(19) United States
(12) Patent Application Publication
(10) Pub. No.: US 2022/0041613 A1
(43) Pub. Date: Feb. 10, 2022

(54) TYROSINE KINASE INHIBITOR COMPOSITIONS, METHODS OF MAKING AND METHODS OF USE

(71) Applicant: Black Diamond Therapeutics, Inc., Cambridge, MA (US)

(72) Inventors: Alexander FLOHR, Basel (CH); Alexander MAYWEG, Stony Brook, NY (US); George TRAINOR, Stony Brook, NY (US); David M. EPSTEIN, Philadelphia, PA (US); Matthew O'CONNOR, Massapequa Park, NY (US); Elizabeth BUCK, Huntington, NY (US)

(21) Appl. No.: 17/280,023

(22) PCT Filed: Sep. 24, 2019
(86) PCT No.: PCT/US2019/052790

§ 371 Date: Mar. 25, 2021

Related U.S. Application Data
(60) Provisional application No. 62/903,598, filed on Sep. 20, 2019.
(60) Provisional application No. 62/736,291, filed on Sep. 25, 2018.

(51) Int. Cl. C07D 491/08; C07D 403/12; A61K 45/06

(52) U.S. Cl. CPC C07D 491/08; A61K 45/06; C07D 403/12

(57) ABSTRACT

The present disclosure relates to new compounds of formula I

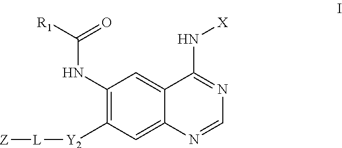

and pharmaceutically acceptable salts and stereoisomers thereof, as inhibitors of receptor tyrosine kinases (RTK), in particular extracellular mutants of ErbB-receptors. The disclosure also relates to methods of preparation these compounds, compositions comprising these compounds, and methods of using them in the treatment of abnormal cell growth in mammals, (e.g., humans).

### RELATED APPLICATIONS

This application claims priority to, and the benefit of, U.S. Application Nos. 62/903,598, filed Sep. 20, 2019, and 62/736,291, filed Sep. 25, 2018, the entire contents of each of which are incorporated herein by reference.

### FIELD OF THE DISCLOSURE

The present disclosure relates to new compounds as inhibitors of receptor tyrosine kinases (RTK), in particular oncogenic mutants of ErbB-receptors. The disclosure also relates to methods of preparing the disclosed compounds, compositions comprising the compounds, and methods of using them in the treatment of abnormal cell growth in mammals, (e.g., humans).

### BACKGROUND

Mutations affecting either the intracellular catalytic domain or extracellular ligand binding domain of an ErbB receptor can generate oncogenic activity (the ErbB protein family consists of 4 members including ErbB-1, also named epidermal growth factor receptor (EGFR) and Erb-2, also named HER2 in humans). ErbB inhibitors are a known treatment for a number of cancers. However, not every patient is responsive satisfactorily to this treatment. Thus, there is a long-felt need in the art for new therapies that are able to address the variable responsiveness of cancer patients to known therapies. The present disclosure provides compositions and methods for treating cancer in patients with these oncogenic mutations without the variable responsiveness observed when patients having these ErbB mutants are treated using the existing standard of care.

### SUMMARY

In some aspects, the present disclosure is directed toward a compound or a pharmaceutically acceptable salt or stereoisomer thereof of formula I

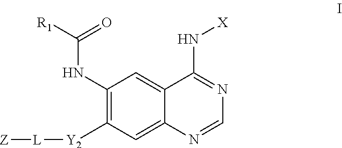

wherein L is a covalent bond, straight chain or branched C₁₋₄ alkyl, or

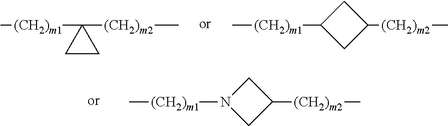

wherein m1 and m2 are independently of each other 0, 1, 2, 3, or 4;
Y² is a covalent bond, —O—, —NH—, —NCH₃—, or —C≡C—;
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl;
R¹ is —CR_{b}═CHRₐ, —C≡CH or —C≡C—CH₃; wherein Rₐ and R_{b} are independently of each other H, hal, or —CH₂—O—CH₃; and
X is a group of formula (i)a

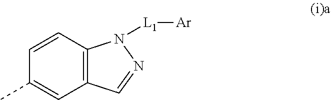

wherein Ar is 6 membered aryl or N-heteroaryl, which is unsubstituted or substituted with one or more of a group selected from halogen, C₁₋₆alkyl, C₁₋₆alkoxy, —CF₃ or —OCF₃;
L¹ is a covalent bond or straight chain or branched C₁₋₃alkyl, which is unsubstituted or substituted with hal, (e.g., a covalent bond or —CH₂—).

In some embodiments, Ar of the compound of formula (i)a or a pharmaceutically acceptable salt or stereoisomer thereof is a group of formula (i)b

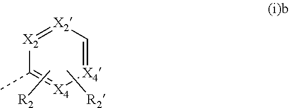

wherein X², X^{2′}, X⁴, and X^{4′} are independently of each other —N═ or —CH═; and R² and R^{2′} are independently of each other H, C₁₋₆ alkyl, hal, —CF₃, or —OCF₃, with the proviso that at least two of X², X^{2′}, X⁴ and X^{4′} are —CH═.

In some embodiments, R² and R^{2′} are bound to X-groups being —CH═. In some embodiments, 2, 3 or all of X², X^{2′}, X⁴ and X^{4′} are —CH═ and thus Ar of formula (i)b is selected from phenyl, pyridine, pyridazine, pyrimidine and pyrazine, (e.g., phenyl, pyridinyl or pyrazinyl; e.g., phenyl).

In some embodiments, group X is a group of formula (ii)a,

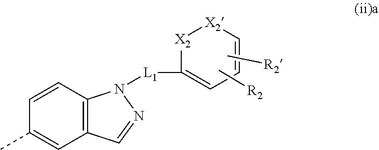

wherein X² and X^{2′} are independently of each other —N═ or —CH═;
R² and R^{2′} are independently of each other H, C₁₋₆ alkyl, hal, —CF₃, or —OCF₃;
L¹ is a covalent bond or straight chain or branched C₁₋₃alkyl, which is unsubstituted or substituted with hal.

In some embodiments, X has the following formula (ii)b, (e.g., (ii)c or (ii)c′)

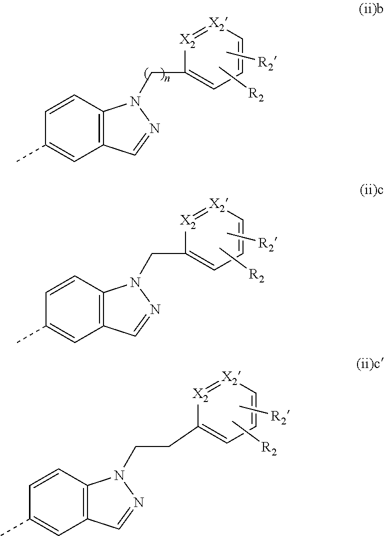

wherein X and X^{2′} are independently of each other —N═ or —CH═; R² and R^{2′} are independently of each other H, C₁₋₆alkyl, hal, —CF₃, or —OCF₃; and n is 0 or 1.

In some embodiments, both X² and X^{2′} are —CH═. In some embodiments, X² is —CH═ and X^{2′} is —N═ or X^{2′} is —CH═ and X² is —N═. In some embodiments, both X² and X^{2′} are —N═. In some embodiments, X has the following formula (ii)d, (ii)e, (ii)f

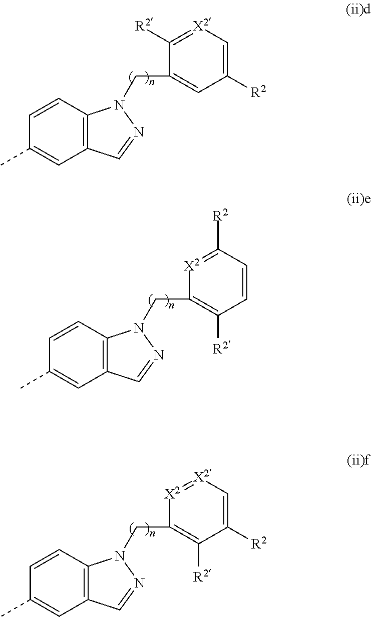

wherein X² and X^{2′} are independently of each other —N═ or —CH═; R² and R^{2′} are independently of each other H, C₁₋₆ alkyl, hal, —CF₃, or —OCF₃; and n is 1 or 2.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl (e.g., H, hal or —CH₃). In some embodiments, R² is H or hal. In some embodiments, R^{2′} is H.

In some embodiments, group X has the following formulas

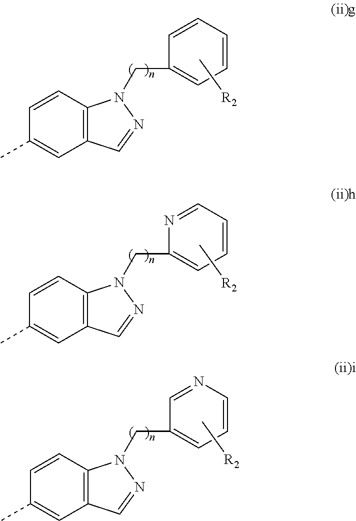

wherein R² is H, C₁₋₆ alkyl, or hal (e.g., H, —CH₃, F, or Cl); and n is 1 or 2.

In some embodiments, —(NR⁶R⁷) ring systems include

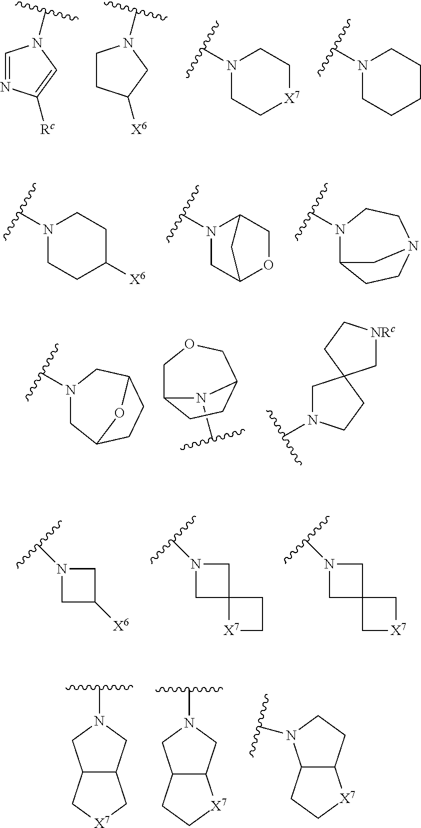

wherein R^{c} is H, C₁₋₄ alkyl, or oxetane; X⁶ is H, —CH₃, —OH, —OCH₃, —OCF₃, —N(CH₃)₂, F, or Cl; X⁷ is —O—, —NH— or —N(CH₃)—.

In some embodiments, —(CHR⁶R⁷) ring systems include

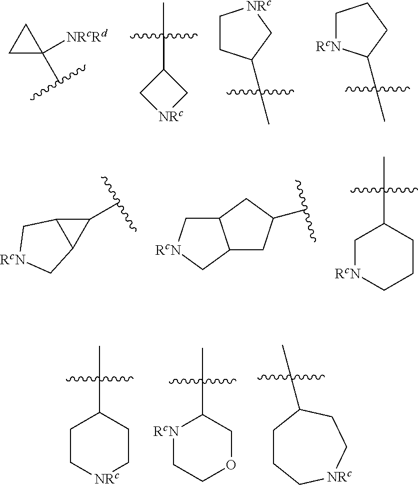

wherein R^{c} is H, C₁₋₄ alkyl, or oxetane; and R^{d} is H or C₁₋₄ alkyl.

In some embodiments Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered (e.g., 6-8-membered heterocycloalkyl), wherein the 3 to 9-membered heterocycloalkyl is a monocycle or a bridged bicycle and is unsubstituted or substituted with C₁₋₄ alkyl.

In some embodiments, ring systems of group Z include

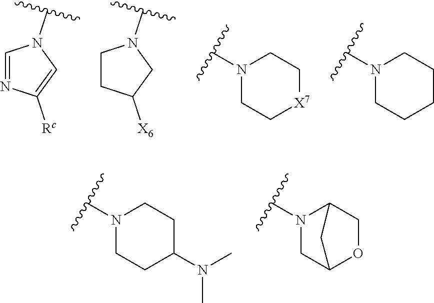

wherein R^{c} is H, C₁₋₄ alkyl, or oxetane; X⁶ is H, —CH₃, —OH, —OCH₃, —OCF₃, —N(CH₃)₂, F, or Cl (e.g., H or —CH₃); X⁷ is —O—, —NH— or —N(CH₃)—.

In some embodiments, the compound of formula I is not a compound wherein X is formula (i)a with L₁ being —CH₂— and Ar being 3-fluorobenzyl, R₁ is CH₂═CH—, Y₂ is O, L is propyl and Z is 4-morpholino, namely N-{4-[1-(3-fluoro-benzyl)-1H-indazole-5-ylamino]-7-[3-(4-morpholino)propoxy]-quinazolin-6-yl}-acrylamide.

In some embodiments, the present disclosure is directed toward a compound or a pharmaceutically acceptable salt or stereoisomer thereof of formula II or III

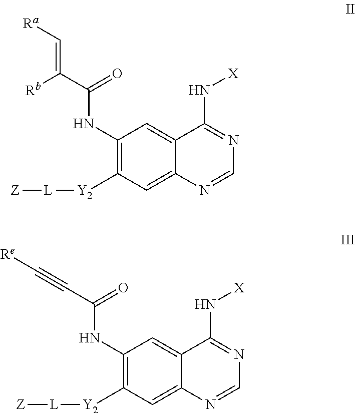

wherein L is a covalent bond, straight chain or branched C₁₋₄ alkyl, or

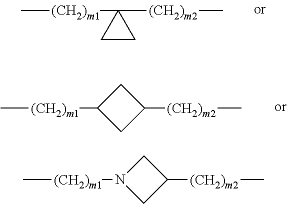

wherein m1 and m2 are independently of each other 0, 1, 2, 3, or 4, (e.g., covalent bond, straight chain or branched C₁₋₄ alkyl);
Y² is a covalent bond, —O—, —NH—, —NCH₃—, or —C≡C—;
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl;
Rₐ and R_{b} are independently of each other H, hal, or —CH₂—O—CH₃, (e.g., H); Re is H or methyl; and X is a group of formula (ii)a

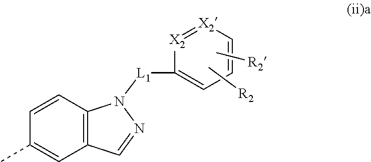

wherein X² and X^{2′} are independently of each other —N═ or —CH═;
L¹ is a covalent bond or straight chain or branched C₁₋₃alkyl, which is unsubstituted or substituted with hal;
R² and R^{2′} are independently of each other H, C₁₋₆ alkyl, hal, —CF₃, or —OCF₃, (e.g., H or hal).

In some embodiments, the compound of formula II is not a compound wherein X is formula (i)a with L₁ being —CH₂— and Ar being 3-fluorobenzyl, Rₐ, R_{b} are H, Y₂ is O, L is propyl and Z is 4-morpholino, namely N-{4-[1-(3-fluoro-benzyl)-1H-indazole-5-ylamino]-7-[3-(4-morpholino)propoxy]-quinazolin-6-yl}-acrylamide.

In some embodiments, the present disclosure is directed toward a compound or a pharmaceutically acceptable salt or stereoisomer thereof of formula IV

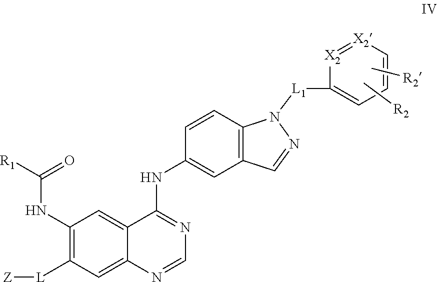

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, —OCF₃;
L is a covalent bond, straight chain or branched C₁₋₄ alkyl, or

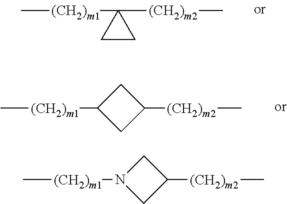

wherein m1 and m2 are independently of each other 0, 1, 2, 3, or 4, (e.g., L is a covalent bond, straight chain or branched C₁₋₄ alkyl);
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, the present disclosure is directed toward a compound or a pharmaceutically acceptable salt or stereoisomer thereof of formula VII

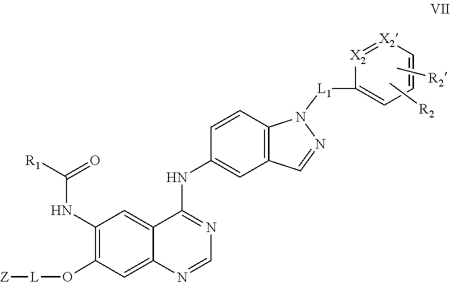

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
L is a covalent bond, straight chain or branched C₁₋₄ alkyl, or

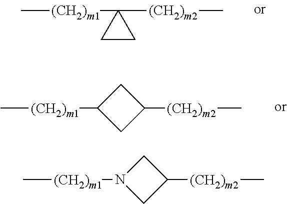

wherein m1, m2 are independently of each other 0, 1, 2, 3, or 4, (e.g., L is a covalent bond, straight chain or branched C₁₋₄ alkyl);
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, the compound of formula VII is not a compound wherein L₁ is —CH₂—, X², X^{2′} are —CH═, R² is 3-fluoro, R^{2′} is H, R₁ is CH₂═CH—, L is propyl and Z is 4-morpholino, namely N-{4-[1-(3-fluoro-benzyl)-1H-indazole-5-ylamino]-7-[3-(4-morpholino)propoxy]-quinazolin-6-yl}-acrylamide.

In some embodiments, the present disclosure is directed toward a compound or a pharmaceutically acceptable salt or stereoisomer thereof of formula X

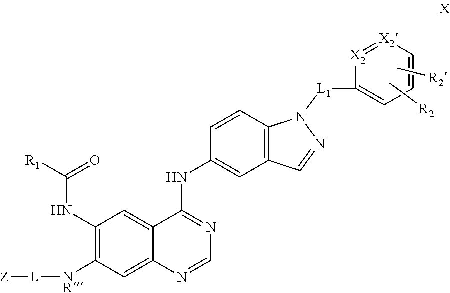

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—C₃;
R² and R^{2′} are independently of each other H, C₁₋₆ alkyl, hal, —CF₃, or —OCF₃;
L is a covalent bond, straight chain or branched C₁₋₄ alkyl, or

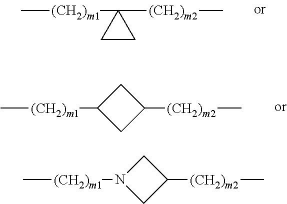

wherein m1 and m2 are independently of each other 0, 1, 2, 3, or 4, (e.g., L is a covalent bond, straight chain or branched C₁₋₄ alkyl);
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, the present disclosure is directed toward a compound or a pharmaceutically acceptable salt or stereoisomer thereof of formula XIII

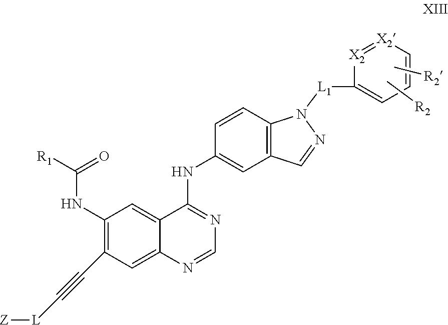

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
L is a covalent bond, straight chain or branched C₁₋₄ alkyl, or

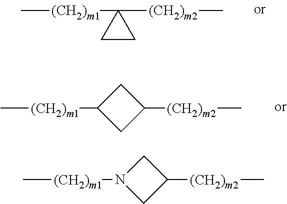

wherein m1 and m2 are independently of each other 0, 1, 2, 3, or 4, (e.g., L is a covalent bond, straight chain or branched C₁₋₄ alkyl);
Z is —(NR⁴R⁵), wherein R⁴ and R⁵ are independently of each other H, C₁₋₆ alkyl, cyclopropyl, cyclobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, —(NR⁶R⁷) and —(CHR⁶R⁷) are selected from

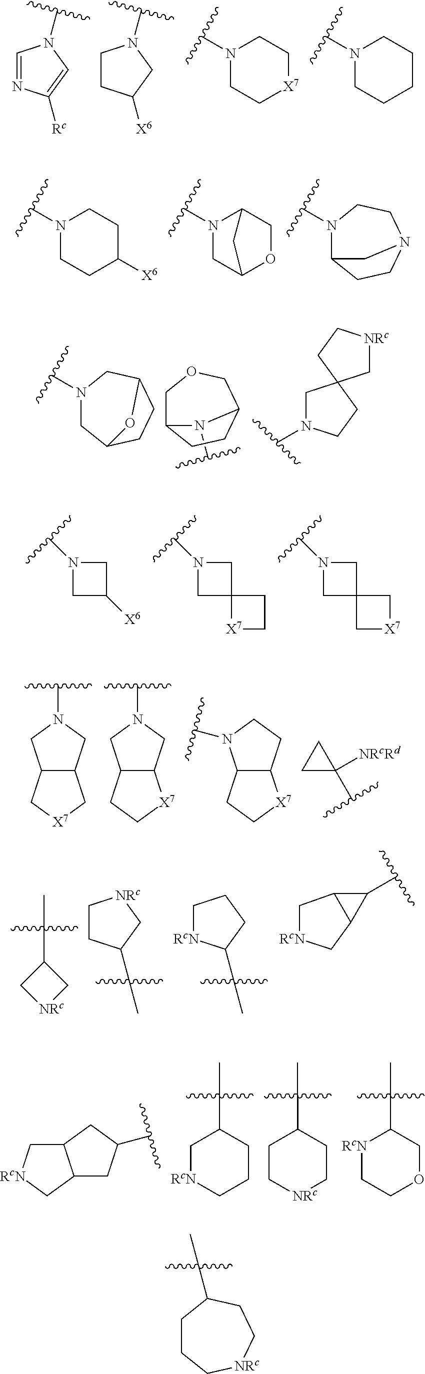

wherein R^{c} is H, C₁₋₄ alkyl, or oxetane; X⁶ is H, —CH₃, —OH, —OCH₃, —OCF₃, —N(CH₃)₂, F, or Cl; X⁷ is —O—, —NH—, or —N(CH₃)—; and R^{d} is H or C₁₋₄ alkyl.

In some embodiments, the disclosure provides a composition comprising a compound of the disclosure or a pharmaceutically acceptable salt or stereoisomer thereof. In some embodiments, the composition further comprises a pharmaceutically acceptable carrier. In some embodiments, the composition further comprises a second therapeutically active agent.

In some embodiments, the second therapeutically active agent comprises a non-Type I inhibitor. In some embodiments, the non-Type I inhibitor comprises a small molecule Type II inhibitor.

In some embodiments, the disclosure provides a composition of the disclosure for use in the treatment of cancer.

In some embodiments, the disclosure provides a use of a composition of the disclosure for treating cancer, comprising administering to a subject a therapeutically-effective amount of the composition.

In some embodiments, the disclosure provides a method of treating cancer in a subject, comprising administering to a subject a therapeutically effective amount of a composition of the disclosure.

In some embodiments, the disclosure provides a method of treating cancer in a subject, comprising administering to a subject a therapeutically effective amount of a composition of the disclosure, wherein the cancer is characterized by expression of an oncogenic variant of an epidermal growth factor receptor (EGFR). In some embodiments, the cancer, a tumor or a cell thereof expresses the oncogenic variant of an EGFR. In some embodiments, the oncogenic variant of EGFR is an allosteric variant of EGFR.

In some embodiments of the methods of treating cancer of the disclosure, including those wherein the cancer is characterized by expression of an oncogenic variant and the oncogenic variant of EGFR is an allosteric variant of EGFR, the oncogenic variant of an EGFR comprises an EGFR variant III (EGFR-Viii) mutation.

In some embodiments of the methods of treating cancer of the disclosure, including those wherein the cancer is characterized by expression of an oncogenic variant and the oncogenic variant of EGFR is an allosteric variant of EGFR, the oncogenic variant of an EGFR comprises a substitution of a valine (V) for an alanine (A) at position 289 of SEQ ID NO: 1.

In some embodiments of the methods of treating cancer of the disclosure, including those wherein the cancer is characterized by expression of an oncogenic variant and the oncogenic variant of EGFR is an allosteric variant of EGFR, the oncogenic variant of an EGFR comprises a modification of a structure of the EGFR, wherein the oncogenic variant of an EGFR is a capable of forming a covalently linked dimer, wherein the covalently linked dimer is constitutively active and wherein the covalently linked dimer enhances an activity of EGFR when contacted to a Type I ErbB inhibitor. In some embodiments, the modification of the structure of the EGFR comprises a modification of one or more of a nucleic acid sequence, an amino acid sequence, a secondary structure, a tertiary structure, and a quaternary structure. In some embodiments, the oncogenic variant comprises a mutation, a splicing event, a post-translational process, a conformational change or any combination thereof. In some embodiments, the modification of the structure of the EGFR occurs within a first cysteine rich (CR1) and/or second cysteine rich (CR2) region of EGFR. In some embodiments, the first cysteine rich (CR1) and/or second cysteine rich (CR2) region of EGFR comprises amino acid residues T211-R334 and/or C526-S645 of SEQ ID NO: 1, respectively. In some embodiments, the oncogenic variant of an EGFR generates a physical barrier to formation of a disulfide bond within the CR1 and/or the CR2 region. In some embodiments, the oncogenic variant of an EGFR removes a physical barrier to formation of a disulfide bond within the CR1 and/or the CR2 region. In some embodiments, the the oncogenic variant of an EGFR comprises one or more free or unpaired Cysteine (C) residues located at a dimer interface of the EGFR. In some embodiments, the oncogenic variant of an EGFR comprises one or more free or unpaired Cysteine (C) residues at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1. In some embodiments, the modification occurs within 10 angstroms or less of an intramolecular disulfide bond at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1.

In some embodiments of the methods of treating cancer of the disclosure, including those wherein the cancer is characterized by expression of an oncogenic variant and the oncogenic variant of EGFR is mutation of EGFR, a nucleotide sequence encoding the oncogenic variant of an EGFR comprises a deletion or a substitution of a sequence encoding exon 19 or a portion thereof. In some embodiments, the deletion or the substitution comprises one or more amino acids that encode an adenosine triphosphate (ATP) binding site. In some embodiments, the ATP binding site comprises amino acids E746 to A750 of SEQ ID NO: 1. In some embodiments, the ATP binding site or the deletion or substitution thereof comprises K858 of SEQ ID NO: 1. In some embodiments, the deletion comprises K858 of SEQ ID NO: 1. In some embodiments, an arginine (R) is substituted for the lysine (K) at position 858 (K858R) of SEQ ID NO: 1. In some embodiments, an arginine (R) is substituted for the leucine (L) at position 858 (L858R) of SEQ ID NO: 1.

In some embodiments of the methods of treating cancer of the disclosure, including those wherein the cancer is characterized by expression of an oncogenic variant and the oncogenic variant of EGFR is an allosteric variant of EGFR, a nucleotide sequence encoding the oncogenic variant of an EGFR comprises an insertion within a sequence encoding exon 20 or a portion thereof. In some embodiments, the sequence encoding exon 20 or a portion thereof comprises a sequence encoding KEILDEAYVMASVDNPHVCAR (SEQ ID NO: 7). In some embodiments, the sequence encoding exon 20 or a portion thereof comprises a sequence encoding a C-helix, a terminal end of the C-helix or a loop following the C-helix. In some embodiments, the insertion comprises the amino acid sequence of ASV, SVD, NPH, or FQEA. In some embodiments, the sequence encoding exon 20 or a portion thereof comprises one or more of: (a) an insertion of the amino acid sequence ASV between positions V769 and D770 of SEQ ID NO: 1; (b) an insertion of the amino acid sequence SVD between positions D770 and N771 of SEQ ID NO: 1; (c) an insertion of the amino acid sequence NPH between positions H773 and V774 of SEQ ID NO: 1; (d) an insertion of the amino acid sequence FQEA between positions A763 and Y764 of SEQ ID NO: 1; (e) an insertion of the amino acid sequence PH between positions H773 and V774 of SEQ ID NO: 1; (f) an insertion of the amino acid G between positions D770 and N771 of SEQ ID NO: 1; (g) an insertion of the amino acid H between positions H773 and V774 of SEQ ID NO: 1; (h) an insertion of the amino acid sequence HV between positions V774 and C775 of SEQ ID NO: 1; (i) an insertion of the amino acid sequence AH between positions H773 and V774 of SEQ ID NO: 1; (j) an insertion of the amino acid sequence SVA between positions A767 and S768 of SEQ ID NO: 1; (k) a substitution of the amino acid sequence GYN for the DN between positions 770 and 771 of SEQ ID NO: 1; (l) an insertion of the amino acid H between positions N771 and P772 of SEQ ID NO: 1; (m) an insertion of the amino acid Y between positions H773 and V774 of SEQ ID NO: 1; (n) an insertion of the amino acid sequence PHVC between positions C775 and R776 of SEQ ID NO: 1; (o) a substitution of the amino acid sequence YNPY for the H at position 773 of SEQ ID NO: 1; (p) an insertion of the amino acid sequence DNP between positions P772 and H773 of SEQ ID NO: 1; (q) an insertion of the amino acid sequence VDS between positions S768 and V769 of SEQ ID NO: 1; (r) an insertion of the amino acid H between positions D770 and N771 of SEQ ID NO: 1; (s) an insertion of the amino acid N between positions N771 and P772 of SEQ ID NO: 1; (t) an insertion of the amino acid sequence PNP between positions P772 and H773 of SEQ ID NO: 1; (u) a substitution of the amino acid sequence GSVDN for the DN between positions 770 and 771 of SEQ ID NO: 1; (v) a substitution of the amino acid sequence GYP for the NP between positions 771 and 772 of SEQ ID NO: 1; (w) an insertion of the amino acid G between positions N771 and P772 of SEQ ID NO: 1; (x) an insertion of the amino acid sequence GNP between positions P772 and 1773 of SEQ ID NO: 1; (y) an insertion of the amino acid sequence GSV between positions V769 and D770 of SEQ ID NO: 1; (z) a substitution of the amino acid sequence GNPHVC for the VC between positions 774 and 775 of SEQ ID NO: 1; (aa) an insertion of the amino acid sequence LQEA between positions A763 and Y764 of SEQ ID NO: 1; (bb) an insertion of the amino acid sequence GL between positions D770 and N771 of SEQ ID NO: 1; (cc) an insertion of the amino acid Y between positions D770 and N771 of SEQ ID NO: 1; (dd) an insertion of the amino acid sequence NPY between positions H773 and V774 of SEQ ID NO: 1; (ee) an insertion of the amino acid sequence TH between positions H773 and V774 of SEQ ID NO: 1; (ff) a substitution of the amino acid sequence KGP for the NP between positions 771 and 772 of SEQ ID NO: 1; (gg) a substitution of the amino acid sequence SVDNP for the NP between positions 771 and 772 of SEQ ID NO: 1; (hh) an insertion of the amino acid sequence NN between positions N771 and P772 of SEQ ID NO: 1; (ii) an insertion of the amino acid T between positions N771 and P772 of SEQ ID NO: 1; and (jj) a substitution of the amino acid sequence STLASV for the SV between positions 768 and 769 of SEQ ID NO: 1.

In some embodiments of the methods of treating cancer of the disclosure, including those wherein the cancer is characterized by expression of an oncogenic variant and the oncogenic variant of EGFR is an allosteric variant of EGFR, the oncogenic variant of an EGFR comprises EGFR-Vii, EGFR-Vvi, EGFR-R222C, EGFR-R252C, EGFR-R252P, EGFR-R256Y, EGFR-T263P, EGFR-Y270C, EGFR-A289T, EGFR-A289V, EGFR-A289D, EGFR-H304Y, EGFR-G331R, EGFR-P596S, EGFR-P596L, EGFR-P596R, EGFR-G598V, EGFR-G598A, EGFR-G614D, EGFR-C620Y, EGFR-C614W, EGFR-C628F, EGFR-C628Y, EGFR-C636Y, EGFR-G645C, EGFR-A660, EGFR-A768 or any combination thereof.

In some embodiments, the disclosure provides a method of treating cancer in a subject, comprising administering to a subject a therapeutically effective amount of a composition of the disclosure, wherein the cancer is characterized by expression of one or more of: (a) a wild type human epidermal growth factor receptor 2 (HER2) receptor or (b) an oncogenic variant of a HER-2 receptor. In some embodiments, the cancer, a tumor, or a cell thereof expresses one or more of: (a) a wild type human epidermal growth factor receptor 2 (HER2) receptor or (b) an oncogenic variant of a HER-2 receptor. In some embodiments of the methods of treating cancer of the disclosure, including those wherein cancer is characterized by characterized by expression of a wild type HER2 receptor, the wild type HER2 receptor comprises the amino acid sequence of SEQ ID NO: 2, 3, 4, 5, or 6.

In some embodiments of the methods of treating cancer of the disclosure, including those wherein cancer is characterized by expression of an oncogenic variant of a HER2 receptor, the oncogenic variant of the HER2 receptor is an allosteric variant of the HER2 receptor.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of a phenylalanine (F) for a serine (S) at position 310 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of a tyrosine (Y) for a serine (S) at position 310 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of a glutamine Q) for an arginine (R) at position 678 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of a leucine (L) for a valine (V) at position 777 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of a methionine (M) for a valine (V) at position 777 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of an isoleucine (I) for a valine (V) at position 842 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER-2 receptor comprises a substitution of an alanine (A) for a leucine (L) at position 755 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of a proline (P) for a leucine (L) at position 755 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises a substitution of a serine (S) for a leucine (L) at position 755 of SEQ ID NO: 2 or 5.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, a nucleotide sequence encoding the oncogenic variant of a HER2 receptor comprises an insertion within a sequence encoding exon 20 or a portion thereof. In some embodiments, the sequence encoding exon 20 or a portion thereof comprises a sequence encoding KEILDEAYVMAGVGSPYVSR(SEQ ID NO: 8). In some embodiments, the sequence encoding exon 20 or a portion thereof comprises a sequence encoding a C-helix, a terminal end of the C-helix or a loop following the C-helix. In some embodiments, the insertion comprises the amino acid sequence of GSP or YVMA. In some embodiments, the sequence encoding exon 20 or a portion thereof comprises one or more of: (a) an insertion of the amino acid sequence YVMA between positions A775 and G776 of SEQ ID NO: 2; (b) an insertion of the amino acid sequence GSP between positions P780 and Y781 of SEQ ID NO: 2; (c) an insertion of the amino acid sequence YVMA between positions A771 and Y772 of SEQ ID NO: 2; (d) an insertion of the amino acid sequence YVMA between positions A775 and G776 of SEQ ID NO: 2; (e) an insertion of the amino acid V between positions V777 and G778 of SEQ ID NO: 2; (f) an insertion of the amino acid V between positions V777 and G778 of SEQ ID NO: 2; (g) a substitution of the amino acid sequence AVGCV for the GV between positions 776 and 777 of SEQ ID NO: 2; (h) a substitution of the amino acid sequence LC for the G between position 776 of SEQ ID NO: 2; (i) a substitution of the amino acid sequence LCV for the G between position 776 of SEQ ID NO: 2; (j) an insertion of the amino acid sequence GSP between positions V777 and G778 of SEQ ID NO: 2; (k) a substitution of the amino acid sequence PS for the LRE between positions 755 and 757 of SEQ ID NO: 2; (l) a substitution of the amino acid sequence CPGSP for the SP between positions 779 and 780 of SEQ ID NO: 2; (m) an insertion of the amino acid C between positions V777 and G778 of SEQ ID NO: 2; (n) a substitution of the amino acid sequence VVMA for the AG between positions 775 and 776 of SEQ ID NO: 2; (o) a substitution of the amino acid sequence VV for the G at position 776 of SEQ ID NO: 2; (p) a substitution of the amino acid sequence AVCV for the GV between positions 776 and 777 of SEQ ID NO: 2; (q) a substitution of the amino acid sequence VCV for the GV between positions 776 and 777 of SEQ ID NO: 2; (r) an insertion of the amino acid G between positions G778 and S779 of SEQ ID NO: 2; (s) a substitution of the amino acid sequence PK for the LRE between positions 755 and 757 of SEQ ID NO: 2; (t) an insertion of the amino acid V between positions A775 and G776 of SEQ ID NO: 2; (u) an insertion of the amino acid sequenceYAMA between positions A775 and G776 of SEQ ID NO: 2; (v) a substitution of the amino acid sequence CV for the G at position 776 of SEQ ID NO: 2; (w) a substitution of the amino acid sequence AVCGG for the GVG between positions 776 and 778 of SEQ ID NO: 2; (x) a substitution of the amino acid sequence CVCC for the GVG between positions 776 and 778 of SEQ ID NO: 2; (y) a substitution of the amino acid sequence VVVG for the GVG between positions 776 and 778 of SEQ ID NO: 2; (z) a substitution of the amino acid sequence SVGG for the GVGS between positions 776 and 779 of SEQ ID NO: 2; (aa) a substitution of the amino acid sequence VVGES for the GVGS between positions 776 and 779 of SEQ ID NO: 2; (bb) a substitution of the amino acid sequence AVGSGV for the GV between positions 776 and 777 of SEQ ID NO: 2; (cc) a substitution of the amino acid sequence CVC for the GV between positions 776 and 777 of SEQ ID NO: 2; (dd) a substitution of the amino acid sequence HVC for the GV between positions 776 and 777 of SEQ ID NO: 2; (ee) a substitution of the amino acid sequence VAAGV for the (V between positions 776 and 777 of SEQ ID NO: 2; (It) a substitution of the amino acid sequence VAGV for the GV between positions 776 and 777 of SEQ ID NO: 2; (gg) a substitution of the amino acid sequence VVV for the GV between positions 776 and 777 of SEQ ID NO: 2; (hh) an insertion of the amino acid sequence FPG between positions G778 and S779 of SEQ ID NO: 2; (ii) an insertion of the amino acid sequence GS between positions S779 and P780 of SEQ ID NO: 2; (jj) a substitution of the amino acid sequence VPS for the VLRE between positions 754 and 757 of SEQ ID NO: 2; (kk) an insertion of the amino acid E between positions V777 and G778 of SEQ ID NO: 2; (ll) an insertion of the amino acid sequence MAGV between positions V777 and G778 of SEQ ID NO: 2; (mm) an insertion of the amino acid S between positions V777 and G778 of SEQ ID NO: 2; (nn) an insertion of the amino acid sequence SCV between positions V777 and G778 of SEQ ID NO: 2; and (oo) an insertion of the amino acid sequence LMAY between positions Y772 and V773 of SEQ ID NO: 2.

In some embodiments of the methods of treating cancer of the disclosure, including those wherein cancer is characterized by expression of an oncogenic variant of a HER2 receptor and wherein the oncogenic variant of the HER2 receptor is an allosteric variant of the HER2 receptor, the oncogenic variant of a HER2 receptor comprises HER2-Δ16 (i.e. a HER2 variant that lacks Exon 16). HER2-C311R, HER2-S310F, p95-HER2-M611 (i.e. a HER2 variant wherein the amino acid encoding the protein begins at M611 of a wild type HER2 sequence, including SEQ ID NO: 2) or any combination thereof.

In some embodiments, the disclosure provides a method of treating cancer in a subject, comprising administering to a subject a therapeutically effective amount of the composition of the disclosure, wherein the cancer is characterized by expression of an oncogenic variant of a HER-4 receptor. In some embodiments, the oncogenic variant of the HER-4 receptor is an allosteric variant of the HER4 receptor. In some embodiments, the oncogenic variant of a HER4 receptor comprises deletion of exon 16 (HER4-Δ16).

In some embodiments of the methods of treating cancer of the disclosure, the administration is systemic. In some embodiments, the administration oral. In some embodiments, the administration is intravenous.

In some embodiments of the methods of treating cancer of the disclosure, the administration is local. In some embodiments, the administration intratumoral, intraocular, intraosseus, intraspinal or intracerebroventricular.

In some embodiments of the methods of treating cancer of the disclosure, the subject or the cancer is insensitive or resistant to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.

In some embodiments of the methods of treating cancer of the disclosure, the subject or the cancer has an adverse reaction to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291. In some embodiments, the adverse reaction is an activation of the oncogenic variant of an EGFR and wherein the oncogenic variant comprises a mutation in an extracellular domain of the receptor. In some embodiments, the adverse reaction is an activation of the oncogenic variant of a HER-2 Receptor and wherein the oncogenic variant comprises a mutation in an extracellular domain of the receptor.

In some embodiments of the methods of treating cancer of the disclosure, the cancer, a tumor, or a cell thereof expresses an oncogenic variant of an EGFR, wherein the sequence encoding the oncogenic variant of the EGFR comprises a deletion of exon 20 or a portion thereof and wherein the the cancer, the tumor or the cell thereof does not comprise a second oncogenic variation in a sequence other than exon 20 of EGFR. In some embodiments, the second oncogenic variation comprises a sequence encoding one or more of an EGFR kinase domain (KD), BRAF, NTRK, and KRAS.

In some embodiments of the methods of treating cancer of the disclosure, the cancer, a tumor or a cell thereof expresses an oncogenic variant of an EGFR, wherein the sequence encoding the oncogenic variant of the EGFR comprises a deletion of exon 20 or a portion thereof and wherein the the cancer, the tumor or the cell thereof does not comprise a marker indicating responsiveness to immunotherapy.

In some embodiments of the methods of treating cancer of the disclosure, the cancer comprises a solid tumor. In some embodiments, the cancer is a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC) or any subtype thereof. In some embodiments, the cancer is a glioblastoma (GBM) or any subtype thereof. In some embodiments, the cancer is a breast cancer or any subtype thereof. In some embodiments, the cancer is a lung cancer or any subtype thereof.

In some embodiments of the methods of treating cancer of the disclosure, the therapeutically effective amount reduces a severity of a sign or symptom of the cancer. In some embodiments, the sign of the cancer comprises a tumor grade and wherein a reduction of the severity of the sign comprises a decrease of the tumor grade. In some embodiments, the sign of the cancer comprises a tumor metastasis and wherein a reduction of the severity of the sign comprises an elimination of the metastasis or a reduction in the rate or extent of the metastasis. In some embodiments, the sign of the cancer comprises a tumor volume and wherein a reduction of the severity of the sign comprises an elimination of the tumor or a reduction in the volume. In some embodiments, the symptom of the cancer comprises pain and wherein a reduction of the severity of the sign comprises an elimination or a reduction in the pain.

In some embodiments of the methods of treating cancer of the disclosure, the therapeutically effective amount induces a period of remission.

In some embodiments of the methods of treating cancer of the disclosure, the therapeutically effective amount improves a prognosis of the subject.

In some embodiments of the methods of treating cancer of the disclosure, the subject is a participant or a candidate for participation in in a clinical trial or protocol thereof. In some embodiments, the subject is excluded from treatment with a Type I inhibitor. In some embodiments, the Type I inhibitor comprises gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 or AZD9291.

In some embodiments of the methods of treating cancer of the disclosure, the method further comprises treating the subject with a Non-Type I inhibitor.

In some embodiments of the methods of treating cancer of the disclosure, the composition further comprises a Non-Type I inhibitor.

In some embodiments of the methods of treating cancer of the disclosure, the Non-Type I inhibitor comprises a Type II small molecule inhibitor. In some embodiments, the Type II small molecule inhibitor comprises neratinib, AST-1306, HKI-357, or lapatinib.

In some embodiments, the disclosure provides a method of treating cancer in a subject comprising administering to the subject a Non-Type I inhibitor or a potent Type I inhibitor, wherein the subject comprises an allosteric variant of an EGFR or an allosteric variant of a HER2-receptor. In some embodiments, the Non-Type I ErbB inhibitor comprises a Type TT small molecule inhibitor. In some embodiments, the Non-Type I ErbB inhibitor or potent Type I inhibitor comprises AMG-595, rindopepimut, sapitinib, afatinib, neratinib, AST-1306, HKI-357, or lapatinib. In some embodiments, the cancer comprises a solid cancer. In some embodiments, the cancer comprises a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC) or any subtype thereof. In some embodiments, the cancer comprises a glioblastoma (GBM) or any subtype thereof. In some embodiments, the cancer comprises a breast cancer or any subtype thereof. In some embodiments, the cancer comprises a lung cancer or any subtype thereof.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 is an illustration of the structure of EGFR and a group of 20 genomic mutations affecting the CR1 or CR2 regions of EGFR and which are expressed in (GBM tumors. Mutations are highlighted within the crystal structure for the ectodoinain of EGFR (1IVO). Mutations are noted as magenta spheres. EGF ligand is shown in green, and the EGFR protomers are shown in grey and orange. See also Table 2.

FIG. 2 is a schematic depiction of an expression pattern for EGFR splicing events and mutations in the CR1 and CR2 regions for a group of 164 GBM tumors. One tumor, TCGA.878, expressing four variants (EGFR-Viii, EGR-A289T, EGFR-A289V, and EGFR-A289D, is noted. More than 65% of GBM tumors express EGFR ectodomain variants affecting the CR1/2 regions.

FIG. 3 is a graph depicting exemplary ectodomain variants of ErbB receptors that are transforming. The proliferation of BaF3 cells expressing EGFR-Viii, EGFR-Vii, or EGFR-A289V, or vector alone (parental), cultured in the absence of IL-3. The proliferation of parental BaF3 cells cultured in the presence of IL-3 is shown as a control.

FIG. 4 is a an illustration of the structure of EGFR and exemplary free cysteines that are formed at the extracellular dimer interface of EGFR as a result of genomic mutations and alternative splicing events in cancer. Arrows note the positions of free cysteines predicted to be generated as a result of the events EGFR-A289V, EGFR-Viii, EGFR-Vii, and EGFR-Vvi. Positions are mapped onto the crystal structure of the ectodomain of EGFR (1IVO). EGF ligand is shown in green, and EGFR protomers are shown in grey and orange.

FIG. 5A is a series of photographs of Western blots depicting the expression of total and phosphorylated monomeric EGFR versus covalent EGFR dimers for EGFR-Viii, EGFR-Vii, EGFR-Vvi, and EGFR-A289V, detected by resolving proteins under non-reducing conditions. The data demonstrate that EGFR-Viii, EGFR-Vii, EGFR-Vvi, and EGFR-A289V exist as covalently activated dimers.

FIG. 5B is a graph depicting the quantitation of results from FIG. 5A and the quantitation of percentage of receptor that exists as covalent dimer for total versus phosphorylated receptor.

FIG. 6 is a pair of photographs of Western blots depicting the effect of EGF treatment on levels of monomeric and dimeric phosphorylated EGFR for EGFR-Vii and EGFR-Vvi. In contrast to EGFR-Viii, EGF further potentiates the formation of active covalent dimers for EGFR-Vii and EGFR-Vvi.

FIG. 7A is a series of photographs of Western blots depicting the effect of 100 nM erlotinib treatment on levels of monomeric and dimeric EGFR levels in cells expressing EGFR-Viii, EGFR-Vii, EGFR-Vvi, or EGFR-A289V. Monomeric and dimeric EGFR levels were detected by resolving proteins under non-reducing conditions. The data demonstrate that Type I inhibitors enhance the formation of covalent dimers for all covalently-activated EGFR variants.

FIG. 7B is a pair of photographs of Western blots depicting the effect of varying concentrations of erlotinib on monomeric and dimeric EGFR levels in cells expressing EGFR-Vii. Monomeric and dimeric EGFR levels were detected by resolving proteins under non-reducing conditions.

FIG. 7C is a graph quantifying the data presented in FIG. 7B. The data demonstrate that erlotinib induces a dose dependent increase in covalently dimerized receptor.

FIG. 8 is a series of photographs of Western blots depicting the effect of a panel of Type I and Type II inhibitors on dimeric and monomeric EGFR levels for cells expressing EGFR-Vii and EGFR-A289V. Monomeric and dimeric EGFR levels were detected by resolving proteins under non-reducing conditions. The data demonstrate that Type I, but not Type II, ErbB inhibitors enhance the formation of covalent dimers for covalently-activated EGFR variants.

FIG. 9 is a series of photographs of Western blots depicting the effect of 100 nM erlotinib treatment on monomeric and dimeric EGFR levels for two EGFR variants. Monomeric and dimeric EGFR levels were detected by resolving proteins under non-reducing conditions. The data demonstrate that both EGFR-A660 and EGFR-A768 can exist as covalent dimers and covalent dimer is potentiated following treatment with erlotinib.

FIG. 10A is a series of photographs of Western blots depicting the effect of varying concentrations of erlotinib on monomeric and dimeric levels of phosphorylated EGFR in cells expressing EGFR-Viii, EGFR-Vii, and EGFR-A289V. Monomeric and dimeric EGFR levels were detected by resolving proteins under non-reducing conditions. The data demonstrate that sub-saturating concentrations of erlotinib stimulate the phosphorylation of covalently dimerized splice-activated EGFR isoforms.

FIG. 10B is a series of photographs of Western blots depicting the effect of varying concentrations of erlotinib treatment, followed by 30 minute washout, on total and phosphorylated EGFR levels in cells expressing EGFR-Vii or EGFR-Vvi. Proteins were resolved under non-reducing conditions. The data demonstrate that erlotinib paradoxically enhances the phosphorylation of covalent dimers for EGFR-Vii and EGFR-Vvi.

FIG. 11A is a graph depicting the effect of DNMSO, 37 nM erlotinib, or 100 nM erlotinib on the proliferation of BaF3 cells expressing EGFR-Viii. Proliferation data were collected at multiple time points over a three day period. The data demonstrate that sub-saturating concentrations of erlotinib result in paradoxical stimulation of proliferation in cells expressing splice-activated EGFR.

FIG. 11B is a graph depicting the effect of varying concentrations of erlotinib on the proliferation of BaF3 cells expressing EGFR-Viii, EGFR-Vii or EGFR-A289V. Proliferation was assessed at 72 hours after erlotinib dosing. The data demonstrate that sub-saturating concentrations of erlotinib paradoxically stimulate the growth of BaF3 cells driven by EGFR-Viii, EGFR-Vii, and EGFR-A289V.

FIG. 12 is a series of graphs depicting the effect of 12.5 nM or 1 uM of WZ8040, WZ3146, or WZ4002 on the proliferation of BaF3 cells expressing EGFR-Viii. Proliferation data were collected at multiple time points over a three day period. The data demonstrate that sub-saturating concentrations of WZ8040, WZ3146 or WZ4002 result in paradoxical stimulation of proliferation in cells expressing EGFR-Viii.

FIG. 13A is an illustration of the structure of EGFR and exemplary free cysteines are formed at the extracellular dimer interface of HER2 receptors as a result of genomic mutations and alternative splicing events in cancer. Arrows point to positions of free cysteines generated by the Δ16 splice event or C311R or S310F mutations.

FIG. 13B is a pair of graphs demonstrating that HER2 and HER4 splice variants are transforming. The proliferation of BaF3 cells expressing HER4-WT (JMA), HER4Δ16 (JMC), and HER2Δ16, or vector alone (parental), cultured in the absence of IL-3. The proliferation of parental BaF3 cells cultured in the presence of IL-3 is shown as a control.

FIG. 14 is a series of photographs of Western blots depicting the expression of dimeric and monomeric levels of phosphorylated HER2 or HER4 receptors in cells expressing each variant. Monomeric and dimeric EGFR levels were detected by resolving proteins under non-reducing conditions. The data demonstrate that multiple HER2 and HER4 splicing events and mutations in the CR1 and CR2 regions result in covalently active dimers.

FIG. 15A is a series of photographs of Western blots depicting the effect of the Type I HER2 inhibitor sapitinib or the Type I HER4 inhibitor afatinib on levels of dimerized receptors for cells expressing HER2-Δ16, HER2-C311R, HER2-S310F, or HER4Δ16. Monomeric and dimeric HER2 and HER4 levels were detected by resolving proteins under non-reducing conditions. The data demonstrate that Type I inhibitors induce the formation of covalent dimers for covalently-activated HER2 and HER4 isoforms.

FIG. 15B a series of photographs of Western blots and corresponding graphs depicting the effect of varying concentrations of sapitinib or afatinib on the levels of dimerized HER2 or HER2 in cells expressing HER2-Δ16 or HER4-Δ16. Monomeric and dimeric HER2 and HRE4 levels were detected by resolving proteins under non-reducing conditions. The data demonstrate that Type I inhibitors induce a dose dependent increase in covalently dimerized receptors for HER2 and HER4 variants.

FIG. 16 is a graph depicting the effect of varying concentrations of sapitinib on the proliferation of BaF3-HER2-Δ16 cells. The data demonstrate that sub-saturating concentrations of the Type I inhibitor sapitinib paradoxically stimulate the proliferation of BaF3-HER2Δ16 cells.

FIGS. 17A-C are a series of graphs demonstrating that expression levels of ErbB splice variants can be measured by isoform selective PCR. The expression levels of EGFR-Viii (A), EGFR-Vii (B), and EGFR-Vvi (C) in cells engineered to express the respective splice-variant as compared to cells that do not express the respective splice-variant. Primers and probes used to detect each variant are listed. Primers and probes used to detect EGFRVIII are identified as SEQ ID NO: 9 (forward), SEQ ID NO: 10 (probe) and SEQ ID NO: 11 (reverse). Primers and probes used to detect EGFRVii are identified as SEQ ID NO: 12 (forward), SEQ ID NO: 13 (probe) and SEQ ID NO: 14 (reverse). Primers and probes used to detect EGFRVvi are identified as SEQ ID NO: 15 (forward), SEQ ID NO: 16 (probe) and SEQ ID NO: 17 (reverse).

FIG. 18 is a graph showing the fraction of the maximum proliferation of cells having, for example, the EGFR-Vii mutation with NT-113, a potent Type I covalent inhibitor. NT-113 induces dimerization for covalently activated ErbB receptors. In contrast to reversible Type I inhibitors, and other covalent Type I inhibitors, there is no evidence for increased cellular proliferation in response to NT-113. Therefore, in contrast to reversible Type I inhibitors, and other covalent Type I inhibitors, NT-113 represents a potent Type I covalent molecule that could be used to treat tumors driven by covalently-activated ErbB receptors.

FIG. 19 is a table providing potency values for representative marketed ErbB inhibitors against EGFR and HER2 receptor variants. The data show that these cpds lack potency and selectivity against allo-HER2 mutations. These compounds also lack potency and selectivity against ErbB Exon 20 ins mutants and ErbB Exon 20 deletion mutants. Potency values reflect cellular anti-proliferative activity (IC50, nM). EGFR-WT=A431 (+H292); HER2-WT=BT474; H4006 EGFR19del; all mutants are BaF3 transformants. Green boxes depict greater than a 10-fold selective inhibition of oncogenic mutants versus WT-EGFR and red boxes depict less than a 10-fold selective inhibition of oncogenic mutants versus WT-EGFR.

FIG. 20 is a table providing potency values for representative marketed ErbB inhibitors against EGFR and HER2 receptor variants. The data show that these cpds lack potency and selectivity against ErbB Exon 20 ins mutants and ErbB Exon 20 deletion mutants. Potency values reflect cellular anti-proliferative activity (IC50, nM). EGFR-WT=A431 (+H292); HER2-WT==BT474; H4006=EGFR19del; all mutants are BaF3 transformants. Green boxes depict greater than a 10-fold selective inhibition of oncogenic mutants versus WT-EGFR and red boxes depict less than a 10-fold selective inhibition of oncogenic mutants versus WT-EGFR.

FIG. 21 is a graph showing the effect of Compound No. 3 on tumors with HER mutant signaling and corresponding Compound No. 3 plasma levels in vivo.

### DETAILED DESCRIPTION

The present disclosure relates to new compounds useful as inhibitors of receptor tyrosine kinases (RTK), in particular oncogenic mutants of ErbB-receptors. In some embodiments of the disclosure, oncogenic mutants of ErbB-receptors are also allosteric mutants of ErbB-receptors.

In some embodiments of the disclosure, allosteric mutants may comprise or consist of an ErbB receptor variant having a mutation in a sequence outside of an ATP-binding site.

In some embodiments of the disclosure, allosteric mutants may comprise or consist of an ErbB receptor variant having a mutation in a sequence within one or more of exon 19, exon or a C1-C2 extracellular dimerization interface.

Mutations affecting either the intracellular catalytic domain or extracellular ligand binding domain of an ErbB receptor can generate oncogenic activity (the ErbB protein family consists of 4 members including ErbB-1, also named epidermal growth factor receptor (EGFR) and Erb-2, also named HER2 in humans). Extracellular mutants of ErbB receptors in cancer, including EGFR-Viii (also EGFR-V3) and HER2-S310F, are constitutively activated in the absence of ligand, exhibit sustained signaling that is resistant to downregulation, and are both transforming and tumorigenic (Nishikawa, Ji et al. 1994, 2013, Francis, Zhang et al. 2014). Their expression is associated with metastasis and with poor long term overall survival.

In glioblastomna (also glioblastoma multiforma or GBM), EGFR-Viii is expressed by 20% of tumors (Sugawa, Ekstrand et al. 1990, Brennan, Verhaak et al. 2013). Expression of EGFR-Viii in GBM tends to be mutually exclusive with expression of other RTK oncogenes, which are co-expressed with EGFR variants in only 7% of GBM tumors (Furnari, Cloughesy et al. 2015). These data demonstrate how EGFR-Viii in GBM has a dominant and mutually exclusive expression pattern compared with other oncogenic drivers. EGFR-Viii is also expressed by approximately 30% of SCCHN tumors (Sok, Coppelli et al. 2006, Keller, Shroyer et al. 2010, Wheeler, Suzuki et al. 2010, Tinhofer, Klinghamrnmer et al. 2011, Wheeler, Egloff et al. 2015) and 10% of squamous NSCLC (Ji, Zhao et al. 2006, Sasaki, Kawano et al. 2007), and is associated with resistance to current therapeutics including the anti-EGFR antibody cetuximab (Sok, Coppeili et al. 2006, Tinhofer, Klinghammer et al. 2011). Normal tissues do not express this oncogenic receptor variants.

HER2-S310F is the most common mutation of HER2 expressed in human tumors, expressed by approximately 0.5% of all tumors. HER2-S310F expression is mutually exclusive with expression of HER2 amplification. HER2-S310F is highly oncogenic, transforming BaF3 cells (a murine interleukin-3 (IL-3) dependent pro-B cell line) to IL-3 independence and promoting tumor growth in vivo.

Short insertions of within Exon 20 of EGFR and HER2 are expressed by lung adenocarcinoma tumors and other tumor groups. ErbB Exon 20 insertion mutants are expressed by 4-5% of lung adenocarcinoma tumors. Examples include HER2-YVMA, EGFR-SVD, and EGFR-NPH. These ErbB Exon 20 insertion mutants are highly oncogenic, transforming BaF3 cells to IL-3 independence and promoting tumor growth in vivo.

ErbB inhibitors are a known treatment for a number of cancers. However, not every patient is responsive satisfactorily to this treatment. Thus, there is a long-felt need in the art for new therapies that are able to address the variable responsiveness of cancer patients to known therapies. The present disclosure is able to overcome some of these drawbacks of the standard of care, as it existed prior to the development of the compositions and methods disclosed herein.

### Definitions

Unless specified otherwise the following general definitions apply to all compounds of the disclosure according to the description.

The term “compound of the disclosure.” as used herein, refers to compounds represented by formulae I to XV and any of the examples disclosed herein.

It is understood that “independently of each other” means that when a group is occurring more than one time in any compound, its definition on each occurrence is independent from any other occurrence.

It is further understood that a dashed line (or a wave being transverse to a bond) depicts the site of attachment of a residue (i.e. a partial formula).

It is also understood that a group defined as being a “covalent bond” refers to a direct linkage between its two neighbouring groups.

The following definitions regarding group Z apply to each of the embodiments cited hereinafter: the term “3 to 6-membered heterocycloalkyl” in combination with —(NR⁴R⁵), refers to a non-aromatic or partially aromatic ring system having 3, 4, 5, or 6 ring atoms independently selected from C, N, O, and S, (e.g., C, N, and O), the number of N atoms being 0, 1, 2 and the number of O and S atoms each being 0, 1, 2. Examples of 3 to 6-membered heterocycloalkyl groups include oxiranyl, thiaranyl, aziridinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

A “partially aromatic” ring system is a ring system with one or more unsaturations, which are not fully conjugated over the whole ring system.

The term “3 to 6-membered heteroaryl” in combination with —(NR⁶R⁷), —(CHR⁶R⁷), refers to a (fully) aromatic ring system having 3, 4, 5, or 6 ring atoms, (e.g., 5 ring atoms, selected from C, N, O, and S, or selected from C, N, and O, or selected from C and N, with the number of N atoms being 0, 1, 2 or 3 and the number of O and S atoms each being 0, 1 or 2). Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl and imidazolyl.

The term “3 to 9-membered heterocycloalkyl” in combination with or —(NR⁶R⁷), —(CHR⁶R⁷), refers to a non-aromatic or partially aromatic ring system having 3, 4, 5, 6, 7, 8, or 9 ring atoms selected from C, N, O, or S, (e.g., C, N, or O, the number of N atoms being 0, 1, 2 or 3 and the number of O and S atoms each being 0, 1 or 2). The term “monocycle” in connection with a 3 to 9-membered heterocycloalkyl refers to the 3 to 9 ring atoms forming a single ring. Examples of such monocycles include oxiranyl, thiaranyl, aziridinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, oxepanyl, thiepanyl, azepanyl, diazepanyl, oxazepanyl and the like. In some embodiments, mnonocycles include azetidinyl, pyrrolidinyl piperidinyl, piperazinyl, morpholinyl.

The term “fused bicycle” in connection with a 3 to 9-membered heterocycloalkyl refers to the 3 to 9 ring atoms selected from C, N, O, and S, forming two or three rings (e.g., two rings) that are sharing two adjacent atoms (i.e. one bond) and at least one ring in the fused ring system contains one or more heteroatoms, (e.g., 1, 2 or 3 heteroatoms selected from N, O, and S). Some non-limiting examples of the fused heterobicyclyl group include 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like.

The term “bridged bicycle” in connection with a 3 to 9-membered heterocycloalkyl refers to the 3 to 9 ring atoms forming a ring system that has a carbocyclyl or heterocyclyl, wherein two non-adjacent atoms of the ring are connected (bridged) by at least one (e.g., one or two) atoms selected from C, N, O, and S, (e.g., C, N, or O), with the proviso that at least one heteroatom is present. Examples of such bridged ring systems include bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl, having one or two heteroatoms selected from N and O).

The term “spirobicycle” connection with a 3 to 9-membered heterocycloalkyl refers to the 3 to 9 ring atoms forming a ring system that has two rings each of which are independently selected from a carbocyclyl or a heterocyclyl, wherein the two rings share one atom. Examples of such spiro ring systems include spiropentanyl, spiro[2.3]hexanyl spiro[3.3]heptanyl, spiro[3.4]octanyl, spiro[4.4]nonanyl, spiro[3.5]nonanyl, spiro[4.5]decanyl, (e.g., spiro[3.3]heptanyl, spiro[4.4]nonanyl), having one or two heteroatoms selected from N and O. In some embodiments, examples include diazaspiro[3.3]heptanyl oxa-azaspiro[3.3]heptanyl, diazaspiro[4.4]nonanyl, oxa-azaspiro[4.4]nonanyl.

The term “halogen” or “hal” as used herein may be fluoro, chloro, bromo or iodo (e.g. fluoro or chloro).

The term “alkyl” as used herein refers to a fully saturated branched or unbranched hydrocarbon moiety. The term “C₁₋₄alkyl” refers to a fully saturated branched or unbranched hydrocarbon moiety having 1, 2, 3 or 4 carbon atoms. Representative examples of alkyl include, but are not limited to, methyl, ethyl, n-propyl, iso-propyl, n-butyl, sec-butyl, iso-butyl, tert-butyl. In connection with group L, the term “straight chain or branched C₁₋₄ alkyl” is —CH₂—, —(CH₂)₂—, —(CH₂)₃—, —(CH₂)₄—, —C(CH₃)₂—, or —CH₂—C(CH₃)₂—.

According to the methods of the disclosure, exemplary subjects are mammals. In some embodiments, exemplary subjects are human. Exemplary subjects may be male or female. Exemplary subjects may be of any age (fetal, neonatal, child, adolescent, or adult) In some embodiments, the subject is an adult. Exemplary subjects may be healthy, for example, healthy subjects of the disclosure may participate in a clinical trial in which one or more steps of the methods of the disclosure are performed. In certain embodiments, exemplary subjects may have at least one benign or malignant tumor. In some embodiments, exemplary subjects have at least one form or type of cancer. Subjects of the methods of the disclosure may be patients diagnosed with cancer, patients undergoing treatment for cancer, potential participants in a research and/or clinical study, and/or participants selected for inclusion in or exclusion from a research and/or clinical study.

According to the methods of the disclosure, the term “mammal” refers to any mammal, including humans, domestic and farm animals, and zoo, sports, or pet animals, such as dogs, cats, cattle, horses, sheep, pigs, goats, rabbits, etc. (e.g. human).

The term “prevention” or “preventing” refers to reducing or eliminating the onset of the symptoms or complications of a disease (e.g., cancer). In some embodiments, such prevention comprises the step of administering a therapeutically effective amount of a compound disclosed herein (e.g., a compound of Formula I or a pharmaceutically acceptable salt thereof) or a pharmaceutical composition disclosed herein (e.g., a pharmaceutical composition containing a compound of Formula I or a pharmaceutically acceptable salt thereof) to a subject in need thereof (e.g., a mammal (e.g., a human).

The term “treatment” or “treating” is intended to encompass therapy and cure. In some embodiments, such treatment comprises the step of administering a therapeutically effective amount of a compound disclosed herein (e.g., a compound of Formula I or a pharmaceutically acceptable salt thereof) or a pharmaceutical composition disclosed herein (e.g., a pharmaceutical composition containing a compound of Formula I or a pharmaceutically acceptable salt thereof) to a subject in need thereof (e.g., a mammal (e.g., a human). In some embodiments, the term “reating” or “treatment” refers to therapeutic treatment measures; wherein the object is to slow down (lessen) the targeted pathologic condition or disorder. Those in need of treatment include those already with the disorder as well as those prone to have the disorder. For example, when treating cancer according to a method of the disclosure, a subject or mammal is successfully “treated” for cancer if, after receiving a therapeutic amount of an ErbB inhibitor according to the methods of the present disclosure, the patient shows observable and/or measurable reduction in or absence of one or more of the following: reduction in the number of cancer cells or absence of the cancer cells; reduction in the proliferation or survival of cancer cells; and/or relief to some extent, one or more of the symptoms associated with the specific infection; reduced morbidity and mortality, and improvement in quality of life issues. The above parameters for assessing successful treatment and improvement in the disease are readily measurable by routine procedures familiar to a physician.

According to the methods of the disclosure, subjects having a mutation of the disclosure may be treated for cancer by administering a therapeutically-effective amount of a composition of the disclosure, a Type II ErbB inhibitor, an EGFR-Viii selective agent/inhibitor or the NT-113 Type I inhibitor. The term “therapeutically effective amount” refers to an amount of a composition of the disclosure, a Type II ErbB inhibitor, an EGFR-Viii selective agent/inhibitor or the NT-113 Type I inhibitor effective to “treat” a disease or disorder (e.g. cancer) in a subject or mammal. See preceding definition of “treating.”

According to the methods of the disclosure, a Type II ErbB inhibitor may include a small molecule. A “small molecule” is defined herein to have a molecular weight below about 1500 Daltons.

According to the methods of the disclosure, mutations may be detected by analyzing either nucleic acid or amino acid sequences from a subject. Nucleic acid and/or amino acid sequences may be isolated prior to sequence analysis.

The terms “nucleic acid” and “polynucleotide” are used interchangeably herein to refer to single- or double-stranded RNA, DNA, or mixed polymers. Polynucleotides may include genomic sequences, extra-genomic and plasmid sequences, and smaller engineered gene segments that express, or may be adapted to express polypeptides.

An “isolated nucleic acid” is a nucleic acid that is substantially separated from other genome DNA sequences as well as proteins or complexes such as ribosomes and polymerases, which naturally accompany a native sequence. The term embraces a nucleic acid sequence that has been removed from its naturally occurring environment, and includes recombinant or cloned DNA isolates and chemically synthesized analogues or analogues biologically synthesized by heterologous systems. A substantially pure nucleic acid includes isolated forms of the nucleic acid. This refers to the nucleic acid as originally isolated and does not exclude genes or sequences later added to the isolated nucleic acid.

The term “polypeptide” is used in its conventional meaning, i.e., as a sequence of amino acids. The polypeptides are not limited to a specific length of the product. Peptides, oligopeptides, and proteins are included within the definition of polypeptide, and such terms may be used interchangeably herein unless indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof.

An “isolated polypeptide” is one that has been identified and separated and/or recovered from a component of its natural environment. In some embodiments, the isolated polypeptide will be purified (1) to greater than 95% by weight of polypeptide as determined by the Lowry method (e.g. more than 99% by weight), (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or silver stain. Isolated polypeptide includes the polypeptide in situ within recombinant cells since at least one component of the polypeptide's natural environment will not be present. In some embodiments, the isolated polypeptide will be prepared by at least one purification step.

A “native sequence” polynucleotide is one that has the same nucleotide sequence as a polynucleotide derived from nature. A “native sequence” polypeptide is one that has the same amino acid sequence as a polypeptide (e.g. EGFR) derived from nature (e.g., from any species). Such native sequence polynucleotides and polypeptides can be isolated from nature or can be produced by recombinant or synthetic means.

A polynucleotide “variant,” as the term is used herein, is a polynucleotide that differs from a disclosed polynucleotide herein in one or more substitutions, deletions, additions and/or insertions.

A polypeptide “variant,” as the term is used herein, is a polypeptide that differs from a disclosed polypeptide herein in one or more substitutions, deletions, additions and/or insertions, or inversions. Such variants may be naturally occurring, non-naturally occurring, or may be synthetically generated.

EGFR mutations (or variants) of the disclosure may comprise one or more substitutions, deletions, additions and/or insertions, or inversions of the amino acid sequence that are alter the function of the resultant protein. Mutations may be detected, for example, by comparison or alignment of a nucleic or amino acid sequence with a wild type sequence.

When comparing polynucleotide and polypeptide sequences, two sequences are said to be “identical” if the sequence of nucleotides or amino acids in the two sequences is the same when aligned for maximum correspondence, as described below. Comparisons between two sequences are performed by comparing the sequences over a comparison window to identify and compare local regions of sequence similarity. A “comparison window” as used herein, refers to a segment of at least about 20 contiguous positions, (e.g. 30 to about 75 or 40 to about 50), in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CAIBOS 4:11-17; Robinson, E. D. (1971) Comb. Theor 11:105; Santou, N. Nes, M. (1987) Mol. Biol. Evol. 4:406-425; Sneath, P. H. A and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (CAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the present disclosure. Software for performing BLAST analyses is publicly available through the National Center for Biotechnology Information.

In some embodiments, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less (e.g. 5 to 15 percent, or 10 to 12 percent), as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

Sequences

A wild type EGFR sequence of the disclosure may comprise or consist of the amino acid
sequence of:

| | |
|---|---|
| 1 | mrpsgtagaa llallaalcp asraleekkv cqgtsnkltq lgtfedhfls lqrmfnncev |
| 61 | vlgnleityv qrnydlsflk tiqevagyvl ialntverip lenlqiirgn myyensyala |
| 121 | vlsnydankt glkelpmrnl qeilhgavrf snnpalcnve siqwrdivss dflsnmsmdf |
| 181 | qnhlgscqkc dpscpngscw gageencqkl tkiicaqqcs grcrgkspsd cchnqcaagc |
| 241 | tgpresdclv crkfrdeatc kdtcpplmly npttyqmdvn pegkysfgat cvkkcprnyv |
| 301 | vtdhgscvra cgadsyemee dgvrkckkce gpcrkvcngi gigefkdsls inatnikhfk |
| 361 | nctsisgdlh ilpvafrgds fthtppldpq eldilktvke itgflliqaw penrtdlhaf |
| 421 | enleiirgrt kqhgqfslav vslnitslgl rslkeisdgd viisgnknlc yantinwkkl |
| 481 | fgtsgqktki isnrgensck atgqvchalc spegcwgpep rdcvscrnvs rgrecvdkck |
| 541 | llegeprefv enseciqchp eclpqamnit ctgrgpdnci qcahyidgph cvktcpagvm |
| 601 | genntlvwky adaghvchlc hpnctygctg pglegcptng pkipsiatgm vgalllllvv |
| 661 | algiglfmrr rhivrkrtlr rllqerelve pltpsgeapn qallrilket efkkikvlgs |
| 721 | gafgtvykgl wipegekvki pvaikelrea tspkankeil deayvmasvd nphverllgi |
| 781 | cltstvqlit qlmpfgclld yvrehkdnig sqyllnwcvq iakgmnyled rrlvhrdlaa |
| 841 | rnvlvktpqh vkitdfglak llgaeekeyh aeggkvpikw malesilhri ythqsdvwsy |
| 901 | gvtvwelmtf gskpydgipa seissilekg erlpqppict idvymimvkc wmidadsrpk |
| 961 | freliiefsk mardpqrylv iqgdermhlp sptdsnfyra lmdeedmddv vdadeylipq |
| 1021 | qgffsspsts rtpllsslsa tsnnstvaci drngiqscpi kedsflqrys sdptgalted |
| 1081 | siddtflpvp eyinqsvpkr pagsvqnpvy hnqplnpaps rdphyqdphs tavgnpeyln |
| 1141 | tvqptcvnst fdspahwaqk gshqisldnp dyqgdffpke akpngifkgs taenaeylrv |
| 1201 | apqssefiga (SEQ ID NO: 1, corresponding to epidermal growth factor receptor |

[Homo sapiens] and Genbank Accession No. CAA25240).
A wild type HER2 Receptor sequence of the disclosure may comprise or consist of the
amino acid sequence of:

| | |
|---|---|
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldng |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpgptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll dideteyhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedigpaspl dstfyrslle dddmgdlvda |
| 1021 | eeylvpqqgf fcpdpapgag gmvhhrhrss strsgggdlt lglepseeea prsplapseg |
| 1081 | agsdvfdgdl gmgaakglqs lpthdpsplq rysedptvpl psetdgyvap ltcspqpeyv |
| 1141 | nqpdvrpqpp spregplpaa rpagatlerp ktispgkngv vkdvfafgga venpeyltpq |
| 1201 | ggaapqphpp pafspafdnl yywdqdpper gappstfkgt ptaenpeylg ldvpv (SEQ ID |

NO: 2, corresponding to receptor tyrosine-protein kinase erbB-2 isoform a precursor
[Homo sapiens] and (GenBank Accession No. NP_004439).
A wild type HER2 Receptor sequence of the disclosure may comprise or consist of the
amino acid sequence of:

| | |
|---|---|
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltiqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | thscvdlddk gcpaeqrasp ltsiisavvg illvvvlgvv fgilikrrqq kirkvtmrrl |
| 661 | lqetelvepl tpsgampnqa qmrilketel rkvkvlgsga fgtvykgiwi pdgenvkipv |
| 721 | aikvlrents pkankeilde ayvmagvgsp yvsrllgicl tstvqlvtql mpygclldhv |
| 781 | renrgrlgsq dllnwcmqia kgmsyledvr lvhrdlaarn vlvkspnhvk itdfglarll |
| 841 | dideteyhad ggkvpikwma lesilrrrft hqsdvwsygv tvwelmtfga kpydgipare |
| 901 | ipdllekger lpqppictid vymimvkcwm idsecrprfr elvsefsrma rdpqrfvviq |
| 961 | nedlgpaspl dstfyrslle dddmgdlvda eeylvpqqgf fcpdpapgag gmvhhrhrss |
| 1021 | strsgggdlt lglepseeea prsplapseg agsdvfdgdl gmgaakglqs lpthdpsplq |
| 1081 | rysedptvpl psetdgyvap ltcspqpeyv nqpdvrpqpp spregplpaa rpagatlerp |
| 1141 | ktlspgkngv vkdvfaggga venpeyltpq ggaapqphpp pafspafdnl yywdqdpper |
| 1201 | gappstfkgt ptaenpeylg ldvpv (SEQ ID NO: 3, corresponding to receptor tyrosine- |

protein kinase erbB-2 isoform b [Homo sapiens] and GenBank Accession No.
NP_001005862).
A wild type HER2 Receptor sequence of the disclosure may comprise or consist of the
amino acid sequence of:

| | |
|---|---|
| 1 | mprgswkpqv ctgtdmklrl paspethldm lrhlyqgcqv vqgnleltyl ptnaslsflq |
| 61 | diqevqgyvl iahnqvrqvp lqrlrivrgt qlfednyala vldngdplnn ttpvtgaspg |
| 121 | glrelqlrsl teilkggvli qrnpqlcyqd tilwkdifhk nnqlaltlid tnrsrachpc |
| 181 | spmckgsrcw gessedcqsl trtvcaggca rckgplptdc cheqcaagct gpkhsdclac |
| 241 | lhfnhsgice lhcpalvtyn tdtfesmpnp egrytfgasc vtacpvnyls tdvgsctlvc |
| 301 | plhnqevtae dgtqrcekcs kpcarvcygl gmehlrevra vtsaniqefa gckkifgsla |
| 361 | flpesfdqdp asntaplqpe qlqvfetlee itgylyisaw pdslpdlsvf qnlqvirgri |
| 421 | lhngaysltl qglgiswlgl rslrelgsgl alihhnthlc fvhtvpwdql frnphqallh |
| 481 | tanrpedecv geglachqlc arghcwgpqp tqcvncsqfl rgqecveecr vlqglpreyv |
| 541 | narhclpchp ecqpqngsvt cfgpeadqcv acahykdppf cvarcpsgvk pdlsympiwk |
| 601 | fpdeegacqp cpincthscv dlddkgcpae qraspltsii savvgillvv vlgvvfgili |
| 661 | krrqqkirky tmrrllqete lvepltpsga mpnqaqmril ketelrkvkv lgsgafgtvy |
| 721 | kgiwipdgen vkipvaikvl rentspkank eildeayvma gvgspyvsrl lgicltstvq |
| 781 | lvtqlmpygc lldhvrenrg rlgsqdllnw cmqiakgmsy ledvrlvhrd laarnvlvks |
| 841 | pnhvkitdfg larlldidet eyhadggkvp ikwmalesil rrrfthqsdv wsygvtvwel |
| 901 | mtfgakpydg ipareipdll ekgerlpqpp ictidvymim vkcwmidsec rprfrelvse |
| 961 | fsrmardpqr fvviqnedlg paspldstfy rslledddmg dlvdaeeylv pqqgffcpdp |
| 1021 | apgaggmvhh rhrssstrsg ggdltlglep seeeaprspl apsegagsdv fdgdlgmgaa |
| 1081 | kglqslpthd psplqrysed ptvplpsetd gyvapltcsp qpeyvnqpdv rpqppspreg |
| 1141 | plpaarpaga tlerpktlsp gkngvvkdvf afggavenpe yltpqggaap qphpppafsp |
| 1201 | afdnlyywdq dppergapps tfkgtptaen peylgldvpv (SEQ ID NO: 4, corresponding |

to receptor tyrosine-protein kinase erbB-2 isoform c [Homo sapiens] and GenBank
Accession No. NP_001276865).
A wild type HER2 Receptor sequence of the disclosure may comprise or consist of the
amino acid sequence of:

| | |
|---|---|
| 1 | melaalcrwg lllallppga astqvctgtd mklrlpaspe thldmlrhly qgcqvvqgnl |
| 61 | eltylptnas lsflqdiqev qgyvliahnq vrqvplqrlr ivrgtqlfed nyalavldhg |
| 121 | dplnnttpvt gaspgglrel qlrslteilk ggvliqrnpq lcyqdtilwk difhknnqla |
| 181 | ltlidtnrsr achpcspmck gsrcwgesse dcqsltrtvc aggcarckgp lptdccheqc |
| 241 | aagctgpkhs dclaclhfnh sgicelhcpa lvtyntdtfe smpnpegryt fgascvtacp |
| 301 | ynylstdvgs ctlvcplhnq evtaedgtqr cekcskpcar vcyglgmehl revravtsan |
| 361 | iqefagckki fgslaflpes fdgdpasnta plqpeqlqvf etleeitgyl yisawpdslp |
| 421 | dlsvfqnlqv irgrilhnga ysltlqglgi swlglrslre lgsglalihh nthlcfvhtv |
| 481 | pwdqlfrnph qallhtanrp edecvgegla chqlcarghc wgpqptqcvn csqflrgqec |
| 541 | veecrvlqgl preyvnarhc lpchpecqpq ngsvtcfgpe adqcvacahy kdppfcvarc |
| 601 | psgvkpdlsy mpiwkfpdee gacqpcpinc thscvdlddk gcpaeqrasp ltsiisavvg |
| 661 | illvvvlgvv fgilikrrqq kirkytmrrl lqetelvepl tpsgampnqa qmrilketel |
| 721 | rkvkvlgsga fgtvykgiwi pdgenvkipv aikvlrents pkankeilde ayvmagvgsp |
| 781 | yvsrllgicl tstvqlvtql mpygclldhv renrgrlgsq dllnwcmqia kgmsyledvr |
| 841 | lvhrdlaarn vlvkspnhvk itdfglarll didetevhad ggkvpikwma lesilrrrft |
| 901 | hqsdvwsygv tvwelmtfga kpydgipare ipdllekger lpqppictid vymimvkcwm |
| 961 | idsecrprfr elvsefsrma rdpqrfvviq nedlgpaspl dstfyrslle dddmgdlvda |
| 1021 | eeylvpqqgff fcpdpapgag gmvhhrhrss strnm (SEQ ID NO: 5, corresponding to |

receptor tyrosine-protein kinase erbB-2 isoform d precursor [Homo sapiens] and GenBank
Accession No. NP_001276866).
A wild type HER2 Receptor sequence of the disclosure may comprise or consist of the
amino acid sequence of:

| | |
|---|---|
| 1 | mklrlpaspe thldmlrhly qgcqvvqgnl eltylptnas lsflqdiqev qgyvliahnq |
| 61 | vrqvplqrlr ivrgtqlfed nyalavldng dplnnttpvt gaspgglrel qlrslteilk |
| 121 | ggvliqrnpq lcyqdtilwk difhknnqla ltlidtnrsr achpcspmck gsrcwgesse |
| 181 | dcqsltrtvc aggcarckgp lptdccheqc aagctgpkhs dclaclhfnh sgicelhcpa |
| 241 | lvtyntdtfe smpnpegryt fgascvtacp ynylstdvgs ctlvcplhnq evtaedgtqr |
| 301 | cekcskpcar vcyglgmehl revravtsan iqefagckki fgslaflpes fdgdpasnta |
| 361 | plqpeqlqvf etleeitgyl yisawpdslp dlsvfqnlqv irgrilhnga ysltlqglgi |
| 421 | swlglrslre lgsglalihh nthlcfvhtv pwdqlfrnph qallhtanrp edecvgegla |
| 481 | chqlcarghc wgpgptqcvn csqflrgqec veecrvlqgl preyvnarhc lpchpecqpq |
| 541 | ngsvtcfgpe adqcvacahy kdppfcvarc psgvkpdlsy mpiwkfpdee gacqpcpinc |
| 601 | ths (SEQ ID NO: 6, corresponding to receptor tyrosine-protein kinase erbB-2 |

isoform e [Homo sapiens] and GenBank Accession No. NP_001276867).

Based on the definitions given throughout the application the skilled person knows which combinations are synthetically feasible and realistic, e.g. combinations of groups leading to heteroatoms directly linked to each other are not contemplated.

### COMPOUNDS OF THE PRESENT DISCLOSURE

In some aspects, the present disclosure is directed toward a compound or a pharmaceutically acceptable salt or stereoisomer thereof of formula I

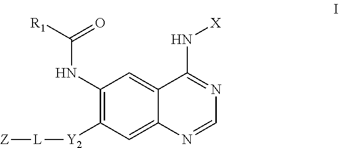

wherein L is a covalent bond, straight chain or branched C₁₋₄ alkyl, or

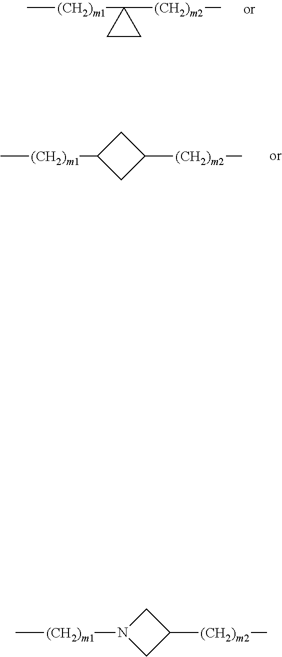

wherein m1 and m2 are independently of each other 0, 1, 2, 3, or 4;
Y² is a covalent bond, —O—, —NH—, —NCH₃—, or —C≡C—;
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle, or a combination thereof and is unsubstituted or substituted with C₁₋₄ alkyl, hal, —OR′, or —NR′R″, wherein R′ and R″ are independently of each other H or C₁₋₄ alkyl;
R¹ is —CR_{b}═CHRₐ, —C≡CH, or —C≡C—CH₃; wherein Rₐ and R_{b} are independently of each other H, hal, or —CH₂—O—CH₃; and
X is a group of formula (i)a

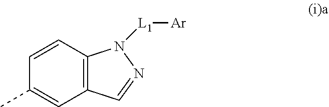

wherein Ar is 6 membered aryl or N-heteroaryl, which is unsubstituted or substituted with one or more of a group selected from halogen, C₁₋₆alkyl, C₁₋₆alkoxy, —CF₃, and —OCF₃;
L¹ is a covalent bond or straight chain or branched C₁₋₃alkyl, which is unsubstituted or substituted with hal, (e.g., a covalent bond or —CH₂—).

In some embodiments, the compound of formula I is not a compound wherein X is formula (i)a with L₁ being —CH₂— and Ar being 3-fluorobenzyl, R₁ is CH₂═CH—, Y₂ is O, L is propyl and Z is 4-morpholino, namely N-{4-[1-(3-fluoro-benzyl)-1H-indazole-5-ylamino]-7-[3-(4-morpholino)propoxy]-quinazolin-6-yl}-acrylamide.

In some embodiments, Ar of the compound of formula (i)a or a pharmaceutically acceptable salt or stereoisomer thereof is a group of formula (i)b

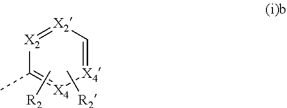

wherein X², X^{2′}, X⁴ and X^{4′} are independently of each other —N═ or —CH═; and R² and R^{2′} are independently of each other H, C₁₋₆ alkyl, hal, —CF₃, or —OCF₃, with the proviso that at least two of X², X^{2′}, X⁴ and X^{4′} are —CH═.

In some embodiments, R² and R^{2′} are bound to X-groups being —CH═.

In some embodiments, 2, 3, or all of X², X^{2′}, X⁴ and X^{4′} are —CH═ and thus Ar of formula (i)b is selected from phenyl, pyridine, pyridazine, pyrimidine and pyrazine, (e.g., phenyl, pyridinyl, and pyrazinyl or phenyl).

In some embodiments, Ar of formula (i)b is a phenyl group a (e.g., a1)

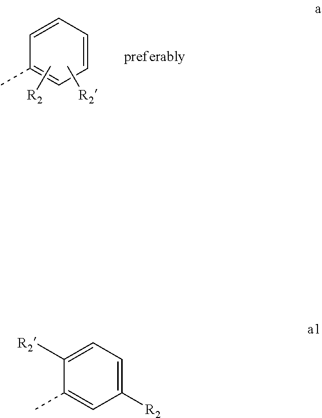

In some embodiments, Ar of formula Ia′ is one of groups b or c (e.g., b1 or c1), wherein the pyridine is linked in ortho- or meta-position to the ring nitrogen

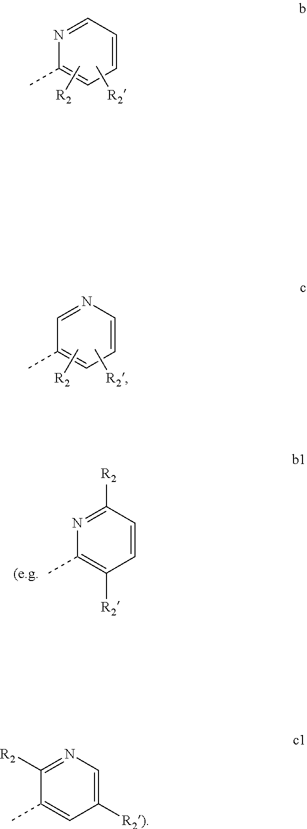

In some embodiments, Ar of formula (i)b is one of groups d or e (e.g., d1 or e1), wherein the pyrimidine is linked in ortho- or meta-position to the ring nitrogens

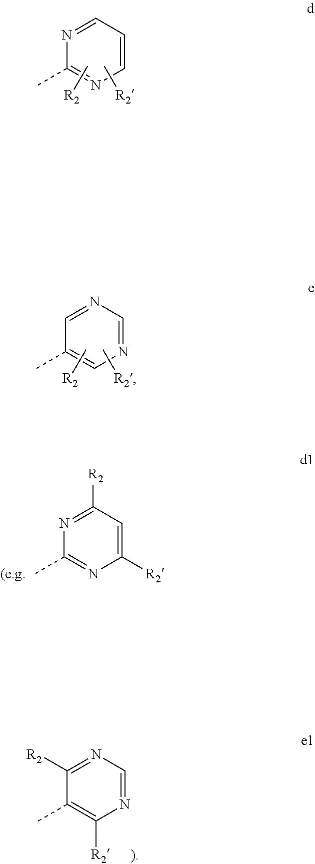

In some embodiments, Ar of formula (i)b is group f (e.g., f1). In some embodiments, Ar of formula (i)b is a pyrazine group g (e.g., g1)

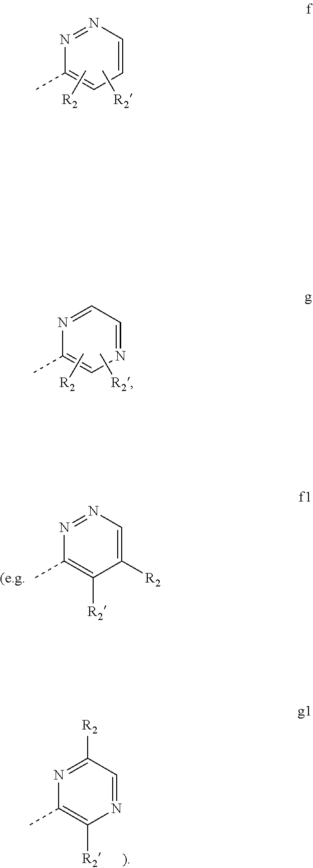

In some embodiments, X⁴ and X^{4′} are —CH═. In some embodiments, Ar groups are a, wherein X², X^{2′}, X⁴ and X^{4′} are —CH═; or b, wherein X^{2′}, X⁴ and X^{4′} are —CH═ and X² is —N═; or c wherein X^{2′} is —N═ and X², X⁴ and X^{4′} are —CH═; or ring f wherein X² and X^{2′} are —N═ and X⁴ and X^{4′} are —CH═, (e.g. groups a or b or c, or group a).

In some embodiments of the compound of formula I, L¹ forms the linker between the indazole bicycle and Ar. In some embodiments, L¹ is a covalent bond. In some embodiments, L¹ is —CH₂— or —CH(CH₃)— or —CH(hal)-. In some embodiments, L¹ is —CH₂—CH₂—, —CH₂—CH(CH₃)— or —CH₂—CH(hal)-. In some embodiments, L¹ is —CH₂—, —CH₂—CH₂—. In some embodiments, L¹ is —CH₂—.

In some embodiments, R⁴ and R⁵ are independently of each other H, C₁₋₄ alkyl, cyclopropyl, or tetrahydrofuryl, (e.g., H or C₁₋₄ alkyl; or CH₃).

In some embodiments, group Z is as defined above. In some embodiments of a compound of formula I, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e g, C, N, and O). In some embodiments, the number of N atoms is 0, 1, or 2. In some embodiments, the number of O and S atoms each is 0, 1, or 2. Examples of 3 to 6-membered heterocycloalkyl groups include oxiranyl thiaranyl, aziridinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl, 1,4-dithianyl, 1,3-dioxane, 1,3-dithianyl, piperazinyl, thiomorpholinyl, piperidinyl, morpholinyl, and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of a compound of formula I, a 3 to 6-membered heteroaryl (in combination with —(NR⁶R⁷) or —(CHR⁶R⁷)) refers to a (fully) aromatic ring system having 3, 4, 5, or 6 ring atoms, (e.g. 3, 4, 5 ring atoms), independently selected from C, N, O, and S, (e.g., C, N, and O or C and N). In some embodiments, the number of N atoms is 0, 1, 2 or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of a compound of formula I, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g., C N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziridinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl, 1,4-dithianyl, 1,3-dioxane, 1,3-dithianyl, piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanoyl, bicyclo[2.2.2]octanyl, bicyclo[3.1.1]hepanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl, (e.g., spiro[3.3]heptanyl, spiro[4.4]nonanyl), having one or two heteroatoms selected from N and O, (e.g., diazaspiro[3.3]heptanyl, oxa-azaspiro[3.3]heptanyl, diazaspiro[4.4]nonanyl, oxa-azaspiro[4.4]nonanyl).

In some embodiments, Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered (e.g., 6-8-membered heterocycloalkyl), wherein the 3 to 9-membered heterocycloalkyl is a monocycle or a bridged bicycle and is unsubstituted or substituted with C₁₋₄ alkyl.

In some embodiments, —(NR⁶R⁷) ring systems include

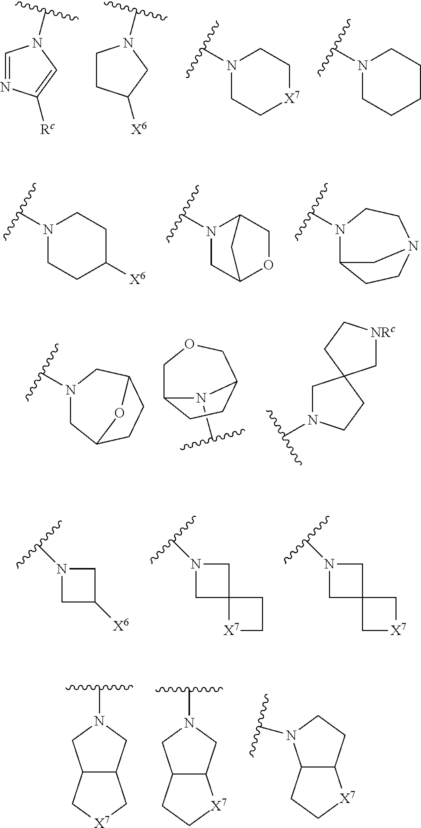

wherein R^{c} is H, C₁₋₄ alkyl, or oxetane; X⁶ is H, —CH₃, —OH, —OCH₃, —OCF₃, —N(CH₃)₂, F, or Cl; X⁷ is —O—, —NH— or —N(CH₃)—.

In some embodiments, —(CHR⁶R⁷) ring systems include

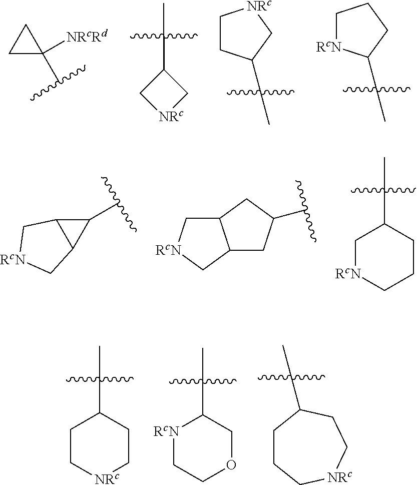

wherein R^{c} is H, C₁₋₄ alkyl, or oxetane; and R^{d} is H or C₁₋₄ alkyl.

In some embodiments, ring systems of group Z include

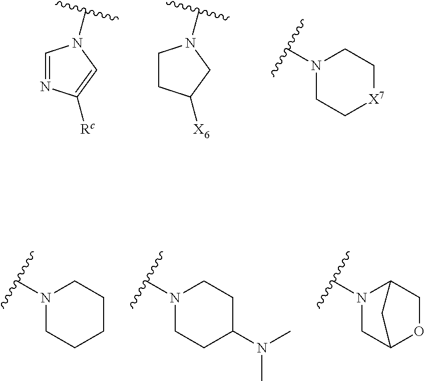

wherein R^{c} is H, C₁₋₄ alkyl or oxetane; X⁶ is H, —CH₃, —OH, —OCH₃, —OCF₃, —N(CH₃)₂, F, or Cl, (e.g., H or —CH₃); X⁷ is —O—, —NH—, or —N(CH₃)—.

In some embodiments, the ring systems of group Z include

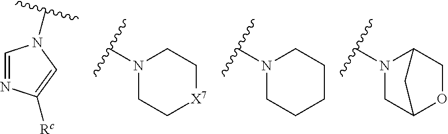

wherein R^{c} is H, C₁₋₄ alkyl, or oxetane; and X⁷ is —O—, —NH—, or —N(CH₃)—.

In some embodiments of a compound of formula I, the following variations of group R¹ are included. In some embodiments, R¹ is —CR_{b}═CHRₐ, wherein Rₐ and R_{b} are independently of each other H, hal, or —CH₂—O—CH₃. In some embodiments, R¹ is —CH═CH₂. In some embodiments, R¹ is —CH═CH-hal or —C(hal)=CH₂. In some embodiments, R¹ is —CH═CH—CH₂—O—C₃. In some embodiments, R¹ is —C≡CH or —C≡C—CH₃.

In some embodiments, groups L and Y², link group Z to the quinazoline core. In some embodiments, Y² is covalent bond. In some embodiments, Y² is —O—. In some embodiments, Y² is —NH— or —NCH₃—. In some embodiments, Y² is —C≡C—.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂— or —CH₂—C(CH)₂—). In some embodiments, L is

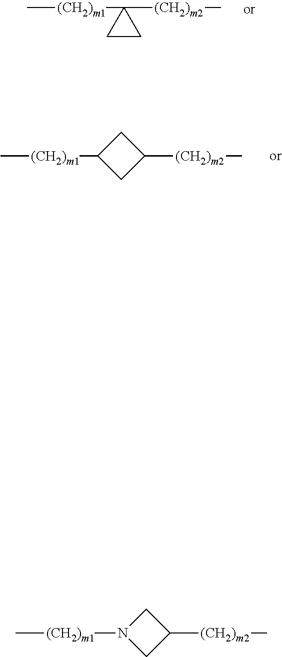

wherein m1 and m2 are independently of each other 0, 1, 2, 3, or 4, (e.g. 0, 1, or 2). In some embodiments, m2 is 0 and m1 is 0, 1, or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄— or —C(CH₃)₂—). In some embodiments, L is —CH₂—, —(CH₂)₂— or —C(CH₃)₂—.

In some embodiments, the present disclosure is directed toward a compound or a pharmaceutically acceptable salt or stereoisomer thereof of formula I

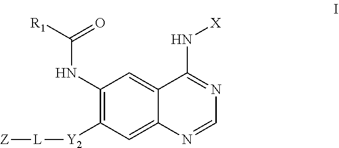

wherein L is a covalent bond, straight chain or branched C₁₋₄ alkyl, or

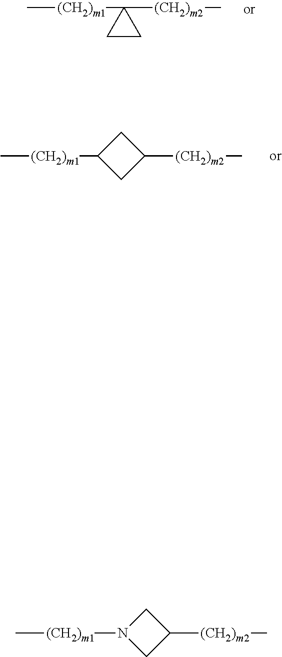

wherein m1 and m2 are independently of each other 0, 1, 2, 3, or 4;
Y² is a covalent bond, —O—, —NH—, —NCH₃—, or —C≡C—;
Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered (e.g., 6-8-membered heterocycloalkyl, wherein the 3 to 9-membered heterocycloalkyl is a monocycle or a bridged bicycle and is unsubstituted or substituted with C₁₋₄ alkyl;
R¹ is —CR_{b}═CHRₐ, —C≡CH, or —C≡C—CH₃ (e.g., —CR_{b}═CHRₐ); wherein Rₐ and R^{b} are independently of each other H, hal, or —CH₂—O—CH₃; and
X is a group of formula (ii)a,

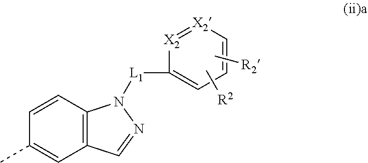

wherein X² and X^{2′} are independently of each other —N═ or —CH═;
R² and R^{2′} are independently of each other H, C₁₋₆ alkyl, hal, —CF₃, or —OCF₃;
L¹ is a covalent bond or straight chain or branched C₁₋₃alkyl, which is unsubstituted or substituted with hal.

In some embodiments of a compound of formula I, the following variations of group R¹ are included, which apply to each of the embodiments cited above. In some embodiments, R¹ is —CR_{b}═CHRₐ, wherein Rₐ and R_{b} are independently of each other H, hal, or —CH₂—O—CH₃. In some embodiments, R¹ is —CH═CH₂. In some embodiments, R¹ is —CH═H-hal or —C(hal)=CH₂. In some embodiments, R¹ is —CH═CH—CH₂—O—CH₃. In some embodiments, R¹ is —C≡CH or —C≡C—CH₃.

In some embodiments, X has the following formula (ii)b, (e.g. (ii)c or (ii)c′)

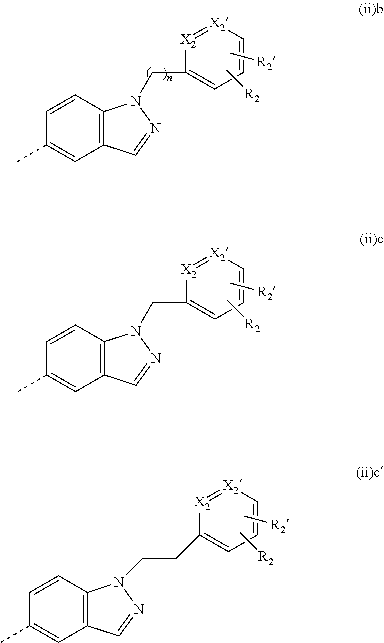

wherein X² and X^{2′} are independently of each other —N═ or —CH═; R² and R^{2′} are independently of each other H, C₁₋₆ alkyl, hal, —CF₃, or —OCF₃; and n is 0 or 1.

In some embodiments, both X² and X^{2′} are —CH═.

In some embodiments, X² is —CH═ and X^{2′} is —N═ or X^{2′} is —CH═ and X² is —N═.

In some embodiments, both X² and X^{2′} are —N═.

In some embodiments, R² and R^{2′} are independently of each other H, C₁₋₆ alkyl, or hal, (e.g., R² is H or hal) and R^{2′} is H.

In some embodiments, X has the following formula (ii)d, (ii)e, (ii)f

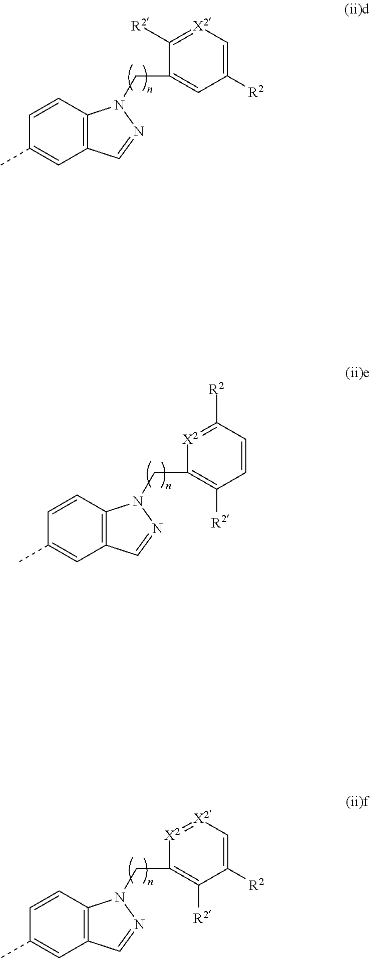

wherein X² and X^{2′} are independently of each other —N═ or —CH═; R² and R^{2′} are independently of each other H, C₁₋₆ alkyl, hal, —CF₃, or —OCF₃; and n is 1 or 2.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal or —CH₃). In some embodiments, R² is H or hal. In some embodiments, R^{2′} is H.

In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments of a compound of the disclosure, group X is

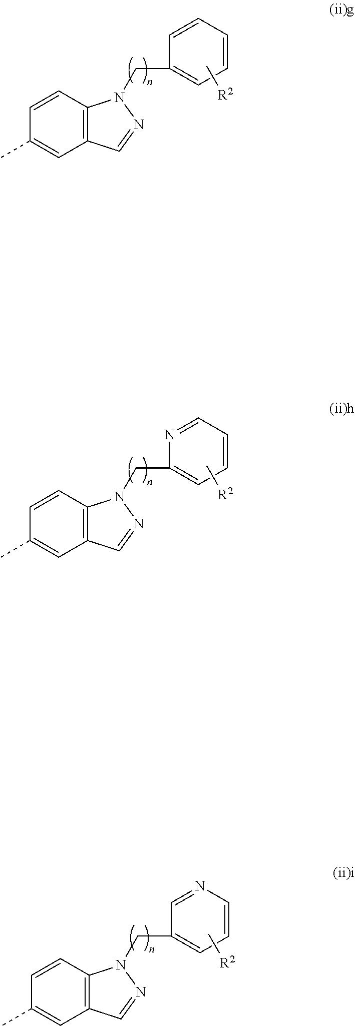

wherein R is H, C₁₋₆ alkyl, or hal (e.g., H, —CH₃, F, or Cl); and n is 1 or 2.

In some embodiments, R⁴ and R⁵ are independently of each other H, or C₁₋₄ alkyl (e.g., methyl).

In some embodiments of a compound of formula I, a 3 to 6-membered heteroaryl refers to a (fully) aromatic ring system having 3, 4, 5, or 6 ring atoms, (e.g., 5 ring atoms), selected from C, N, O, and S, (e g. C, N, and O, or C and N), with the number of N atoms being 0, 1, 2 or 3, (e.g., 0 or 1), and the number of O and S atoms each being 0, 1 or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrroyl, imidazolyl.

In some embodiments of a compound of formula I, a 3 to 9-membered heterocycloalkyl refers to a non-aromatic or partially aromatic ring system having 3 to 9, (e.g., 5 to 7 ring atoms) independently selected from C, N, O, and S, (e.g. C, N, or O), the number of N atoms being 0, 1, 2, or 3, (e.g., 0 or 1), and the number of O and S atoms each being 0, 1 or 2. Examples of a 3 to 8-membered heterocycloalkyl include monocycles and bridged bicycles Monocycles include oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl morpholinyl); bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O.

In some embodiments, Z is —(NR⁴R⁵), wherein R⁴ and R⁵ are independently of each other H, C₁₋₆ alkyl, or —(NR⁶R⁷), wherein R⁶ and R⁷ form together with the atom to which they are attached to 5-membered heteroaryl containing 0, 1, 2, or 3 N atoms and 0, 1, or 2 O atoms or a 5 to 7-membered heterocycloalkyl containing 0, 1, 2, or 3 N atoms and 0, 1, or 2 O atoms, wherein the 5 to 7-membered heterocycloalkyl is a monocycle or a bridged bicycle and is unsubstituted or substituted with C₁₋₄ alkyl, hal, —OR′, or —NR′R″, wherein R′ and R″ are independently of each other H or C₁₋₄ alkyl.

In some embodiments, —(NR⁶R⁷) ring systems include

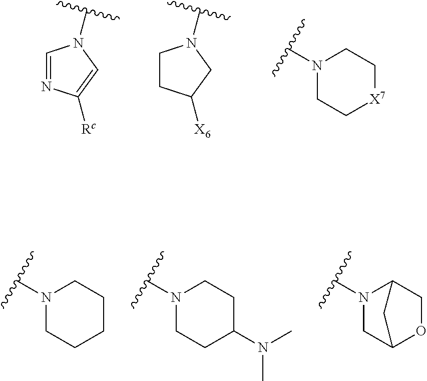

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X is —O—, —NH—, or —N(CH₃)—.

In some embodiments, Y² is covalent bond. In some embodiments, Y² is —O—. In some embodiments, Y² is —NH— or NCH₃—. In some embodiments, Y² is —C≡C—.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, —CH₂—, —(CH₂)₂—, or —C(CH₃)₂—. In some embodiments, L is

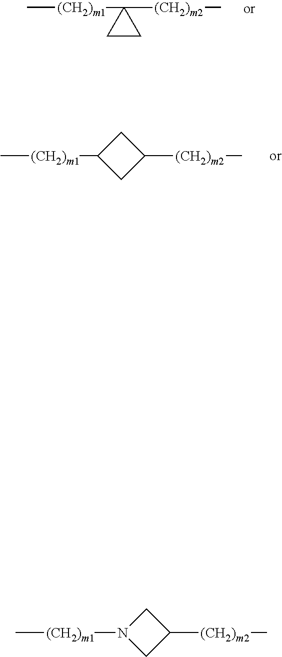

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

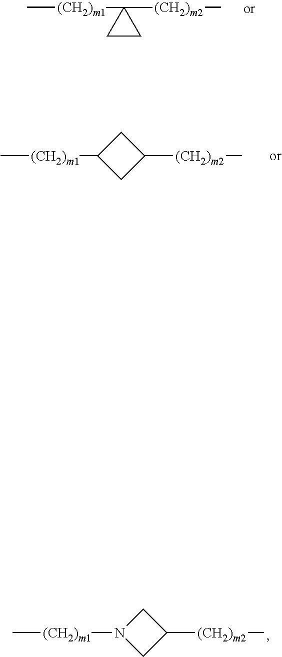

wherein m1 and m2 are independently of each other 0, 1, 2, 3, or 4, (e.g., 0, 1, or 2). In some embodiments, m2 is 0 and m1 is 0, 1, or 2. In some embodiments, m1 and m2 are 1. In some embodiments, m1 and m2 are 2.

In some embodiments, the present disclosure is directed toward a compound or a pharmaceutically acceptable salt or stereoisomer thereof of formula II or III

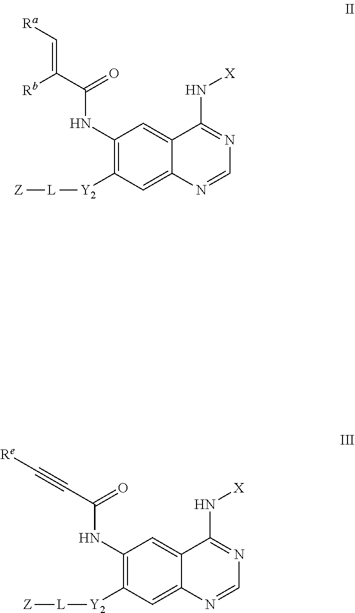

wherein L is a covalent bond, straight chain or branched C₁₋₄ alkyl, or

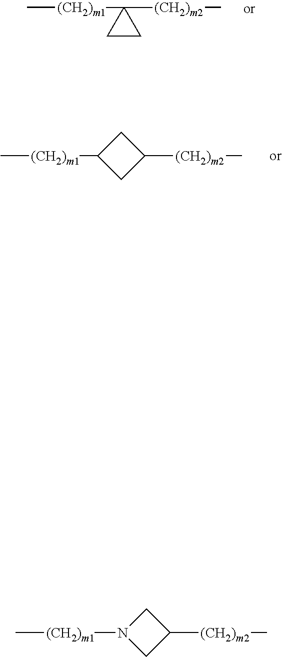

wherein m1 and m2 are independently of each other 0, 1, 2, 3, or 4, (e.g., a covalent bond, straight chain or branched C₁₋₄ alkyl);
Y² is a covalent bond, —O—, —NH—, —NCH₃—, or —C≡C—;
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl;
Rₐ and R_{b} are independently of each other H, hal, or —CH₂—O—CH₃, (e.g., H); Rₑ is H or methyl; and X is a group of formula (ii)a

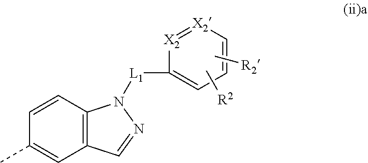

wherein X² and X^{2′} are independently of each other —N═ or —CH═;
L¹ is a covalent bond or straight chain or branched C₁₋₃alkyl, which is unsubstituted or substituted with hal;
R² and R^{2′} are independently of each other H, C₁₋₆ alkyl, hal, —CF₃, or —OCF₃, (e.g., H or hal).

In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-. In some embodiments, L¹ is —CH₂—, —CH₂—CH₂—. In some embodiments, L¹ is —CH₂—.

In some embodiments, the compound of formula II is not a compound wherein X is formula (i)a with L₁ being —CH₂— and Ar being 3-fluorobenzyl. R₁ is CH₂═CH—, Y₂ is O, L is propyl and Z is 4-morpholino, namely N-{4-[1-(3-fluoro-benzyl)-1H-indazole-5-ylamino]-7-[3-(4-morpholino)propoxy]-quinazolin-6-yl}-acrylamide. In some embodiments, X has the following formula (ii)b, (e.g., (ii)c or (ii)c′)

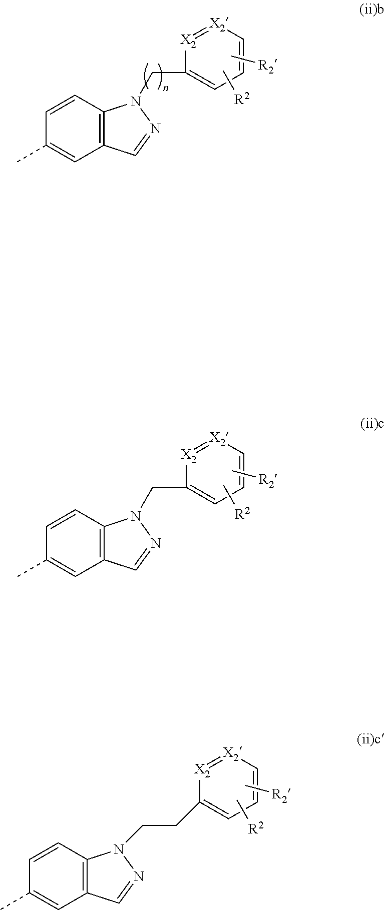

wherein X² and X^{2′} are independently of each other —N═ or —CH═; R² and R^{2′} are independently of each other H, C₁₋₆ alkyl, hal, —CF₃, or —OCF₃; and n is 1 or 2.

In some embodiments, both X² and X^{2′} are —CH═. In some embodiments, X² is —CH═ and X^{2′} is —N═ or X^{2′} is —CH═ and X² is —N═. In some embodiments, both X² and X^{2′} are —N═.

In some embodiments, R² and R^{2′} are independently of each other H, C₁₋₆ alkyl, or hal, (e.g. H, —CH₃, F, or Cl and H or F).

In some embodiments, X has the following formula (ii)d, (ii)e, (ii)f

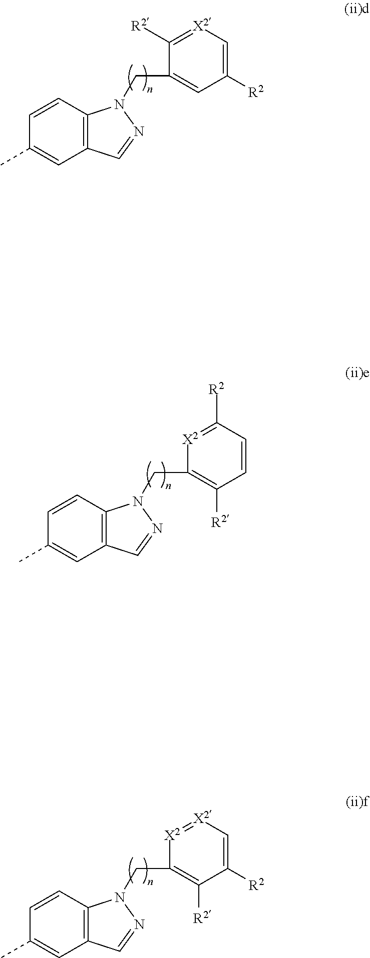

wherein X² and X^{2′} are independently of each other —N═ or —CH═; R² and R^{2′} are independently of each other H, C₁₋₆ alkyl, hal, —CF₃, or —OCF₃; and n is 1 or 2.

In some embodiments, both X² and X^{2′} are —CH═. In some embodiments, X² is —CH═ and X^{2′} is −N═ or X^{2′} is —CH═ and X² is —N═. In some embodiments, both X² and X^{2′} are —N═.

In some embodiments, R² and R^{2′} are independently of each other H, C₁₋₆ alkyl, or hal, (e.g., H, —CH₃, F, or Cl; and H or F).

In some embodiments of a compound of formula II or III, group X has the following formula (ii)g, (ii)h, (ii)i

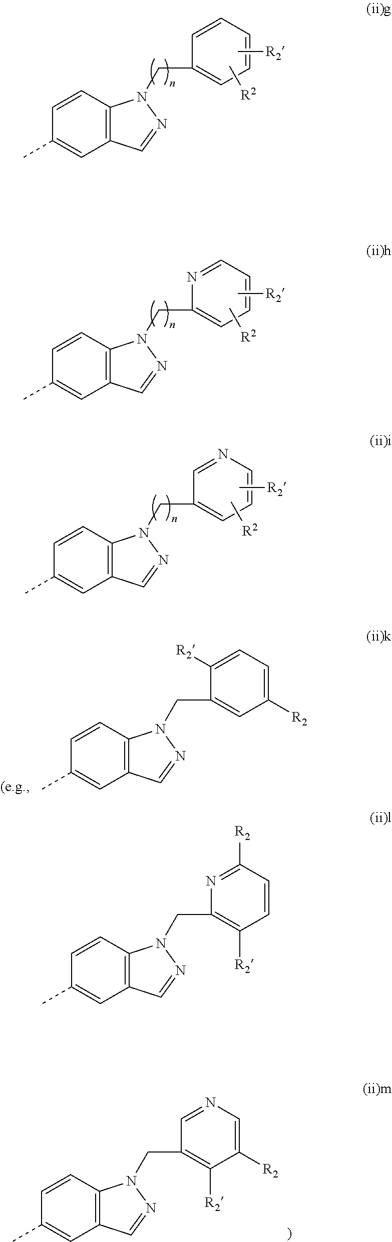

wherein R² is H, C₁₋₆ alkyl, or hal, (e.g. H, —CH₃, F, or Cl); and n is 1 or 2.

In some embodiments of a compound of formula II or III, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the number of N atoms is 0, 1, or 2. In some embodiments, the number of O and S atoms each is 0, 1, or 2. Examples of 3 to 6-membered heterocycloalkyl groups include oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl, 1,4-dithianyl, 1,3-dioxane, 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of a compound of formula II or III, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, 5 ring atoms), independently selected from C, N, O, and S, (e.g., C N, and O, or C and N). In some embodiments, the number of N atoms is 0, 1, 2, or 3.

In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of a compound of formula II or III, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g., C, N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl, 1,4-dithianyl, 1,3-dioxane, 1,3-dithianyl, piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl, (e.g., spiro[3.3]heptanyl, spiro[4.4]nonanyl), having one or two heteroatoms selected from N and O, (e.g., diazaspiro[3.3]heptanyl oxa-azaspiro[3.3]heptanyl, diazaspiro[4.4]nonanyl oxa-azaspiro[4.4]nonanyl).

In some embodiments, group Z of a compound of formula II or III is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered (e.g., 6-8-membered heterocycloalkyl), wherein the 3 to 9-membered heterocycloalkyl is a monocycle or a bridged bicycle and is unsubstituted or substituted with C₁₋₄ alkyl.

In some embodiments, —(NR⁶R⁷) ring systems include

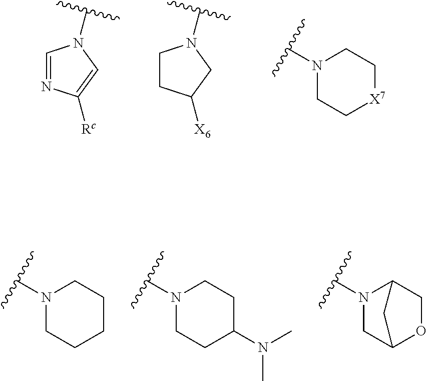

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, the ring systems of group Z include

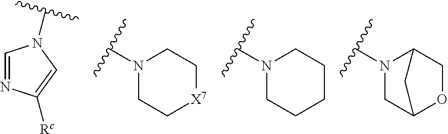

wherein R^{c} is H, C₁₋₄ alkyl, or oxetane; and X⁷ is —O—, —NH—, or —N(CH₃)—.

In some embodiments, Y² is covalent bond. In some embodiments, Y² is —O—. In some embodiments, Y² is —NH— or NCH₃—. In some embodiments, Y² is —C≡C—.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—.

In some embodiments, the compounds of formula II are of formula IIa

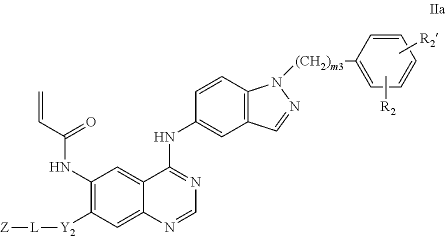

wherein L is a covalent bond, straight chain or branched C₁₋₄ alkyl, or

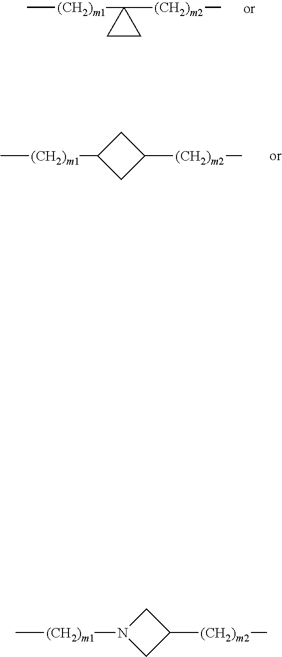

wherein m1 and m2 are independently of each other 0, 1, 2, 3, or 4, (e.g., a covalent bond, straight chain or branched C₁₋₄ alkyl);
m3 is 1 or 2, (e.g., 1);
Y² is a covalent bond, —O—, —NH—, —NCH₃—, or —C≡C—;
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl; and
R² and R^{2′} are independently of each other H, C₁₋₆ alkyl, hal, —CF₃, or —OCF₃, (e.g., H or hal).

In some embodiments of a compound of formula IIa, L is straight chain or branched C₁₋₄ alkyl, (e.g., —CH₂—, —(CH₂)₂—, —(CH₂)₃—, —(CH₂)₄— or —C(CH₃)₂—). In some embodiments, L is —CH₂—, —(CH₂)₂—, or —C(CH₃)₂—.

In some embodiments of a compound of formula IIa, Y² is —O—, —NH—, —NMe-, or —C≡C—, (e.g., —O—, —NMe- or —C≡C—).

In some embodiments of a compound of formula IIa, Z is —(NR⁴R⁵), wherein R⁴ and R⁵ are independently of each other H or C₁₋₄ alkyl, (e.g., Me). In some embodiments, Z is —(NR⁶R⁷) wherein R⁶ and R⁷ form together with the nitrogen to which they are attached to a 3 to 9-membered heterocycloalkyl, wherein the 3 to 9-membered heterocycloalkyl is a monocycle or a fused-, bridged-, or spiro-bicycle or a combination thereof, (e.g., a monocycle or a bicycle). In some embodiments, the 3 to 9-membered heterocycloalkyl is a bicycle. In some embodiments, the —(NR⁶R⁷) ring system includes

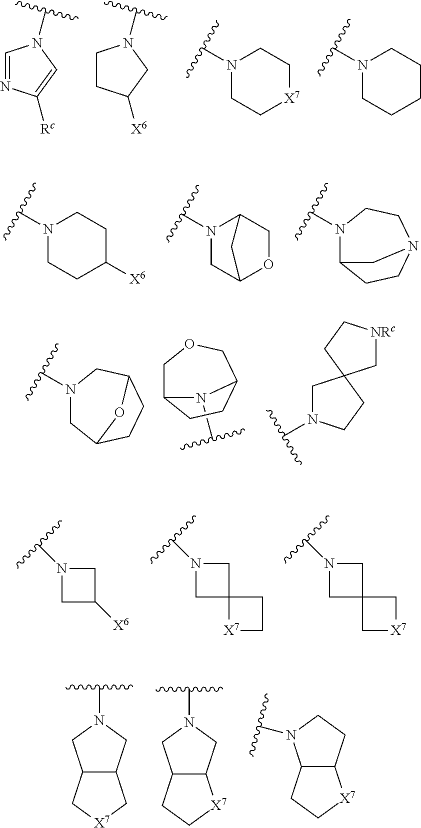

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—. In some embodiments, the —(NR⁶R⁷) ring system includes

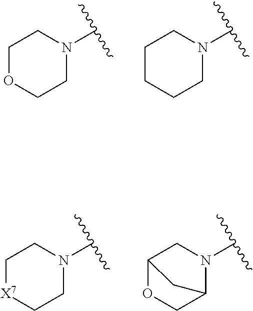

wherein X⁷ is —NH— or —N(CH₃)—.

In some embodiments, the compounds of formula II or IIa are of formula IIb

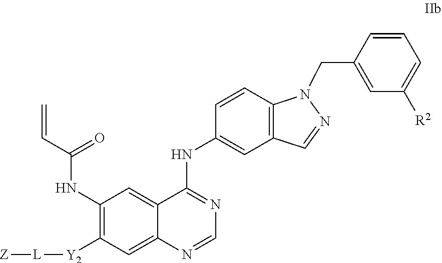

wherein L is a covalent bond, straight chain or branched C₁₋₄ alkyl, or

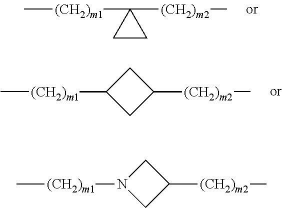

wherein m1 and m2 are independently of each other 0, 1, 2, 3, or 4, (e.g., a covalent bond, straight chain or branched C₁₋₄ alkyl);
Y² is a covalent bond, —O—, —NH—, —NCH₃, or —C≡C—;
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl; and
R² is C₁₋₆ alkyl, hal, —CF₃, or —OCF₃, (e.g., H or hal).

In some embodiments of a compound of formula IIb, L is straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—.

In some embodiments of a compound of formula IIb, Y² is —O—, —NH—, —NMe-, or —C≡C—.

In some embodiments of a compound of formula IIb, R² is F.

In some embodiments of a compound of formula IIb, Z is —(NR⁴R⁵), wherein R⁴ and R⁵ are independently of each other H or C₁₋₄ alkyl, (e.g., Me). In some embodiments, Z is —(NR⁶R⁷) wherein R⁶ and R⁷ form together with the nitrogen to which they are attached to a 3 to 9-membered heterocycloalkyl, wherein the 3 to 9-membered heterocycloalkyl is a monocycle or a fused-, bridged-, or spiro-bicycle or a combination thereof, (e.g., a monocycle or a bicycle). In some embodiments, the 3 to 9-membered heterocycloalkyl is a bicycle. In some embodiments, the —(NR⁶R⁷) ring system includes

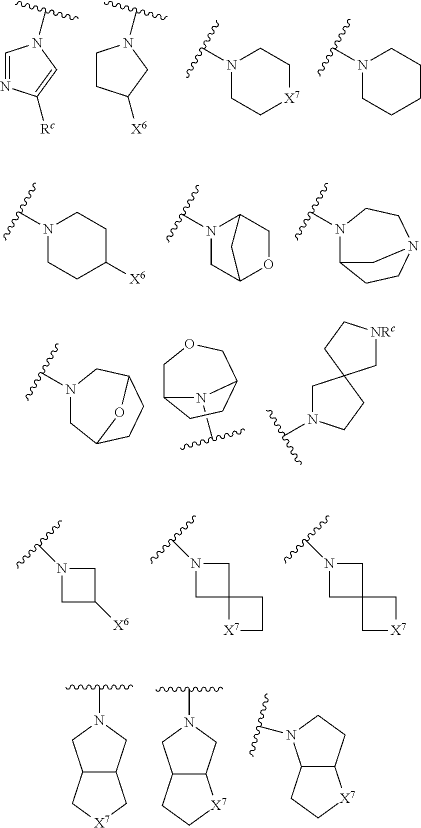

wherein R, is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—. In some embodiments, the —(NR⁶R⁷) ring system includes

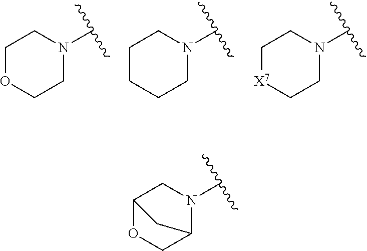

wherein X⁷ is —NH— or —N(CH₃)—.

In some embodiments, the compounds of formula II, IIa or IIb are of formula IIc, IId or IIe

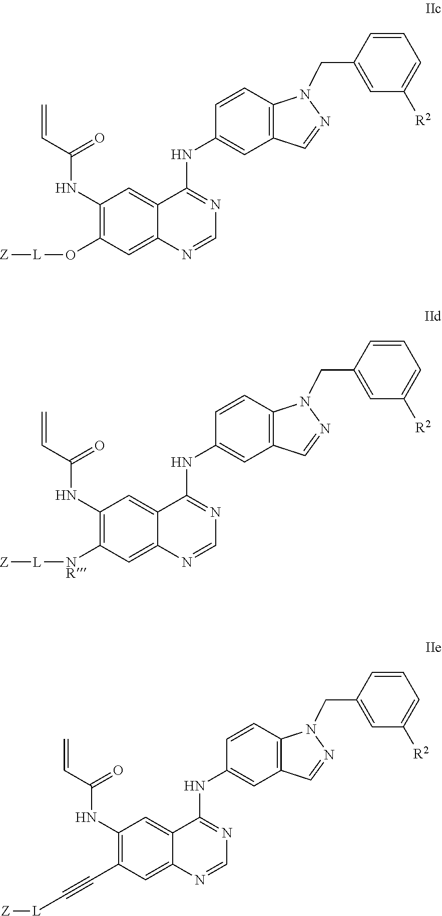

wherein L is a covalent bond, straight chain or branched C₁₋₄ alkyl, or

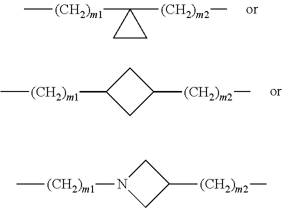

wherein m1 and m2 are independently of each other 0, 1, 2, 3, or 4, (e.g., a covalent bond, straight chain or branched C₁₋₄ alkyl);

### R′″ is H or Me;

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷) or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl; and
R² is C₁₋₆ alkyl, hal, —CF₃, or —OCF₃, (e.g., H or hal).

In some embodiments of a compound of formula IIc, IId or IIe, L is straight chain or branched C₁₋₄ alkyl, (e.g., —CH₂—, —(CH₂)₂—, —(CH₂)₄—, or —C(CH₃)₂—). In some embodiments, L is —CH₂—, —(CH₂)₂—, or —C(CH₃)₂—.

In some embodiments of a compound of formula IIc, IId or IIe, R² is F.

In some embodiments of a compound of formula IIc, IId or IIe, Z is —(NR⁴R⁵), wherein R⁴ and R⁵ are independently of each other H or C₁₋₄ alkyl, (e.g., Me). In some embodiments, Z is —(NR⁶R⁷) wherein R⁶ and R⁷ form together with the nitrogen to which they are attached to a 3 to 9-membered heterocycloalkyl, wherein the 3 to 9-membered heterocycloalkyl is a monocycle or a fused-, bridged-, or spiro-bicycle or a combination thereof, (e.g., a monocycle or a bicycle). In some embodiments, the 3 to 9-membered heterocycloalkyl is a bicycle. In some embodiments, the —(NR⁶R⁷) ring system includes

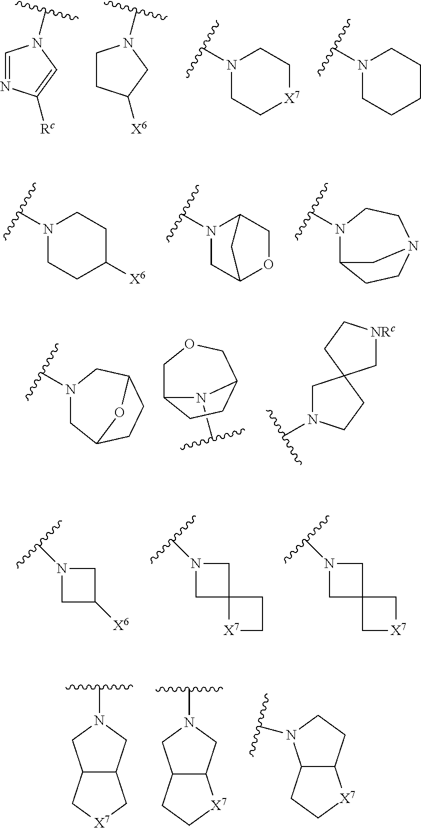

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—. In some embodiments, the —(NR⁶R⁷) ring system includes

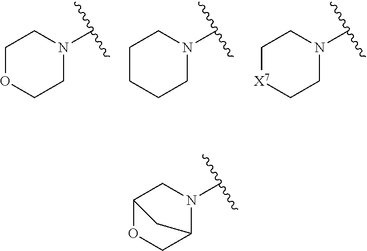

wherein X⁷ is —NH— or —N(CH₃)—.

In some embodiments, the present disclosure is directed toward a compound or a pharmaceutically acceptable salt or stereoisomer thereof of formula I above wherein Y² is covalent bond, having the following formula IV

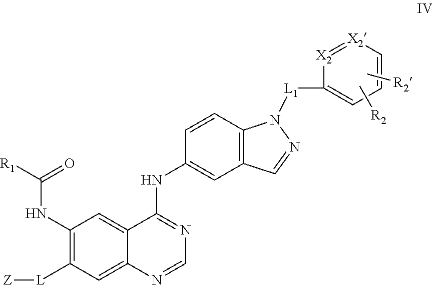

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH, or —C≡C—CH₃—, wherein Rₐ and R_{b} are independently of each other H, hal, or —CH₂—O—CH₃;
R² and R^{2′} are independently of each other H, C₁₋₆ alkyl, hal, —CF₃, or —OCF₃;
L is a covalent bond, straight chain or branched C₁₋₄ alkyl, or

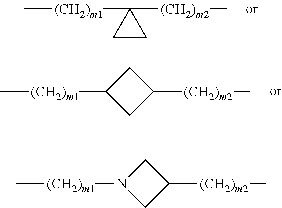

wherein m1 and m2 are independently of each other 0, 1, 2, 3, or 4, (e.g., L is a covalent bond, straight chain or branched C₁₋₄ alkyl);
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═. In some embodiments, R² and R^{2′} are independently of each other H, hal, or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═—CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)—, —(CH₂)₃—, —(CH₃)₄—, —C(CH₃)₂—, or —CH₂—C(CH₃)₂—) In some embodiments, L is —CH₂—, —(CH₂)₂—, or —C(CH₃)₂—. In some embodiments, L is

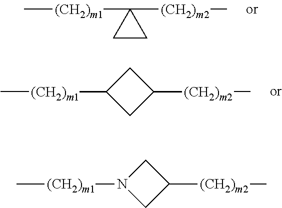

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g. —CH₂—, —(CH₂)₂—, —(CH₂)₃—, —(CH₂)₄—, —C(CH₃)₂—, or —CH₂—C(CH₃)₂—). In some embodiments, L is

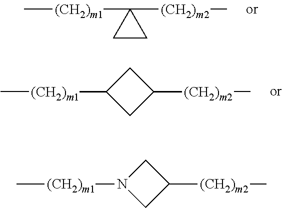

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, L¹ is a covalent bond. In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-.

In some embodiments, L¹ is —CH₂—, —CH₂—CH₂—. In some embodiments, —CH₂—.

In some embodiments, compound IV has the following formula IV-1

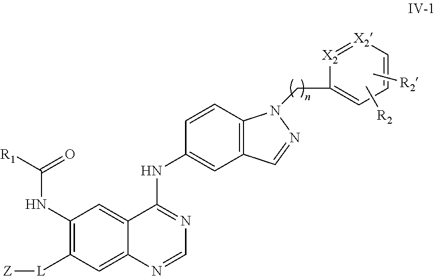

(e.g. one of the following formulas IV-1a or IV-1b)

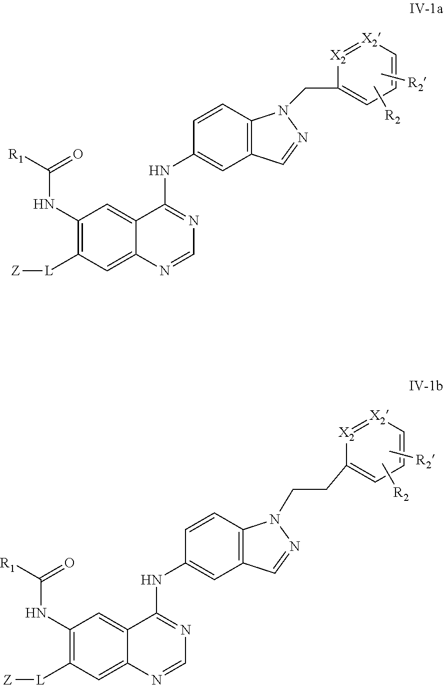

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
s R² and R^{2′} are independently of each other H, C₁₋₆ alkyl, hal, —CF₃, or —OCF₃;
n is 0, 1, 2, 3, (e.g., 1 or 2);
L is a covalent bond, straight chain or branched C₁₋₄ alkyl, or

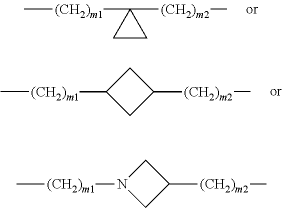

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

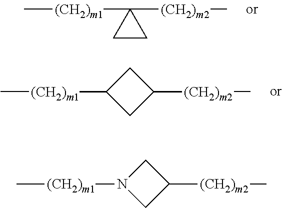

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

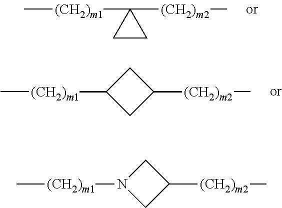

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, compound IV has one of the following formulas

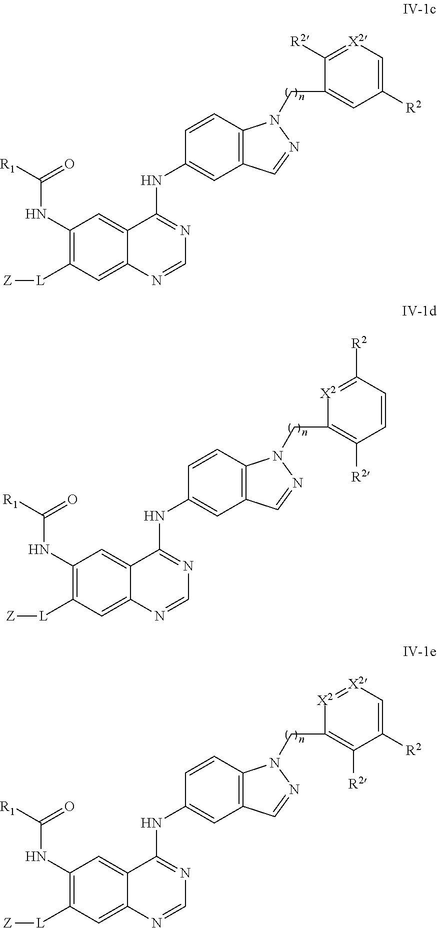

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

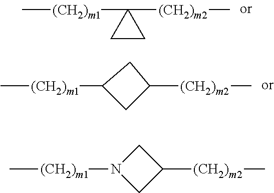

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal, or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

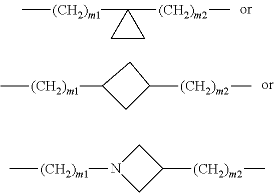

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g. —CH₂—, —(CH₂)₂—, —(CH₂)₃—, —(CH₂)₄—, —C(CH₃)₂—, or CH₂—C(CH₃)₂—). In some embodiments, L is

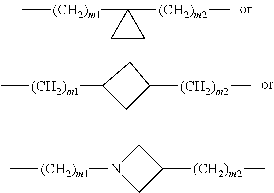

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, compound IV has one of the following formulas

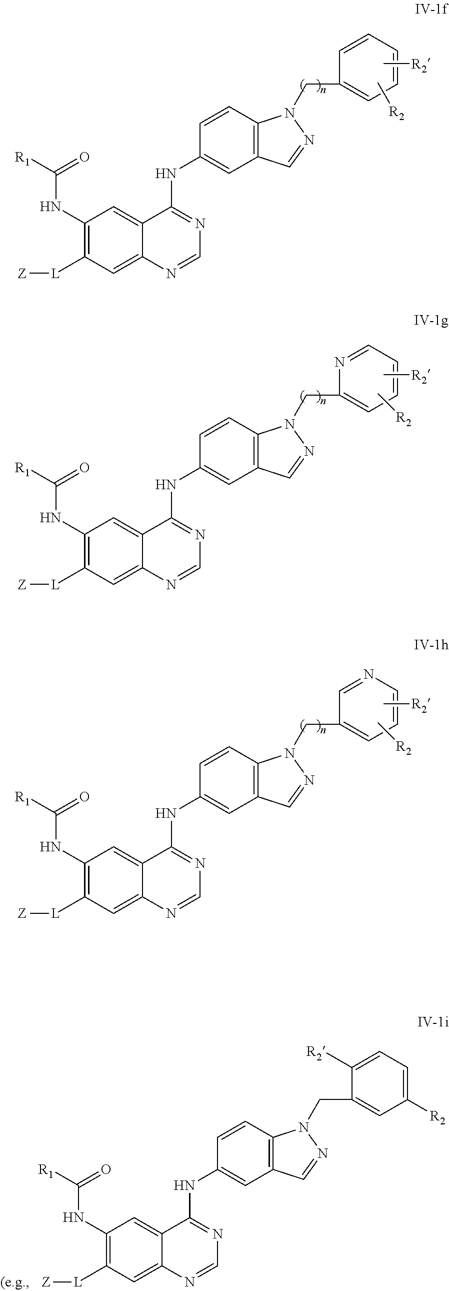

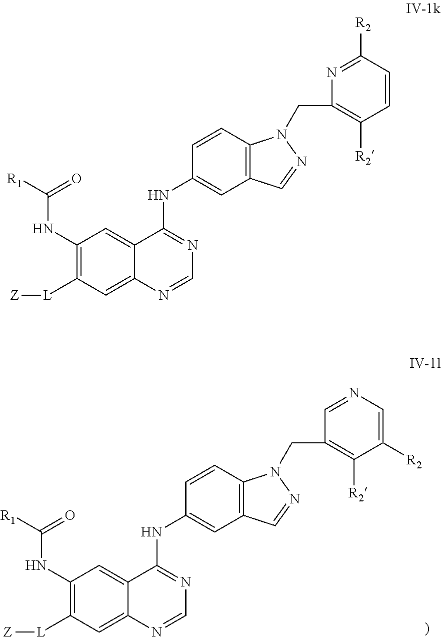

wherein R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

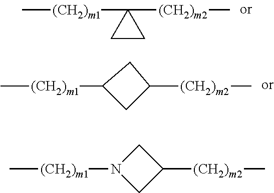

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR₆R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H4 or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═—CH—CH₂—O—C₃H. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—) In some embodiments, L is

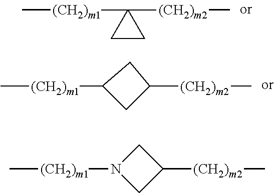

wherein m1 and m2 are independently of each other 0, 1, 2, 3, or 4, (e.g., 0, 1, or 2). In some embodiments, m2 is 0 and m1 is 0, 1, or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, of a compound of formula IV, IV-1, and IV-1a to IV-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the number of N atoms is 0, 1, or 2.

In some embodiments, the number of O and S atoms each is 0, 1, or 2. Examples of 3 to 6-membered heterocycloalkyl groups include oxiranyl, thiaranyl, aziradinyl, oxetanyl, thiatanyl, azetidinyl, pyrrolidinyl tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of each compound of formula IV, IV-1, and IV-1a to IV-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, 5 ring atoms), independently selected from C, N, O, and S, (e.g., C, N, and O or C and N. In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of a compound of formula IV, IV-1, and IV-1a to IV-1l, a 3 to 9-membered heterocycloalkyl (in combination with —(NR⁶R⁷) or —(CHR⁶R⁷) refers to a non-aromatic or partially aromatic ring system having 3 to 9 ring atoms independently selected from C, N, O, and S, (e.g., C, N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl, (e.g., spiro[3.3]heptanyl, spiro[4.4]nonanyl), having one or two heteroatoms selected from N and O, (e.g., diazaspiro[3.3]heptanyl oxa-azaspiro[3.3]heptanyl, diazaspiro[4.4]nonanyl, oxa-azaspiro[4.4]nonanyl).

In some embodiments, —(NR⁶R⁷) ring systems include

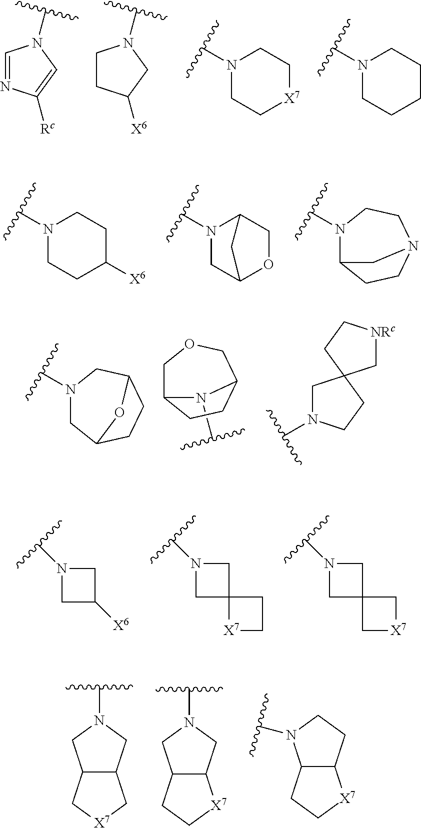

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CHR⁶R⁷) ring systems include

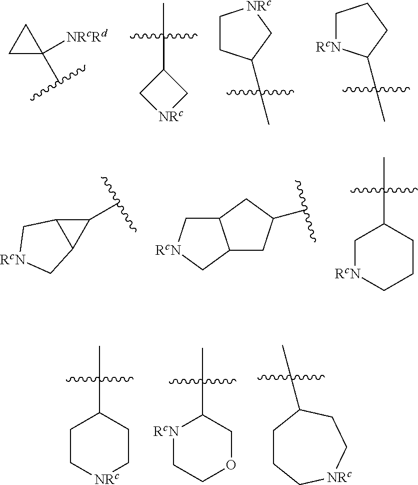

wherein R^{c} is H, C₁₋₄ alkyl, or oxetane; and R^{d} is H or C₁₋₄ alkyl.

In some embodiments of each compound of formula IV, IV-1, and TV-1a to IV-1l, Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl).

In some embodiments, ring systems of group Z include

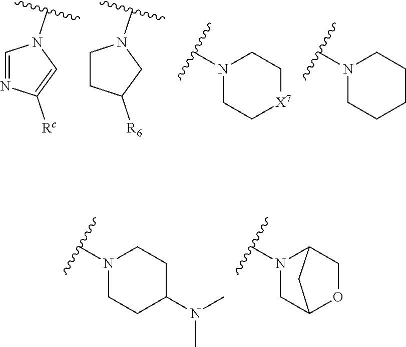

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, ring systems of group Z include

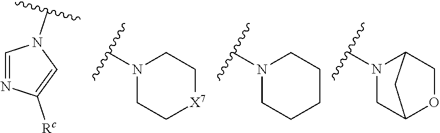

wherein R^{c} is H, C₁₋₄ alkyl, or oxetane; and X⁷ is —O—, —NH—, or —N(CH₃)—.

In some embodiments a compound of formula IV has the formula V or VI

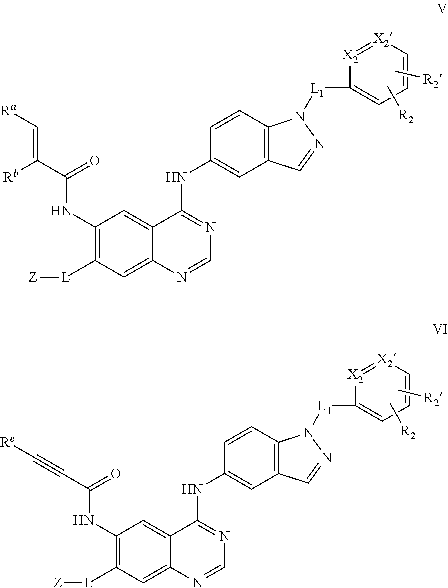

wherein X² and X^{2′} are independently of each other —N═ or —CH═;
L¹ is a covalent bond or straight chain or branched C₁₋₃alkyl, which is unsubstituted or substituted with hal;
R² and R^{2′} are independently of each other H, C₁₋₆ alkyl, hal, —CF₃, or —OCF₃;
Rₐ and R_{b} are independently of each other H, hal, or —CH₂—O—CH₃; and Rₑ is H or methyl.

In some embodiments, L is a covalent bond, straight chain or branched C₁₋₄ alkyl, or

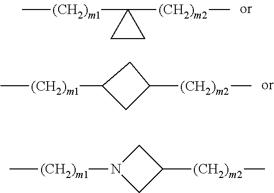

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, or —(NR⁶R⁷), —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′ or —NR′R″, wherein R′ and R″ are independently of each other H1 or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal, or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-. In some embodiments, L¹ is —CH₂—, or —CH₂—CH₂—, (e.g., —CH₂—).

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂— or —C(CH₃)₂—. In some embodiments, L is

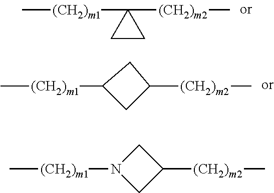

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

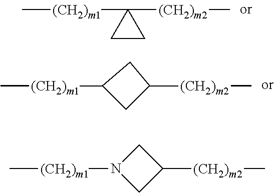

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula V or VI has the formula V-1 or VI-1, (e.g., V-1a, V-1b or VI-1a, VI-1b)

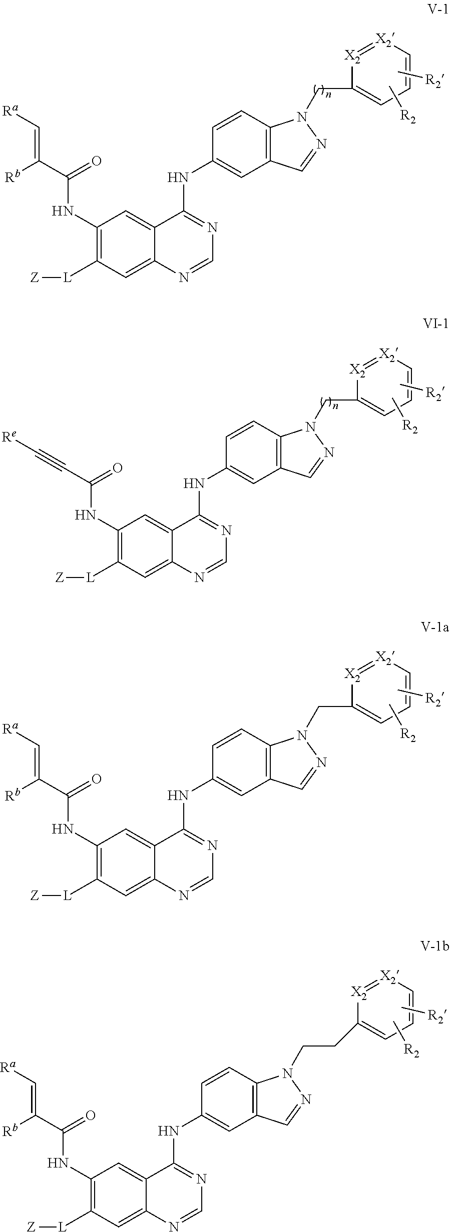

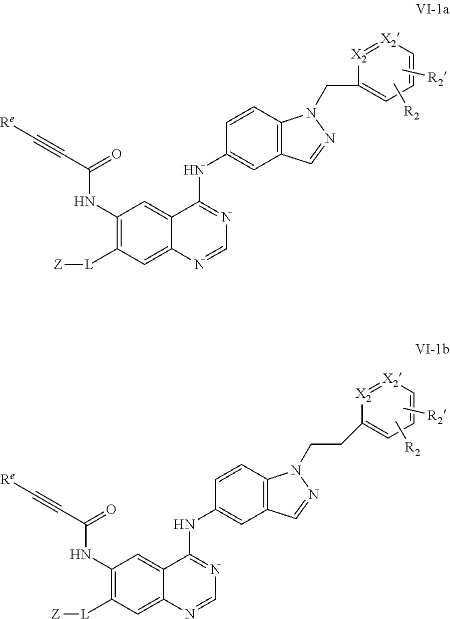

wherein X² and X^{2′} are independently of each other —N or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

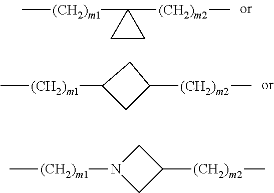

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 1s 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, both X² and X^{2′} are —CH═. In some embodiments, X² is —N═ and X^{2′} is —CH═ or XT is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

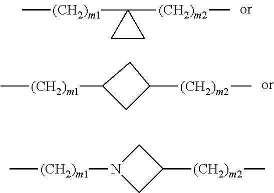

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

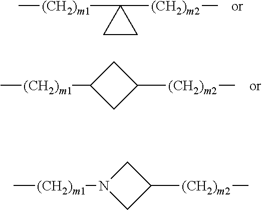

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula V-1 and VI-1 have the formula V-1c, V-1d, V-1e and VI-1c, VI-1d, VI-1e

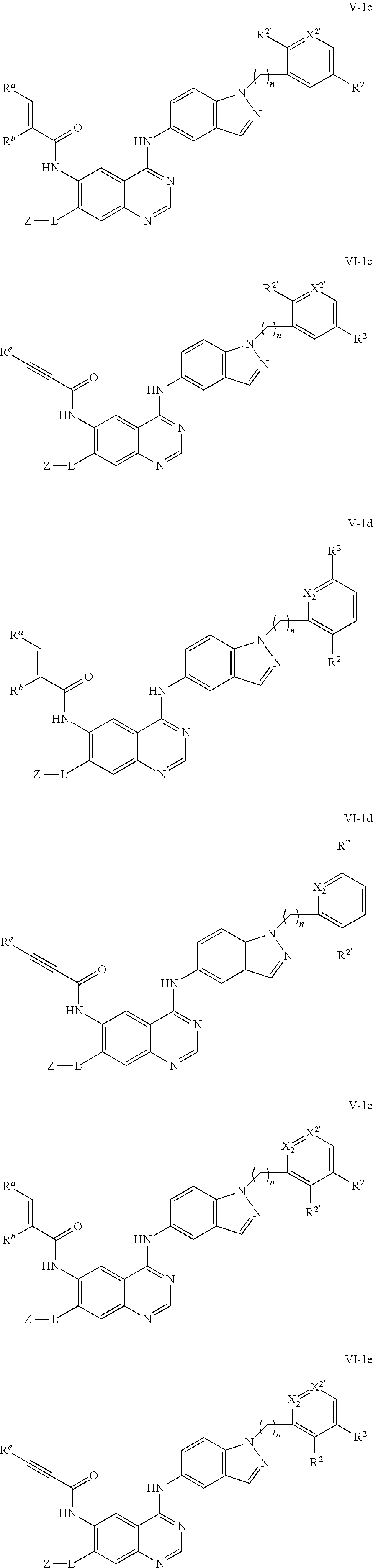

wherein X² and X^{2′} are independently of each other —N═ or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, or 3, (e.g., 1 or 2);
L is a covalent bond, straight chain or branched C₁₋₄ alkyl, or

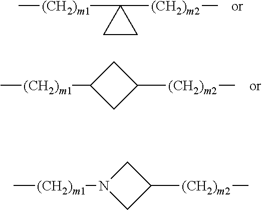

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal, or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

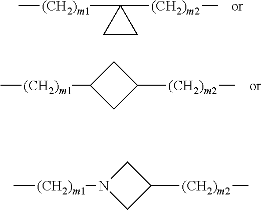

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

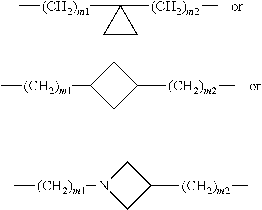

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula V-1 and VI-1 have the formula V-1f, V-1g, V-1h and VI-1f, VI-1g, VI-1h

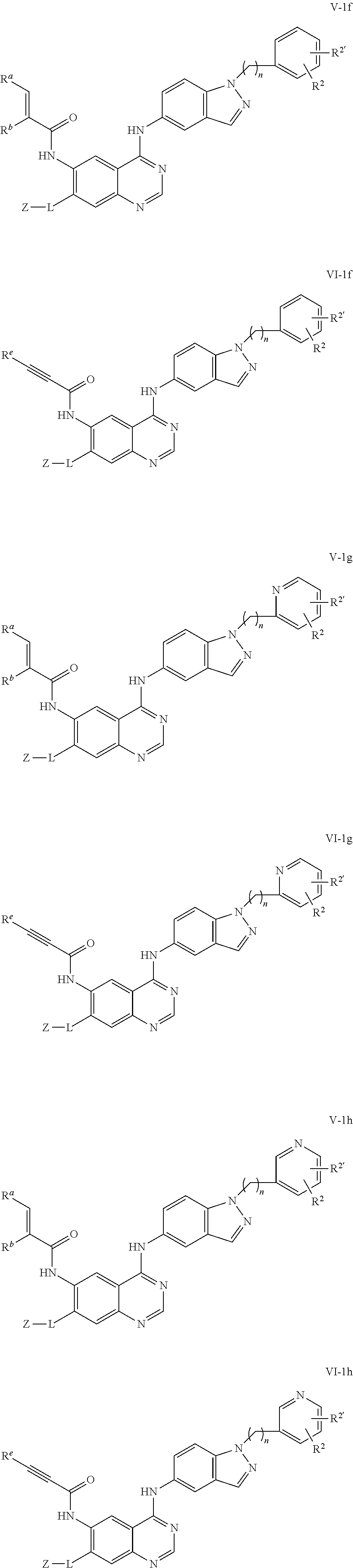

wherein Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

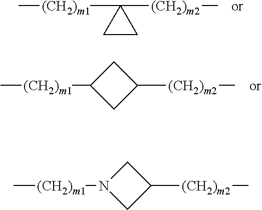

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, R² and R^{2′} are independently of each other H, hal or C₁₋₆ alkyl, (e.g., H, hal or —CH₃). In some embodiments, R² is H or hal. In some embodiments, R^{2′} is H. In some embodiments, R² and R^{2′} are H. In Some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

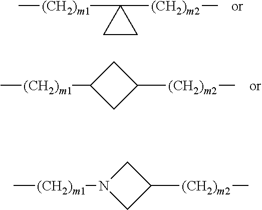

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

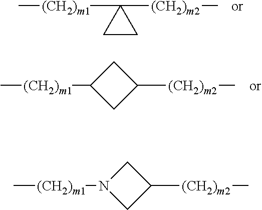

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula V-1 and VI-1 have the formula V-1i, V-1k, V-1l and VI-1i, VI-1k, VI-1l

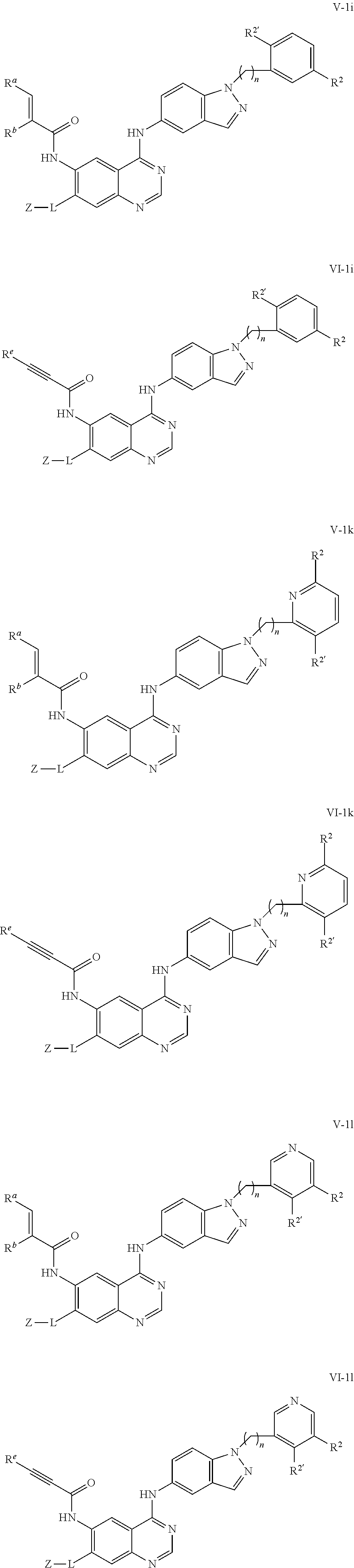

wherein Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

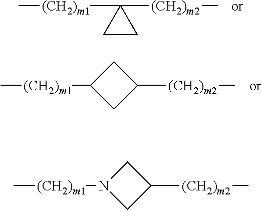

wherein m1 and 2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′RR″, wherein R′ and R″ are independently of each other H or C₁₋₄ alkyl.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In Some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

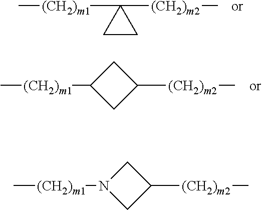

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

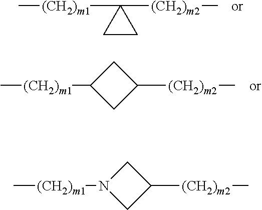

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments of each compound of formula V, V-1, V-1a to V-1l, and VI, VI-1, VI-1a to VI-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the number of N atoms is 0, 1, or 2. In some embodiments, the number of O and S atoms each is 0, 1, or 2. Examples of 3 to 6-membered heterocycloalkyl groups include oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of each compound of formula V, V-1, V-1a to V-1l, and VI, VI-1, VI-1a to VI-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, 5 ring atoms), independently selected from C, N, O and S, (e.g., C, N, and O, or C and N). In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of each compound of formula V, V-1, V-1a to V-1l, and VI, VI-1, VI-1a to VI-1l, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g., C, N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl, (e.g., spiro[3.3]heptanyl, spiro[4.4]nonanyl), having one or two heteroatoms selected from N and O, (e.g., diazaspiro[3.3]heptanyl, oxa-azaspiro[3.3]heptanyl, diazaspiro[4.4]nonanyl, oxa-aspiro[4.4]nonanyl).

In some embodiments, —(NR⁶R⁷) ring systems include

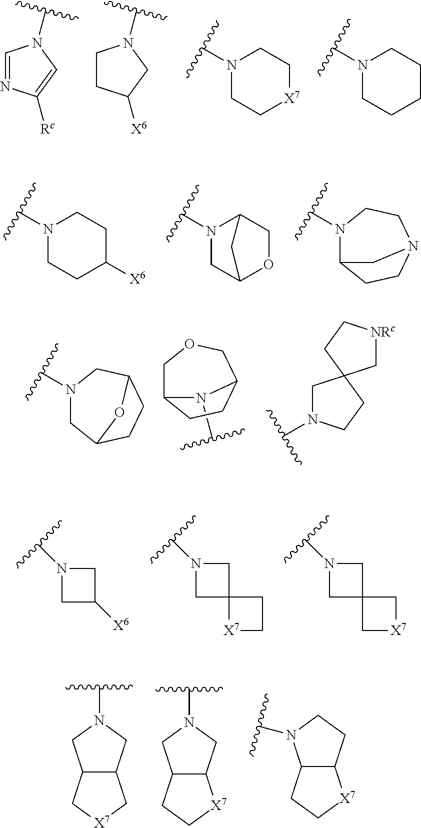

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CH R⁶R⁷) ring systems include

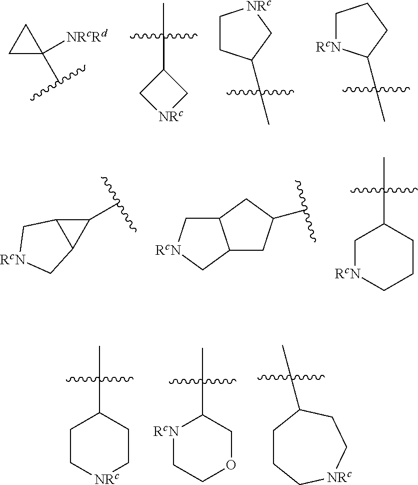

wherein R^{c} is H, C₁₋₄ alkyl, or oxetane; and R^{d} is H or C₁₋₄ alkyl.

In some embodiments of each compound of formula V, V-1. V-1a to V-1l, and VI, VI-1, VI-1a to VI-1l, Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl) include the ring systems as defined above.

In some embodiments, ring systems of group Z include

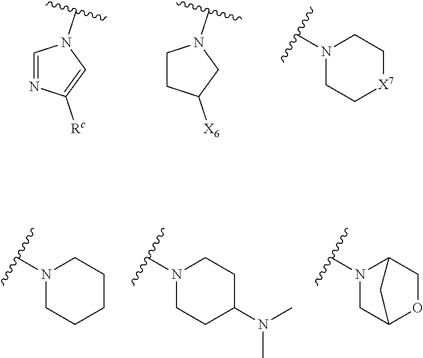

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, ring systems of group Z include

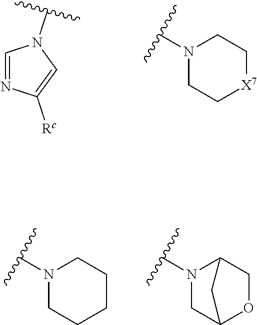

wherein R^{c} is H, C₁₋₄ alkyl, or oxetane; and X⁷ is —O—, —NH—, or —N(CH₃)—.

In some embodiments, the present disclosure is directed toward a compound or a pharmaceutically acceptable salt or stereoisomer thereof of formula I above wherein Y² is —O—, having the following formula VII

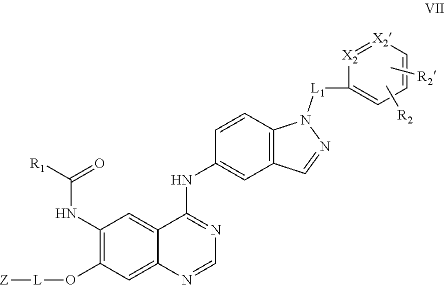

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
L is a covalent bond, straight chain or branched C₁₋₄ alkyl, or

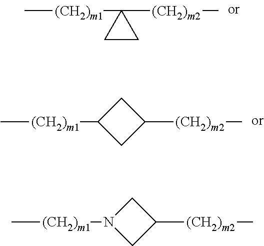

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl,
with the proviso that when R¹ is —CH═CH₂; X², X^{2′}, R² and R^{2′} form m-fluorophenyl, L is methylene and L is propylene in a compound of formula VII (including VII-1, VII-1a to VII-1l and VIII, VIII-1, VIII-1a to VIII-1l), Z cannot be N-linked morpholine.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

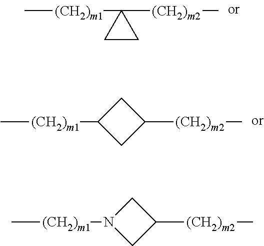

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

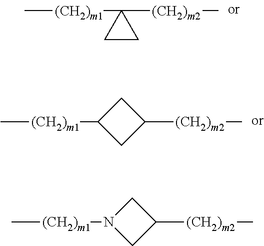

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, L¹ is a covalent bond. In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-.

In some embodiments, L¹ is —CH₂—, —CH₂—CH₂—. In some embodiments, L¹ is —CH₂—.

In some embodiments, the compound of formula VII is not a compound wherein X is formula (i)a with L₁ being —CH₂— and Ar being 3-fluorobenzyl, R₁ is CH₂═CH—, Y₂ is O, L is propyl and Z is 4-morpholino, namely N-{4-[1-(3-fluoro-benzyl)-1H-indazole-5-ylamino]-7-[3-(4-morpholino)propoxy]-quinazolin-6-yl}-acrylamide.

In some embodiments, compound VII has the following formula VII-1

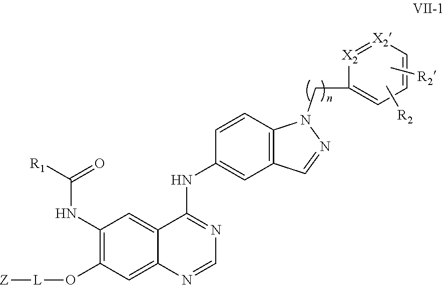

(e.g., one of the following formulas VII-1a or VII-1b

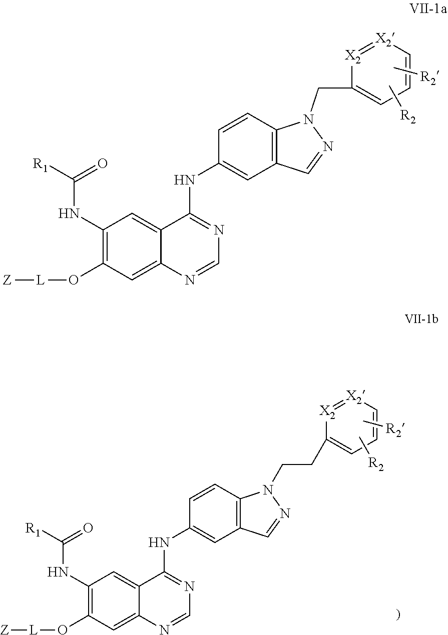

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

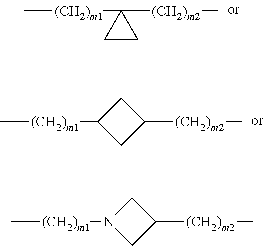

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, the compound is a compound of formula VII-1 or VII-1a, with the proviso that when R¹ is —CH═CH₂; X², X^{2′}, R², R^{2′} form m-fluorophenyl, n is 1 and L is propylene in a compound of formula VII-1 (including VII-1a), Z cannot be N-linked morpholine.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C═CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

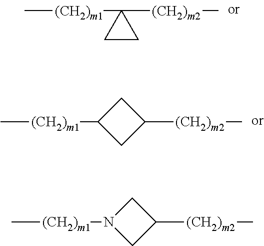

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂— or —CH₂—C(CH₃)₂—). In some embodiments, L is

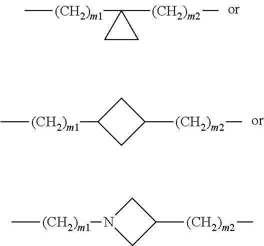

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—).

In some embodiments, compound VII has one of the following formulas

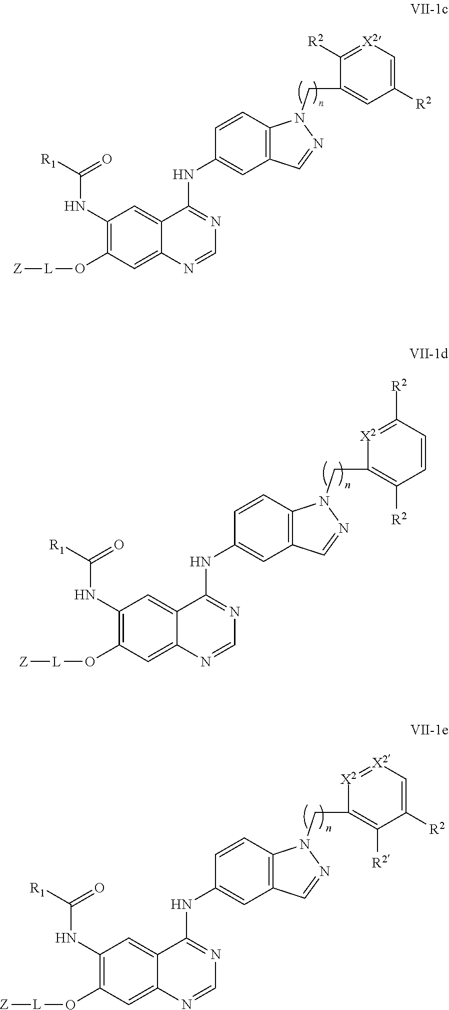

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

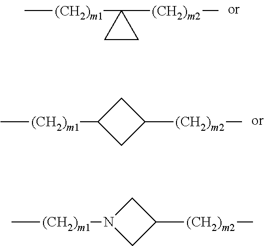

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is —H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

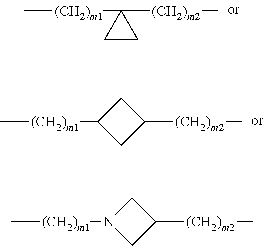

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

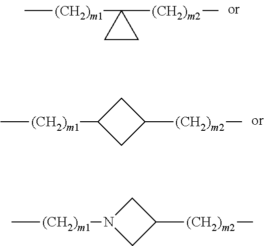

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, compound VII has one of the following formulas

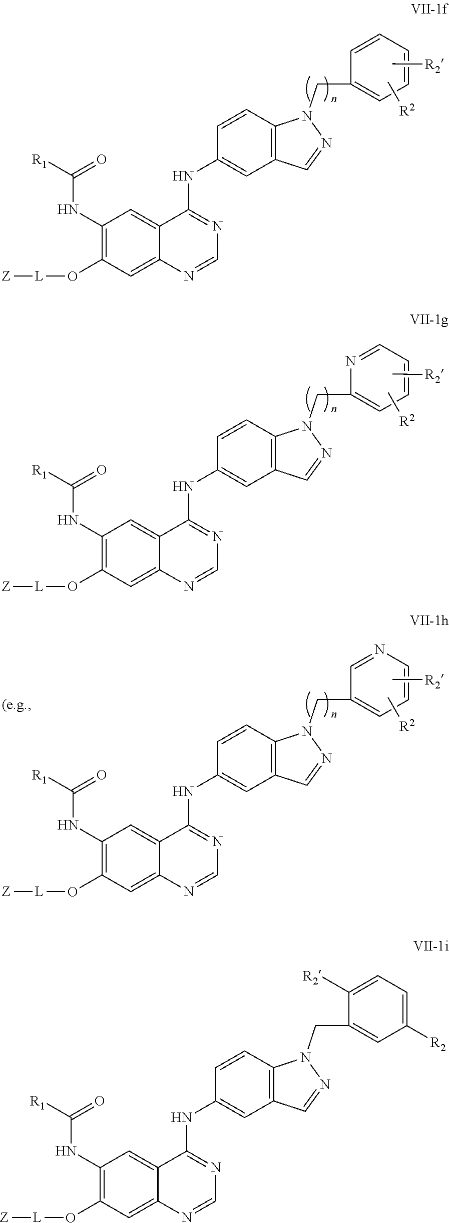

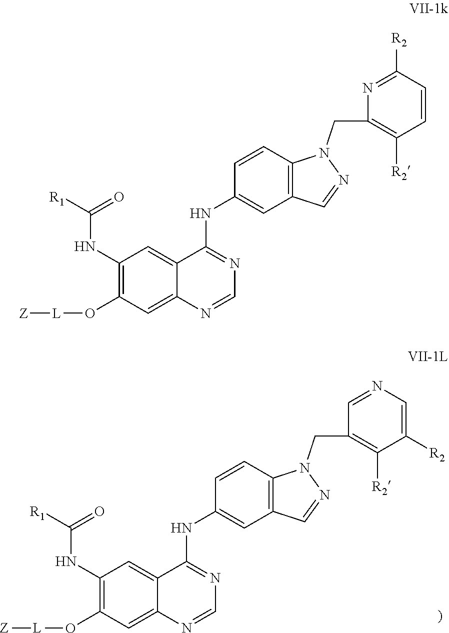

wherein R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

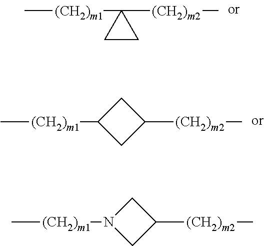

s wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′ or —NR′R″, wherein R′ and R″ are independently of each other H1 or —C₁₋₄ alkyl.

In some embodiments, the compound is a compound of formula VII-1f or VII-1i, with the proviso that when R¹ is —CH═CH₂; X², X^{2′}, R², R^{2′} form m-fluorophenyl, n is 1 and L is propylene in a compound of formula VII-1 (including VII-1a), Z cannot be N-linked morpholine.

In some embodiments, both X², X^{2′} are —CH═. In some embodiments X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₂.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—, i In some embodiments, L is

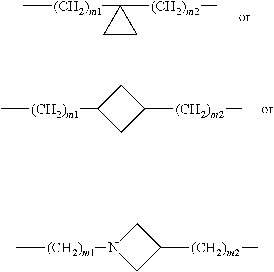

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

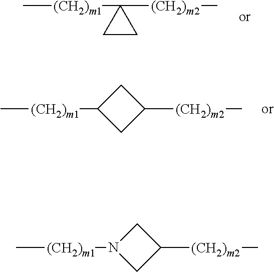

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments of a compound of formula VII, VII-1, and VII-1a to VII-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the number of N atoms is, 1, or 2.

In some embodiments, the number of O and S atoms each is 0, 1, or 2. Examples of 3 to 6-membered heterocycloalkyl groups include oxiranyl, thiaranyl, aziradinyl, oxetanyl, thiatanyl, azetidinyl, pyrrolidin, tetrahydrofuranyl, tetrahydrothiopyranyl, dihydropyranyl, tetrahydropyranyl, 1,3-dioxolanyl, 1,4-dioxanyl, 1,4-oxathianyl, 1,4-dithianyl, 1,3-dioxane, 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of each compound of formula VII, VI-1, and VII-1a to VII-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, 5 ring atoms), independently selected from C, N, O, and S, (e g, C, N and O, or C and N). In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimindinyl, pyrrolyl, thiazolyl, thienyl, and the like. In some embodiments, examples of “heteroaryl” include pyrrolyl, imidazolyl.

In some embodiments of a compound of formula VII, VII-1, and VII-1a to VII-1l, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g. C, N, and O). In some embodiments, the number of N atoms is 0, 1, 2, or 3. In some embodiments, the number of O and S atoms each is 0, 1, or 2. Examples of 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl (e.g., spiro[3.3]heptanyl, spiro[4.4]nonanyl), having one or two heteroatoms selected from N and O, (e.g., diazaspiro[3.3]heptanyl, oxa-azaspiro[3.3]heptanyl, diazaspiro[4.4]nonanyl, oxa-azaspiro[4.4]nonanyl).

In some embodiments, —(NR⁶R⁷) ring systems include

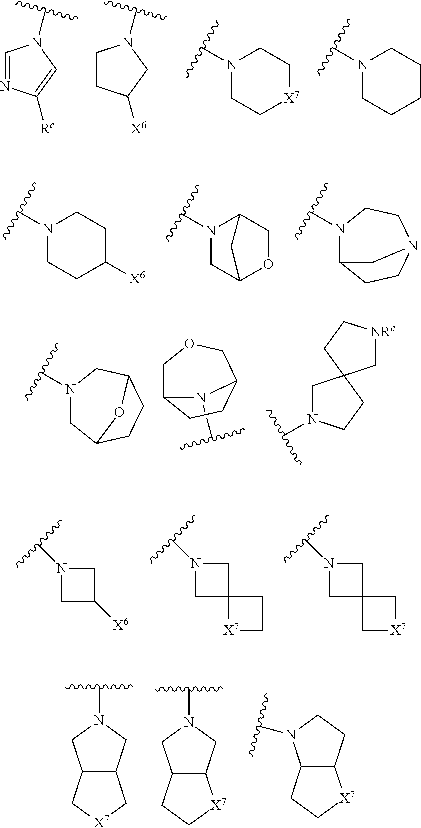

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CHR⁶R⁷) ring systems include

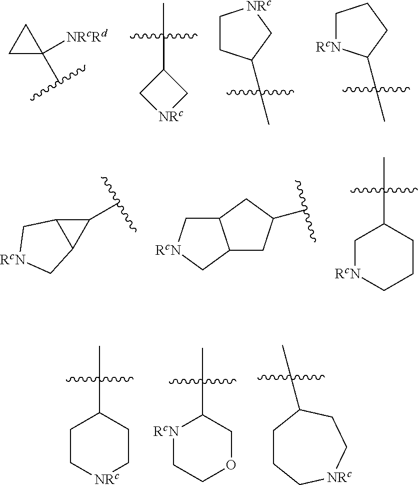

wherein R^{c} is H, C₁₋₄ alkyl, or oxetane; and R^{d} is H or C₁₋₄ alkyl.

In some embodiments of each compound of formula VII, VII-1, and VII-1a to VII-1l, Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl) include the ring systems as defined above.

In some embodiments, ring systems of group Z include

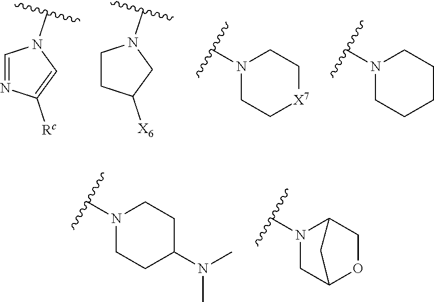

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH—); and X⁷ is —O—, —NH—, or —N(CH)—.

In some embodiments, ring systems of group Z include

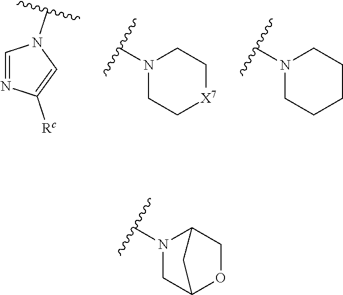

wherein R^{c} is H, C₁₋₄ alkyl, or oxetane; and X⁷ is —O—, —NH—, or —N(CH₃)—.

In some embodiments, a compound of formula VII has the formula VIII or IX

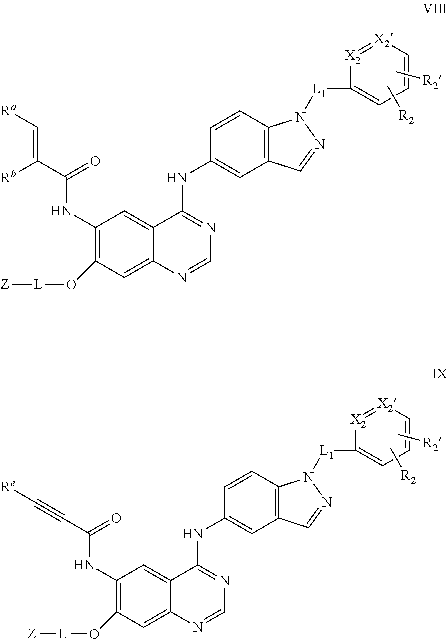

wherein X² and X^{2′} are independently of each other —N═ or —CH═;
L¹ is a covalent bond or straight chain or branched C₁₋₃alkyl, which is unsubstituted or substituted with hal;
R² and R^{2′} are independently of each other H, C₁₋₆ alkyl, hal, —CF₃, or —OCF₃;
Rₐ and R_{b} are independently of each other H, hal, or —CH₂—O—CH₃; and Rₑ is H or methyl.

In some embodiments, L is a covalent bond, straight chain or branched C₁₋₄ alkyl, or

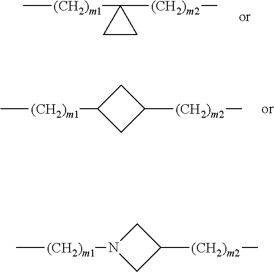

wherein m1 and m2 are independently of each other 0, 1, 2, 3, or 4, (e.g., L is a covalent bond, straight chain or branched C₁₋₄ alkyl); and
Z is —(NR⁴R⁵) wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-. In some embodiments, L¹ is —CH₂—, —CH₂—CH₂—. In some embodiments, L¹ is —CH₂—.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—), In some embodiments, L is —CH₂—, —(CH₂)₂—, or —C(CH₃)₂—. In some embodiments, L is

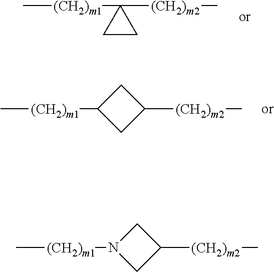

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

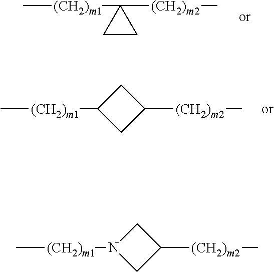

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—.

In some embodiments, a compound of formula VIII or IX has the formula VIII-1 or IX-1, (e.g., VIII-1a, VIII-1b or IX-1a, IX-1b)

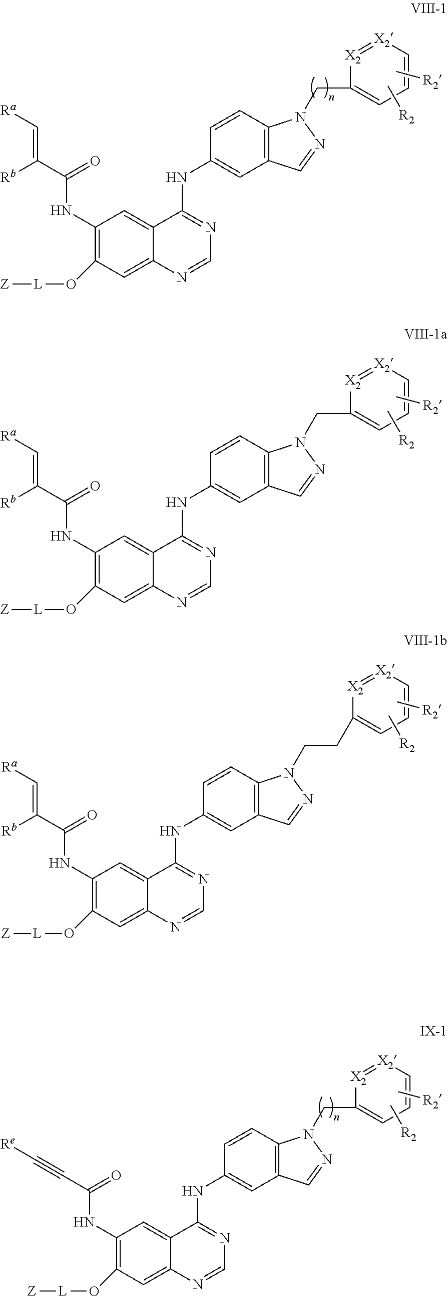

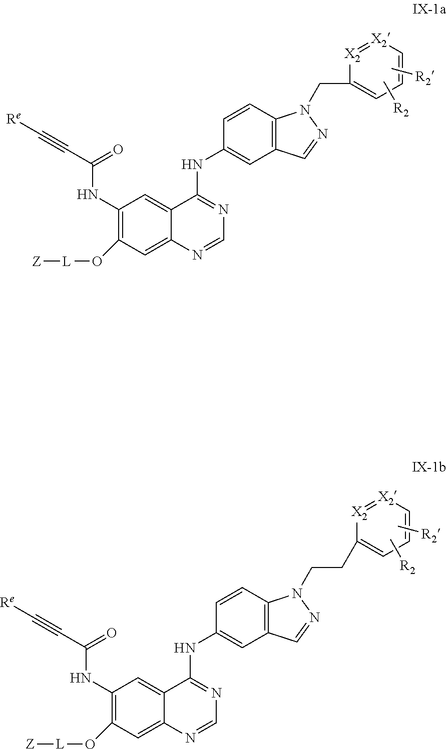

wherein X² and X^{2′} are independently of each other —N═ or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal —CF₃, or —OCF₃;

n is 0, 1, 2, or 3, (e.g., 1 or 2);
L is a covalent bond, straight chain or branched C₁₋₄ alkyl, or

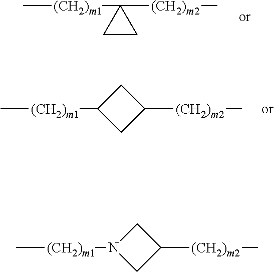

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, the compound is a compound of formula VIII-1 or VIII-1a, with the proviso that when R¹ is —CH═CH₂; X², X^{2′}, R², R^{2′} form m-fluorophenyl, n is 1 and L is propylene in a compound of formula VIII-1 or VIII-1a, Z cannot be N-linked morpholine.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═. In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

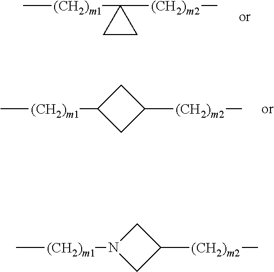

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

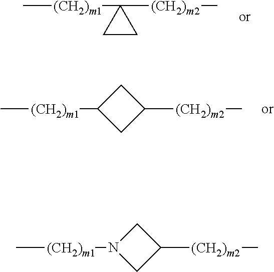

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula VIII-1 and IX-1 have the formula VIII-1c, VIII-1d, VIII-1e and IX-1c, IX-1d, IX-1e

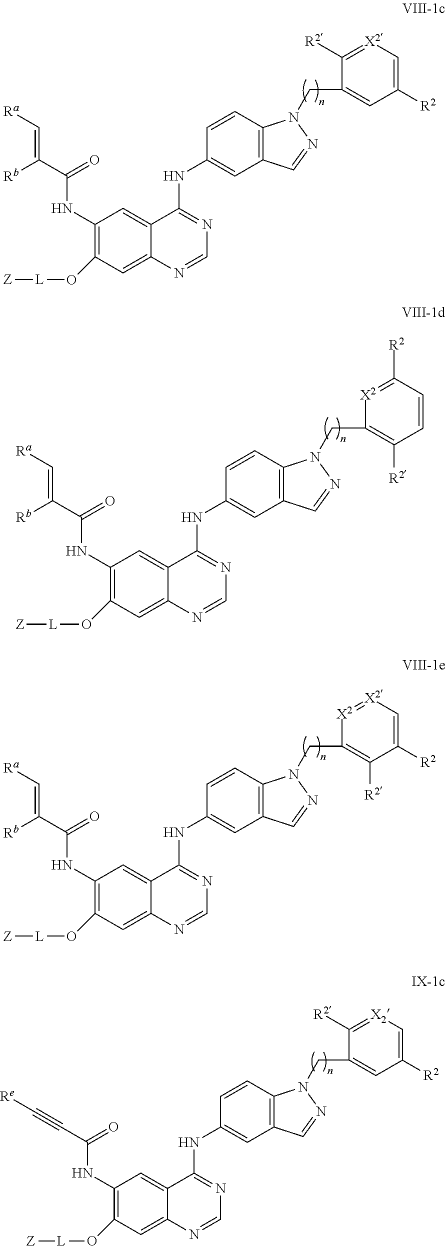

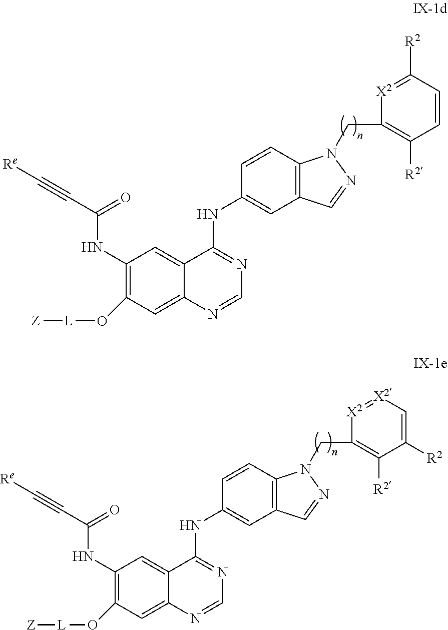

wherein X² and X^{2′} are independently of each other —N═ or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

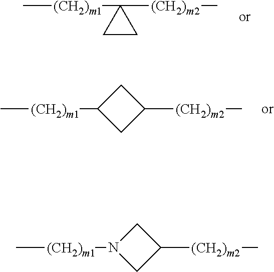

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, the compound is a compound of formula VIII-1c, VIII-1d or VIII-1e, with the proviso that when R^{a} and R^{b}, R^{2′} are H; R² is F; X², X^{2′} are —CH═, n is 1 and L is propylene in a compound of formula VIII-1c, VIII-1d or VIII-1e, Z cannot be N-linked morpholine.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R² is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

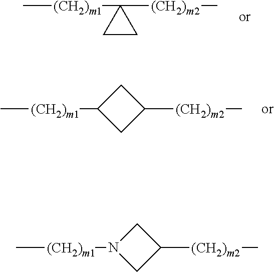

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

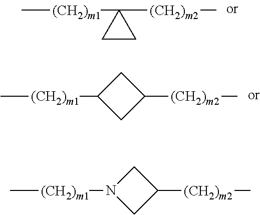

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula VIII-1 and IX-1 have the formula VIII-1f, VIII-1g, VIII-1h and IX-1f, IX-1g, IX-1h

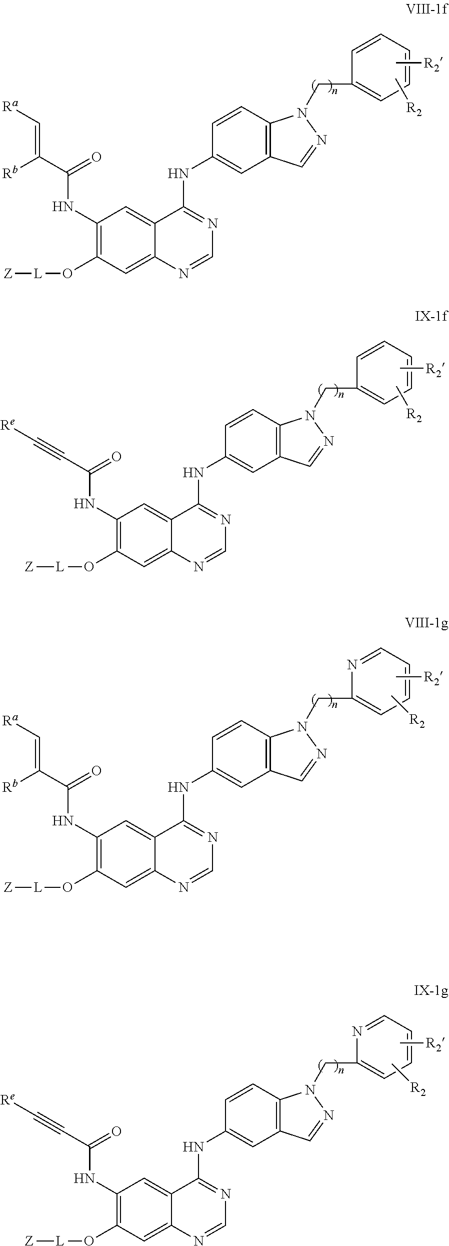

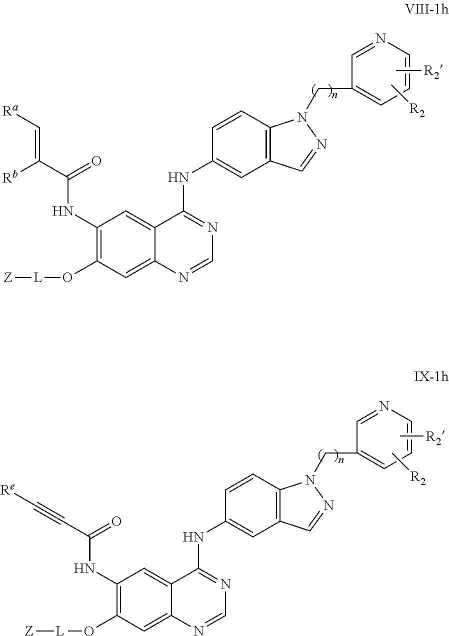

wherein Rₐ and R_{b} are independently of each other H, hal, or —CH₂—O—CH₃; and Rₑ is H or methyl.

In some embodiments, R² and R^{2′} are independently of each other H, C₁₋₆ alkyl, hal —CF₃, or —OCF₃;

n is 0, 1, 2, or 3, (e.g., 1 or 2);
L is a covalent bond, straight chain or branched C₁₋₄ alkyl, or

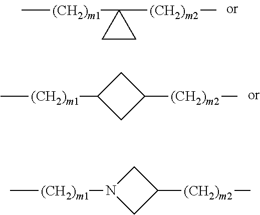

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, the compound is a compound of formula VIII-1f, with the proviso that when R^{a} and R^{b}, R^{2′} are H; R² is 3-F, n is 1 and L is propylene in a compound of formula VIII-1f, Z cannot be N-linked morpholine.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

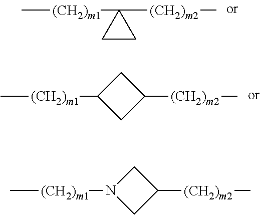

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

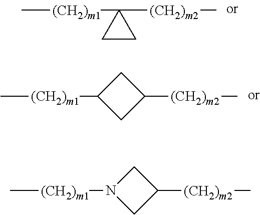

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula VIII-1 and IX-1 have the formula VIII-1i, VIII-1k, VIII-1l and IX-1i, IX-1k, IX-1l

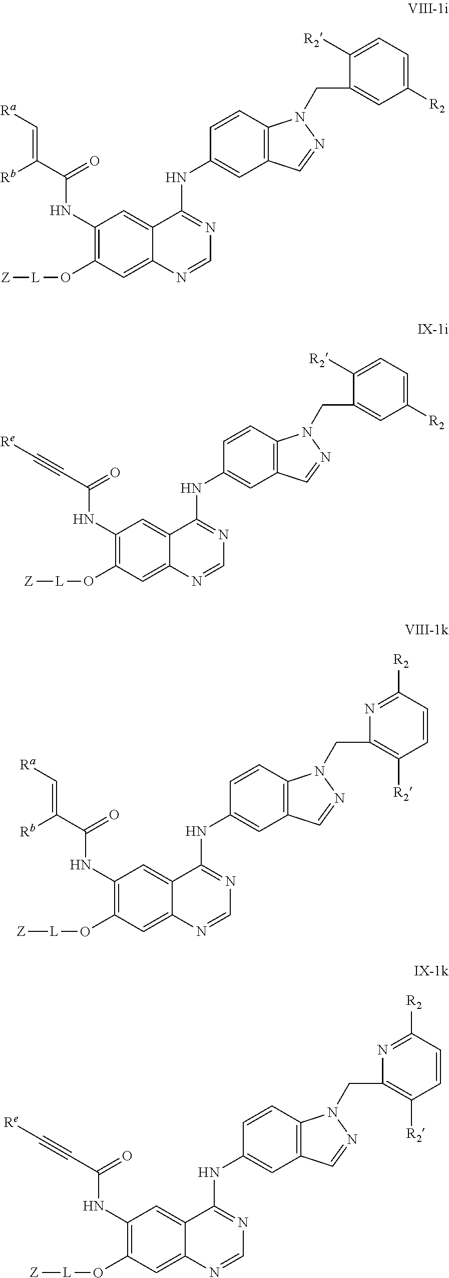

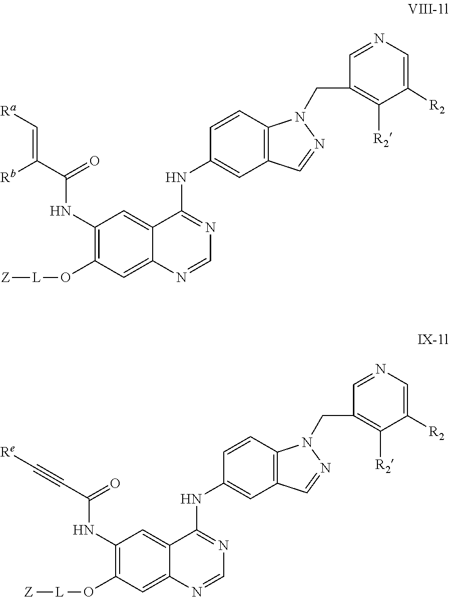

wherein Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

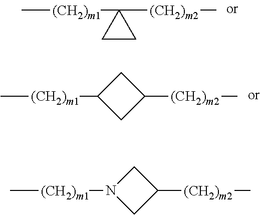

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, the compound is a compound of formula VII-1i, with the proviso that when R^{a} and R^{b}, R^{2′} are H; R² is F, and L is propylene in a compound of formula VIII-1i, Z cannot be N-linked morpholine.

In some embodiments, R² and R^{2′} are independently of each other H, hal or C₁₋₆ alkyl, (e.g., H, hal or —CH₃). In some embodiments, R² is H or hal. In some embodiments, R² is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

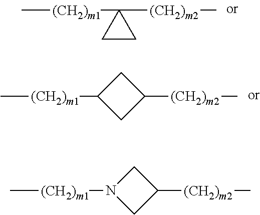

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

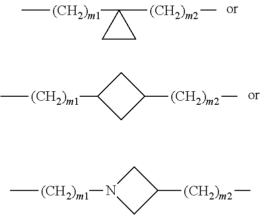

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments of each compound of formula VIII I, VIII-1a to VIII-1l, and IX, IX-1, IX-1a to IX-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g. C, N, and O). In some embodiments, the number of N atoms is 0, 1, or 2. In some embodiments, the number of O and S atoms each is 0, 1, or 2. In some embodiments, the 3 to 6-membered heterocycloalkyl comprises at least one nitrogen atom, (e.g., 1 or 2 nitrogen atoms). Examples of 3 to 6-membered heterocycloalkyl groups include oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl thiomorpholinyl piperidinyl, morpholinyl and the like, (e.g., morpholinyl, piperazinyl and piperidinyl). In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of each compound of formula VIII, VIII-1, VIII-1a to VIII-1l, and IX, IX-1, IX-1a to IX-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, 5 ring atoms), independently selected from C, N, O, and S, (e.g., C, N, and O, or C and N). In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of each compound of formula VIII, VIII-1, VIII-1a to VIII-1l, and IX, IX-1, IX-1a to IX-1l, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g., C, N, and O). In some embodiments, the number of N atoms is 0, 1, 2, or 3 (e.g., 1 or 2). In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decant (e.g., spiro[3.3]heptanyl, spiro[4.4]nonanyl), having one or two heteroatoms selected from N and O, (e.g., diazaspiro[3.3]heptanyl, oxa-azaspiro[3.3]heptanyl, diazaspiro[4.4]nonanyl, oxa-azaspiro[4.4]nonanyl).

In some embodiments —(NR⁶R⁷) ring systems include

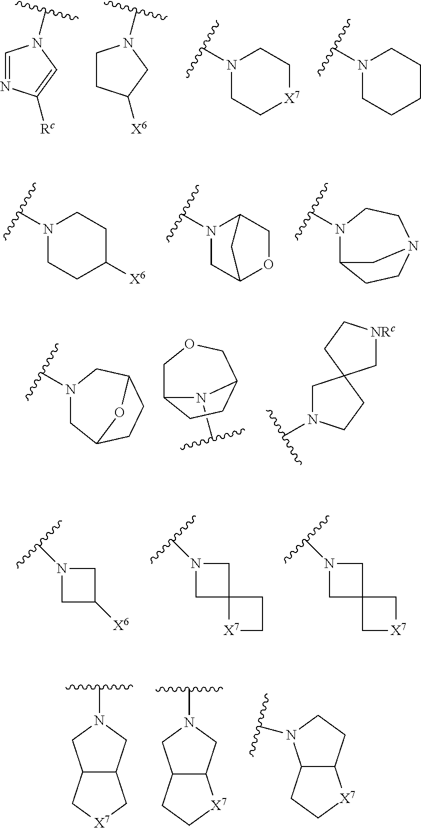

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CHR⁶R⁷) ring systems include

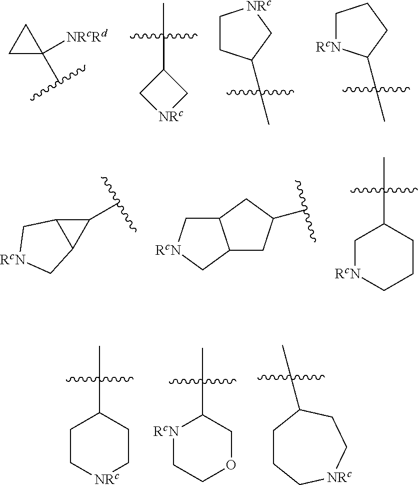

wherein R^{c} is H, C₁₋₄ alkyl, or oxetane; and R^{d} is H or C₁₋₄ alkyl.

In some embodiments of each compound of formula VIII, VIII-1, VIII-1a to VIII-1l, and IX, IX-1, IX-1a to IX-1l, Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl) include the ring systems as defined above.

In some embodiments, ring systems of group Z include

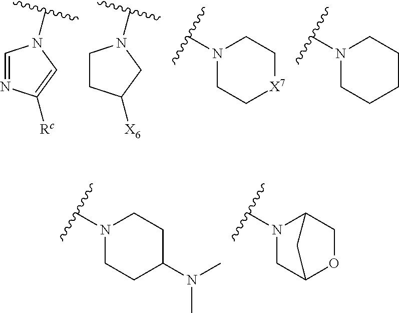

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, ring systems of group Z include

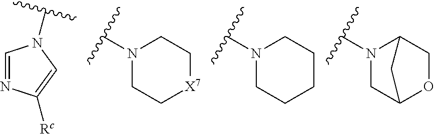

wherein R^{c} is H, C₁₋₄ alkyl, or oxetane; and X⁷ is —O—, —NH—, or —N(CH₃)—.

In some embodiments, the present disclosure is directed toward a compound or a pharmaceutically acceptable salt or stereoisomer thereof of formula I above wherein Y² is —NR′″—, having the following formula X

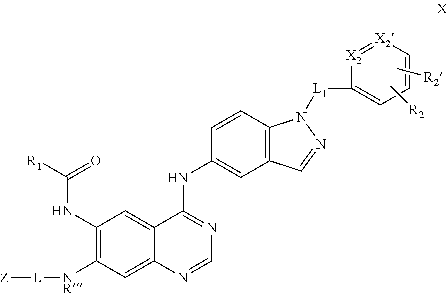

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH, or —C≡C—CH—, wherein Rₐ and R_{b} are independently of each other H, hal, or —CH₂—O—CH₃;
R² and R^{2′} are independently of each other H, C₁₋₆ alkyl, hal, —CF₃, or —OCF₃;
L is a covalent bond, straight chain or branched C₁₋₄ alkyl, or

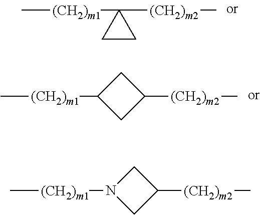

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′ or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃. In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

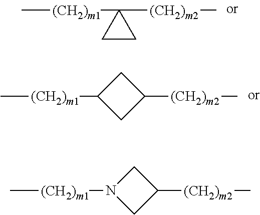

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —C₂, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

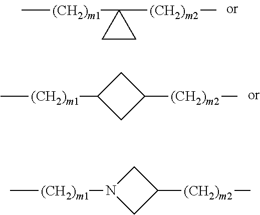

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—).

In some embodiments, L¹ is a covalent bond. In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-.

In some embodiments, L¹ is —CH₂— or —CH₂—CH₂—, (e.g., —CH₂—).

In some embodiments compound X has the following formula X-1

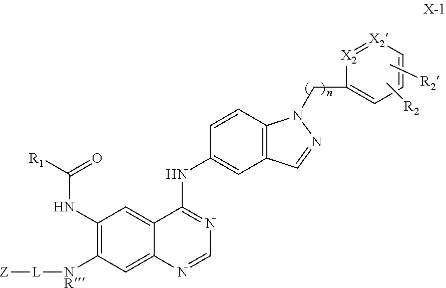

(e.g., one of the following formulas X-1a or X-1b

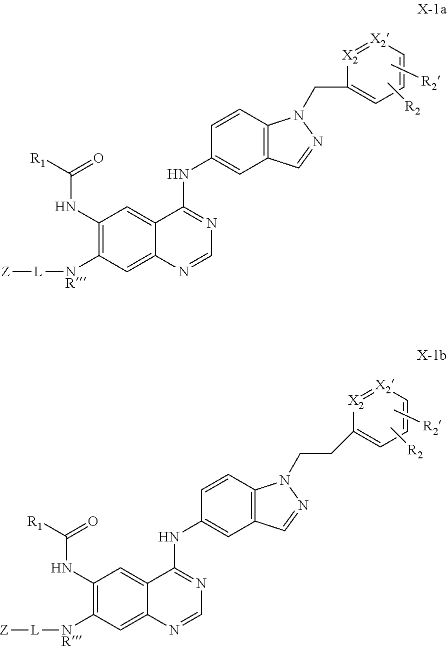

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

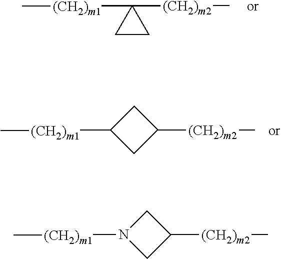

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵) wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

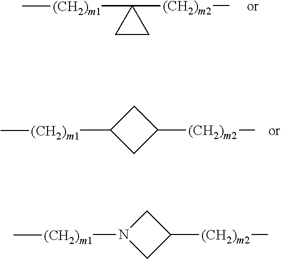

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂— or —C(CH₃)₂—.

In some embodiments, compound X has one of the following formulas

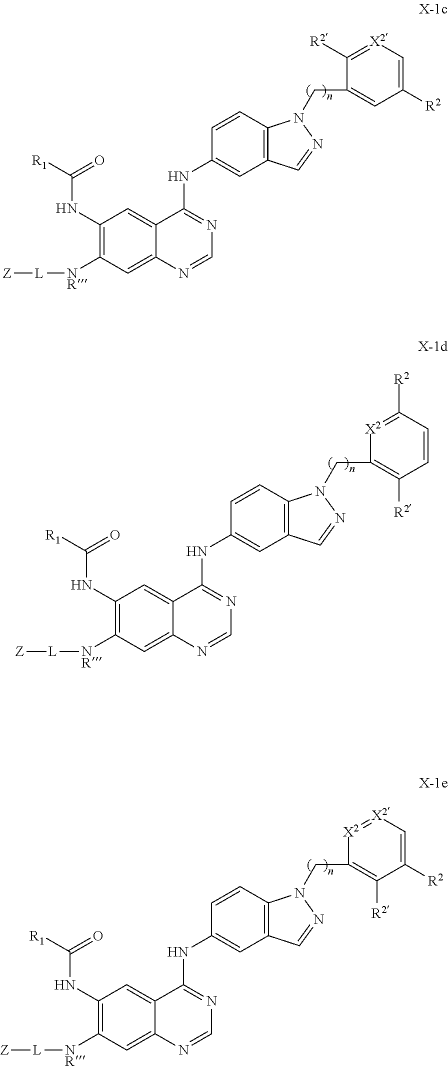

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

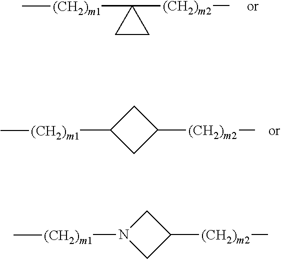

wherein m1 and m22 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁶ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

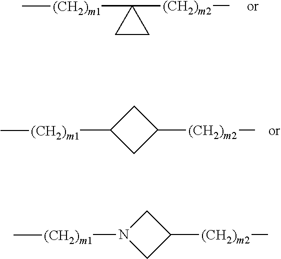

wherein m1 and m2 are independently of each other 0, 1, 2, 3, or 4, (e.g., 0, 1, or 2). In some embodiments, m2 is 0 and m1 is 0 or 1 or 2 In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄— or —C(CH₃)₂—). In some embodiments, L is —CH₂—, —(CH₂)₂— or —C(CH₃)₂—.

In some embodiments, compound X has one of the following formulas

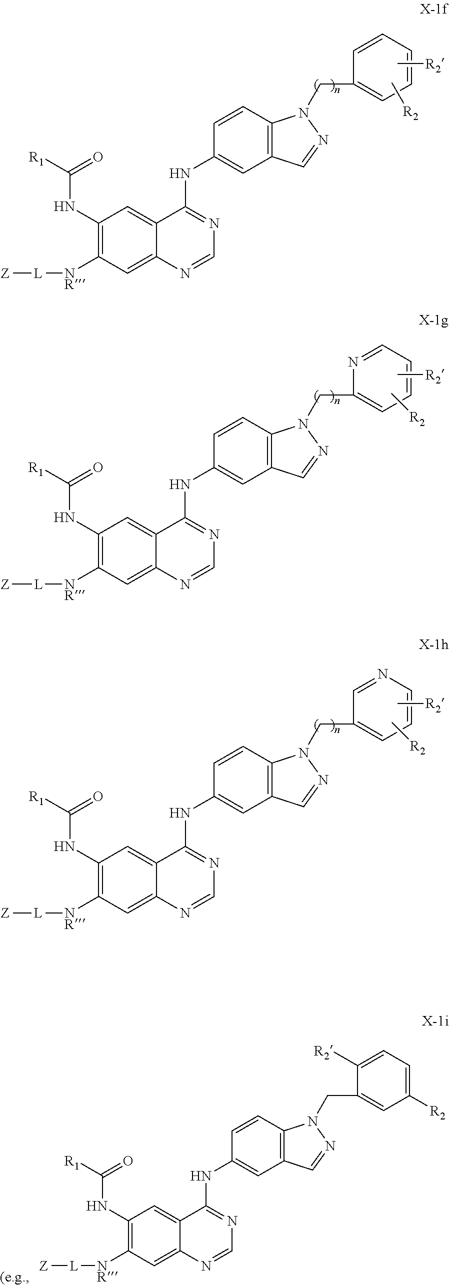

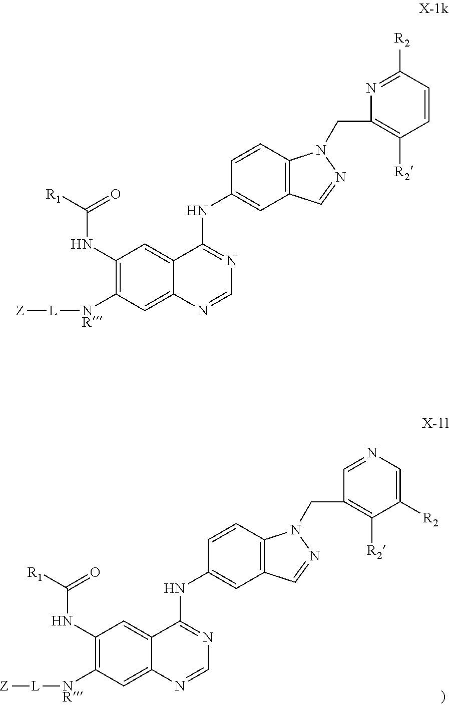

wherein R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

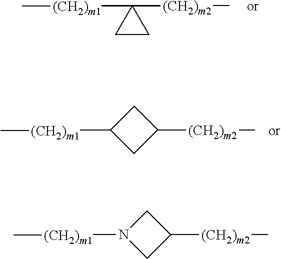

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

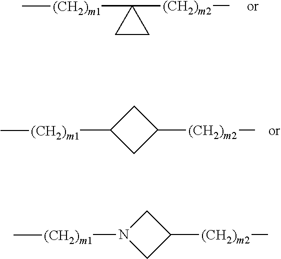

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂— or —C(CH₃)₂—.

In some embodiments of each compound of formula X, X-1, and X-1a to X-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the number of N atoms is 0, 1, 2. In some embodiments, the number of O and S atoms each is 0, 1, 2. Examples of 3 to 6-membered heterocycloalkyl groups include oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl I-4-dithianyl, 1,3-dioxane. 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of each compound of formula X, X-1, and X-1a to X-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, 5 ring atoms), independently selected from C, N, O, and S (e.g., C, N, and O, or C and N). In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of each compound of formula X, X-1, and X-1a to X-1l, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g., C, N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl, (e.g., spiro[3.3]heptanyl, spiro[4.4]nonanyl), having one or two heteroatoms selected from N and O, (e.g., diazaspiro[3.3]heptanyl, oxa-azaspiro[3.3]heptanyl, diazaspiro[4.4]nonanyl, oxa-azaspiro[4.4]nonanyl).

In some embodiments, —(NR⁶R⁷) ring systems include

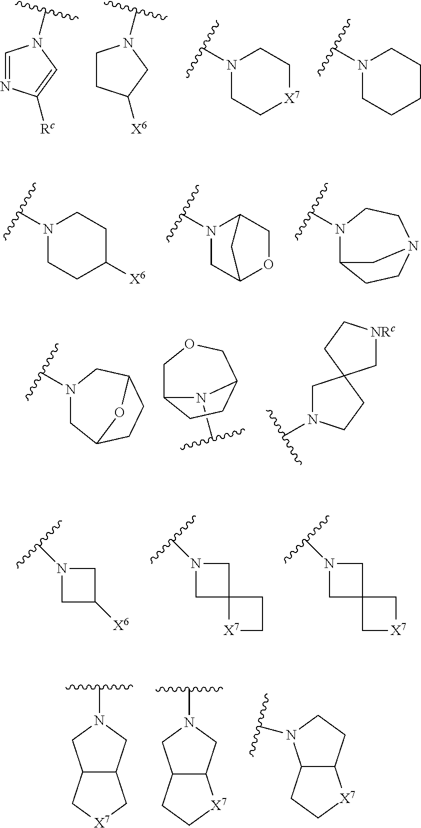

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CR⁶R⁷) ring systems include

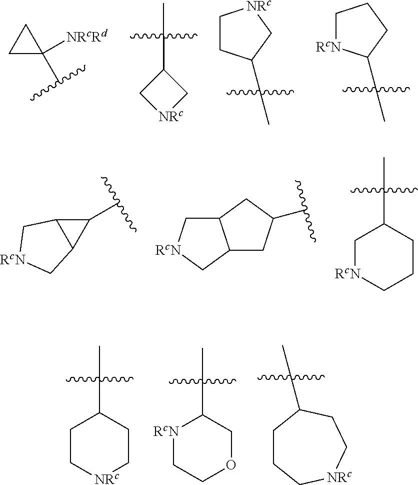

wherein R^{c} is H, C₁₋₄ alkyl, or oxetane; and R^{d} is H or C₁₋₄ alkyl.

In some embodiments of each compound of formula X, X-1, and X-1a to X-1l, Z is —(NR⁴R⁴), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl) include the ring systems as defined above.

In some embodiments, ring systems of group Z include

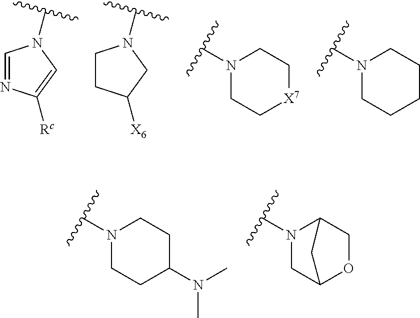

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, ring systems of group Z include

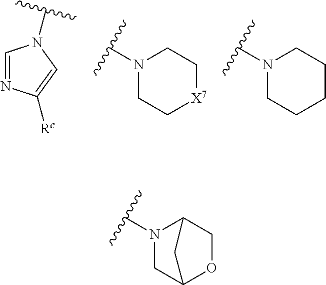

wherein R^{c} is H, C₁₋₄ alkyl, or oxetane; and X⁷ is —O—, —NH—, or —N(CH₃)—.

In some embodiments a compound of formula X has the formula XI or XII

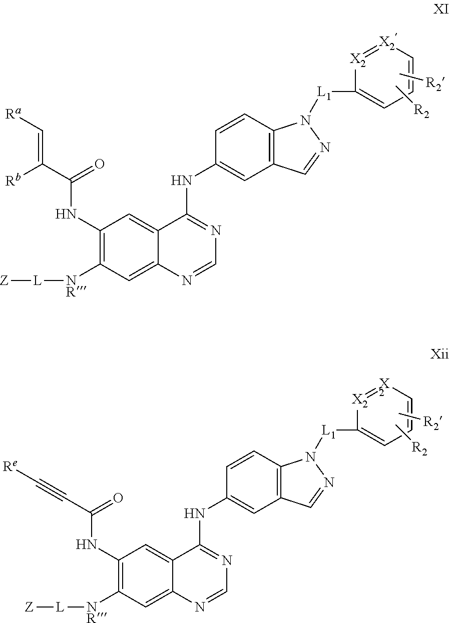

wherein X² and X^{2′} are independently of each other —N═ or —CH═;
L¹ is a covalent bond or straight chain or branched C₁₋₃alkyl, which is unsubstituted or substituted with hal;
R² and R^{2′} are independently of each other H, C₁₋₆ alkyl, hal, —CF₃, or —OCF₃;
Rₐ and R_{b} are independently of each other H, hal, or —CH₂—O—CH₃; and Rₑ is H or methyl.

In some embodiments, L is a covalent bond straight chain or branched C₁₋₄ alkyl, or

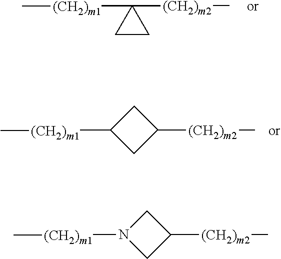

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, w herein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃. In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-. In some embodiments, L¹ is —CH₂— or —CH₂—CH₂—, (e.g., —CH₂—).

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

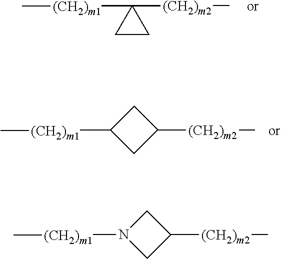

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂— or —C(CH₃)₂—.

In some embodiments, a compound of formula XI or XII has the formula XI-1 or XII-1, (e.g., XI-1a, XI-1b or XI-1a, XII-1b)

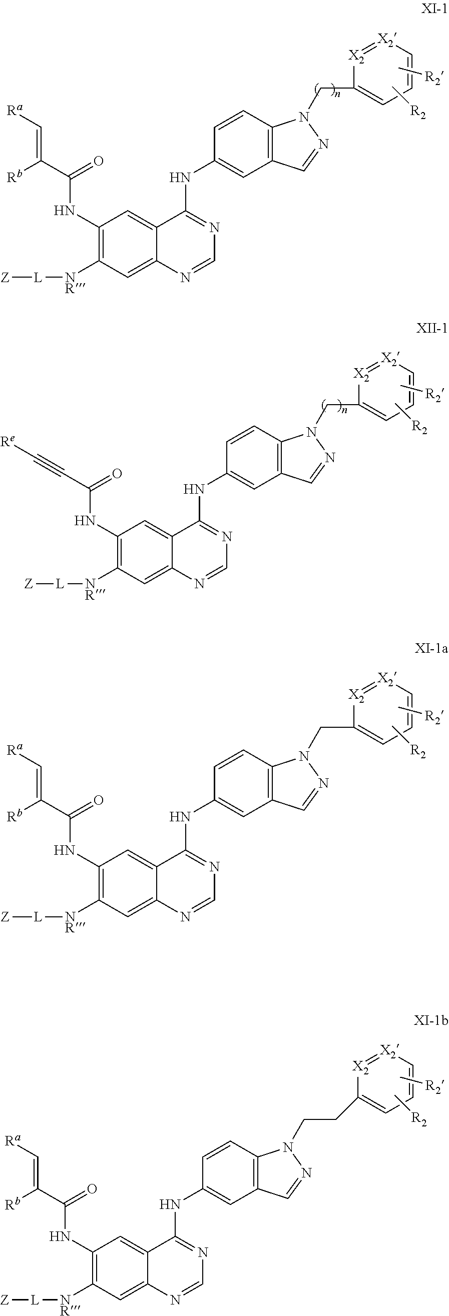

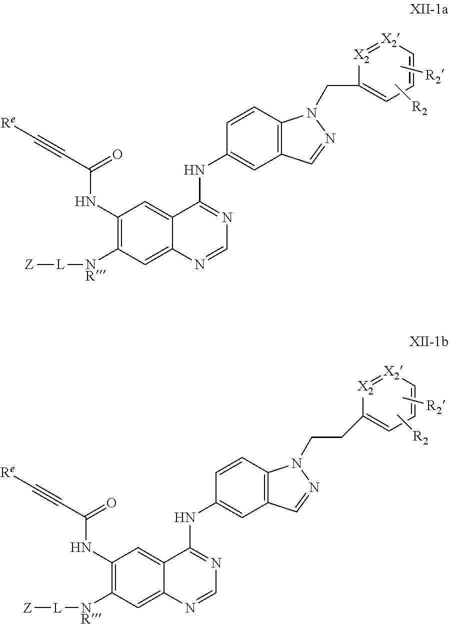

wherein X² and X^{2′} are independently of each other —N═ or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

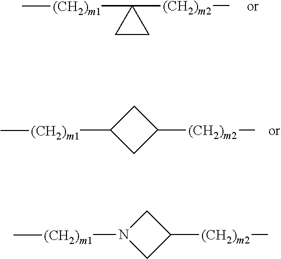

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷, wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

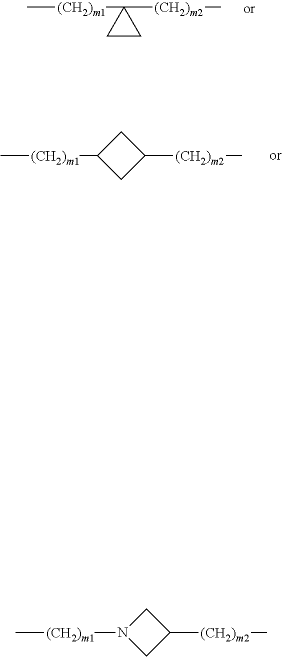

wherein m1 and m2 are independently of each other 0, 1, 2, 3, or 4, (e.g., 0, 1, or 2). In some embodiments, m2 is 0 and m1l is 0 or 1 or 2, In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

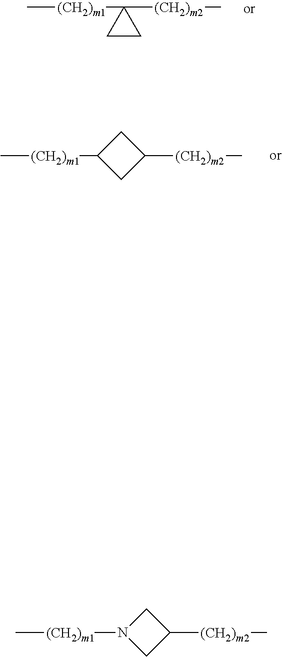

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—).

In some embodiments, a compound of formula XI-1 and XII-1 have the formula XI-1c, XI-1d, XI-1e and XII-1c, XII-1d, XII-1e

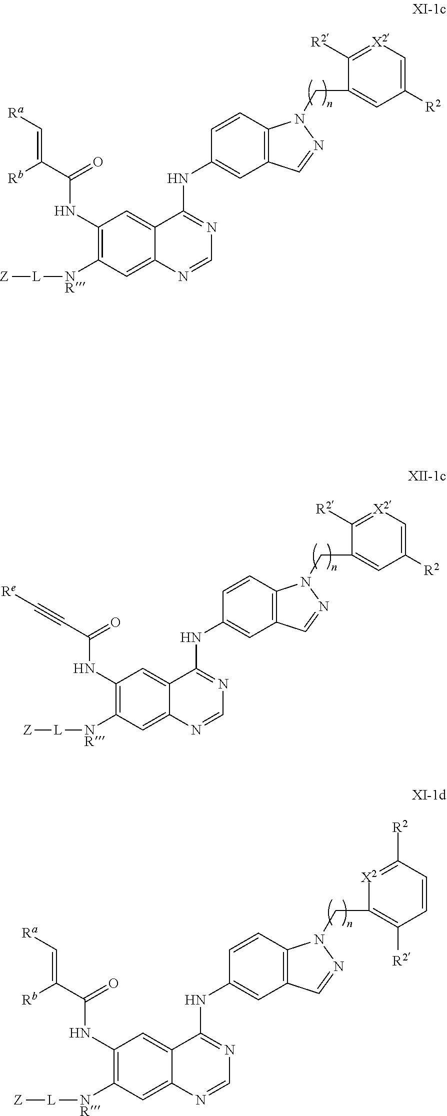

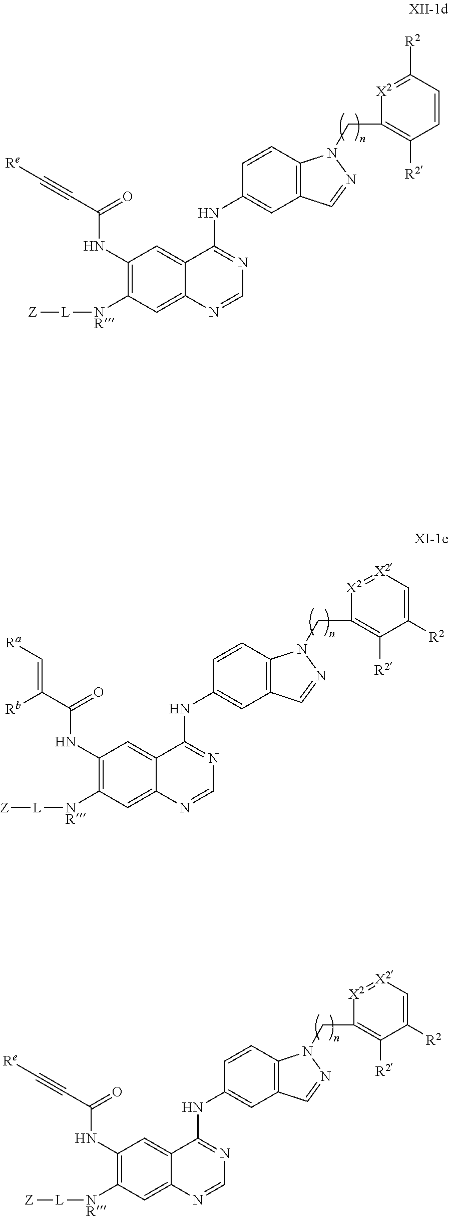

wherein X² and X^{2′} are independently of each other —N═ or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

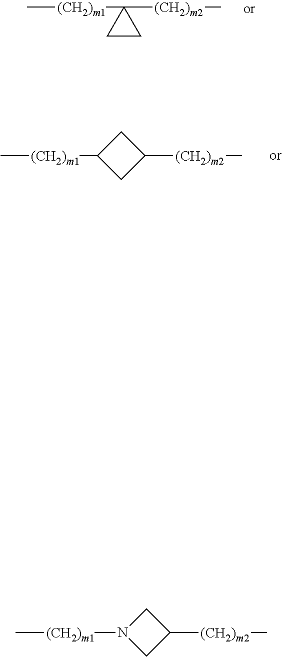

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

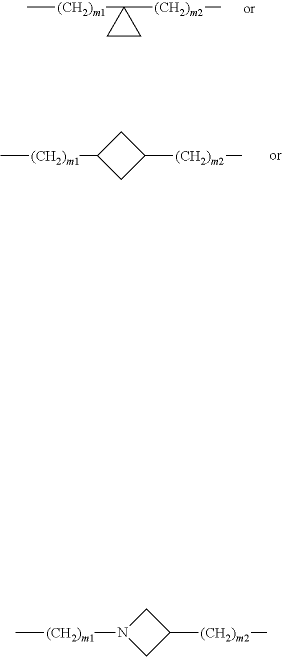

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

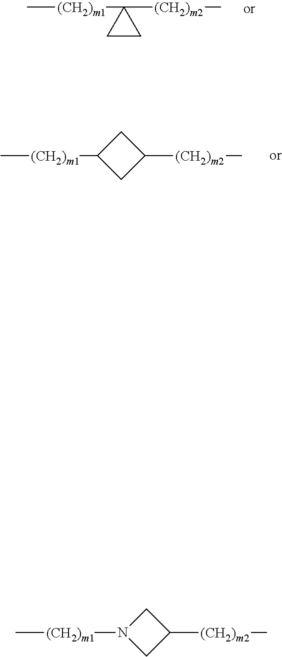

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula XI-1 and XII-1 have the formula XI-1f, XI-1g, XI-1h and XII-1f, XII-1g, XII-1h

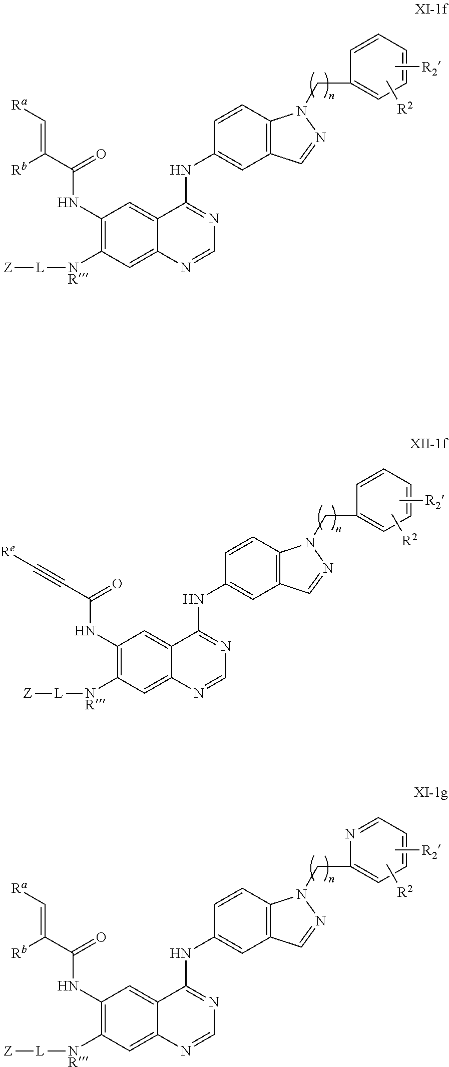

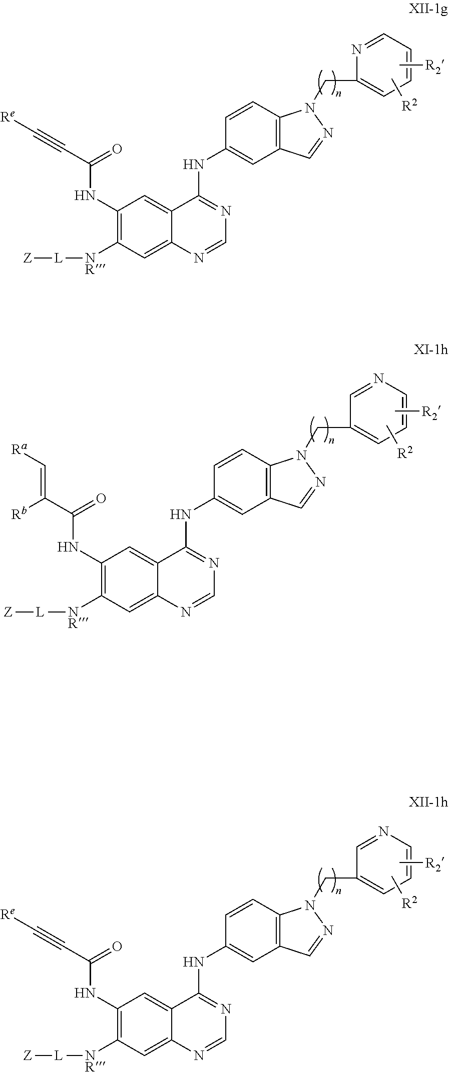

wherein Rₐ and R_{b} are independently of each other H, hal, or —CH₂—O—CH₃; and Rₑ is H or methyl.

In some embodiments, R², R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

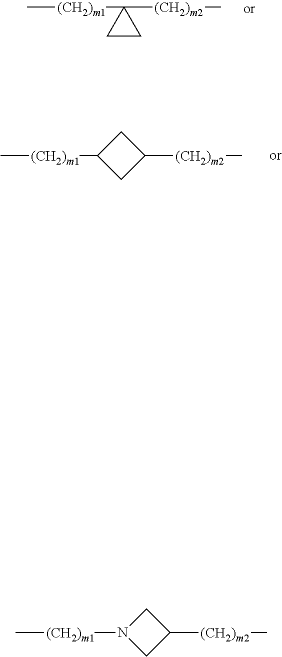

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)—, or —CH₂—C(CH₃)₂—). In some embodiments, L is —CH₂—, —(CH₂)₂—, or —C(CH₃)₂—. In some embodiments, L is

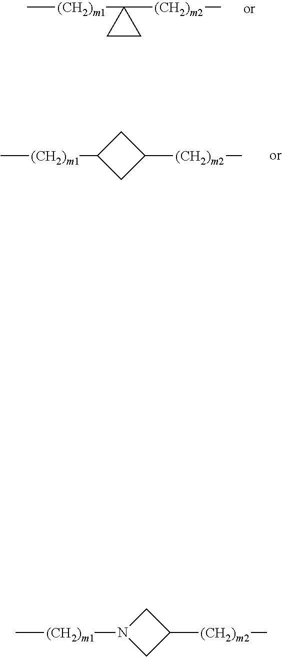

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

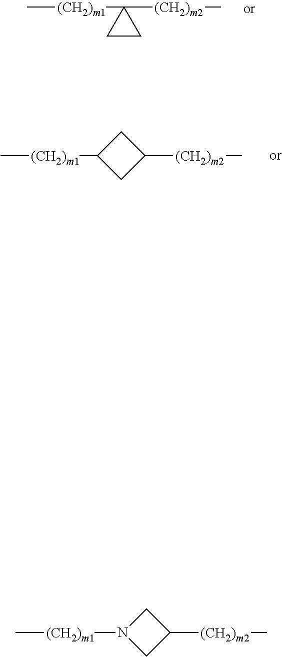

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula XI-1 and XII-1 have the formula XI-1i, XI-1k, XI-1l and XII-1i, XII-1k, XII-1l

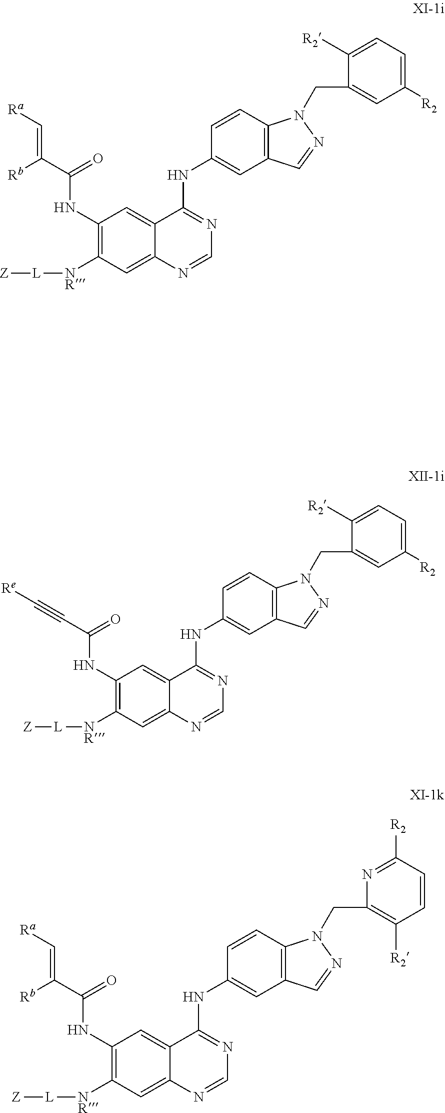

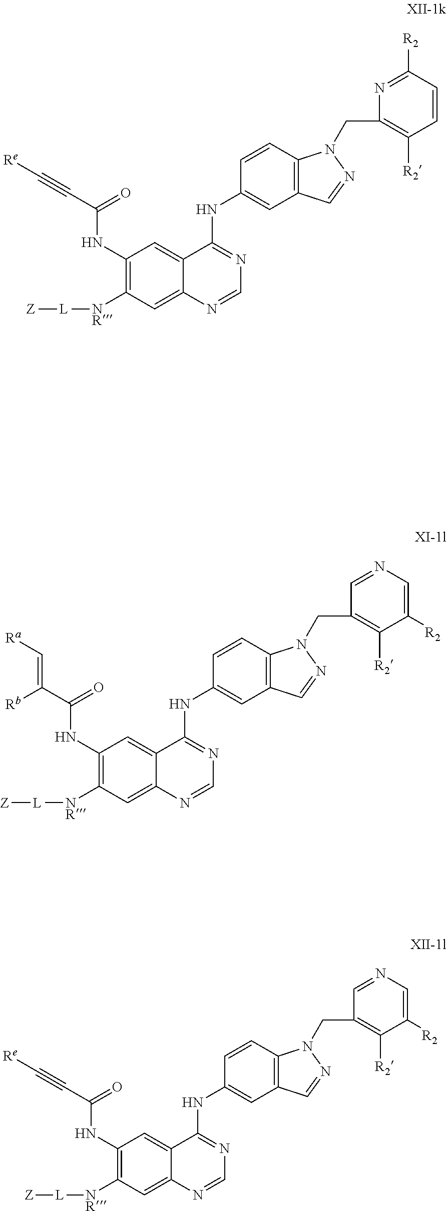

wherein Rₐ and R_{b} are independently of each other H, hal, or —CH₂—O—CH₃; and Rₑ is H or methyl.

In some embodiments, R² and R^{2′} are independently of each other H, C₁₋₆ alkyl hal —CF₃, or —OCF₃;

n is 0, 1, 2, or 3, (e.g., 1 or 2);
L is a covalent bond, straight chain or branched C₁₋₄ alkyl, or

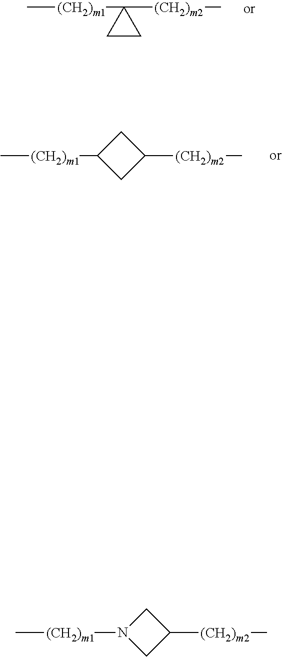

wherein m1 and m2 are independently of each other 0, 1, 2, 3, or 4, (e.g., L is a covalent bond, straight chain or branched C₁₋₄ alkyl);

### R′″ is H or —CH₃; and

Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—), In some embodiments, L is —CH₂—, —(CH₂)₂—, or —C(CH₃)₂—. In some embodiments, L is

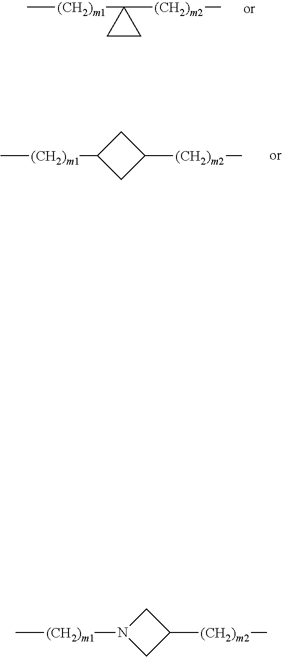

wherein m1 and m2 are independently of each other 0, 1, 2, 3, or 4, (e.g., 0, 1, or 2). In some embodiments, m2 is 0 and m1 is 0 or 1 or 2, In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

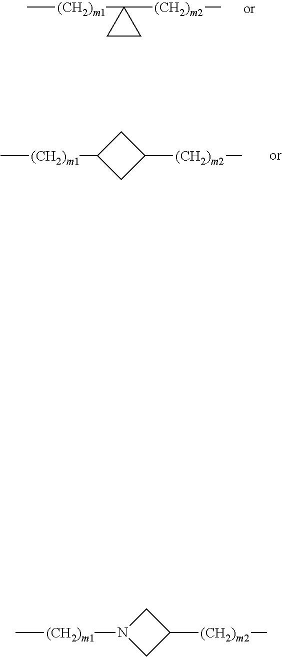

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments of each compound of formula XI, XI-1, XI-1a to XI-1l, and XII, XII-1, XII-1a to XII-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the so number of N atoms is 0, 1, or 2. In some embodiments, the number of O and S atoms each is 0, 1, or 2. Examples of 3 to 6-membered heterocycloalkyl groups include oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In son embodiments of each compound of formula XI, XI-1, XI-1a to XI-1l, and XII, XII-1, XII-1a to XII-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, ring atoms), independently selected from C, N, O, and S, (e.g., C, N, and O, or C and N). In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of each compound of formula XI, XI-1, XI-1a to XI-1l, and XII, XII-1, XII-1a to XII-1l, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g. C, N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl): fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl (e.g., spiro[3.3]heptanyl, spiro[4.4]nonanyl), having one or two heteroatoms selected from N and O (e.g., diazaspiro[3.3]heptanyl, oxa-azaspiro[3.3]heptanyl diazaspiro[4.4]nonanyl, oxa-azaspiro[4.4]nonanyl).

In some embodiments —(NR⁶R⁷) ring systems include

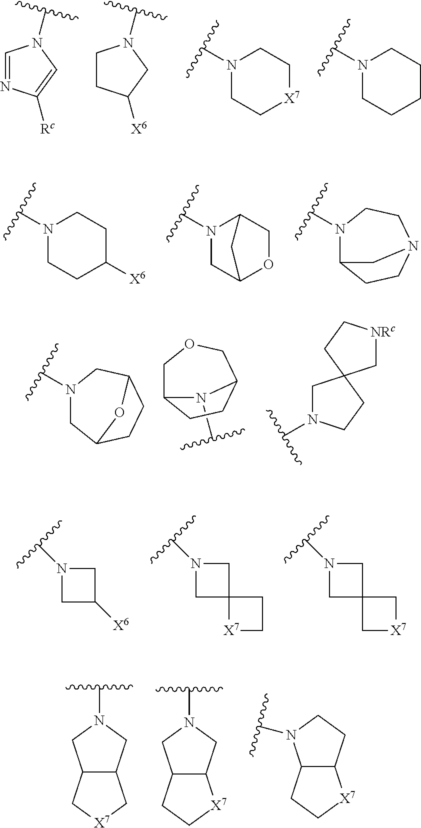

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CHR⁶R⁷) ring systems include

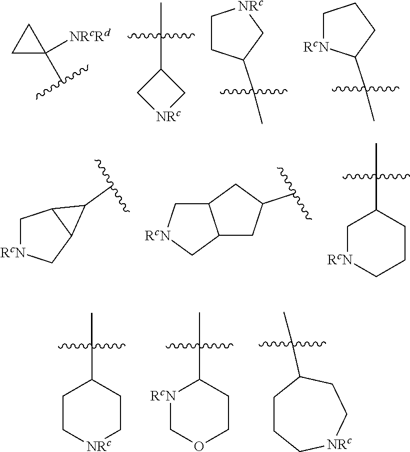

wherein R^{c} is H, C₁₋₄ alkyl, or oxetane; and R^{d} is H or C₁₋₄ alkyl.

In some embodiments of each compound of formula, XI, XI-1, XI-1a to XI-1l, and XII, XII-1, XII-1a to XII-1l, Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl) include the ring systems as defined above.

In some embodiments, ring systems of group Z include

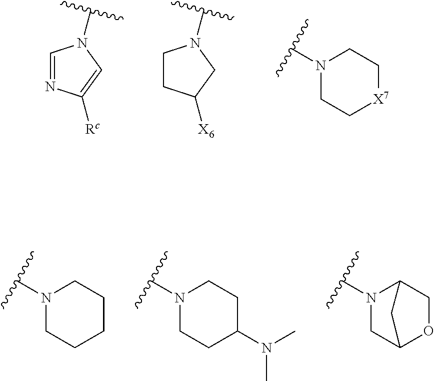

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, ring systems of group Z include

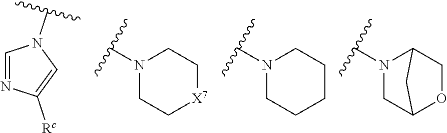

wherein R^{c} is H, C₁₋₄ alkyl, or oxetane; and X⁷ is —O—, —NH—, or —N(CH₃)—.

In some embodiments, the present disclosure is directed toward a compound or a pharmaceutically acceptable salt or stereoisomer thereof of formula I above wherein Y² is —C≡C— having the following formula XIII

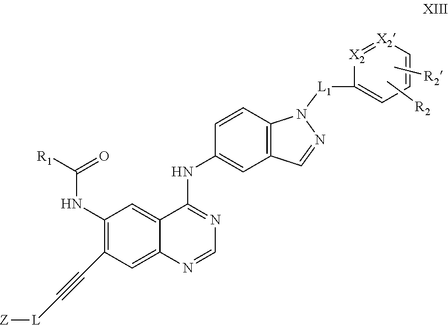

wherein L¹ is a covalent bond or straight chain or branched C₁₋₃alkyl, which is unsubstituted or substituted with hal;
X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
L is a covalent bond, straight chain or branched C₁₋₄ alkyl, or

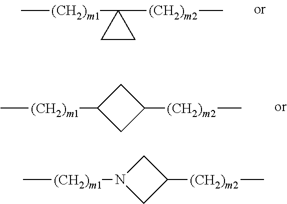

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C═CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₂)₂—), In some embodiments, L is —CH₂—, —(CH₂)₂—, or —C(CH₃)₂— In some embodiments, L is

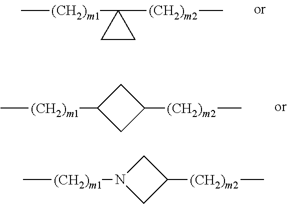

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)—, —(CH₂)₃—, —(CH₂)₄—, or —C(CH)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

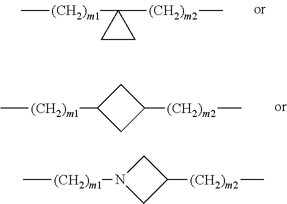

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, L¹ is a covalent bond. In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-.

In some embodiments, L¹ is —CH₂— or —CH₂—CH₂—, (e.g., —CH₂—).

In some embodiments, compound XIII has the following formula XIII-1

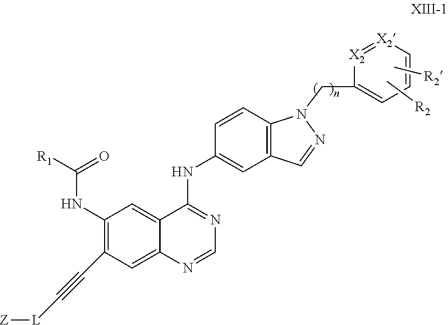

(e.g., one of the following formulas XIII-1a or XIII-1b

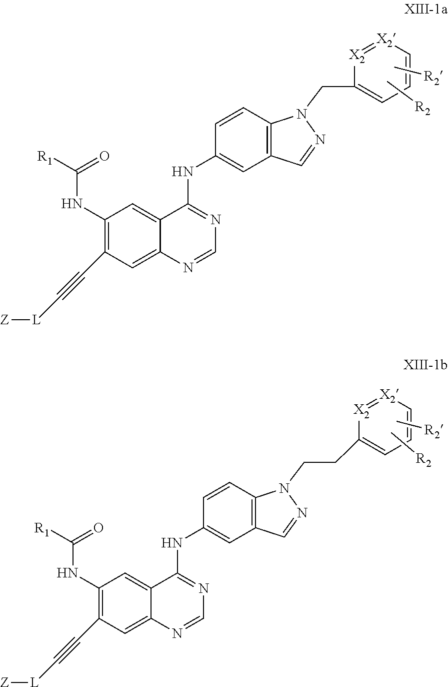

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

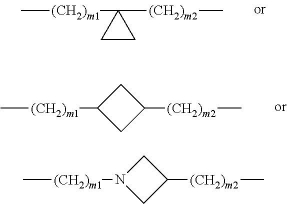

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —CH≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂— In some embodiments, L is

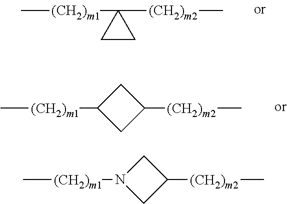

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₁₂—C(CH₃)₂—). In some embodiments, L is

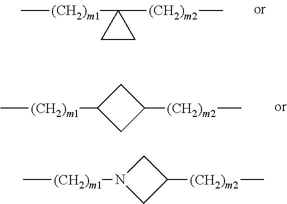

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, compound XIII has one of the following formulas

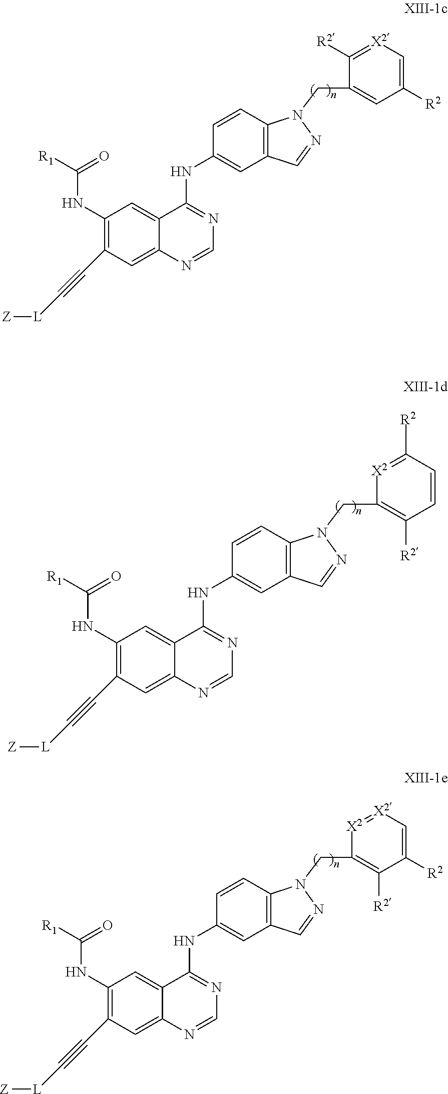

wherein X² and X^{2′} are independently of each other —N═ or —CH═;
R¹ is —CR_{b}═CHRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃, or —OCF₃;
n is 0, 1, 2, or 3, (e.g., 1 or 2);
L is a covalent bond, straight chain or branched C₁₋₄ alkyl, or

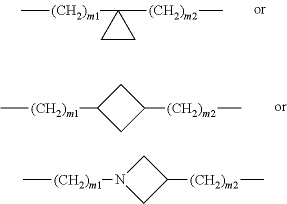

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X is —N═ and X² is —CH═. In some embodiments, both X² and X^{2′} are —N═.

In some embodiments, R² and R^{2′} are independently of each other H, hal or C₁₋₆ alkyl, (e.g., H, hal or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R² is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

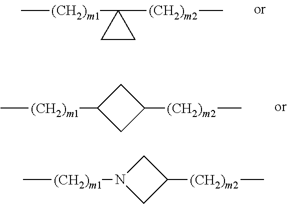

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In is some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

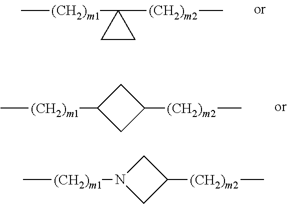

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—).

In some embodiments, compound XIII has one of the following formulas

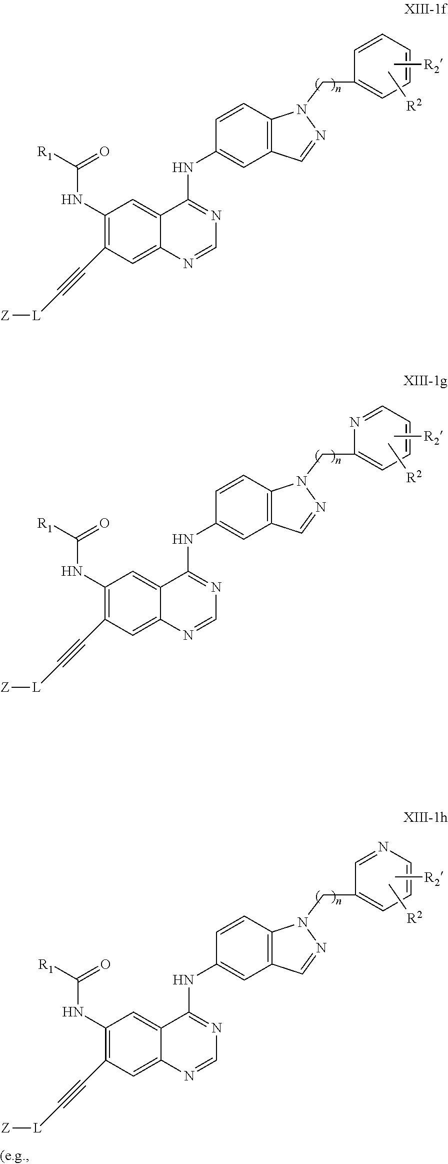

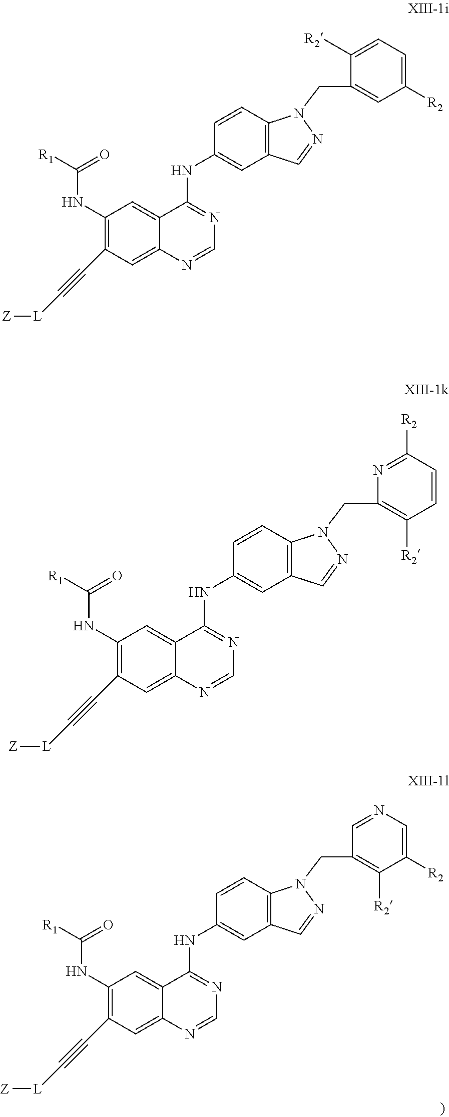

wherein R¹ is —CR_{b}═CRₐ, —C≡CH, or —C≡C—CH₃, wherein Rₐ and R_{b} are independently of each other H, hal, or —CH₂—O—CH₃;
R² and R^{2′} are independently of each other H, C₁₋₆ alkyl, hal, —CF₃— or —OCF₃;
n is 0, 1, 2, or 3, (e.g., 1 or 2).
L is a covalent bond, straight chain or branched C₁₋₄ alkyl, or

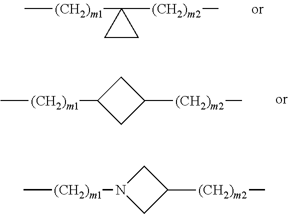

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, and —NR′R″, wherein R′ and R″ are independently of each other H or C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, R¹ is —CH═CH₂. In some embodiments, R¹ is —CH═CH-hal or —C(hal)=CH₂. In some embodiments, R¹ is —CH═CH—CH₂—O—CH₃. In some embodiments, R¹ is —C≡CH or —C≡C—CH₃.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂— In some embodiments, L is

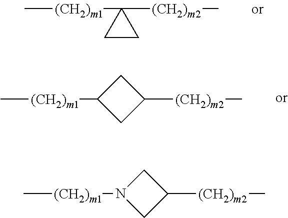

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

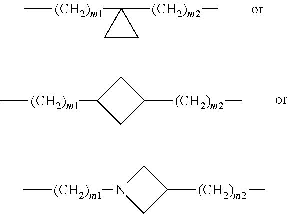

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—).

In some embodiments of each compound of formula XIII, XIII-1, and XIII-1a to XIII-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the number of N atoms is 0, 1, or 2.

In some embodiments, the number of O and S atoms each is 0, 1, or 2. Examples of 3 to 6-membered heterocycloalkyl groups include oxiranyl, thiaranyl, aziradinyl, oxetanyl, thiatanyl, azetidinyl, pyrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of each compound of formula XIII, XIII-1, and XIII-1a to XIII-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, or 5 ring atoms), independently selected from C, N, O, and S, (e.g., C, N, and O, or C and N). In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of each compound of formula XIII, XIII-1, and XIII-1a to XIII-1l, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g., C, N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl, (e.g., spiro[3.3]heptanyl, spiro[4.4]nonanyl), having one or two heteroatoms selected from N and O, (e.g. diazaspiro[3.3]heptanyl, oxa-azaspiro[3.3]heptanyl, diazaspiro[4.4]nonanyl, oxa-azaspiro[4.4]nonanyl).

In some embodiments, —(NR⁶R⁷) ring systems include

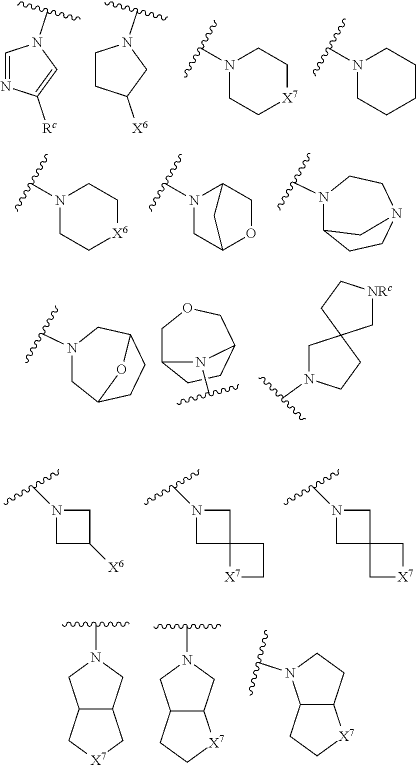

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CH R⁶R⁷) ring systems include

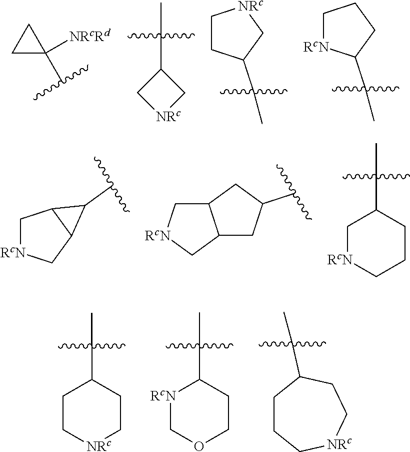

wherein R^{c} is H, C₁₋₄ alkyl, or oxetane; and R^{d} is H or C₁₋₄ alkyl.

In some embodiments of each compound of formula XIII, XIII-1, and XIII-1a to XIII-1l, Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl) include the ring systems as defined above.

In some embodiments, ring systems of group Z include

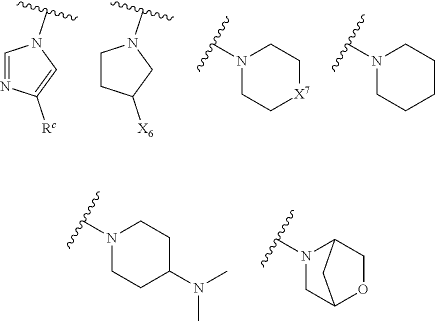

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, ring systems of group Z include

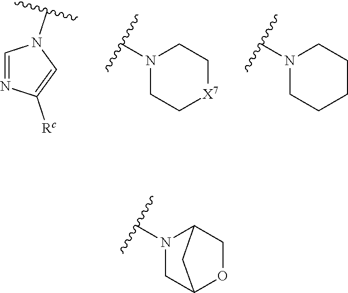

wherein R^{c} is H, C₁₋₄ alkyl, or oxetane; and X⁷ is —O—, —NH—, or —N(CH₃)—.

In some embodiments a compound of formula XII has the formula XIV or XV

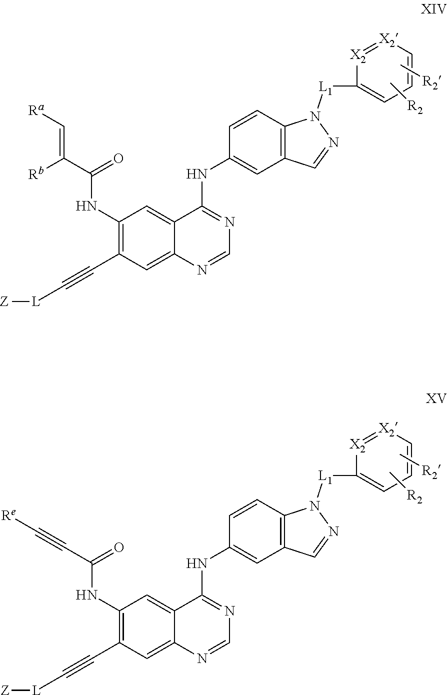

wherein X² and X^{2′} are independently of each other —N═ or —CH═;
L¹ is a covalent bond or straight chain or branched C₁₋₃alkyl, which is unsubstituted or substituted with hal;
R² and R^{2′} are independently of each other H, C₁₋₆ alkyl, hal, —CF₃, or —OCF₃;
Rₐ and R_{b} are independently of each other H, hal, or —CH₂—O—CH₃; and Rₑ is H or methyl.

In some embodiments, L is a covalent bond, straight chain or branched C₁₋₄ alkyl, or

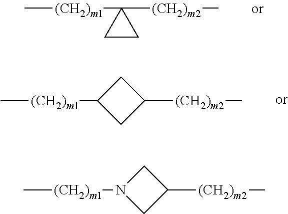

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or C₁₋₄ alkyl.

In some embodiments, both X² and X^{2′} are —CH═. In some embodiments, X² is —N═ and X² is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L¹ is —CH₂—, —CH(CH₃)—, or —CH(hal)-. In some embodiments, L¹ is —CH₂—CH₂—, —CH₂—CH(CH₃)—, or —CH₂—CH(hal)-. In some embodiments, L¹ is —CH₂— or —CH₂—CH₂—, (e.g., —CH₂—).

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

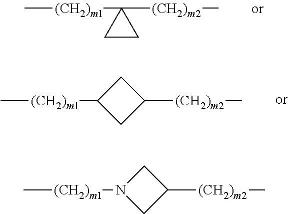

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—.

In some embodiments, a compound of formula XIV or XV has the formula XIV-1 or XV-1, (e.g., XIV-1a, XIV-1b or XV-1a, XV-1b)

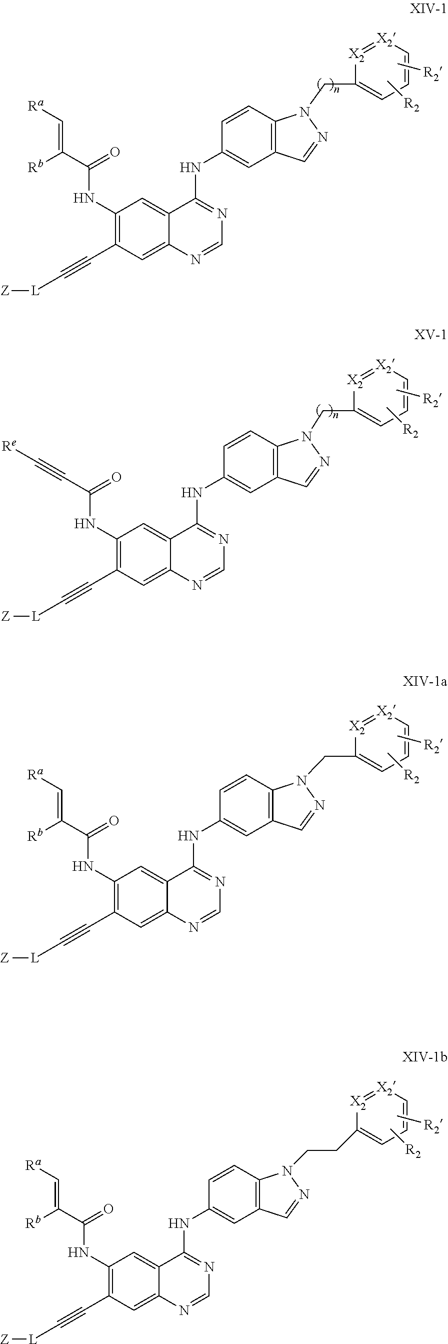

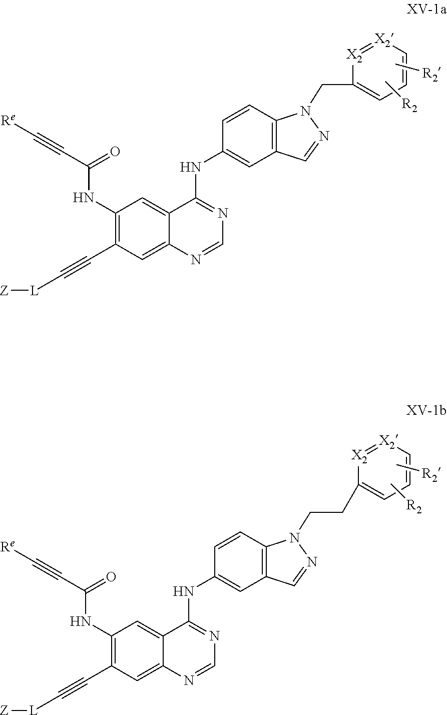

wherein X² and X^{2′} are independently of each other —N═ or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

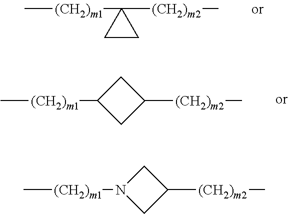

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁶ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—), In some embodiments, L is —CH₂—, —(CH₂)₂—, or —C(CH₃)₂—. In some embodiments, L is

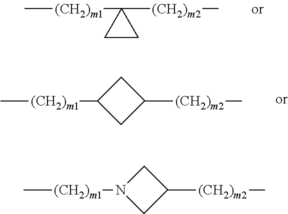

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

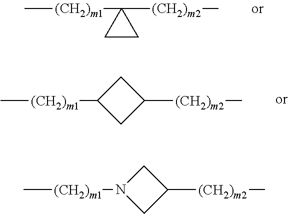

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula XIV-1 and XV-1 have the formula XIV-1c, XIV-1d, XIV-1e and XV-1c, XV-1d, XV-1e

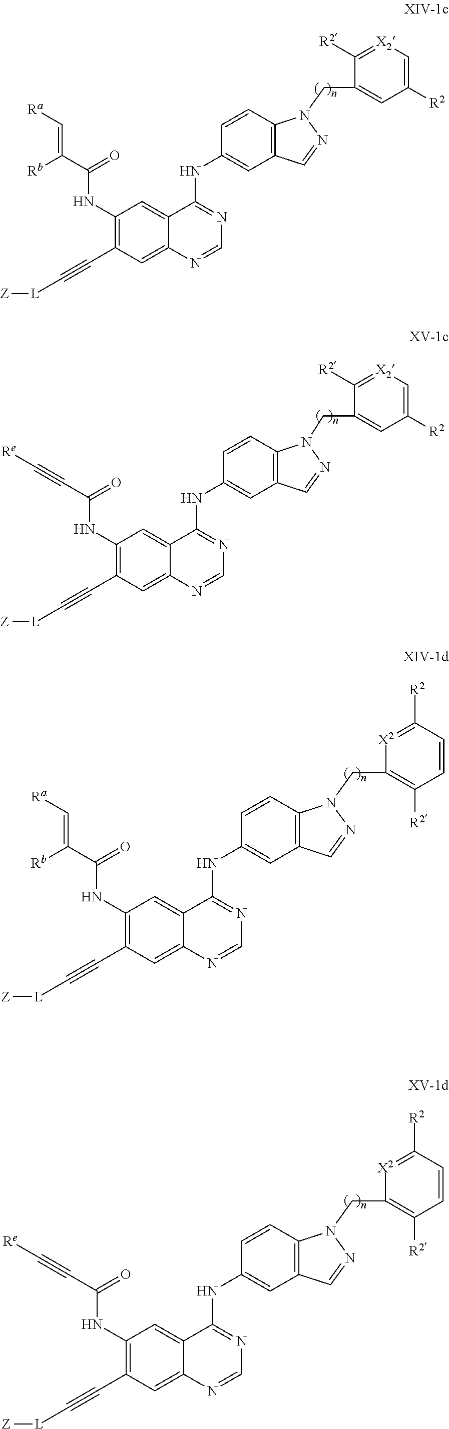

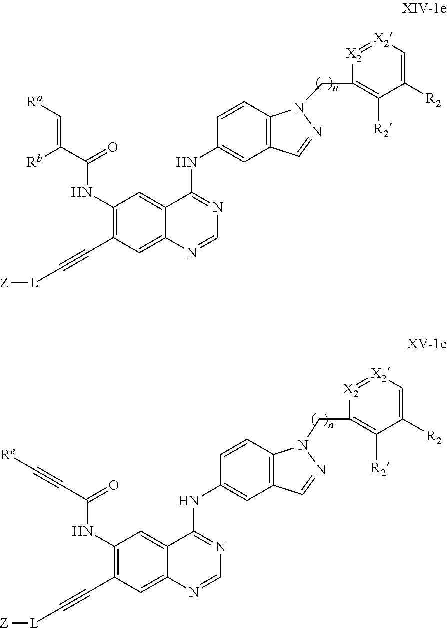

wherein X² and X^{2′} are independently of each other —N═ or —CH═;
Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

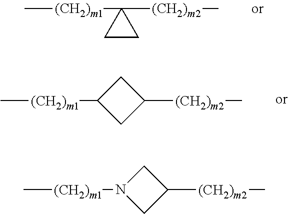

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, both X² and X^{2′} are —CH═. In some embodiments, X² is —N═ and X^{2′} is —CH═ or X^{2′} is —N═ and X² is —CH═. In some embodiments, both X² and X^{2′} are —N═.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

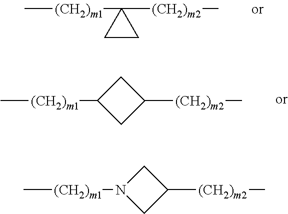

wherein m1 and m2 are independently of each other 0, 1, 2, 3, or 4, (e.g., 0, 1, or 2). In some embodiments, m2 is 0 and m is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

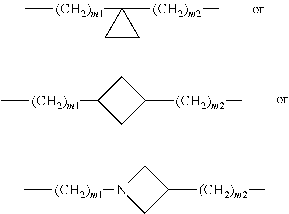

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula XIV-1 and XV-1 have the formula XIV-1f, XIV-1g, XIV-1h and XV-1f, XV-1g, XV-1h

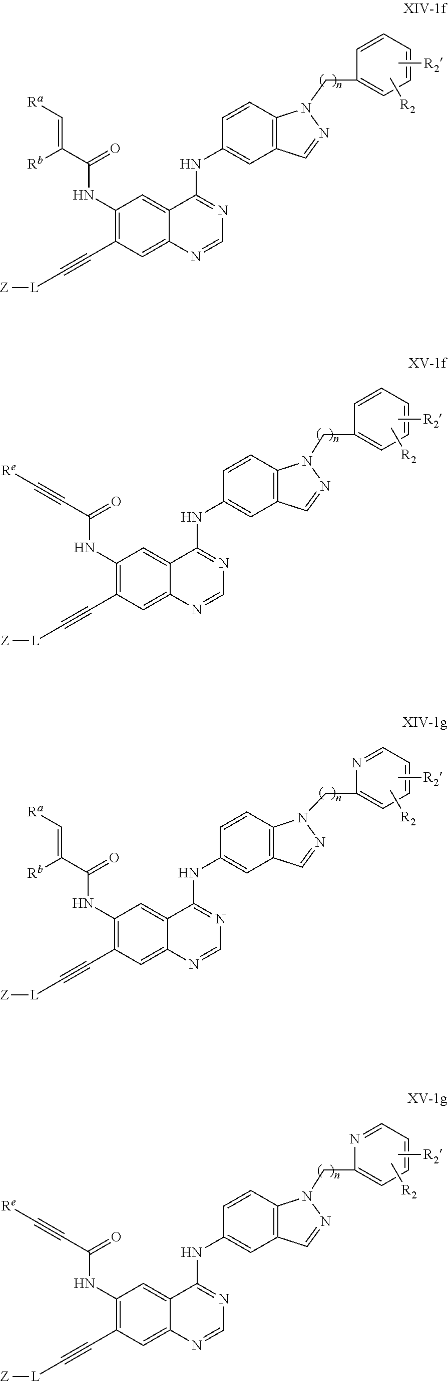

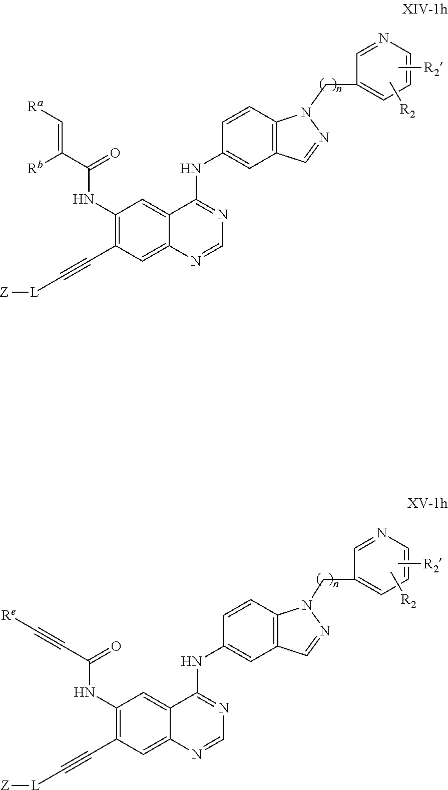

wherein Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

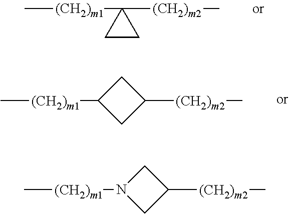

wherein m1 and m2 are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(N⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, R² and R^{2′} are independently of each other H, hal, or C₁₋₆ alkyl, (e.g., H, hal or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is —CH₂—, —(CH₂)₂—, or —C(CH₃)₂—. In some embodiments, L is

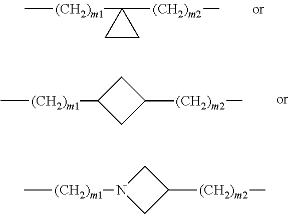

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—. In some embodiments, L is —CH₂—, —(CH₂)₂—, or —C(CH₃)₂—.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

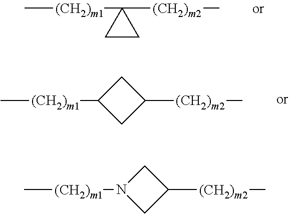

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments, a compound of formula XIV-1 and XV-1 have the formula XIV-1i, XIV-1k, XIV-1l and XV-1i, XV-1k, XV-1l

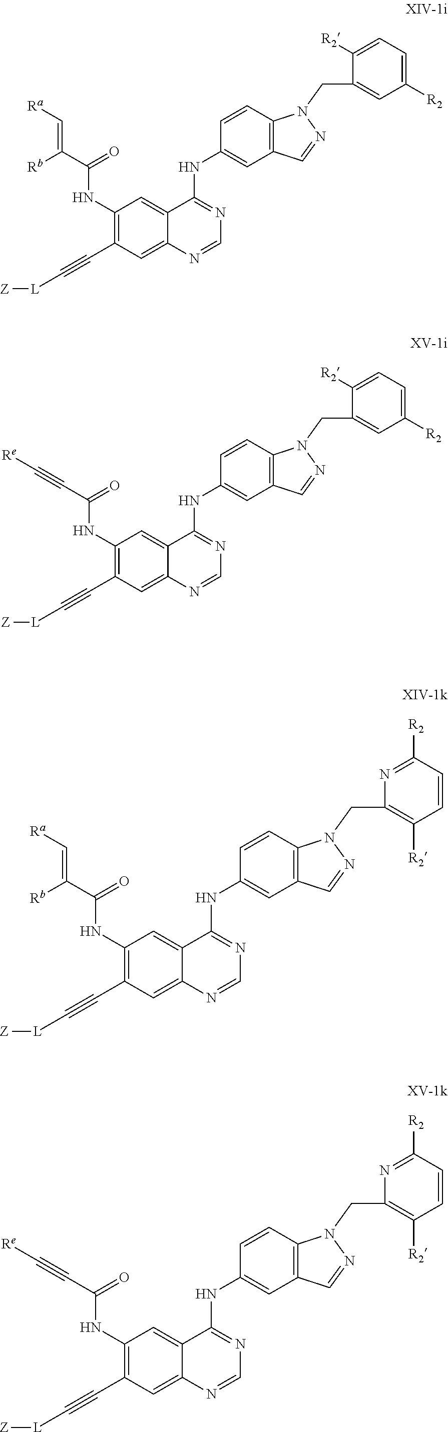

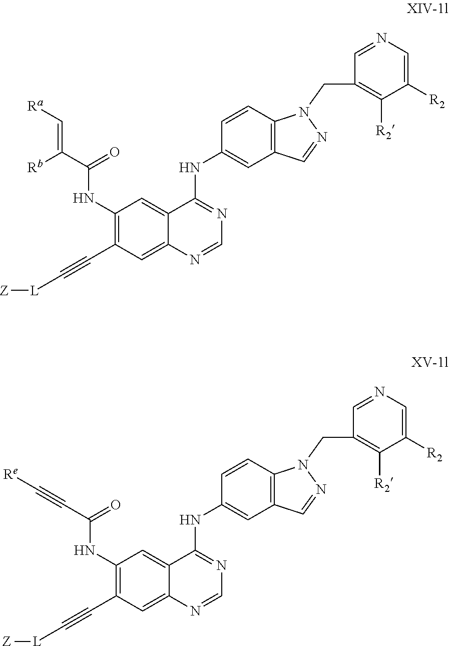

wherein Rₐ and R_{b} are independently of each other H, hal, or —CH₂—O—CH₃; and Rₑ is H or methyl.

In some embodiments, R² and R^{2′} are independently of each other H, C₁₋₆ alkyl, hal, —CF₃, or —OCF₃;

n is 0, 1, 2, or 3, (e.g., 1 or 2);
L is a covalent bond, straight chain or branched C₁₋₄ alkyl, or

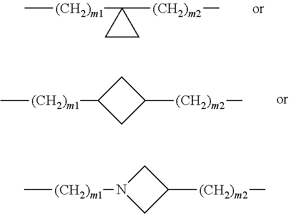

wherein m1 and R² are independently of each other 0, 1, 2, 3, or 4, (e.g., L is a covalent bond, straight chain or branched C₁₋₄ alkyl); and
Z is —(NR⁴R⁵), wherein R⁴ and R⁵ are independently of each other H, C₁₋₆ alkyl, cyclopropyl, cylobutyl, 3 to 6-membered heterocycloalkyl, —(NR⁶R⁷), or —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged-, or spiro-bicycle or a combination thereof and is unsubstituted or substituted with C₁₋₄ alkyl, hal, —OR′, or —NR′R″, wherein R′ and R″ are independently of each other H or —C₁₋₄ alkyl.

In some embodiments, R² and R^{2′} are independently of each other H, hal or C₁₋₆ alkyl, (e.g., H, hal, or —CH₃). In some embodiments, R² is H or hal. In some embodiments, R^{2′} is H. In some embodiments, R² and R^{2′} are H. In some embodiments, R² and R^{2′} are hal. In some embodiments, R² is hal and R^{2′} is H. In some embodiments, R² is H and R^{2′} is hal.

In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂— or —CH₂—C(CH₃)₂—). In some embodiments, L is —CH₂—, —(CH₂)₂—, or —C(CH₃)₂—. In some embodiments, L is

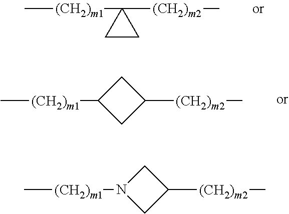

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g. —CH₂—, —(CH₂)₂—, —(CH₂)₃—, —(CH₂)₄—, or —C(CH₃)₂—). In some embodiments, L is —CH₂—, —(CH₂)₂—, or —C(CH₃)₂—. In some embodiments, L is a covalent bond. In some embodiments, L is straight chain or branched C₁₋₄ alkyl, (e.g., —CH₂—, —(CH₂)₂—, —(CH₂)₃—, —(CH₂)₄—, —C(CH₃)₂—, or —CH₂—C(CH₃)₂—). In some embodiments, L is

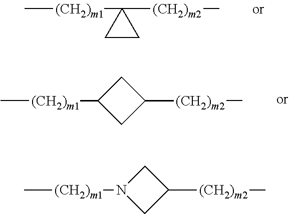

wherein m1 and m2 are independently of each other 0, 1, 2, 3, or 4, (e.g., 0, 1, or 2). In some embodiments, m2 is 0 and m1 is 0 or 1 or 2. In some embodiments, m1 and m2 are 1. In some embodiments, m1 and m2 are 2.

In some embodiments, L is a covalent bond or straight chain or branched C₁₋₄ alkyl, (e.g., —CH₂—, —(CH₂)₂—, —(CH₂)₃—, —(CH₂)₄—, or —C(CH₃)₂—).

In some embodiments of each compound of formula XIV, XIV-1, XIV-1a to XIV-1l, and XV, XV-1, XV-1a to XV-1l, a 3 to 6-membered heterocycloalkyl (in combination with —(NR⁴R⁵)) refers to a non-aromatic or partially aromatic ring system having 3, 4, 5, or 6 ring atoms independently selected from C, N, O, and S, (e.g., C, N, and O). In some embodiments, the number of N atoms is 0, 1, or 2. In some embodiments, the number of O and S atoms each is 0, 1, or 2. Examples of 3 to 6-membered heterocycloalkyl groups include oxiranyl, thiaranyl, aziradinyl, oxetanyl, thiatanyl azetidinyl, pyrrolidinyl, tetrahydrofuranyl, tetrahydrothiopyranyl, dihydropyranyl, tetrahydropyranyl, 1,3-dioxolanyl, 1,4-dioxanyl, 1,4-oxathianyl 1,4-dithianyl, 1,3-dioxane, 1,3-dithianyl, piperazinyl, thiomorpholinyl, piperidinyl, morpholinyl and the like. In some embodiments, 3 to 6-membered heterocycloalkyl include 5-membered heterocycloalkyl having 1 or 2 O-atoms, such as oxiranyl, oxetanyl, tetrahydrofuranyl, dioxanyl.

In some embodiments of each compound of formula XIV, XIV-1, XIV-1a to XIV-1l, and XV, XV-1, XV-1a to XV-1l, a 3 to 6-membered heteroaryl (in combination with —(NR⁶R⁷) or —(CHR⁶R⁷)) refers to a (fully) aromatic ring system having 3, 4, 5, or 6 ring atoms, (e.g., 3, 4, or 5 ring atoms), independently selected from C, N, O, and S, (e.g. C, N, and O, or C and N).

In some embodiments, the number of N atoms is 0, 1, 2, or 3. In some embodiments, the number of O and S atoms each is 0, 1, or 2. Examples of “heteroaryl” include furyl, imidazolyl, isoxazolyl, oxazolyl, pyrazinyl, pyrazolyl (pyrazyl), pyridazinyl, pyridinyl, pyrimidinyl, pyrrolyl, thiazolyl, thienyl, and the like. In some embodiments, examples of “heteroaryl” include pyrrolyl, imidazolyl.

In some embodiments of each compound of formula XIV, XIV-1, XIV-1a to XIV-1l, and XV, XV-1, XV-1a to XV-1l, a 3 to 9-membered heterocycloalkyl (in combination with —(NR⁶R⁷) or —(CHR⁶R⁷)) refers to a non-aromatic or partially aromatic ring system having 3 to 9 ring atoms independently selected from C, N, O, and S, (e.g. C, N, and O). In some embodiments, the number of N atoms is 0, 1, 2, or 3. In some embodiments, the number of O and S atoms each is 0, 1, or 2. Examples of a 3 to 9-membered heterocycloalkyl (in combination with —(NR⁶R⁷) or —(CHR⁶R⁷)) include monocycles such as oxiranyl, thiaranyl, aziradinyl, oxetanyl, thiatanyl, azetidinyl, pyrrolidinyl, tetrahydrofuranyl, tetrahydrothiopyranyl, dihydropyranyl, tetrahydropyranyl, 1,3-dioxolanyl 1,4-dioxanyl, 1,4-oxathianyl 1,4-dithianyl, 1,3-dioxane, 1,3-dithianyl piperazinyl, thiomorpholinyl, piperidinyl, morpholinyl, oxepanyl, thiepanyl, azepanyl, diazepanyl, oxazepanyl, (e.g., azetidinyl, pyrrolidinyl, piperidinyl, piperazinyl, morpholinyl); fused ring systems, such as 3-azabicyclo[3.1.0]hexane, 3-azabicyclo[3.3.0]octyl, 3,7-diazabicyclo[3.3.0]octyl, 3-aza-7-oxabicyclo[3.3.0]octyl, 2,6-diazabicyclo[3.3.0]octyl, 2,7-diazabicyclo[3.3.0]octyl, 2,8-diazabicyclo[4.3.0]nonyl, 3-oxa-8-azabicyclo[4.3.0]nonyl, 2-oxa-8-azabicyclo[4.3.0]nonyl, 2,8-diaza-5-oxabicyclo[4.3.0]nonyl, 4,9-diazabicyclo[4.3.0]nonyl, 2,9-diazabicyclo[4.3.0]nonyl, 3,8-diazabicyclo[4.3.0]nonyl, 3,7-diazabicyclo[4.3.0]nonyl, 3,9-diazabicyclo[4.3.0]nonyl, 3-oxa-8-azabicyclo[4.3.0]nonyl, 3-thia-8-azabicyclo[4.3.0]nonyl, and the like; bridged ring systems such as bicyclo[3.3.1]nonanyl, bicyclo[3.2.1]octanyl, bicyclo[2.2.2]octanyl, bicyclo[3.1.1]heptanyl, bicyclo[2.2.1]heptanyl, (e.g., bicyclo[3.2.1]octanyl, bicyclo[2.2.1]heptanyl), having one or two heteroatoms selected from N and O; spiro ring systems such as spiropentanyl, spiro[2.3]hexanyl spiro[3.3]heptanyl, spiro[3.4]octanyl, spiro[4.4]nonanyl, spiro[3.5]nonanyl, spiro[4.5]decanyl (e.g., spiro[3.3]heptanyl spiro[4.4]nonanyl), having one or two heteroatoms selected from N and O, (e.g., diazaspiro[3.3]heptanyl, oxa-azaspiro[3.3]heptanyl, diazaspiro[4.4]nonanyl oxa-azaspiro[4.4]nonanyl).

In some embodiments —(NR⁶R⁷) ring systems include

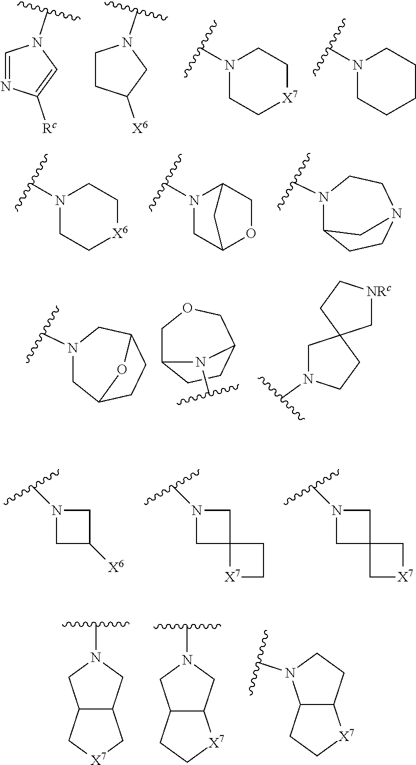

wherein R^{c} is H, C₁₋₄ alkyl, or oxetane; X⁶ is H, —CH₃, —OH, —OCH₃, —OCF₃, —N(CH₃)₂, F, or Cl; and X⁷ is —O—, —NH—, or —N(CH₃)—.

In some embodiments, —(CHR⁶R⁷) ring systems include

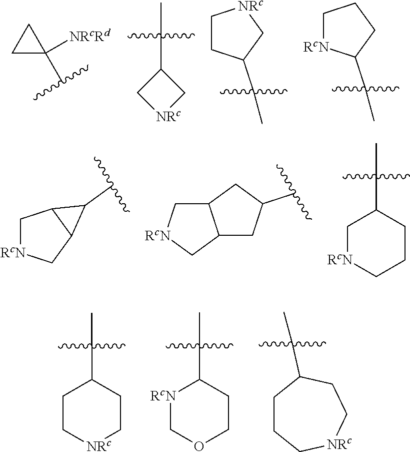

wherein R^{c} is H, C₁₋₄ alkyl, or oxetane; and R^{d} is H or C₁₋₄ alkyl.

In some embodiments of each compound of formula XIV, XIV-1, XIV-1a to XIV-1l, and XV, XV-1, XV-1a to XV-1l Z is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, (e.g., 5-membered heteroaryl) or 3 to 9-membered, (e.g., 6-8-membered heterocycloalkyl), wherein the 3 to 9-membered heterocycloalkyl is a monocycle or a bridged bicycle and is unsubstituted or substituted with C₁₋₄ alkyl, wherein 3 to 6-membered, (e.g., 5-membered heteroaryl) or 3 to 9-membered, (e.g., 6-8-membered heterocycloalkyl) include the ring systems as defined above.

In some embodiments, ring systems of group Z include

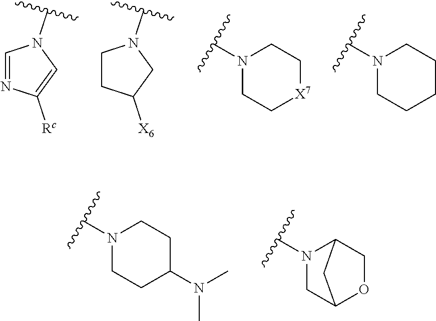

wherein R^{c} is H, C₁₋₄ alkyl, or oxetane; X⁶ is H, —CH₃, —OH, —OCH₃, —OCF₃, —N(CH₃)₂, F, or Cl, (e.g., H or —CH₃); and X⁷ is —O—, —NH—, or —N(CH₃)—.

In some embodiments, ring systems of group Z include

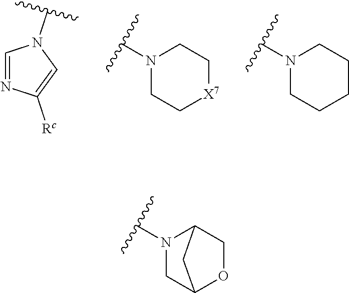

wherein R^{c} is H, C₁₋₄ alkyl, or oxetane; and X⁷ is —O—, —NH—, or —N(CH₃)—.

In some embodiments, the compound is selected from the compounds described in Table I, pharmaceutically acceptable salts thereof, and stereoisomers thereof.

In some embodiments, the compound is selected from the compounds described in Table I and pharmaceutically acceptable salts thereof.

In some embodiments, the compound is selected from the compounds described in Table I.

TABLE I

| Compound | No. |
|---|---|
| 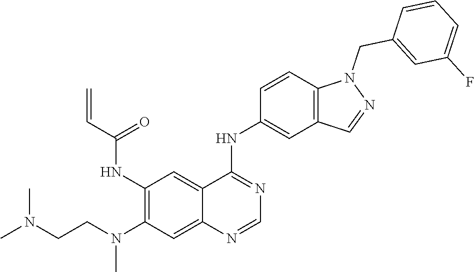 [SMILES: C=CC(=O)Nc1cc2c(Nc3ccc4c(cnn4Cc4cccc(F)c4)c3)ncnc2cc1N(C)CCN(C)C] | 1 |
| 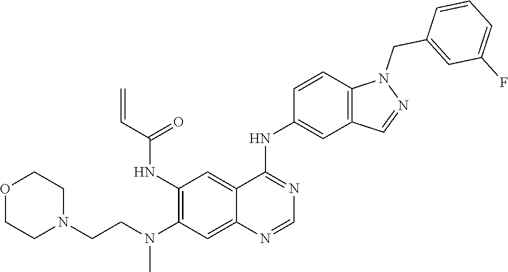 [SMILES: C=CC(=O)Nc1cc2c(Nc3ccc4c(cnn4Cc4cccc(F)c4)c3)ncnc2cc1N(C)CCN1CCOCC1] | 2 |
| 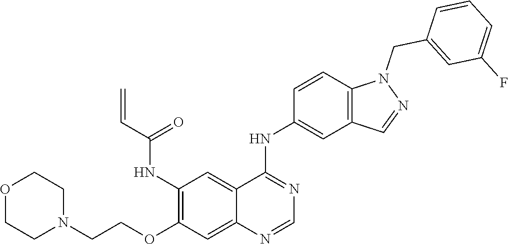 [SMILES: C=CC(=O)Nc1cc2c(Nc3ccc4c(cnn4Cc4cccc(F)c4)c3)ncnc2cc1OCCN1CCOCC1] | 3 |
| 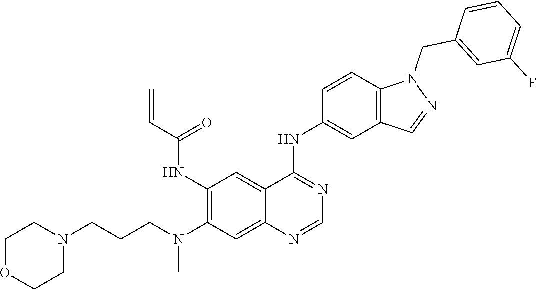 [SMILES: C=CC(=O)Nc1cc2c(Nc3ccc4c(cnn4Cc4cccc(F)c4)c3)ncnc2cc1N(C)CCCN1CCOCC1] | 4 |
| 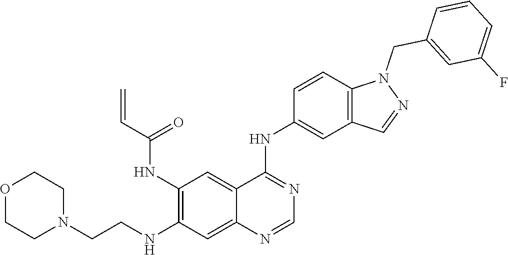 [SMILES: C=CC(=O)Nc1cc2c(Nc3ccc4c(cnn4Cc4cccc(F)c4)c3)ncnc2cc1NCCN1CCOCC1] | 5 |
| 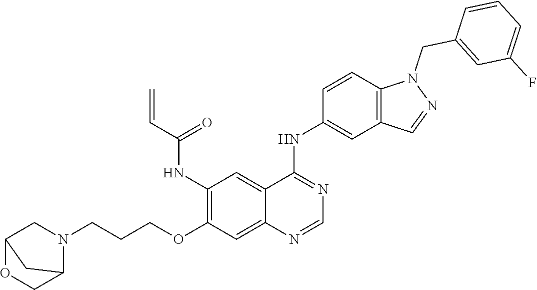 [SMILES: C=CC(=O)Nc1cc2c(Nc3ccc4c(cnn4Cc4cccc(F)c4)c3)ncnc2cc1OCCCN1CC2CC1CO2] | 6 |
| 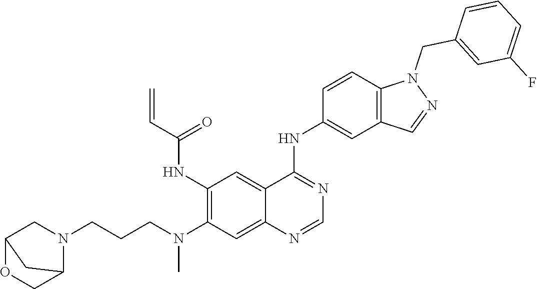 [SMILES: C=CC(=O)Nc1cc2c(Nc3ccc4c(cnn4Cc4cccc(F)c4)c3)ncnc2cc1N(C)CCCN1CC2CC1CO2] | 7 |
| 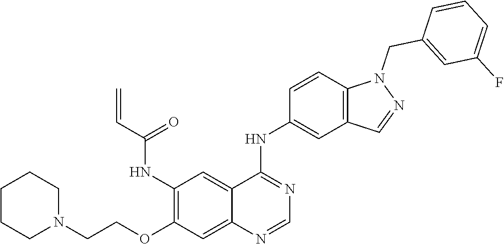 [SMILES: C=CC(=O)Nc1cc2c(Nc3ccc4c(cnn4Cc4cccc(F)c4)c3)ncnc2cc1OCCN1CCCCC1] | 8 |
| 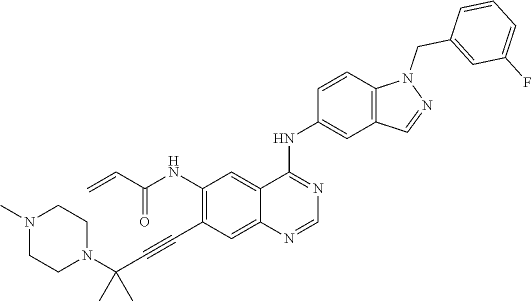 [SMILES: C=CC(=O)Nc1cc2c(Nc3ccc4c(cnn4Cc4cccc(F)c4)c3)ncnc2cc1C#CC(C)(C)N1CCN(C)CC1] | 9 |
| 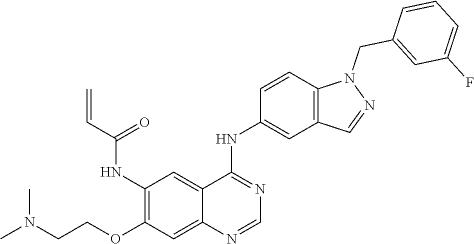 [SMILES: C=CC(=O)Nc1cc2c(Nc3ccc4c(cnn4Cc4cccc(F)c4)c3)ncnc2cc1OCCN(C)C] | 10 |

The compounds of the disclosure can contain one or more asymmetric centers in the molecule. A compound without designation of the stereochemistry is to be understood to include all the optical isomers (e.g., diastereomers, enantiomers, etc) in pure or substantially pure form, as well as mixtures thereof (e.g. a racemic mixture, or an enantiomerically enriched mixture). It is well known in the art how to prepare such optically active forms (e.g. by resolution of the racemic form by recrystallization techniques, by synthesis from optically-active starting materials, by chiral synthesis, by chromatographic separation using a chiral stationary phase, and other methods).

The compounds can be isotopically-labeled compounds, for example, compounds including various isotopes of hydrogen, carbon, nitrogen, oxygen, phosphorous, fluorine, iodine, or chlorine. The disclosed compounds may exist in tautomeric forms and mixtures and separate individual tautomers are contemplated. In addition, some compounds may exhibit polymorphism.

The compounds of the disclosure include the free form as well as the pharmaceutically acceptable salts and stereoisomers thereof. The pharmaceutically acceptable salts include all the typical pharmaceutically acceptable salts. The pharmaceutically acceptable salts of the present compounds can be synthesized from the compounds of this disclosure which contain a basic or acidic moiety by conventional chemical methods, see e.g. Berge et al, “Pharmaceutical Salts,” J. Pharm. Sci, 1977:66:1-19.

For example, conventional pharmaceutically acceptable salts for a basic compound include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like. Conventional pharmaceutically acceptable salts for an acidic compound include those derived from inorganic bases include aluminum, ammonium, calcium, copper, ferric, ferrous, lithium, magnesium, manganic salts, manganous, potassium, sodium, zinc and the like. Salts derived from pharmaceutically acceptable organic bases include salts of primary, secondary and tertiary amines, substituted amines including naturally occurring substituted amines, cyclic amines and basic ion exchange resins, such as arginine, betaine caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

The compounds of the disclosure may exist in solid, i.e. crystalline or noncrystalline form (optionally as solvates) or liquid form. In the solid state, it may exist in, or as a mixture thereof. In crystalline solvates, solvent molecules are incorporated into the crystalline lattice during crystallization. The formation of solvates may include non-aqueous solvents such as, but not limited to, ethanol, isopropanol, DMSO, acetic acid, ethanolamine, or ethyl acetate, or aqueous solvents such as water (also called “hydrates”). It is common knowledge that crystalline forms (and solvates thereof) may exhibit polymorphism, i.e. exist in different crystalline structures known as “polymorphs”, that have the same chemical composition but differ in packing, geometrical arrangement, and other descriptive properties of the crystalline solid state. Polymorphs, therefore, may have different physical properties such as shape, density, hardness, deformability, stability, and dissolution properties, and may display different melting points, IR spectra, and X-ray powder diffraction patterns, which may be used for identification. Such different polymorphs may be produced, for example, by changing or adjusting the reaction conditions or reagents, during preparation of the compound of the disclosure.

### Syntheses of Compounds

In some embodiments, the present disclosure provides methods of preparation of the compounds of the present disclosure. In some embodiments, the compounds are prepared according to the syntheses shown in schemes A to D hereinafter.

In some embodiments, the present disclosure provides a method of preparing a compound of the present disclosure.

In some embodiments, the present disclosure provides a method of a compound, comprising one or more steps as described herein.

In some embodiments, the present disclosure provides a compound obtainable by, or obtained by, or directly obtained by a method for preparing a compound as described herein.

In some embodiments, the present disclosure provides an intermediate as described herein, being suitable for use in a method for preparing a compound as described herein.

The compounds of the present disclosure can be prepared by any suitable technique known in the art. Processes for the preparation of these compounds are described in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilized.

It will be appreciated that during the synthesis of the compounds of the disclosure in the processes defined herein, or during the synthesis of certain starting materials, it may be desirable to protect certain substituent groups to prevent their undesired reaction. The skilled chemist will appreciate when such protection is required, and how such protecting groups may be put in place, and later removed. For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule. Thus, if reactants include, for example, groups such as amino, carboxy or hydroxy it may be desirable to protect the group in some of the reactions mentioned herein.

As will be understood by the person skilled in the art of organic synthesis, compounds of the present disclosure are readily accessible by various synthetic routes, some of which are exemplified in the accompanying examples. The skilled person will easily recognize which kind of reagents and reactions conditions are to be used and how they are to be applied and adapted in any particular instance—wherever necessary or useful in order to obtain the compounds of the present disclosure. In some embodiments, some of the compounds of the present disclosure can readily be synthesised by reacting other compounds of the present disclosure under suitable conditions, for instance, by converting one particular functional group being present in a compound of the present disclosure, or a suitable precursor molecule thereof, into another one by applying standard synthetic methods, like reduction, oxidation, addition or substitution reactions; those methods are well known to the skilled person.

Likewise, the skilled person will apply—whenever necessary or useful—synthetic protecting (or protective) groups; suitable protecting groups as well as methods for introducing and removing them are well-known to the person skilled in the art of chemical synthesis and are described, in more detail, in, e.g., P.G.M. Wuts, T.W. Greene, “Greene's Protective Groups in Organic Synthesis”, 4th edition (2006) (John Wiley & Sons).

General routes for the preparation of a compound of the application are described in the general procedures A-D:

### General Procedure A:

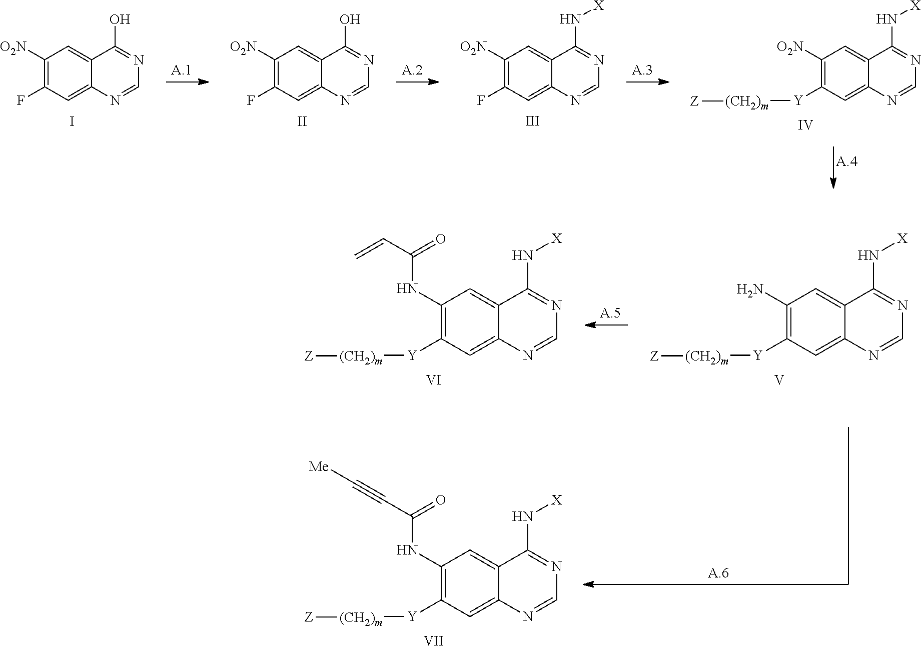

### Step A.1:

A solution of 7-fluoro-6-nitro-quinazolin-4-ol (5.00 g, 23.9 mmol, 1.00 eq) in thionyl chloride (20.0 mL) was added dimethyl formamide (174 mg, 2.39 mmol, 183 uL, 0.10 eq).

The reaction was stirred at 80° C. for 10 h. The reaction mixture was concentrated under reduced pressure to give 4-chloro-7-fluoro-6-nitroquinazoline (6.00 g, crude) as an off-white solid. The product was taken to next step without purification.

### Step A.2:

A mixture of 4-chloro-7-fluoro-6-nitroquinazoline (2.4 g, 10.55 mmol, 1 eq) and the free amine H₂N—X (1 eq) in isopropyl alcohol was heated at 80° C. for 1 h. The reaction mixture was concentrated under reduced pressure to give a residue. The residue was triturated with ethyl acetate to give amine III.

### Step A.3:

To a solution of amine III (1 eq) and the NH or OH nucleophile Z—(CH₂)ₘ—YH (1.1 eq) in acetonitrile was added cesium carbonate (2 eq) or DBU (2 eq) and optionally potassium iodide (1 eq). Then the mixture was stirred at 80-110° C. for 12 h. The reaction mixture was quenched by addition of water and then extracted with ethyl acetate. The combined organic layers were washed with brine dried over sodium sulfate, filtered and concentrated under reduced pressure to give a residue. The residue was purified by flash silica gel chromatography to give IV.

### Step A.4:

Variant i): A mixture of IV (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine V.

Variant ii): A mixture of IV (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine V.

### Step A.5:

Variant i): To a solution of V (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide VI.

Variant ii): To a solution of V (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide VI.

Variant iii): To a solution of V (1.0 eq) in dimethylformamide was added triethylamine (3.00 eq) and acryloyl chloride (1.20 eq) at 0° C. The reaction mixture was stirred at 0° C. for 1 h and subsequently filtered. The filtrate was purified by prep-HPLC to give acrylamide VI.

### Step A.6:

To a solution of V (1.0 eq), 1-(3-dimethylaminopropyl)-3-ethylcarbodiimide hydrochloride (5.00 eq) and pyridine (5.00 eq) in N,N-dimethylformamide was added but-2-ynoic acid (10.0 eq). The mixture was stirred at 50° C. for 2 h and subsequently concentrated in vacuum. The mixture was purified by prep-HPLC to give ynamide VII.

### General Procedure B:

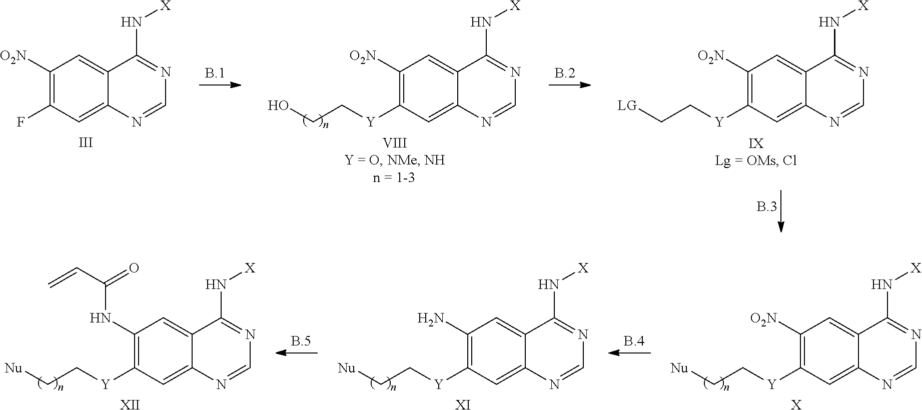

### Step B.1:

To a solution of III, obtained in step A.2 (1.00 eq) and potassium tert-butoxide (4.00 eq) In dimethylsulfoxide (10.0 mL) was added the corresponding diol of aminoalcohol (6.00 eq) dropwise at 20° C. The mixture was stirred at 20° C. for 12 h. The mixture was diluted with water and extracted with ethyl acetate. The combined organic layer was washed with brine and dried over sodium sulfate, filtered and concentrated to give crude product. The crude product was purified by silica gel chromatography to give alcohol VIII.

### Step B.2:

Variant i): To a solution of VIII (1 eq) and triethylamine (4.00 eq) in dichloromethane and dimethylsulfoxide (6:1) was added MsCl (4.00 eq) dropwise at 0° C. The mixture was stirred at 20° C. for 2 h. The mixture was diluted with water and extracted with dichloromethane. The combined organic layer was washed with brine and dried over sodium sulfate, filtered and concentrated to give Mesylate IX.

Variant ii): To a solution of VIII (1.0 eq) in thionyl chloride was added N,N-dimethylformamide (0.1 eq). The mixture was stirred at 90° C. for 3 h. The mixture was cooled to 25° C. and then concentrated in vacuum. The mixture was partitioned between and ethyl acetate. The organic phase was washed with brine, dried with anhydrous sodium sulfate, filtered and concentrated in vacuum. The residue was purified by silica gel chromatography to afford chloride IX.

### Step B.3:

To a solution of IX (1.0 eq) and potassium carbonate (4.00 eq) in dimethylsulfoxide was the corresponding N—H nucleophile (2.0 eq) in one portion at 20° C. The mixture was stirred at 50° C. for 12 h. The mixture was diluted with water and extracted with ethyl acetate. The combined organic layer was washed with brine and dried over sodium sulfate, filtered and concentrated to give crude product. The crude product was purified by prep-HPLC to give X.

### Step B.4:

Variant i): A mixture of X (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine XI.

Variant ii): A mixture of X (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine XI.

### Step B.5:

Variant i): To a solution of XI (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide XII.

Variant ii): To a solution of XI (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide XII.

Variant iii): To a solution of XI (10 eq) in dimethylformamide was added triethylamine (3.00 eq) and acryloyl chloride (1.20 eq) at 0° C. The reaction mixture was stirred at 0° C. for 1 h and subsequently filtered. The filtrate was purified by prep-HPLC to give acrylamide XII.

### General Procedure C:

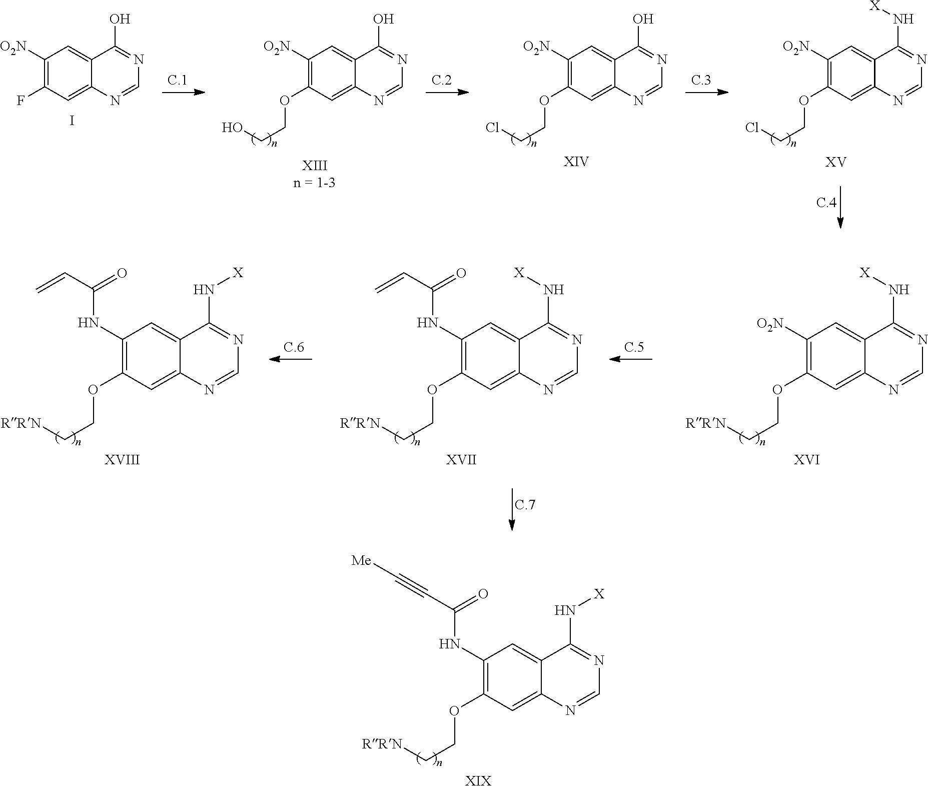

### C.1:

Sodium (3.0 eq) was added to the corresponding diol (18.7 eq) at 25° C. The suspension was stirred at 25° C. for 0.5 h. Alcohol 1 (1.0 eq) was added to the above suspension. The mixture was heated to 70° C. and stirred at 70° C. for 1.5 h. The mixture was cooled to 25° C. and then adjusted to pH=7 with hydrochloric acid (3 M). After filtration, the filter cake was dried under reduced pressure to afford diol XIII.

### Step C.2:

To a solution of diol XIII (1.00 eq) in thionyl chloride (101.0 mL) was added N,N-dimethylformamide (0.1 eq). The mixture was stirred at 90° C. for 3 h. The mixture was cooled to 25° C. and then concentrated in vacuum. The mixture was partitioned between water and ethyl acetate. The organic phase was washed with brine, dried with anhydrous sodium sulfate, filtered and concentrated in vacuum. The residue was purified by silica gel chromatography to afford dichloride XIV.

### Step C.3:

A solution of dichloride XIV (1.0 eq) and H₂N—X (1.50 eq) in propan-2-ol was stirred at 90° C. for 12 h. The mixture was cooled to 25° C. and then concentrated in vacuum. The residue was triturated with methanol, then filtered and dried under reduced pressure to afford XV.

### Step C.4:

To a solution of XV (1.0 eq), potassium iodide (0.1 eq) and tetrabutylammonium iodide (0.1 eq) in toluene was added HNR′R″ (3.00 eq). The mixture was stirred at 110° C. for 12 h. The mixture was cooled to 25° C. and then concentrated in vacuum. The residue was triturated with water and filtered, the filter cake was dried in vacuum to afford XVI.

### Step C.5:

Variant i): A mixture of XVI (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine XVII.

Variant ii): A mixture of XVI (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine XVII.

### Step C.6:

Variant i): To a solution of XVII (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide XVIII.

Variant ii): To a solution of XVII (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide XVIII.

Variant iii): To a solution of XVII (1.0 eq) in dimethylformamide was added triethylamine (3.00 eq) and acryloyl chloride (1.20 eq) at 0° C. The reaction mixture was stirred at 0° C. for 1 h and subsequently filtered. The filtrate was purified by prep-HPLC to give acrylamide XVIII.

### Steps C.7:

To a solution of XVII (1.0 eq), 1-(3-dimethylaminopropyl)-3-ethylcarbodiimide hydrochloride (5.00 eq) and pyridine (5.00 eq) in N,N-dimethylformamide was added but-2-ynoic acid (10.0 eq). The mixture was stirred at 50° C. for 2 h and subsequently concentrated in vacuum. The mixture was purified by prep-HPLC to give ynamide XIX.

### General Procedure D:

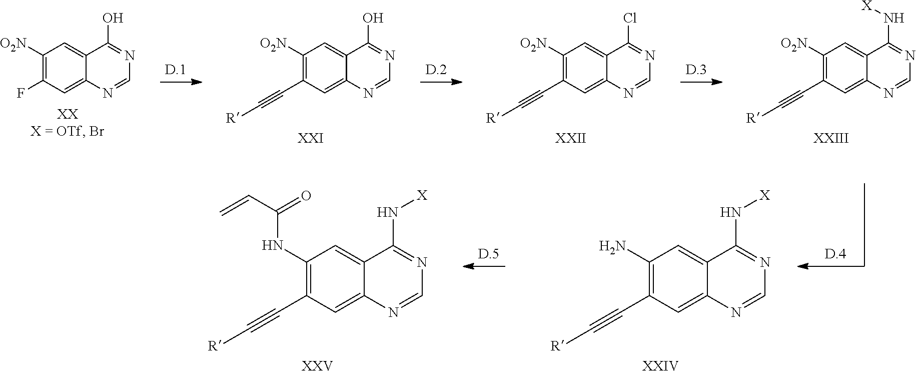

### Step D.1:

To a solution of bromide or triflate XX (1.00 eq) in dimethylsulfoxide was added the corresponding alkyne (1.50 eq), triethylamine (3.00 eq), copper (1) iodide (0.5 eq), tetrakis(triphenylphosphine)palladium (0.05 eq) at 20° C. The mixture was degassed with nitrogen and stirred at 20° C. for 12 h under nitrogen. The mixture was added methanol and filtered, the filter cake was concentrated to give alkyne XXI.

### Step D.2:

To a suspension of alkyne XXI (1.00 eq) in thionyl chloride was added N,N-dimethylformamide (2.0 eq) at 20° C. The mixture was stirred at 90° C. for 0.5 h until the suspension turned to homogenous solution. The solution was concentrated to give chloride XXII.

### Step D.3:

A suspension of chloride XXII (1.0 eq) and H₂N—X in propan-2-ol was stirred at 80° C. for 12 h. The mixture was concentrated to give a residue. And the residue was purified by reverse phase chromatography to give XXIII.

### Step D.4:

Variant i): A mixture of XXIII (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine XXIV.

Variant ii): A mixture of XXIII (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine XXIV.

### Step D.5:

Variant i): To a solution of XXIV (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide XXV.

Variant ii): To a solution of XXIV (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide XXV.

Variant iii) To a solution of XXIV (1.0 eq) in dimethylformamide was added triethylamine (3.00 eq) and acryloyl chloride (1.20 eq) at 0° C. The reaction mixture was stirred at 0° C. for 1 h and subsequently filtered. The filtrate was purified by prep-HPLC to give acrylamide XXV.

### Pharmaceutical Compositions

In some embodiments, the disclosure further provides a pharmaceutical composition comprising a therapeutically-effective amount of one or more of the compounds of the disclosure or a pharmaceutically acceptable salt thereof and one or more pharmaceutically acceptable carriers and/or excipients (also referred to as diluents). The excipients are acceptable in the sense of being compatible with the other ingredients of the formulation and not deleterious to the recipient thereof (i.e., the patient). The term “therapeutically-effective amount” as used herein refers to the amount of a compound (as such or in form of a pharmaceutical composition) of the present disclosure which is effective for producing some desired therapeutic effect.

Pharmaceutical compositions may be in unit dose form containing a predetermined amount of a compound of the disclosure per unit dose. Such a unit may contain a therapeutically effective dose of a compound of the disclosure or salt thereof or a fraction of a therapeutically effective dose such that multiple unit dosage forms might be administered at a given time to achieve the desired therapeutically effective dose. In some embodiments, unit dosage formulations are those containing a daily dose or sub-dose, or an appropriate fraction thereof, of a compound of the disclosure or salt thereof.

The compounds of the present disclosure may be administered by any acceptable means in solid or liquid form, including (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) nasally; (9) pulmonary; or (10) intrathecally.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or poly anhydrides; and (22) other non-toxic compatible substances employed in pharmaceutical compositions.

Such compositions may contain further components conventional in pharmaceutical preparations, e.g. wetting agents, emulsifiers and lubricants, such as sodium lauryl sulfate and magnesium stearate, as well as coloring agents, release agents, coating agents, sweetening flavoring and perfuming agents, preservatives and antioxidants, pH modifiers, bulking agents, and further active agents. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxy anisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Such compositions may be prepared by any method known in the art, for example, by bringing into association the active ingredient with one or more carriers and/or excipients. Different compositions and examples of carriers and/or excipients are well known to the skilled person and are described in detail in, e.g., Remington: The Science and Practice of Pharmacy. Pharmaceutical Press, 2013; Rowe, Sheskey, Quinn: Handbook of Pharmaceutical Excipients. Pharmaceutical Press, 2009. Excipients that may be used in the preparation of the pharmaceutical compositions may include one or more of buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide a composition suitable for an administration of choice.

In some embodiments, the compounds of the present disclosure may be in solid or liquid form and administered by various routes in any convenient administrative form, e.g., tablets, powders, capsules, solutions, dispersions, suspensions, syrups, sprays, suppositories, gels, emulsions, patches, etc.

In solid dosage forms of the disclosure for oral administration (capsules, tablets, pills, dragees, powders, granules, trouches and the like), a compound is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof, (10) coloring agents; and (11) controlled release agents such as crospovidone or ethyl celluose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent. The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, pol ethylene gly cols and fatty acid esters of sorbitan, and mixtures thereof. An oral composition can also include adjuvants such as wetting agents, emulsifying and suspending agents, sweetening, flavoring, coloring, perfuming and preservative agents.

In form of suspensions, a compound may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for rectal or vaginal administration of a compound of the disclosure include a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Other suitable forms include pessaries, tampons, creams, gels, pastes, foams or spray formulations containing such carriers as are known in the art to be appropriate.

Dosage forms for the topical or transdermal administration of a compound of the disclosure include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required. Such ointments, pastes, creams and gels may contain, in addition to a compound of the disclosure, excipients, such as animal and vegetable fats, oils, waxes, paraffins, starch, tragacanth, cellulose derivatives, polyethylene glycols, silicones, bentonites. silicic acid, talc and zinc oxide, or mixtures thereof.

Dosage forms such as powders and sprays for administration of a compound of the disclosure may contain excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Dosage forms such as transdermal patches for administration of a compound of the disclosure may include absorption enhancers or retarders to increase or decrease the flux of the compound across the skin. The rate of such flux can be controlled by either providing a rate controlling membrane or dispersing the compound in a polymer matrix or gel. Other dosage forms contemplated include ophthalmic formulations, eye ointments, powders, solutions and the like. It is understood that all contemplated compositions must be stable under the conditions of manufacture and storage, and preserved against the contaminating action of microorganisms, such as bacteria and fungi.

The dosage levels of a compound of the disclosure in the pharmaceutical compositions of the disclosure may be adjusted in order to obtain an amount of a compound of the disclosure which is effective to achieve the desired therapeutic response for a particular patient, composition, and mode of administration, without being deleterious to the patient. The dosage of choice will depend upon a variety of factors including the nature of the particular compound of the present disclosure used, the route of administration, the time of administration, the rate of excretion or metabolism of the particular compound used, the rate and extent of absorption, the duration of the treatment, other drugs, compounds and/or materials used in combination with the particular compound, the age, sex, weight, condition, general health and prior medical history of the patient being treated, and like factors well known in the medical arts. A medical practitioner having ordinary skill in the art can readily determine and prescribe the effective amount of the pharmaceutical composition required. In some embodiments, a suitable daily dose of a compound of the disclosure will be that amount of the compound which is the lowest dose effective to produce a therapeutic effect. Such an effective dose will generally depend upon the factors described above. In some embodiments, oral, intravenous, intracerebroventricular and subcutaneous doses of the compounds of this disclosure for a patient, when used for the indicated analgesic effects, will range from about 0.0001 to about 100 mg, more usual 0.1 to 100 mg/kg per kilogram of body weight of recipient (patient, mammal) per day. Acceptable daily dosages may be from about 1 to about 1000 mg/day, and for example, from about 1 to about 100 mg/day.

The effective dose of a compound of the disclosure may be administered as two, three, four, five, six or more sub-doses administered separately at appropriate intervals throughout a specified period (per day or per week or per month), optionally, in unit dosage forms. In some embodiments, dosing also depends on factors as indicated above, e.g. on the administration, and can be readily arrived at by one skilled in medicine or the pharmacy art. The compounds of the disclosure inhibit or modulate the activity of a receptor tyrosine kinase, in particular extracellular mutants of ErbB-receptors, such as, but not limited to, EGFR-Viii (also EGFR-V3) and HER2-S310F. Thus, the compounds and compositions of the disclosure can be useful as a medicament, i.e. as a medicament in therapy, (e.g., for the treatment of cancer). Therefore, in a further aspect, the present disclosure provides a method of treatment of a mammal, for example, a human, suffering from cancer. The term “treatment” is intended to encompass prophylaxis, therapy and cure. Such treatment comprises the step of administering a therapeutically effective amount of a compound of Formula I or salt thereof (or of a pharmaceutical composition containing a compound of Formula I or salt thereof) to said mammal, for example, a human.

In some embodiments, the present disclosure is directed toward the use of the compounds of the disclosure or a pharmaceutically acceptable salt or stereoisomer thereof or a pharmaceutical composition thereof for the treatment of cancer in a mammal, for example a human.

Such a use (or method of treatment) of a subject comprises administering to a subject in need of such treatment a therapeutically effective amount of a compound of the disclosure or a pharmaceutically acceptable salt thereof or a pharmaceutical composition thereof by targeting allosteric and/or oncogenic variants of EGFR and HER-2 receptor.

The present disclosure contemplates administration of a compound of the disclosure alone or in combination with one or more additional therapeutic agents, such as other Tyrosine kinase inhibitors: Erlotinib hydrochloride (e.g. Tarceva® by Genentech/Roche), Linifanib (or ABT 869, by Genentech), sunitinib malate (e.g. Sutent® by Pfizer), bosutinib (or SKI-606, described in U.S. Pat. No. 6,780,996), dasatinib (e.g. Sprycel® by Bristol-Myers Squibb), armala (e.g. pazopanib, e.g. Votrient® by GlaxoSmithKline), imatinib and imatinib mesylate (e.g. Gilvec® and Gleevec® by Novartis); Vascular Endothelial Growth Factor (VEG) receptor inhibitors (Bevacizumab, or Avastin® by Genentech/Roche), axitinib, (or AGO 3736, described in WO 01/002369), Brivanib Alaninate (or BMS-582664), motesanb (or AMG-706, described in PCT WO 02/066470), pasireotide (e.g. SOM230, described in WO 02/010192), sorafenib (e.g. Nexavar®); HER2 receptor inhibitors: Trastuzumab (e.g. Herceptin® by Genentech/Roche), neratinib (or HKI-272, described WO 05/028443), lapatinib or lapatinib ditosylate (e.g. Tykerb® by GlaxoSmithKline); CD20 antibodies: Rituximab (e.g. Riuxan® and MabThera® by Genentech/Roche), tositumomab (e.g. Bexxar® by GlaxoSmithKline), ofatumumab (e.g. Arzerra® by GlaxoSmithKline); Bcr/Abl kinase inhibitors: nilotinib hydrochloride (e.g. Tasigna® by Novartis); DNA Synthesis inhibitors: Capecitabine (e.g. Xeloda® by Roche), gemcitabine hydrochloride (e.g. Gemzar® by Eli Lilly and Company), nelarabine (or Arranon® and Atriance® by GlaxoSmithKline); Antineoplastic agents: oxaliplatin (e.g. Eloxatin® ay Sanofi-Aventis described in U.S. Pat. No. 4,169,846); Epidermal growth factor receptor (EGFR) inhibitors: Gefitinib (or Iressa®), Afatinib (or Tovok® by Boehringer Ingelheim), cetuximab (e.g. Erbitux® by Bristol-Myers Squibb), panitumumab (e.g. Vectibix® by Amgen); HER dimerization inhibitors: Pertuzumab (e.g. Omnitarg®, by Genentech); Human Granulocyte colony-stimulatingfactor (G-CSF) modulators: Filgrastim (e.g. Neupogen® by Amgen); Immunomodulators: Afutuzumab (by Roche®), pegfilgrastir (e.g. Neulasta® by Amgen), lenalidornide (e.g. CC-5013, e.g. Revlimid®), thalidomide (e.g. Thalomid®); (m) CD40 inhibitors: Dacetuzumab (e.g. SGN-40 or huS2C6, by Seattle Genetics, Inc); Pro-apoptotic receptor agonists (PARAs): Dulanermin (e.g. AMG-951, by Amgen/Genentech); Hedgehog antagonists: Vismodegib (or GDC-0449, described in WO 06/028958); PI3K inhibitors: Pictilisib (or GDC-0941 described in WO 09/036082 and WO 09/055730), Dactolisib (or BEZ 235 or NVP-BEZ 235, described in WO 06/122806); Phospholipase A2 inhibitors: Anagrelide (e.g. Agrylin®); BCL-2 inhibitors: Navitoclax (or ABT-263, described in WO 09/155386); Mitogen-activated protein kinase kinase (MEK) inhibitors: XL-518 (Cas No. 1029872-29-4, by ACC Corp.); Aromatase inhibitors: Exemestane (e.g. Aromasin® by Pfizer), letrozole (e.g. Femara® by Novartis), anastrozole (e.g. Arimidex®); Topoisomerase I inhibitors: Irinotecan (e.g. Camptosar® by Pfizer), topotecan hydrochloride (e.g. Hycamtin® by GlaxoSmithKline); Topoisomerase II inhibitors: etoposide (e.g. VP-16 and Etoposide phosphate, e.g. Toposar®, VePesid® and Etopophos®), teniposide (e.g. VM-26, e.g. Vumon®); mrTOR inhibitors: Temsirolimus (e.g. Torisel® by Pfizer), ridaforolimus (formally known as deferolimus, (or AP23573 and MK8669, described in WO 03/064383), everolimus (e.g. Afinitor® by Novartis); Osteoclastic bone resorption inhibitors: zoledronic acid (or Zometa® by Novartis); CD33 Antibody Drug Conjugates: Gertuzumab ozogamicin (e.g. Mylotarg® by Pfizer/Wyeth); CD22 Antibody Drug Conjugates: Inotuzumab ozogamicin (also referred to as CMC-544 and WAY-207294, by Hangzhou Sage Chemical Co., Ltd.); CD20 Antibody Drug Conjugates: Ibritumomab tiuxetan (e.g. Zevalin®); Somatostain analogs: octreotide (e.g. octreotide acetate, e.g. Sandostatin® and Sandostatin LAR®); Synthetic Interleukin-11 (IL-11): oprelvekin (e.g. Neumega® by Pfizer/Wyeth); Synthetic erythropoietin: Darbepoetin alfa (e.g. Aranesp® by Amgen); Receptor Activator for Nuclear Factor kappa B (RANK) inhibitors: Denosumab (e.g. Prolia® by Amgen); Thrombopoietin mimetic peptibodies: Romiplostim (e.g. Nplate® by Aigen; Cell growth stimulators: Palifermin (e.g. Kepivance® by Amgen); Anti-Insulin-like Growth Factor-1 receptor (IGF-1R) antibodies: Figitumumab (e.g. CP-751,871, by ACC Corp), robatumumab (CAS No. 934235-44-6); Anti-CS1 antibodies: Elotuzumab (HuLuc63, CAS No. 915296-00-3); CD52 antibodies: Alentuzumab (e.g. Campath®); CTLA-4 inhibitors: Tremelimunab (IgG2 monoclonal antibody by Pfizer, formerly known as ticilimumab, CP-675,206), ipilimumab (CTLA-4 antibody, e.g. MDX-010, CAS No. 477202-00-9); Histone deacetylase inhibitors (HDI): Voninostat (e.g. Zolinza® by Merck); Alkylating agents: Temozolomide (e.g. Temodar® and Temodal® by Schering-Plough/Merck), dactinomycin (e.g. actinomycin-D and e.g. Cosmegen®), melphalan (e.g. L-PAM, L-sarcolysin, and phenylalanine mustard. e.g. Alkeran®), altretamine (e.g. hexamethylmelamine (HMM), e.g. Hexalen®), carmustine (e.g. BiCNU®), bendamustine (e.g. Treanda®), busulfan (e.g. Busulfex® and Myleran®), carboplatin (e.g. Paraplatin®), lomustine (e.g. CCNU, e.g. CeeNU®), cisplatin (e.g. CDDP, e.g. Platinol® and Platinol®-AQ), chlorambucil (e.g. Leukeran®), cyclophosphanride (e.g. Cytoxan® and Neosar®), dacarbazine (e.g. DTIC, DIC and imidazole carboxamide, e.g. DTIC-Dome®), altretamine (e.g. hexamethylmelamine (HMM) e.g. Hexalen®), ifosfamide (e.g. Ifex®), procarbazine (e.g. Matulane®), mechloretharine (e.g. nitrogen mustard, mustine and mechloroethamine hydrochloride, e.g. Mustargen®), streptozocin (e.g. Zanosar®), thiotepa (e.g. thiophosphoamide, TESPA and TSPA, e.g. Thioplex®; Biologic response modifiers: bacillus calmette-guerin (e.g. theraCys® and TICE® BCG), denileukin diftitox (e.g. Ontak®); Anti-tumor antibiotics: doxorubicin (e.g. Adriamycin® and Rubex®), bleomycin (e.g. Lenoxane®), daunorubicin (e.g. dauorubicin hydrochloride, daunomycin, and rubidomycin hydrochloride, e.g. Cerubidine®), daunorubicin liposomal (daunorubicin citrate liposome, e.g. DaunoXome®), mitoxantrone (e.g. DHAD, e.g. Novantrone®), epirubicin (e.g. Ellence™), idarubicin (e.g. Idamycin®, Idamycin PFS®), mitomycin C (e.g. Mutamycin®); Anti-microtubule agents: Estramustine (e.g. Emcyl®); Cathepsin K inhibitors: Odanacatib (or MK-0822, by Lanzhou Chon Chemicals, ACC Corp., and ChemieTek, described in WO 03/075836); Epothilone B analogs: Ixabepilone (e.g. Lxempra® by Bristol-Myers Squibb); Heat Shock Protein (HSP) inhibitors: Tanespimycin (17-allylamino-17-demethoxygeldanamycin, e.g. KOS-953 and 17-AAG, by SIGMA, described in U.S. Pat. No. 4,261,989); TpoR agonists: Eltrombopag (e.g. Promacta® and Revolade® by GlaxoSmithKline); Anti-mitotic agents: Docetaxel (e.g. Taxotere® by Sanofi-Aventis); Adrenal steroid inhibitors: aminoglutethimide (e.g. Cytadren®); Anti-androgens: Nilutamide (e.g. Nilandron® and Anandron®), bicalutamide (sold under tradename Casodex®), flutamide (e.g. Fulexin™); Androgens: Fluoxymesterone (e.g. Halotestin®); Proteasome inhibitors: Bortezomib (e.g. Velcade®); CDK1 inhibitors: Alvocidib (e.g. flovopirdol or HMR-1275, described in U.S. Pat. No. 5,621,002); Gonadotropin-releasing hormone (GnRH) receptor agonists: Leuprolide or leuprolide acetate (e.g. Viadure® by Bayer AG, Eligard® by Sanofi-Aventis and Lupron® by Abbott Lab); Taxane anti-neoplastic agents: Cabazitaxel, larotaxel; 5HT1a receptor agonists: Xaliproden (or SR57746, described in U.S. Pat. No. 5,266,573); HPC vaccines: Cervarix® sold by GlaxoSmithKline, Gardasil® sold by Merck; Iron Chelating agents: Deferasinox (e.g. Exjade® by Novartis); Anti-metabolites: Claribine (2-chlorodeoxyadenosine, e.g. Leustatin®), 5-fluorouracil (e.g. Adrucil®), 6-thioguanine (e.g. Purinethol®), pemetrexed (e.g. Alimta®), cytarabine (e.g. arabinosylcytosine (Ara-C), e.g. CYtosar-U®), cytarabine liposomal (e.g. Liposomal Ara-C, e.g. DepoCyt™), decitabine (e.g. Dacogen®), hydroxyurea (e.g. Hydrea®, Droxia™ and Mylocel™), fludarabine (e.g. Fludara®), floxuridine (e.g. FUDR®), cladribine (e.g. 2-chlorodeoxyadenosine (2-CdA) e.g. Leustatin™), methotrexate (e.g. amethopterin, methotrexate sodim (MTX), e.g. Rheumatrex® and Trexall™), pentostatin (e.g. Nipent®); Bisphosphonates: Pamidronate (e.g. Aredia®), zoledronic acid (e.g. Zometa®); Demethylating agents: 5-azacitidine (e.g. Vidaza®), decitabine (e.g. Dacogen®); Plant Alkaloids: Paclitaxel protein-bound (e.g. Abraxane®), vinblastine (e.g. vinblastine sulfate, vincaleukoblastine and VLB, e.g. Alkaban-AQ® and Velban®), vincristine (e.g. vincristine sulfate, LCR, and VCR, e.g. Oncovin® and Vincasar Pfs®), vinorelbine (e.g. Navelbine®), paclitaxel (e.g. Taxol and Onxal™); Retinoids: Alitretinoin (e.g. Panretin®), tretinoin (all-trans retinoic acid, e.g. ATRA, e.g. Vesanoid®), Isotretinoin (13-cis-retinoic acid, e.g. Acccutane®, Annesteem®, Claravis®, Clarus®, Decutan®, Isotane®, Izotech®, Oratane®, Isotret®, and Sotret®), bexarotene (e.g. Targretin®); Glucocorticosteroids: Hydrocortisone (e.g. cortisone, hydrocortisone sodium succinate, hydrocortisone sodium phosphate, and e.g. Ala-Cort®, Hydrocortisone Phosphate, Solu-Cortef®, Hydrocort Acetate® and Lanacort®), dexamethasone, prednisolone (e.g. Delta-Cortel®, Orapred®, Pediapred® and Prelone®), prednisone (e.g. Deltasone®, Liquid Red®, Meticorten® and Orasone®), methylprednisolone (e.g. 6-Methylprednisolone, Methylprednisolone Acetate, Methylprednisolone Sodium Succinate, e.g. Duralone®, Medralone®, Medrol®, M-Prednisol® and Solu-Medrol®); Cytokines: interleukin-2 (e.g. aldesleukin and IL-2, e.g. Proleukin®), interleukin-11 (e.g. oprevelkin, e.g. Neumega®), alpha interferon alfa (e.g. IFN-alpha, e.g. Intron® A, and Roferon-A®); Leutinizing hormone releasing hormone (LHRH) agonists: Goserelin (e.g. Zoladex®); Progesterones: megestrol (e.g. megestrol acetate, e.g. Megace®); Miscellaneous cytotoxic agents: Arsenic trioxide (e.g. Trisenox®), asparaginase (e.g. L-asparaginase, Erwinia L-asparaginase, e.g. Elspar® and Kidrolase®); Anti-nausea drugs: NK-1 receptor antagonists: Casopitant (e.g. Rezonic® and Zunrisa® by GlaxoSmithKline); and Cytoprotective agents: Amifostine (e.g. Ethyol®), leucovorin (e.g. calcium leucovorin, citrovorum factor and folinic acid).

### Biological Assays

Compounds designed, selected and/or optimised by methods described above, once produced, can be characterised using a variety of assays known to those skilled in the art to determine whether the compounds have biological activity. For example, the molecules can be characterised by conventional assays, including but not limited to those assays described below, to determine whether they have a predicted activity, binding activity and/or binding specificity.

In some embodiments, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Potent Inhibition

Compounds and compositions of the disclosure are potent inhibitors of one or more oncogenic variants of an EGFR. Alternatively, or in addition, compounds and compositions of the disclosure are potent inhibitors of one or more of a wild type HER-2 receptor or an oncogenic variant of a HER-2 receptor. In some embodiments, the oncogenic variant of a HER-2 receptor is an allosteric variant of a HER-2 receptor.

Tables A and B assign each compound a potency code: A, B, C, D, E, F, G, H, I, J or K. According to the code, A represents an IC50 value ≤5 nM. B represents an IC50 value >5 nM and ≤10 nM. C represents an IC50 value >10 nM and ≤20 nM. D represents an IC50 value >20 nM and ≤30 nM. E represents an IC50 value >30 nM and ≤50 nM. F represents an IC50 value >50 nM and ≤100 nM. G represents an IC50 value >100 nM and ≤200 nM. H represents an IC50 value >200 nM and ≤300 nM. I represents an IC50 value >300 nM and ≤500 nM. J represents an IC50 value >500 nM and ≤1000 nM. K represents an IC50 value >1000 nM.

TABLE A
Activity for Inhibiting EGFR

| Compound No. | EGFR WT | EGFR V3 | EGFR NPH | EGFR SVD |
|---|---|---|---|---|
| 1 | J | F | | |
| 2 | K | E | | |
| 3 | H | C | C | C |
| 4 | J | E | | |
| 5 | H | G | | |
| 6 | G | E | | |
| 7 | I | G | | |
| 8 | G | D | E | E |
| 9 | I | E | E | E |
| 10 | G | D | E | E |

TABLE B
Activity for Inhibiting HER2

| Compound No. | HER2 WT | HER2 S310F | HER2 YVMA |
|---|---|---|---|
| 1 | D | G | |
| 2 | D | G | |
| 3 | A | B | D |
| 4 | C | G | |
| 5 | B | G | |
| 6 | A | C | |
| 7 | C | G | |
| 8 | B | C | G |
| 9 | B | C | F |
| 10 | A | D | F |

In some embodiments, the compound is capable of inhibiting a mutant EGFR (e.g., EGFR-Viii, EGFR-NPH, or EGFR-SVD).

In some embodiments, the compound exhibits an IC₅₀ value of about 100 nM or less, about 80 μM or less, about 60 nM or less, about 50 nM or less, about 40 nM or less, about 30 nM or less, about 20 nM or less, about 10 nM or less, or about 5 nM or less for inhibiting a mutant EGFR (e.g., EGFR-Viii, EGFR-NPH, or EGFR-SVD).

In some embodiments, the compound exhibits an IC50 value of about 100 nM or less for inhibiting EGFR-Viii. In some embodiments, the compound is selected from the group consisting of 1, 2, 3, 4, 6, 8, 9, and 10, or a pharmaceutically acceptable salt or stereoisomer thereof.

In some embodiments, the compound exhibits an IC₅₀ value of about 50 nM or less for inhibiting EGFR-Viii. In some embodiments, the compound is selected from the group consisting of 2, 3, 4, 6, 8, 9, and 10, or a pharmaceutically acceptable salt or stereoisomer thereof.

In some embodiments, the compound exhibits an IC₅₀ value of about 30 nM or less for inhibiting EGFR-Viii. In some embodiments, the compound is selected from the group consisting of 3, 8, and 10, and a pharmaceutically acceptable salt and a stereoisomer thereof.

In some embodiments, the compound exhibits greater inhibition of a mutant EGFR (e.g. EGFR-Viii, EGFR-NPH, or EGFR-SVD) relative to wild-type EGFR.

In some embodiments, the compound exhibits at least about 2-fold, about 3-fold, about 5-fold, about 10-fold, about 20-fold, about 30-fold, about 50-fold, or about 100-fold greater inhibition of a mutant EGFR (e.g., EGFR-Viii, EGFR-NPH, or EGFR-SVD) relative to wild-type EGFR.

In some embodiments, the compound exhibits at least about 5-fold greater inhibition of EGFR-Viii relative to wild-type EGFR. In some embodiments, the compound is selected from the group consisting of 1, 2, 3, 4, 8, 9, and 10, or a pharmaceutically acceptable salt or stereoisomer thereof.

In some embodiments, the compound exhibits at least about 10-fold greater inhibition of EGFR-Viii relative to wild-type EGFR. In some embodiments, the compound is selected from the group consisting of 1, 2, 3, 4, and 9, or a pharmaceutically acceptable salt or stereoisomer thereof.

In some embodiments, the compound exhibits at least about 20-fold greater inhibition of EGFR-Viii relative to wild-type EGFR. In some embodiments, the compound is selected from the group consisting of 2, and 4, or a pharmaceutically acceptable salt or stereoisomer thereof.

In some embodiments, the compound is capable of inhibiting wild-type HER2 or a mutant HER2 (e.g., HER2-S310F or HER2-YVMA).

In some embodiments, the compound exhibits an IC₅₀ value of about 100 nM or less, about 80 nM or less, about 60 nM or less, about 50 nM or less, about 40 nM or less, about 30 nM or less, about 20 nM or less, about 10 nM or less, or about 5 nM or less for inhibiting wild-type HER2 or a mutant HER2 (e.g., HER2-S310F or HER2-YVMA).

In some embodiments, the compound exhibits an IC50 value of about 100 nM or less, about 80 nM or less, about 60 nM or less, about 50 nM or less, about 40 nM or less, about 30 nM or less, about 20 nM or less, about 10 nM or less, or about 5 nM or less for inhibiting wild-type HER2.

In some embodiments, the compound exhibits an IC₅₀ value of about 20 nM or less for inhibiting wild-type HER2. In some embodiments, the compound is selected from the group consisting of 3, 4, 5, 6, 7, 8, 9, and 10, or a pharmaceutically acceptable salt or stereoisomer thereof.

In some embodiments, the compound exhibits an IC₅₀ value of about 10 nM or less for inhibiting wild-type HER2. In some embodiments, the compound is selected from the group consisting of 3, 5, 6, 8, 9, and 10, or a pharmaceutically acceptable salt or stereoisomer thereof.

In some embodiments, the compound exhibits an IC₅₀ value of about 6 nM or less for inhibiting wild-type HER2. In some embodiments, the compound is selected from the group consisting of 3, 6, and 10, or a pharmaceutically acceptable salt or stereoisomer thereof.

In some embodiments, the compound exhibits an IC₅₀ value of about 100 nM or less, about 80 μM or less, about 60 μM or less, about 50 μM or less, about 40 μM or less, about 30 μM or less, about 20 μM or less, about 10 μM or less, or about 5 μM or less for inhibiting a mutant HER2 (e.g., HER2-S310F or HER2-YVMA).

In some embodiments, the compound exhibits an IC₅₀ value of about 50 nM or less for inhibiting HER2-S310F. In some embodiments, the compound is selected from the group consisting of 3, 6, 8, 9, and 10, or a pharmaceutically acceptable salt or stereoisomer thereof.

In some embodiments, the compound exhibits an IC₅₀ value of about 20 nM or less for inhibiting HER2-S310F. In some embodiments, the compound is selected from the group consisting of 3, 6, 8, and 9, or a pharmaceutically acceptable salt or stereoisomer thereof.

### Paradoxic ErbB Receptor Activation

Although the mechanisms described herein apply to any form of cancer in which these EGFR variants of the disclosure are expressed, the prevalence of these variants in glioblastoma (GBM) are provide by way of example. Other cancers expressing the EGFR variants of the disclosure include, but are not limited to, solid cancers, epithelial cancers and/or cancers of epithelial origin, bladder cancer, breast cancer, cervical cancer, colorectal cancer, endometrial cancer, gastric cancer, glioblastoma (GBM), head and neck cancer, lung cancer, and non-small cell lung cancer (NSCLC).

In GBM tumors EGFR is frequently the target of genomic mutations and alternative splicing events that result in alteration of the extracellular dimer interface. Many tumors express more than one aberrant isoform. The disclosure provides the mechanism of activation for the most commonly occurring variants, EGFR-Viii, EGFR-Vii, EGFR-Vvi, and EGFR-A289V. Although each isoform is the result of a distinct ectodomain alteration, all are activated by a common mechanism involving covalent ligand-independent dimerization.

AMG-595 (Amgen) is an EGFR-Viii isoform selective antibody that has no activity against wild type EGFR or other splice-activated variants. Rindopepimut (Celldex) is a vaccine the produces an immunological response selectively against tumor cells expressing EGFR-Viii but not wild type EGFR or other splice-activated isoforms. Other EGFR isoforms expressed in GBM tumors (EGFR-Vii and EGFR-Vvi) are constitutively active covalent receptors and their expression may limit the breadth and duration of treatment benefit for an ErbB inhibitor that is selective only for EGFR-Viii. Therefore, it may be useful to exclude patients whose tumors express EGFR-Vii, EGFR-Vvi, or EGFR ectodomain point mutants from treatment with an EGF R-Viii selective therapy.

The heterogenetic expression pattern for multiple ectodomain variants of ErbB receptors in tumors indicates that a small molecule inhibitor that inhibits all variants may be preferred.

The family of covalently-activated EGFR isoforms responds very differently to small molecule ErbB inhibitors compared to EGFR catalytic domain mutations observed in NSCLC. Importantly, Type I inhibitors, including erlotinib, all induce the formation of covalent EGFR dimers and increase EGFR phosphorylation at sub-saturating concentrations, an activity that is further enhanced when ErbB inhibitor is washed away. This manifests in paradoxical activation of proliferation at sub-saturating concentrations.

The discovery of paradoxical activation of proliferation at sub-saturating concentrations of Type I ErbB inhibitors is further demonstrated for a series of extracellular variants of HER2, prevalent in a number of cancers including breast and bladder. All variants existed as covalently activated receptors, and levels of covalent dimers increased following treatment with Type I inhibitors including sapitinib and afatinib. As with covalently-activated EGFR variants, sub-saturating doses of Type I inhibitors paradoxically increased phosphorylation of HER2 variants, increasing the proliferation of cells expressing them.

In contrast to Type I inhibitors, the disclosure demonstrates that Non-Type I (e.g. Type IT) inhibitors including neratinib are devoid of paradoxical activation for cells expressing ErbB ectodomain variants. Neratinib is found to exemplify a molecule that is both potent and selective for each member of the covalently-activated EGFR family versus wild type EGFR.

In some embodiments, the disclosure provides a structure/functional relationship for predicting how structural variations affecting receptor regions distal to the active site can confer dramatically different responses to small molecule active site inhibitors. The disclosure described herein of paradoxical activation of covalently-activated ErbB receptor variants by Type I inhibitors has important clinical implications. The data of the disclosure provide a mechanistic explanation for the failed clinical studies for Type I inhibitors in tumor types where expression of covalently-activated ErbB receptors is prevalent. This includes erlotinib and gefitinib in GB tumors, erlotinib in SCCHN tumors, and sapitinib in breast tumors. In some embodiments, the disclosure provides methods of using tumor expression levels for covalently-activated ErbB receptors as exclusion criteria for treating patients with a Type I ErbB receptor inhibitor therapeutic.

### Glioblastoma

Glioblastoma (GBM), grade IV astrocytoma, is the most common form of brain cancer. The outcome for this disease is dismal. Surgery followed by a therapeutic regimen of radiation and temozolomide is standard of care, however this produces a median overall survival (OS) of only 14.6 months and few patients survive for five years. There has been little progress made in extending survival for GBM patients over the past decade. Although bevacizumab showed an improved progression free survival benefit in the recurrent setting, the addition of bevacizumab to standard of care therapy in the front-line setting did not result in an OS benefit.

EGFR is the most frequently altered oncogene in GBM. In addition to EGFR gene amplification, many tumors express variants generated by aberrant splicing or genomic mutation. The first recognized variant is EGFR-Viii, resulting from truncation of exons 2-7 and expressed by approximately 30% of GBM tumors. EGFR-Viii is oncogenic. EGFR-Viii is constitutively activated in the absence of EGF ligand, exhibiting sustained signaling that is resistant to downregulation. Therefore, EGFR-V Iii is both transforming and tumorigenic. Expression of EGFR-Viii is associated with poor long term overall survival in GBM. RNA sequencing data has revealed that EGFR-Viii is just one of several aberrantly spliced variants of EGFR expressed in GBM tumors. Two others result in truncation of exons 12-13 and 14-15 (EGFR-Vii). Like EGFR-Viii, EGFR-Vii is both transforming and tumorigenic. In addition to splice variants, (IBM tumors also express a collection of EGFR point mutations including C620Y and A289V, which are transforming and tumorigenic. The complex landscape of EGFR alterations in GBM is further compounded by the observation that many tumors express more than one receptor variant.

Because the expression of multiple EGFR variants in GBM gives rise to transforming and tumorigenic activity and because EGFR is the most frequently altered oncogene present in GBM tumors, EGFR is an especially attractive target for small molecule ErbB inhibitors. Following the success for small molecule EGFR therapeutics against NSCLC tumors harboring activating mutations in EGFR (erlotinib, gefitinib, and afatinib), these drugs were tested in GBM. Despite intense clinical investigation of this group of ErbB inhibitors in GBM, involving >30 clinical trials and >1500 patients, all failed to produce any benefit, even for those tumors that expressed EGFR-Viii. Some evidence suggests that erlotinib promoted disease progression. A phase 2 study evaluating erlotinib in combination with radiation and temozolomide showed median PFS (mPFS) and median OS (mOS) of 2.8 months and 8.6 months, as compared to 6.9 months and 14.6 months for patients receiving radiation and temozolomide alone. Another randomized phase II trial with erlotinib showed that patients who received erlotinib, including those whose tumors expressed EGFR-Viii, performed worse by a number of parameters than those patients who received standard of care therapy. The clinical failures for ErbB inhibitors such as erlotinib in GBM tumors has cast doubt on the role of EGFR as a driver of tumor growth in GBM and led to inquiry as to why ErbB inhibitors that were so effective in treating EGFR mutations in lung cancer were so ineffective in treating EGFR variants in GBM.

A distinctive feature for the EGFR variants expressed in GBM is their location within the extracellular domain. This is in contrast to activating mutations of EGFR found in lung cancer, which often reside in the intracellular catalytic domain. EGFR is composed of four extracellular domains (two ligand binding domains and two cysteine rich regions), a transmembrane domain, and an intracellular catalytic domain. Ligand binding promotes dimerization of the extracellular cysteine rich domains (CR1 and CR2), an event that confers dimerization of the intracellular domain and activation of receptor catalytic activity. Nearly all EGFR splicing events and mutations in GBM affect the extracellular region. (e.g., two cysteine rich regions (CR1 and CR2) that form the extracellular dimer interface). The CR regions contain >40 cysteine residues, all of which form intramolecular disulfide bonds. In EGFR-Viii, truncation of exons 2-7 results in partial loss of sequence encoding the CR1 region. A consequence is loss of one cysteine from the Cys295-Cys307 pair, leaving Cys307 as a free unpaired cysteine. For EGFR-Viii, this cysteine can form an intermolecular disulfide bond with another EGFR monomer to drive a covalently dimerized and constitutively activated receptor. Mutation of Cysteine 307 to a Serine (C307S) prevents the formation of covalently dimerized EGFR-Viii and is inactive.

Although several recent preclinical studies have suggested that EGFR kinase inhibitors such as erlotinib are quite ineffective at inhibiting EGFR-Viii, there has been no mechanism proposed for this effect. There is also a lack in current understanding for the mechanism responsible for activation of other ectodomain variants in GBM, including EGFR-Vii and EGFR-A289V. The disclosure provides a mechanism of receptor activation and impact on ErbB inhibitor activity for a group of four of the most common ectodomain variants in GBM EGFR-Viii, EGFR-Vii, EGFR-delta 12-13, and EGFR-A289V.

The disclosure demonstrates that like EGFR-Viii, an additional group of commonly occurring EGFR variants in GBM (EGFR-Vii, EGFR-Vvi, and EGFR-A289V) all exist as constitutively active covalent dimers and together form a family of EGFR isoforms that are activated by this common mechanism. Furthermore, the disclosure shows that the propensity of these variants to covalently dimerize is coupled to the conformation of the intracellular catalytic site, conferring distinct activity for classes of small molecules inhibitors binding to this distal site. Inhibitors that stabilize the active conformation of the kinase (Type I inhibitors, including erlotinib) induce the formation of covalent dimers for all covalently-activated EGFR isoforms. This is associated with the propensity of Type I inhibitors to increase EGFR phosphorylation at sub-saturating concentrations and to paradoxically stimulate the proliferation of cells expressing covalently-activated EGFR isoforms. Neither enhanced dimerization nor paradoxical activation of EGFR is seen with small molecule inhibitors that stabilize the inactive kinase conformation (Type II inhibitors, including lapatinib and neratinib). Examples of Type II inhibitors were identified that were potent inhibitors of covalently-activated EGFR isoforms and which were selective for this family compared to WT-EGFR.

Similar to the mutations identified for EGFR, the disclosure identifies a group of splice events and mutations affecting the CR domains of HER2 and HER4. The disclosure demonstrates that this group of splice events and mutations affecting the CR domains of HER2 and HER4 exists as covalent dimers and are paradoxically activated by agents with a Type I binding mode. These data provide a mechanistic explanation for the failure of multiple clinical trials involving Type I inhibitors, including >30 clinical trials of Type I ErbB inhibitors in GBM. Collectively these data indicate that tumors expressing covalently-activated EGFR isoforms should be excluded from treatment with Type I ErbB inhibitors such as erlotinib because of paradoxical activation. These data further demonstrate the utility for optimizing Type II ErbB inhibitors against the covalently-activated ErbB family.

### Clinical Trials Using Type I ErbB Inhibitors

In some embodiments, methods of the disclosure identify subjects expressing ErbB family receptor variants in one or more cancer cells or cancer cell types of the subject. Identification of a subject as having a variant of the disclosure may be used as either inclusion or exclusion criteria for either a clinical trial to assess the efficacy of an existing or novel cancer treatment or for an approved treatment protocol.

In some embodiments, the methods of the disclosure may be used to exclude patients expressing one or more of the ErbB variants of the disclosure from a clinical trial assessing the safety and/or efficacy of a Type I inhibitor of the disclosure. The ErbB variants of the disclosure are paradoxically activated upon contact with a Type I inhibitor, leading to increased proliferation of the cancer cell. In past and ongoing clinical trials, the patient populations used for these studies had not been screened for expression of an ErbB variant of the disclosure. Consequently, a Type I inhibitor of the disclosure that “failed” a clinical trial by failing to show increased efficacy over a standard treatment or placebo for the treatment of cancer may, in fact, be effective but the results may have been confounded by the inclusion of patients who express an ErbB variant of the disclosure. Because patients who express an ErbB variant of the disclosure may demonstrate increased proliferation of cancer cells when treated with a Type I inhibitor, and, therefore, demonstrate a lack of improvement or even a further progression of the cancer, these patients may prevent approval of cancer therapeutics that could be life-saving for those patients who do not express an ErbB receptor variant of the disclosure. In some embodiments, the methods of the disclosure include identifying a subject as expressing an ErbB receptor variant of the disclosure and excluding this patient from treatment with a Type I inhibitor. In some embodiments, a patient who expresses an ErbB receptor variant of the disclosure may be successfully treated with a Non-Type I inhibitor, including a Type II inhibitor.

In some embodiments, when a patient who expresses an ErbB receptor variant of the disclosure is identified as expressing only the EGFR-Viii splice variant, the patient may be treated with an EGFR-Viii selective inhibitor or may be included in a clinical trial for an EGFR-Viii selective inhibitor. In some embodiments of the methods of the disclosure, the patient should express only the EGFR-Viii splice variant to be treated with an EGFR-Viii selective inhibitor. In some embodiments, if the patient expresses multiple variants, including the EGFR-Viii variant, resulting in a combination of expressed variants, the patient should be excluded from treatment with an EGFR-Viii selective inhibitor, however, this patient may be successfully treated with a Non-Type I selective inhibitor (e.g. a Type II inhibitor).

In some embodiments, should a selective inhibitor target any one or more of the ErbB receptor variants of the disclosure, the identification of expression of the splice variant in a patient may be used as an inclusion criterion for a clinical study or treatment regimen providing that selective inhibitor.

Table I provides a listing of exemplary clinical trials for Type I inhibitors that “failed” when in tumor types that express covalently activated ErbB receptors were included in the study. In some embodiments, the disclosure provides a method of screening or re-screening participants in a clinical trial for expression of one or more covalently activated ErbB receptor variants of the disclosure. In some embodiments, the methods of the disclosure include treating those patients who do not express one or more covalently activated ErbB receptor variants of the disclosure for a first or subsequent attempt with a Type I inhibitor to determine efficacy of the Type I inhibitor in a tumor type or patient that does not express one or more covalently activated ErbB receptor variants of the disclosure. In some embodiments, those patients who are excluded from a first or subsequent treatment with a Type I inhibitor may be treated with a Non-Type I inhibitor of the disclosure, including a Type II inhibitor.

TABLE 1
Listing of clinical trials for Type I inhibitors that failed in tumor
types where expression levels of covalently activated
ErbB receptors is prevalent.

| Type I inhibitor | Tumor Setting | Study |
|---|---|---|
| erlotinib | GBM | Van den Bent et al. J Clin Oncol., |
| | | 2009 |
| erlotinib | GBM | Peereboom et al. J Neuro-oncol., 2010 |
| afatinib | GBM | Reardon et al. Neuro Oncol., 2014 |
| gefitinib | SCCHN | Argiris et al. J Clin Oncol., 2013 |
| erlotinib | SCCHN | Martins et al. J Clin Oncol. 2013 |
| gefitinib | bladder | Petrylak et al. BJU Int. 2010 |
| gefitinib | bladder | Philips et al. Ann Oncol. 2009 |
| sapitinib | breast | NCT00900627/THYME |
| sapitinib | breast | NCT01151215 |

Table 2 provides a listing of exemplary ErbB inhibitors of the disclosure. In some embodiments, methods of the disclosure may include the identification or determination of expression of an ErbB receptor of the disclosure as either an exclusion criteria for treatment or a clinical trial administering a Type I inhibitor or as inclusion criteria for treatment or a clinical trial administering a Non-Type I (e.g. Type II) inhibitor or the NT-113 Type I inhibitor.

TABLE 2
Exemplary ErbB inhibitors

| Inhibitor Name | CAS Number | Type I? |
|---|---|---|
| CUDC-101 | 1012054-59-9 | Yes |
| poziotinib (HM781-36B) | 1092364-38-9 | Yes |
| dacomitinib (PF-299804) | 110813-31-4 | Yes |
| JNJ-26483327 | 1131863-89-2 | Yes |
| WZ 4002 | 1213269-23-8 | Yes |
| WZ 3146 | 1214265-56-1 | Yes |
| WZ8040 | 1214265-57-2 | Yes |
| AP-26113 | 1350848-43-9 | Yes |
| Rociletinib (CO 1686, | 1374640-70-6 | Yes |
| AVL301) | | |
| NT-113 | 1398833-56-1 | Yes |
| AZD9291 | 1421373-65-0 | Yes |
| erlotinib (OSI-744) | 183319-69-9 | Yes |
| gefitinib (ZD1839) | 184475-35-2 | Yes |
| PKI 166 | 187724-61-4 | Yes |
| PD 168393 | 194423-15-9 | Yes |
| BIBX 1382 | 196612-93-8 | Yes |
| vatalanib (CGP79787) | 212141-54-3 | Yes |
| lapatinib | 231277-92-2 | No** |
| pelitinib (EKB-569) | 257933-82-7 | Yes |
| Canertinib (Cl-1033) | 267243-28-7 | Yes |
| afatinib (BIBW2992) | 439081-18-2 | Yes |
| vandetanib (ZD6474) | 443913-73-3 | Yes |
| AEE788 | 497839-62-0 | Yes |
| icotinib (BPI-2009H) | 610798-31-7 | Yes |
| dovitinib | 692737-80-7 | Yes |
| neratinib (HKI-272) | 698387-09-6 | No** |
| AC-480 (BMS-599626) | 714971-09-2 | Yes |
| XL-647 | 781613-23-8 | Yes |
| HKI-357 | 848133-17-5 | No** |
| Sapitinib (AZD8931) | 848942-61-0 | Yes |
| TAK 285 | 871026-44-7 | No** |
| AST 1306 | 897383-62-9 | No** |
| AV-412 | 451493-31-5 | Yes |

*Type I inhibitors of the disclosure are characterized by their mode of kinase inhibition which is described by their ability to target the ATP-binding site in an active conformation to competitively inhibit ATP-binding. Key structural elements have been described including an alignment of specific hydrophobic residues.
**Inhibitors of inactive kinases bind to target in such a manner as to disrupt key structural elements of the active conformation, including specific hydrophobic residues. These non-Type I inhibitors are differentiated from Type I inhibitors by their interaction with target in such a way as to prevent the target adopting an active ATP-binding conformation. Non-Type I inhibitors of the disclosure include, but are not limited to Type II inhibitors. Inhibitors that are not Type I inhibitors in this table are Type II inhibitors.

### Paradoxical Stimulation of Proliferation by Type 1 Inhibitors in Cells Driven by Covalently-Activated ErbB Oncoproteins

Although illustrated through the example of EGFR variants in the diagnosis and treatment of glioblastoma, the methods of the disclosure may include ErbB receptor variants (e.g. EGFR, and HER2 variants) in any cancer in which these variants are expressed. An exemplary collection of these variants is provided in Table 3.

TABLE 3
Exemplary Covalent ErbB Oncoproteins

| Receptor | Event | Event Type | Region | Expression |
|---|---|---|---|---|
| EGFR | Viii | splicing | CR1 (deletion of | GBM, |
| | | | exons 2-7) | NSCLC, |
| | | | | SCCNHN |
| EGFR | Vii | splicing | CR2 (deletion of | GBM |
| | | | exons 14-15) | |
| EGFR | Vvi | splicing | CR2 (deletion of | GBM |
| | | | exons 12-13) | |
| EGFR | delta768 | splicing | CR1 (deletion of | neuroblastoma |
| | | | nucleotides 102- | |
| | | | 769) | |
| EGFR | delta660 | splicing | CR1 (deletion of | SCCHN |
| | | | nucleotide 237 of | |
| | | | exon 2 to 896 of | |
| | | | exon 8) | |
| ErbB2 | delta16 | splicing | CR2 (deletion of | GBM |
| | | | exon 16) | |
| ErbB2 | p95HER2 | splicing/altered | AA 1-611 deletion | Breast |
| | | translation start/ | | |
| | | proteolytic | | |
| | | cleavage | | |

With respect to EGFR and glioblastoma, RNA sequencing of 164 GBM tumors reveals heterogenous expression of multiple ectodomain variants of EGFR. Aberrant splicing, alone or coincident with genomic rearrangement, produces EGFR-Viii (loss of exons 2-7), EGFR-Vii (loss of exons 14-15), and EGFR-Vvi (loss of exons 12-13), Table 4.

TABLE 4

| | Tumor | | | |
|---|---|---|---|---|
| | expression | Exons | | |
| Variant | (prevalence) | spliced out | Position | Free Cys generated |
| EGFR-Vii | GBM (3%) | 14-15 | CR2 | Cys539, Cys628, |
| | | | | Cys636 |
| EGFR-Viii | GBM (20%)/ | 2-7 | CR1 | Cys307 |
| | SCCHN (36%)/ | | | |
| | NSCLC (3%)/ | | | |
| | BrCa (5%) | | | |
| EGFR-Vvi | GBM (32%) | 12-13 | CR2 | Cys555 |
| EGFR- | GBM (16%) | NA | CR1 | ND |
| A289V | | | | |

Prevalence is based on expression levels >1% as reported by TCGA data sets (Brennan et al. (2013) Cell 155(2): 462-477).

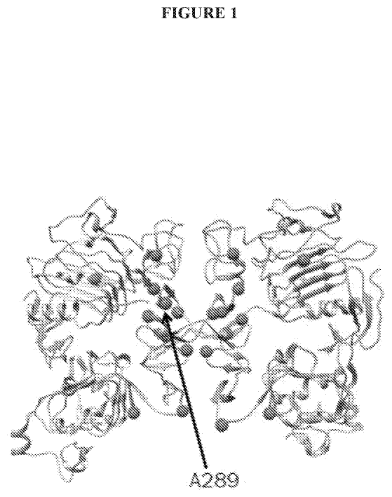

All three ectodomain variants affect the CR1 or CR2 regions and result in loss of exons coding for sequence at the extracellular dimer interface. There is also a series of greater than 20 genomic mutations found in GBM tumors, which also map to the CR1 and CR2 regions at the dimer interface (see, for example, FIG. 1 and Table 5).

TABLE 5

| Mutation | Region |
|---|---|
| R222C | CR1 |
| R252C/P | CR1 |
| R256Y | CR1 |
| T263P | CR1 |
| V270C | CR1 |
| A289T/V/D | CR1 |
| H304Y | CR1 |
| G331R | CR1 |
| P596S/L/R | CR2 |
| G598V/A | CR2 |
| G614D | CR2 |
| C628F/Y | CR2 |
| C636Y | CR2 |
| S645C | CR2 |

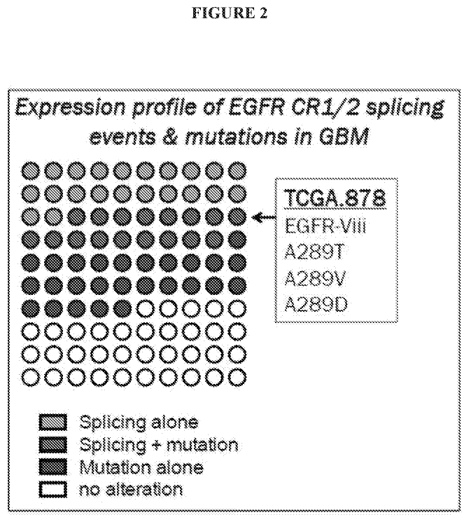

The most common of these affect A289, with A289V being most prevalent. EGFR-Viii is expressed by 20%, Vii by 3% and Vvi by 32% of tumors. Mutations within the extracellular region are observed in 40% of tumors, and at position A289 by 16% of tumors. Expression of at least one variant is observed in 65% of GBM tumors (FIG. 2). Many tumors express multiple variants. This is exemplified by TCGA.878, a GBM tumor expressing EGFR-Viii, A289T, A289V, and A289D (FIG. 2). 69% of tumors expressing EGFR-Viii also co-express at least one other ectodomain variant of EGFR, and several tumors co-expressed all three ectodomain variants. Only 6% of GBM tumors express EGFR-Viii in isolation. Expression of EGFR in GBM tends to be mutually exclusive with expression of other RTK oncogenes, which are co-expressed with EGFR variants in only 7% of GBM tumors. Collectively, these data demonstrate how EGFR alterations in GBM have a dominant and mutually exclusive expression pattern compared with other oncogenic drivers.

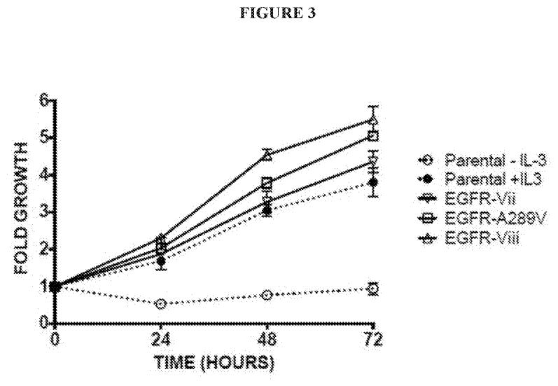

Splicing events and mutations affecting the extracellular ligand binding domain have been shown to be both transforming and tumorigenic. The data of the disclosure confirmed the transforming properties for EGFR-Viii, EGFR-Vii, and EGFR-A289V. When expressed in BaF3 cells all transformed cells to proliferate in the absence of IL-3 (FIG. 3).

The x-ray structure for the ectodomain of wild type EGFR reveals 21 intramolecular disulfide bonds lining the dimer interface at the CR1 and CR2 regions. Exemplary disulfide bonds lining the dimer interface at the CR1 and CR2 regions may occur at one or more regions of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1. Exemplary disulfide bonds lining the dimer interface at the CR1 and CR2 regions of a HER-2 receptor may occur at one or more regions of C199-C212, C220-C227, C224-C235, C236-C244, C240-C252, C255-C264, C268-C295, C299-C311, C315-C331, C334-C338, C342-C367, C511-C520, C531-C540, C544-C560, C563-C576, C567-C584, C587-C596, C600-C623, C626-C634 and C630-C642.

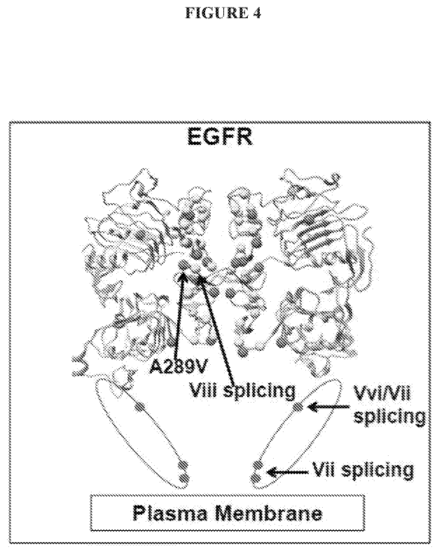

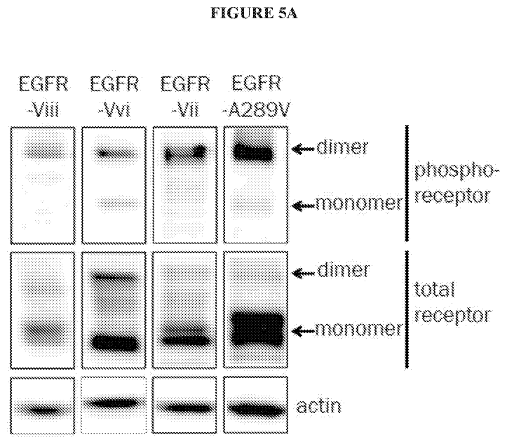

This is a common feature for all ErbB receptors. One of the 11 intramolecular disulfide bonds in the CR1 region of EGFR is formed by Cys295-Cys307, which is disrupted in EGFR-Viii. Loss of sequence coding for part of the CR1 region eliminates Cys295, leaving Cys307 free to form an intermolecular disulfide bond with another EGFR-Viii monomer (FIG. 4). The mutation Cys307-Ser prevents formation of covalent EGFR-Viii dimers and exhibits reduced tumorigenicity in vivo.

Inspection of sequences losses produced by truncations for both EGFR-Vvi and EGFR-Vii reveals that intramolecular disulfide bonds at the CR2 ectodomain dimer interface will be disrupted. Loss of exons 14-15 in EGFR-Vvi will result in disruption of the Cys539-Cys555 bond, leaving Cys555 as a free cysteine, and loss of exons 14-15 in EGFR-Vii will result in disruption of the Cys539-Cys555, Cys620-Cys628 and Cys624-Cys636 bonds, leaving Cys555, Cys628 and Cys636 as free cysteines. Cys555, Cys628, and Cys636 all reside in the CR2 region of the dimerization interface, FIG. 4. Free cysteines generated at these sites could confer the potential for receptors to form covalent dimers, as has been demonstrated for EGFR-Viii.

Most point mutations reside in cysteine rich regions CR1 and CR2 and could also affect disulfide bonds at the ectodomain dimer interface (FIG. 1). Some point mutations introduce new cysteines into the CR1 region (e.g. R252C). Other mutations directly affect cysteines that form intramolecular disulfide bonds in the CR2 region of wild type EGFR (e.g. C624F), and some of these have been shown to promote covalently dimerized receptors in the presence of EGF ligand. Many other mutations do not directly affect cysteine composition within the ectodomain but are situated in close proximity to native intramolecular disulfide bonds at the dimer interface, and offer the potential to disrupt these structures. Mutations that are adjacent to a disulfide bond in the third Ig-like domain of FGFR2 have been shown to disrupt this bond and confer a covalently dimerized and activated receptor. A289, the most common site for mutation in GBM, is less than 10 angstroms from the Cys-295-Cys307 bond, and alterations at this site might disrupt this disulfide, resulting in presentation of free cysteines at the CR1 dimer interface region.

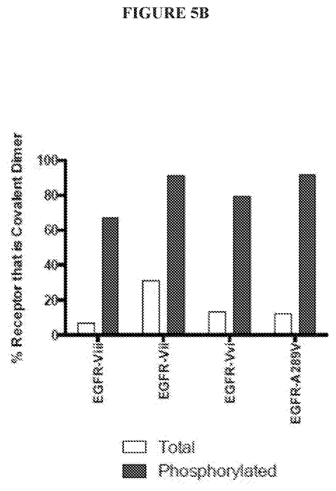

The occurrence of free cysteines at the ectodomain dimer interface for EGFR-Vvi, EGFR-Vii, and EGFR-A289V could give rise to covalent and constitutively active dimers as has been demonstrated for EGFR-Viii. To test this hypothesis, each receptor isoform was expressed in U87-MG tumor cells, which endogenously express only a very low level of wild type EGFR, and evaluated for the phosphorylation of EGFR under non-reducing conditions to allow detection of covalently dimerized versus monomeric receptor. EGFR-Viii, EGFR-Vii, EGFR-Vvi, and EGFR-A289V were all present as covalent and active receptors (FIG. 5). Although covalent dimer represented only a minor fraction of total receptor levels, the majority of phosphorylated and activated receptors were present as covalent dimers. Therefore, distinct rearrangements within the ectodomain generated by genomic alterations and aberrant splicing all produce receptors activated by a common mechanism involving ligand independent covalent dimerization.

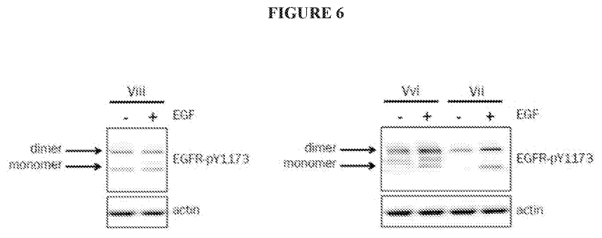

The ability of EGF ligand to modulate the activity for each member of the splice-activated EGFR family was assessed. In EGFR-Viii the ligand binding domain has been mostly truncated because of loss of sequence encoded by exons 27. Consistently, the addition of EGF has no effect on the phosphorylation of monomeric or covalently dimerized EGFR-Viii expressed in U87-MG cells (FIG. 6). The ectodomain truncations for both EGFR-Vii and EGFR-Vvi occur downstream and affect sequence within the CR2 region more proximal to the transmembrane domain. The EGF binding site is intact for both of these variants. In contrast to EGFR-Viii, both EGFR-Vii and EGFR-Vvi have constitutive basal activity for covalent dimers, which can be further enhanced by EGF (FIG. 6).

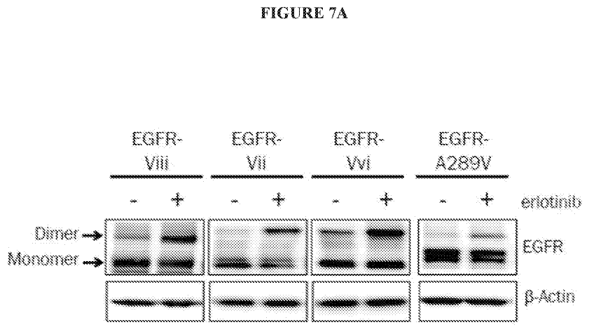

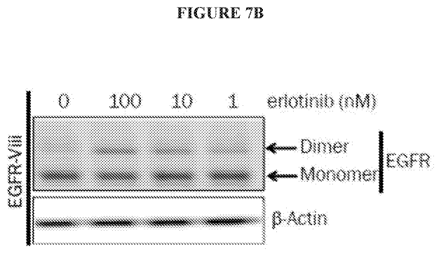

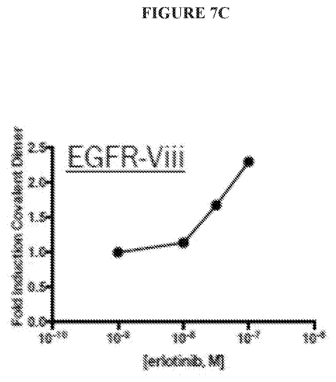

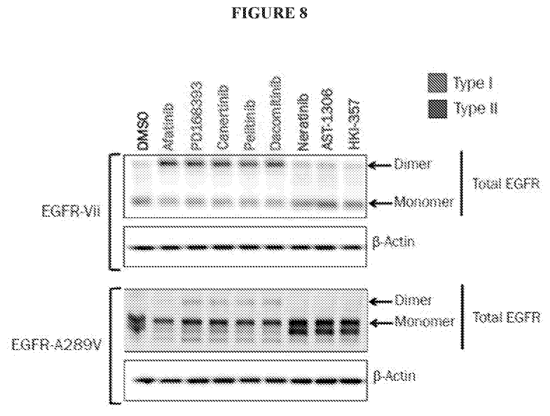

The ability of multiple aberrations of EGFR in GBM to drive constitutive activation indicates that EGFR is an important therapeutic target. However, none of the ErbB inhibitors approved for treatment of EGFR catalytic site mutations in NSCLC proved effective in treating GBM. The experiments of the disclosure sought to establish whether small molecule ErbB inhibitors that have demonstrated clinical activity against oncogenic catalytic mutations expressed in NSCLC might have differential activity against each of the covalently-activated EGFR isoforms. Herein, the data demonstrate that erlotinib enhances the formation of covalent dimers for all three splice-activated EGFR isoforms and EGFR-A289V (FIG. 7A). These effects were dose-dependent (FIG. 7B). This ability of erlotinib to induce covalent dimers for covalently-activated EGFR variants was observed for all Type I ErbB inhibitors, but not Type II inhibitors, and includes molecules with either reversible or covalent binding modes (FIG. 8 and Table 6).

TABLE 6

| Molecule | Binding Mode | Class | Induced dimers |
|---|---|---|---|
| Erlotinib | reversible | Type I | Yes |
| Gefitinib | reversible | Type I | Yes |
| Lapatinib | reversible | Type II | No |
| Afatinib | covalent | Type I | Yes |
| CO-1686 | covalent | Type I | Yes |
| AZD9291 | covalent | Type I | Yes |
| WZ8040 | covalent | Type I | Yes |
| WZ3146 | covalent | Type I | Yes |
| WZ4002 | covalent | Type I | Yes |
| Neratinib | covalent | Type II | No |
| HKI-357 | covalent | Type II | No |
| PD168393 | covalent | Type I | Yes |
| Canertinib | covalent | Type I | Yes |
| Pelitinib | covalent | Type I | Yes |
| Dacomitinib | covalent | Type I | Yes |
| AST-1306 | covalent | Type II | No |

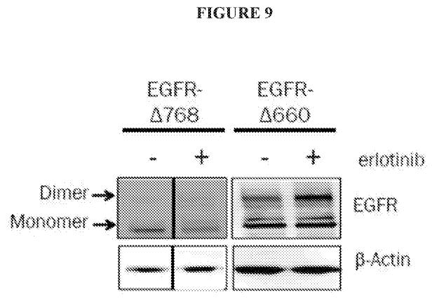

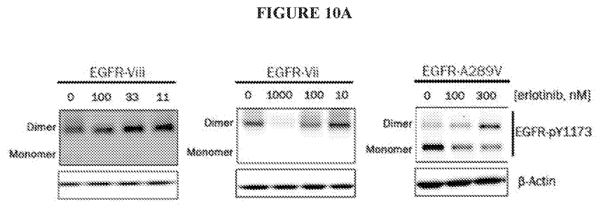

This discovery was extended to two other splice variants that were identified in glioblastomna and head and neck cancers, EGFR-Δ768 and EGFR-Δ660 (FIG. 9 and Table 7). Both receptor isoforms could exist as covalently activated receptors, and erlotinib induced covalent dimerization for both.

TABLE 7

| | Tumor expression | Exons | | Free Cys |
|---|---|---|---|---|
| Variant | (prevalence) | spliced out | Position | generated |
| EGFR-Δ768 | Neuroblastoma (NA) | 2-7 | CR1 | Cys291 |
| | | (partial) | | |
| EGFR-Δ660 | SCCHN (NA) | 2-8 | CR1 | Cys 307 |
| | | (partial) | | |

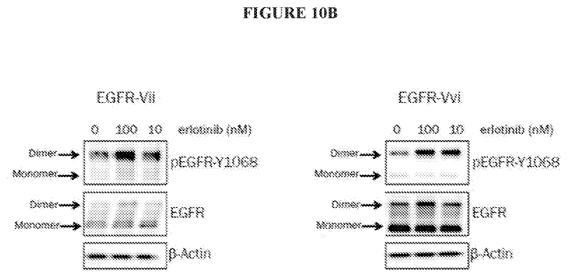

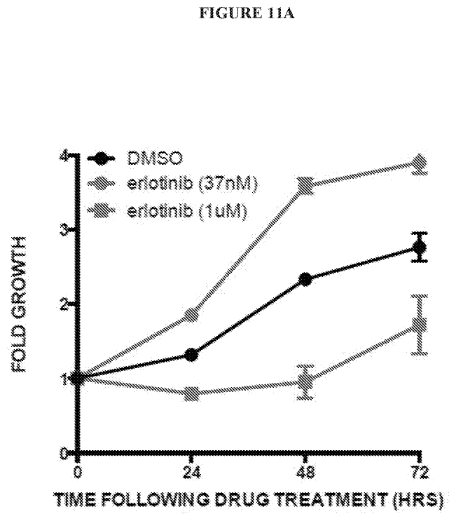

Treatment with sub-saturating concentrations of the Type I ErbB inhibitor erlotinib also results in enhanced phosphorylation of covalently-activated EGFR variants, shown for EGFR-Vii, EGFR-Viii, and EGFR-A289V (FIG. 10A). Further, when cells expressing either EGFR-Vii or EGFR-Vvi are treated with erlotinib, and then washed prior to collection of lysates, all show dramatically enhanced phosphorylation compared to untreated control cells, consistent with increased diner formation in response to the Type I inhibitor (FIG. 10B).

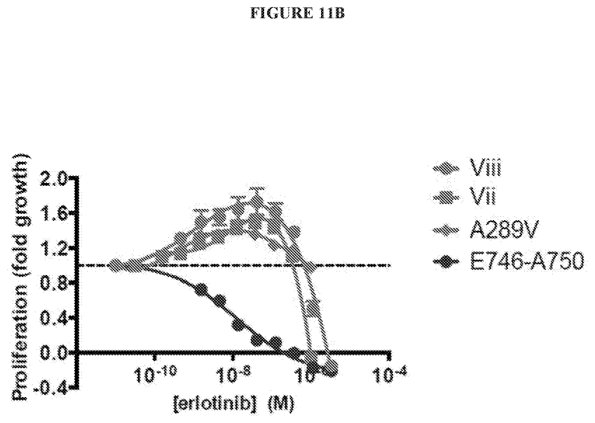

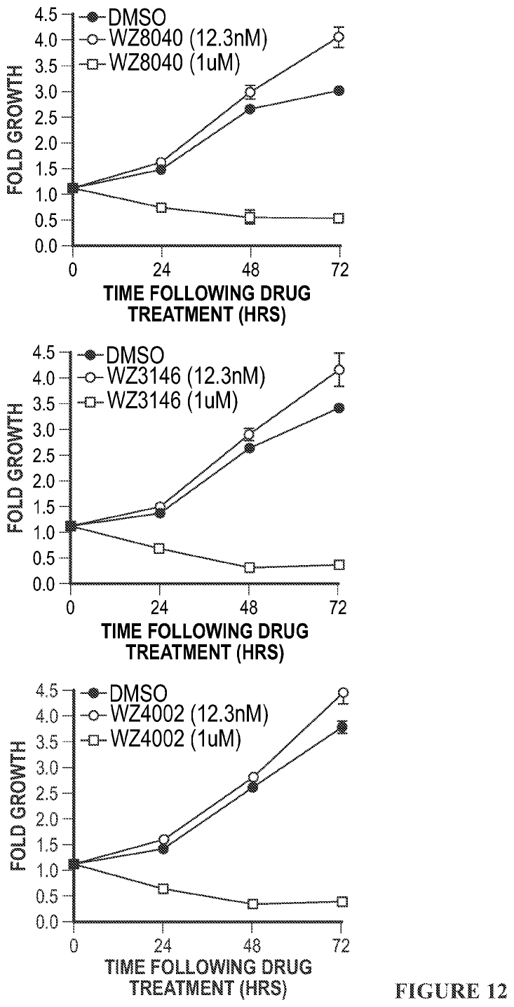

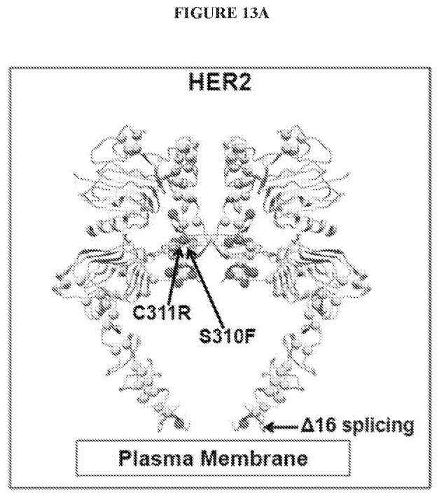

To assess the impact of enhanced EGFR activity evoked by sub-saturating concentrations of erlotinib on cell proliferation, EGFR-Viii, EGFR-Vii, and EGFR-A289V were expressed in BaF3 cells to transform them to IL-3 independence. While high saturating concentrations of erlotinib (1 uM) inhibited proliferation of BaF3-EGFR-Viii cells, lower sub-saturating concentrations (37 nM) stimulated proliferation (FIG. 11A). The biphasic effect of erlotinib on the proliferation of cells expressing covalently-activated EGFR was similarly seen in BaF3 cells expressing EGFR-Vii or EGFR-A2989V, but was not seen in isogenic BaF3 cells expressing the oncogenic EGFR catalytic domain mutation E746-A750 (FIG. 11B), thus demonstrating that paradoxical activation is specific to covalently-activated EGFR isoforms. The biphasic effect on proliferation for cells expressing EGFR-Viii was also seen with the covalent inhibitors WZ8040, WZ4002, and WZ3146, indicating that this behavior exists for small molecules with both reversible and covalent binding modes (FIG. 12). The ability of Type I inhibitors to paradoxically enhance cell proliferation at sub-saturating drug concentrations is fully consistent with the ability of molecules with this type of mechanism to promote the formation of covalently activated dimers.

Mutations and splicing events affecting the CR1 and CR2 regions of the HER2 and HER4 ectodomains are also observed in cancer (Table 8). The most common of these is HER2Δ16, expressed in approximately 50% of breast cancers, but not detected in any normal tissue. HER2Δ16 results from alternative splicing and loss of exon 16, encoding the extracellular juxtamembrane region, producing two free cysteine residues situated at the dimer interface in the CR2 region, Cys626 and Cys630 (Table 8). Compared to HER2-WT, HER2Δ16 is highly tumorigenic. In breast cancer patients, expression of HER-2Δ16 is associated with greater incidence of lymph node involvement and metastatic disease.

TABLE 8

| | | Exons | | |
|---|---|---|---|---|
| | Tumor expression | spliced | | Free Cys |
| Variant | (prevalence) | out | Position | generated |
| HER2-Δ16 | BrCa (52% of | 16 | CR2 | Cys626, Cys630 |
| | HER2+)/GaCa | | | |
| HER2- | GBM (<1%) | NA | CR1 | Cys299 |
| C311R | | | | |
| HER2- | Bladder (5%), Breast | NA | CR1 | ND |
| S310F/Y | (2%), Cervical (1%), | | | |
| | Stomach (1%), | | | |
| | NSCLC (2% | | | |
| | squamous) | | | |

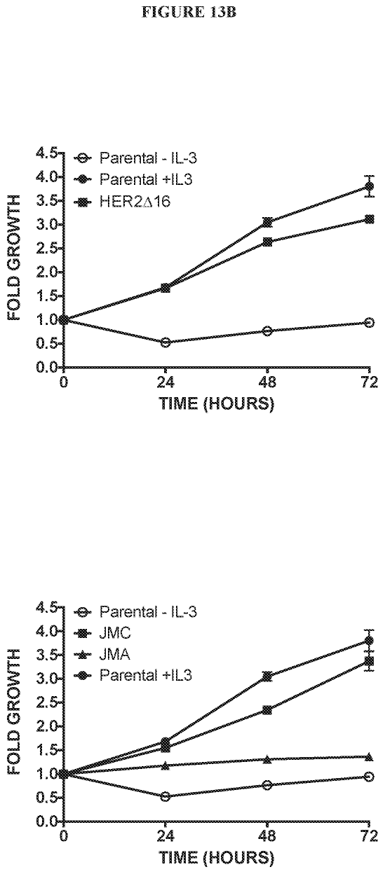

As observed with EGFR, point mutations also occur at the dimer interface of the HER2 CR1 region (Table 8 and FIG. 13). Some mutations introduce novel cysteines or remove one member of a pair of cysteines coordinating an intramolecular disulfide bond. Other mutations, including HER2-S310F/Y, are situated proximal to disulfide bonds and may allosterically disrupt them, as discovered for EGFR-A289V. HER2-S310F/Y mutations are the most frequently occurring HER2 mutations in cancer, expressed by >15% of bladder cancers.

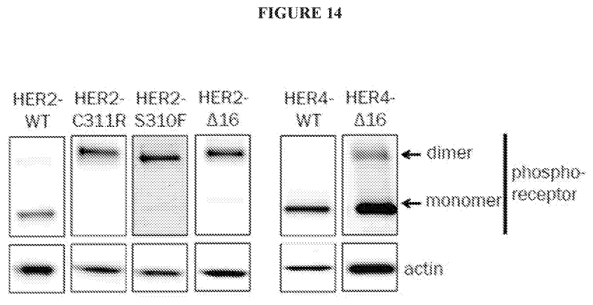

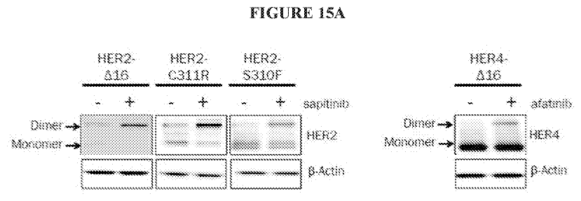

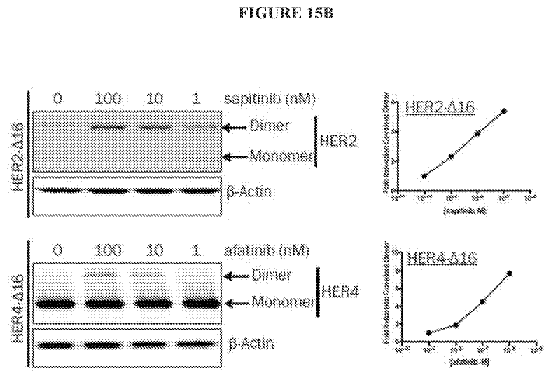

Select extracellular variants of HER2, including HER2-C311R and HER2Δ16, exist as covalently activated dimers. The data of the disclosure demonstrate that other commonly occurring extracellular variants including HER2-S310F also exist as covalently activated receptors (FIG. 14).

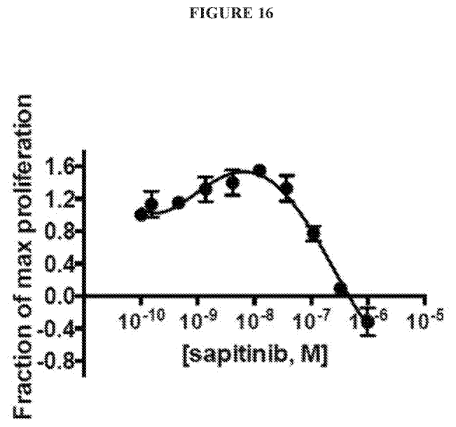

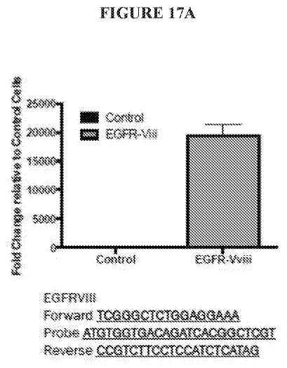

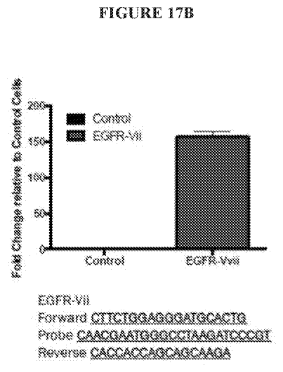

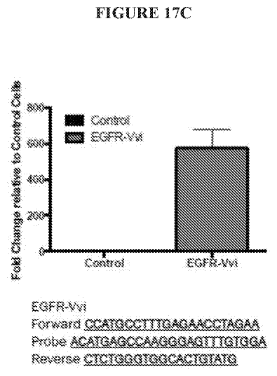

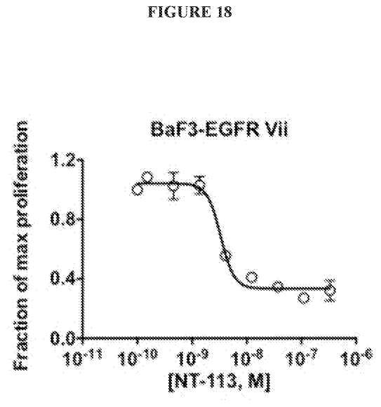

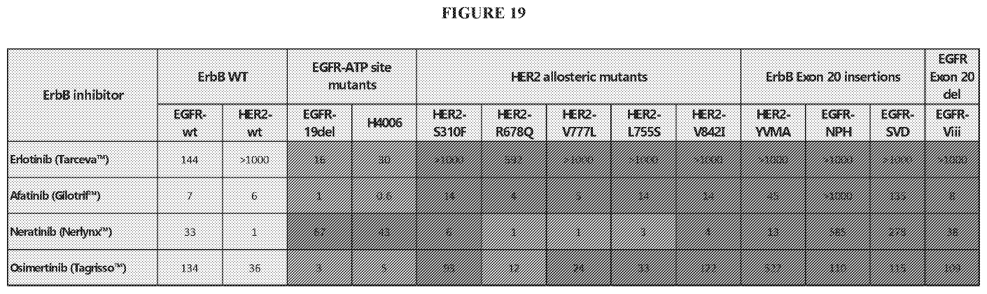

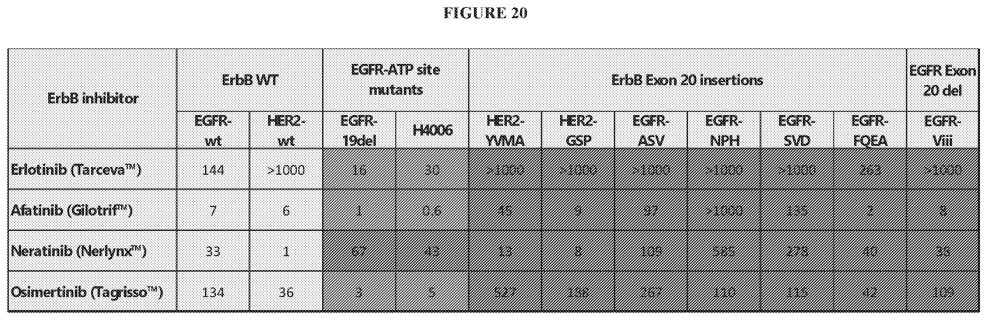

Similar to observations for covalently-activated EGFR variants, Type I inhibitors (sapitinib and afatinib) induce the expression of covalent dimers for HER2 extracellular variants (FIG. 15A). These effects were dose dependent (FIG. 15B). Finally, sapitinib can paradoxically stimulate the proliferation of BaF3 cells driven by HER2-Δ16 (FIG. 16). These data may provide instructive guidelines for the treatment of tumors expressing covalently activated ErbB receptors, including exclusion criteria for Type I inhibitors and a method of treatment for Type II pharmacophores in tumors expressing these variant receptors.

### Methods

Retroviral Production: EGFR mutants were subcloned into pMXs-IRES-Blasticidin (RTV-016, Cell Biolabs, San Diego, Calif.). Retroviral expression vector retrovirus was produced by transient transfection of HEK 293T cells with the retroviral EGFR mutant expression vector pMXs-IRES-Blasticidin (RTV-016, Cell Biolabs), pCMV-Gag-Pol vector and pCMV-VSV-G-Envelope vector. HEK 293T/17 cells were plated in 100 mm collagen coated plate (354450, Corning Life Sciences, Tewksbury, Mass.) (4×10⁵ per plate) and incubated overnight. The next day, retroviral plasmids (3 μg of EGFR mutant, 1.0 μg of pCMV-Gag-Pol and 0.5 μg pCMV-VSV-G) were mixed in 500 μl of Optimem (31985, Life Technologies). The mixture was incubated at room temperature for 5 min and then added to Optimem containing transfection reagent Lipofectamine (11668, Invitrogen) and incubated for 20 minutes. Mixture was then added dropwise to HEK 293T cells. The next day the medium was replaced with fresh culture medium and retrovirus was harvested @ 24 and 48 hrs. Generation of EGFR mutant stable cell lines: BaF3 cells (1.5E5 cells) were infected with 1 ml of viral supernatant supplemented with 8 μg/ml polybrene by centrifuging for 30 min at 1000 rpm. Cells were placed in a 37° C. incubator overnight. Cells were then spun for 5 minutes to pellet the cells. Supernatant was removed and cells re-infected a fresh 1 ml of viral supernatant supplemented with 8 μg/ml polybrene by centrifuging for 30 min at 1000 rpm. Cells were placed in 37° C. incubator overnight. Cells were then maintained in RPMI containing 10% Heat Inactivated FBS, 2% L-glutamine containing 10 ng/ml IL-3. After 48 hours cells were selected for retroviral infection in 10 μg/ml Blasticidin for one week. Blasticidin resistant populations were washed twice in phosphate buffered saline before plating in media lacking IL-3 to select for IL-3 independent growth.

Assay for cell proliferation: BaF3 cell lines were resuspended at 1.3E5 c/ml in RPMI containing 10% Heat Inactivated FBS, 2% L-glutamine and 1% Pen/Strep and dispensed in triplicate (17.5E4 c/well) into 96 well plates. To determine the effect of drug on cell proliferation, cells incubated for 3 days in the presence of vehicle control or test drug at varying concentrations. Inhibition of cell growth was determined by luminescent quantification of intracellular ATP content using CellTiterGilo (Promega), according to the protocol provided by the manufacturer. Comparison of cell number on day 0 versus 72 hours post drug treatment was used to plot dose-response curves. The number of viable cells was determined and normalized to vehicle-treated controls. Inhibition of proliferation, relative to vehicle-treated controls was expressed as a fraction of 1 and graphed using PRISM® software (Graphpad Software, San Diego, Calif.). EC₅₀ values were determined with the same application.

Cellular protein analysis: Cell extracts were prepared by detergent lysis (RIPA, R0278, Sigma, St Louis, Mo.) containing 10 mM Iodoacetamide (786-228, G-Biosciences, St. Louis, Mo.), protease inhibitor (P8340, Sigma, St. Louis, Mo.) and phosphatase inhibitors (P5726, P0044, Sigma, St. Louis, Mo.) cocktails. The soluble protein concentration was determined by micro-BSA assay (Pierce, Rockford Ill.). Protein immunodetection was performed by electrophoretic transfer of SDS-PAGE separated proteins to nitrocellulose, incubation with antibody, and chemiluminescent second step detection. Nitrocellulose membranes were blocked with 5% nonfat dry milk in TBS and incubated overnight with primary antibody in 5% bovine serum albumin. The following primary antibodies from Cell Signaling Technology were used at 1:1000 dilution:phospho-EGFR[Y1173] and total EGFR. β-Actin antibody, used as a control for protein loading, was purchased from Sigma Chemicals. Horseradish peroxidase-conjugated secondary antibodies were obtained from Cell Signaling Technology and used at 1:5000 dilution. Horseradish peroxidase-conjugated secondary antibodies were incubated in nonfat dry milk for 1 hour. SuperSignal chemiluminescent reagent (Pierce Biotechnology) was used according to the manufacturer's directions and blots were imaged using the Alpha Innotech image analyzer and AlphaEaseFC software (Alpha Innotech, San Leandro Calif.).

### Uses of the Compounds and Compositions

In some aspects, the present disclosure is directed to a method of inhibiting an oncogenic variant of an ErbB receptor (e.g., an oncogenic variant of an EGFR), comprising administering the subject in need thereof a therapeutically effective amount of a compound described herein.

In some aspects, the present disclosure is directed to a method of inhibiting an oncogenic variant of an ErbB receptor (e.g., an oncogenic variant of an EGFR), comprising administering the subject in need thereof a composition described herein.

In some aspects, the present disclosure is directed to a method of preventing or treating cancer, comprising administering the subject in need thereof a therapeutically effective amount of a compound described herein.

In some aspects, the present disclosure is directed to a method of preventing or treating cancer, comprising administering the subject in need thereof a composition described herein.

In some aspects, the present disclosure is directed to a method of preventing or treating cancer, comprising: i) identifying a subject candidate as the subject in need of the treatment when that at least one oncogenic variant of an ErbB receptor described herein is present in the subject; and ii) administering the subject in need of the treatment a therapeutically effective amount of a compound described herein.

In some aspects, the present disclosure is directed to a method of preventing or treating cancer comprising: i) identifying a subject candidate as the subject in need of the treatment when that at least one oncogenic variant of an ErbB receptor described herein is present in the subject; and ii) administering the subject in need of the treatment a composition described herein.

In some aspects, the present disclosure is directed to a method of preventing or treating cancer, comprising: i) identifying a subject candidate as the subject in need of the treatment when that at least one oncogenic variant of an ErbB receptor described herein is present in a biological sample from the subject; and ii) administering the subject in need of the treatment a therapeutically effective amount of a compound described herein.

In some aspects, the present disclosure is directed to a method of preventing or treating cancer, comprising: i) identifying a subject candidate as the subject in need of the treatment when that at least one oncogenic variant of an ErbB receptor described herein is present in a biological sample from the subject; and ii) administering the subject in need of the treatment a composition described herein.

In some aspects, the present disclosure is directed to a method of preventing or treating cancer, comprising administering the subject in need thereof a therapeutically effective amount of a compound described herein when that at least one oncogenic variant of an ErbB receptor described herein is identified as being present in the subject.

In some aspects, the present disclosure is directed to a method of preventing or treating cancer, comprising administering the subject in need thereof a compound described herein when that at least one oncogenic variant of an ErbB receptor described herein is identified as being present in the subject.

In some aspects, the present disclosure is directed to a method of preventing or treating cancer, comprising administering the subject in need thereof a therapeutically effective amount of a compound described herein when that at least one oncogenic variant of an ErbB receptor described herein is identified as being present in a biological sample from the subject.

In some aspects, the present disclosure is directed to a method of preventing or treating cancer, comprising administering the subject in need thereof a composition described herein when that at least one oncogenic variant of an ErbB receptor described herein is identified as being present in a biological sample from the subject.

In some aspects, the present disclosure is directed to a compound described herein for use in the inhibition of an oncogenic variant of an ErbB receptor (e.g., an oncogenic variant of an EGFR).

In some aspects, the present disclosure is directed to a compound described herein for use in the prevention or treatment of cancer.

In some aspects, the present disclosure is directed to a composition described herein for use in the inhibition of an oncogenic variant of an ErbB receptor (e.g., an oncogenic variant of an EGFR).

In some aspects, the present disclosure is directed to a composition described herein for use in the prevention or treatment of cancer.

In some aspects, the present disclosure is directed to a compound described herein for use in the prevention or treatment of cancer in a subject, wherein at least one oncogenic variant of an ErbB receptor described herein is present in the subject.

In some aspects, the present disclosure is directed to a composition described herein for use in the prevention or treatment of cancer in a subject, wherein at least one oncogenic variant of an ErbB receptor described herein is present in the subject.

In some aspects, the present disclosure is directed to a compound described herein for use in the prevention or treatment of cancer in a subject, wherein at least one oncogenic variant of so an ErbB receptor described herein is present in a biological sample from the subject.

In some aspects, the present disclosure is directed to a composition described herein for use in the prevention or treatment of cancer in a subject, wherein at least one oncogenic variant of an ErbB receptor described herein is present in a biological sample from the subject.

In some aspects, the present disclosure is directed to use of a compound described herein in the manufacture of a medicament for inhibiting an oncogenic variant of an ErbB receptor (e.g., an oncogenic variant of an EGFR).

In some aspects, the present disclosure is directed to use of a compound described herein in the manufacture of a medicament for preventing or treating cancer.

In some embodiments, the compound is selected from the compounds described in Table 1, pharmaceutically acceptable salts thereof, and stereoisomers thereof.

In some embodiments, the compound is selected from the compounds described in Table 1 and pharmaceutically acceptable salts thereof.

In some embodiments, the compound is selected from the compounds described in Table 1.

In some embodiments, cancer is a solid tumor.

In some embodiments, the cancer is a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC), or any subtype thereof.

In some embodiments, the cancer is glioblastoma (GBM) or any subtype thereof.

In some embodiments, the cancer is glioblastoma.

The disclosure provides a composition comprising a compound of the disclosure or pharmaceutically acceptable salts or stereoisomers thereof. In some embodiments, the composition comprises a pharmaceutically acceptable carrier. In some embodiments, the composition composition comprises a second therapeutically active agent. In some embodiments, the second therapeutically active agent comprises a second compound of the disclosure. In some embodiments, the second therapeutically active agent comprises a non-Type I inhibitor. In some embodiments, the non-Type I inhibitor comprises a Type II inhibitor. In some embodiments, the Type II inhibitor comprises a small molecule inhibitor.

The disclosure provides a composition of the disclosure for use in the treatment of cancer, wherein the cancer or a tumor or a cell thereof expresses an oncogenic variant of an epidermal growth factor receptor (EGFR).

In some embodiments, of the compositions for use in the treatment of cancer of the disclosure, including those wherein the cancer or a tumor or a cell thereof expresses an oncogenic variant of an epidermal growth factor receptor (EGFR), the oncogenic variant of an EGFR is an allosteric variant of EGFR.

In some embodiments, of the compositions for use in the treatment of cancer of the disclosure, cancer or a tumor or a cell thereof expresses an oncogenic variant of an epidermal growth factor receptor (EGFR) and wherein the oncogenic variant of an EGFR is an allosteric variant of EGFR, the oncogenic variant of an EGFR comprises an EGFR variant III (EGFR-Viii) mutation.

In some embodiments of the compositions for use in the treatment of cancer of the disclosure, cancer or a tumor or a cell thereof expresses an oncogenic variant of an epidermal growth factor receptor (EGFR) and wherein the oncogenic variant of an EGFR is an allosteric variant of EGFR, the oncogenic variant of an EGFR comprises a substitution of a valine (V) for an alanine (A) at position 289 of SEQ ID NO: 1.

In some embodiments of the compositions for use in the treatment of cancer of the disclosure, cancer or a tumor or a cell thereof expresses an oncogenic variant of an epidermal growth factor receptor (EGFR) and wherein the oncogenic variant of an EGFR is an allosteric variant of EGFR, the oncogenic variant of an EGFR comprises a modification of a structure of the EGFR, wherein the oncogenic variant of an EGFR is a capable of forming a covalently linked dimer, wherein the covalently linked dimer is constitutively active and wherein the covalently linked dimer enhances an activity of EGFR when contacted to a Type I ErbB inhibitor. In some embodiments, the modification of the structure of the EGFR comprises a modification of one or more of a nucleic acid sequence, an amino acid sequence, a secondary structure, a tertiary structure, and a quaternary structure. In some embodiments, the oncogenic variant comprises a mutation, a splicing event, a post-translational process, a conformational change or any combination thereof. In some embodiments, the modification of the structure of the EGFR occurs within a first cysteine rich (CR1) and/or second cysteine rich (CR2) region of EGFR. In some embodiments, the first cysteine rich (CR1) and/or second cysteine rich (CR2) region of EGFR comprises amino acid residues T211-R334 and/or C526-S645 of SEQ ID NO: 1, respectively. In some embodiments, the oncogenic variant of an EGFR generates a physical barrier to formation of a disulfide bond within the CR1 and/or the CR2 region. In some embodiments, the oncogenic variant of an EGFR removes a physical barrier to formation of a disulfide bond within the CR1 and/or the CR2 region. In some embodiments, the oncogenic variant of an EGFR comprises one or more free or unpaired Cysteine (C) residues located at a dimer interface of the EGFR. In some embodiments, the oncogenic variant of an EGFR comprises one or more free or unpaired Cysteine (C) residues at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1. In some embodiments, the modification occurs within 10 angstroms or less of an intramolecular disulfide bond at a site selected from the group consisting of C190-C199, C194-C207, C215-C223, C219-C231, C232-C240, C236-C248, C25I-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1.

In some embodiments of the compositions for use in the treatment of cancer of the disclosure, cancer or a tumor or a cell thereof expresses oncogenic variant of EGFR and the oncogenic variant of EGFR is a mutation of EGFR, a nucleotide sequence encoding the oncogenic variant of an EGFR comprises a deletion or a substitution of a sequence encoding exon 19 or a portion thereof. In some embodiments, the deletion or the substitution comprises one or more amino acids that encode an adenosine triphosphate (ATP) binding site. In some embodiments, the ATP binding site comprises amino acids E746 to A750 of SEQ ID NO: 1.

In some embodiments, the ATP binding site or the deletion or substitution thereof comprises K858 of SEQ ID NO: 1. In some embodiments, the deletion comprises K858 of SEQ ID NO: 1. In some embodiments, an arginine (R) is substituted for the lysine (K) at position 858 (K858R) of SEQ ID NO: 1. In some embodiments, an arginine (R) is substituted for the leucine (L) at position 858 (L858R) of SEQ ID NO: 1.

In some embodiments of the compositions for use in the treatment of cancer of the disclosure, cancer or a tumor or a cell thereof expresses an oncogenic variant of an epidermal growth factor receptor (EGFR) and wherein the oncogenic variant of an EGFR is an allosteric variant of EGFR, a nucleotide sequence encoding the oncogenic variant of an EGFR comprises an insertion within a sequence encoding exon 20 or a portion thereof. In some embodiments, the sequence encoding exon 20 or a portion thereof comprises a sequence encoding KEILDEAYVMASVDNPHVCAR (SEQ ID NO: 7). In some embodiments, the sequence encoding exon 20 or a portion thereof comprises a sequence encoding a C-helix, a terminal end of the C-helix or a loop following the C-helix. In some embodiments, the insertion comprises the amino acid sequence of ASV, SVD, NPH, or FQEA. In some embodiments, the sequence encoding exon 20 or a portion thereof comprises one or more of: (a) an insertion of the amino acid sequence ASV between positions V769 and D770 of SEQ ID NO: 1; (b) an insertion of the amino acid sequence SVD between positions D770 and N771 of SEQ ID NO: I; (c) an insertion of the amino acid sequence NPH between positions H773 and V774 of SEQ ID NO: 1; (d) an insertion of the amino acid sequence FQEA between positions A763 and Y764 of SEQ ID NO: 1; (e) an insertion of the amino acid sequence PH between positions H773 and V774 of SEQ ID NO: 1; (f) an insertion of the amino acid G between positions D770 and N771 of SEQ ID NO: 1; (g) an insertion of the amino acid H between positions H773 and V774 of SEQ ID NO: 1; (h) an insertion of the amino acid sequence HV between positions V774 and C775 of SEQ ID NO: 1; (i) an insertion of the amino acid sequence AH between positions H773 and V774 of SEQ ID NO: 1; (j) an insertion of the amino acid sequence SVA between positions A767 and S768 of SEQ ID NO: 1; (k) a substitution of the amino acid sequence GYN for the DN between positions 770 and 771 of SEQ ID NO: 1; (l) an insertion of the amino acid H between positions N771 and P772 of SEQ ID NO: 1; (m) an insertion of the amino acid Y between positions H773 and V774 of SEQ ID NO: 1; (n) an insertion of the amino acid sequence PHVC between positions C775 and R776 of SEQ ID NO: 1; (o) a substitution of the amino acid sequence YNPY for the H at position 773 of SEQ ID NO: 1; (p) an insertion of the amino acid sequence DNP between positions P772 and H773 of SEQ ID NO: 1; (q) an insertion of the amino acid sequence VDS between positions S768 and V769 of SEQ ID NO: 1; (r) an insertion of the amino acid H between positions D770 and N771 of SEQ ID NO: 1; (s) an insertion of the amino acid N between positions N771 and P772 of SEQ ID NO: 1; (t) an insertion of the amino acid sequence PNP between positions P772 and H773 of SEQ ID NO: 1; (u) a substitution of the amino acid sequence GSVDN for the DN between positions 770 and 771 of SEQ ID NO: 1; (v) a substitution of the amino acid sequence GYP for the NP between positions 771 and 772 of SEQ ID NO: 1; (w) an insertion of the amino acid G between positions N771 and P772 of SEQ ID NO: 1; (x) an insertion of the amino acid sequence GNP between positions P772 and H773 of SEQ ID NO: 1; (v) an insertion of the amino acid sequence GSV between positions V769 and D770 of SEQ ID NO: 1; (z) a substitution of the amino acid sequence GNPHVC for the VC between positions 774 and 775 of SEQ ID NO: 1; (aa) an insertion of the amino acid sequence LQEA between positions A763 and Y764 of SEQ ID NO: 1; (bb) an insertion of the amino acid sequence GL between positions D770 and N771 of SEQ ID NO: 1; (cc) an insertion of the amino acid Y between positions D770 and N771 of SEQ ID NO: 1; (dd) an insertion of the amino acid sequence NPY between positions H773 and V774 of SEQ ID NO: 1; (ee) an insertion of the amino acid sequence TH between positions H773 and V774 of SEQ ID NO: 1; (ff) a substitution of the amino acid sequence KGP for the NP between positions 771 and 772 of SEQ ID NO: 1; (gg) a substitution of the amino acid sequence SVDNP for the NP between positions 771 and 772 of SEQ ID NO: 1; (hh) an insertion of the amino acid sequence NN between positions N771 and P772 of SEQ ID NO: 1; (ii) an insertion of the amino acid T between positions N771 and P772 of SEQ ID NO: 1; and (jj) a substitution of the amino acid sequence STLASV for the SV between positions 768 and 769 of SEQ ID NO: 1.

In some embodiments of the compositions for use in the treatment of cancer of the disclosure, cancer or a tumor or a cell thereof expresses an oncogenic variant of an epidermal growth factor receptor (EGFR) and wherein the oncogenic variant of an EGFR is an allosteric variant of EGFR, the oncogenic variant of an EGFR comprises EGFR-Vii, EGFR-Vvi, EGFR-R222C, EGFR-R252C, EGFR-R252P, EGFR-R256Y, EGFR-T263P, EGFR-Y270C, EGFR-A289T, EGFR-A289V, EGFR-A289D, EGFR-H304Y, EGFR-G331R, EGFR-P596S, EGFR-P596L, EGFR-P596R, EGFR-G598V, EGFR-G598A, EGFR-G614D, EGFR-C620Y, EGFR-C614W, EGFR-C628F, EGFR-C628Y, EGFR-C636Y, EGFR-G645C, EGFR-A660, EGFR-Δ768 or any combination thereof.

In some embodiments, the disclosure provides a composition of the disclosure for use in the treatment of cancer, wherein the cancer, a tumor or a cell thereof expresses one or more of: (a) a wild type human epidermal growth factor receptor 2 (HER2) receptor or (b) an oncogenic variant of a HER-2 receptor.

In some embodiments of the compositions for use in the treatment of cancer of the disclosure, including those wherein the cancer or a tumor or a cell thereof expresses a wild type HER-2 receptor, the wild type HER2 receptor comprises the amino acid sequence of SEQ ID NO: 2, 3, 4, 5, or 6.

In some embodiments of the compositions for use in the treatment of cancer of the disclosure, including those wherein the cancer or a tumor or a cell thereof expresses an oncogenic variant of a HER-2 receptor, the oncogenic variant of a I-HER2 receptor is an allosteric variant of the HER2 receptor.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of a phenylalanine (F) for a serine (S) at position 310 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of a tyrosine (Y) for a serine (S) at position 310 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of a glutamine (Q) for an arginine (R) at position 678 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of a leucine (L) for a valine (V) at position 777 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of a methionine (M) for a valine (V) at position 777 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the I-ER-2 receptor, the oncogenic variant of a HER2 receptor comprises a substitution of an isoleucine (I) for a valine (V) at position 842 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of an alanine (A) for a leucine (L) at position 755 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of a proline (P) for a leucine (L) at position 755 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises a substitution of a serine (S) for a leucine (L) at position 755 of SEQ ID NO: 2 or 5.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, a nucleotide sequence encoding the oncogenic variant of a HER2 receptor comprises an insertion within a sequence encoding exon 20 or a portion thereof. In some embodiments, the sequence encoding exon 20 or a portion thereof comprises a sequence encoding KEILDEAYVMAGVGSPYVSR(SEQ ID NO: 8). In some embodiments, the sequence encoding exon 20 or a portion thereof comprises a sequence encoding a C-helix, a terminal end of the C-helix or a loop following the C-helix. In some embodiments, the insertion comprises the amino acid sequence of GSP or YVMA. In some embodiments, the sequence encoding exon 20 or a portion thereof comprises one or more of: (a) an insertion of the amino acid sequence YVMA between positions A775 and G776 of SEQ ID NO: 2; (b) an insertion of the amino acid sequence GSP between positions P780 and Y781 of SEQ ID NO: 2; (c) an insertion of the amino acid sequence YVMA between positions A771 and Y772 of SEQ ID NO: 2; (d) an insertion of the amino acid sequence YVMA between positions A775 and G776 of SEQ ID NO: 2: (e) an insertion of the amino acid V between positions V777 and G778 of SEQ ID NO: 2; (f) an insertion of the amino acid V between positions V777 and (G778 of SEQ ID NO: 2; (g) a substitution of the amino acid sequence AVGCV for the GV between positions 776 and 777 of SEQ ID NO: 2; (h) a substitution of the amino acid sequence LC for the G between position 776 of SEQ ID NO: 2; (i) a substitution of the amino acid sequence LCV for the G between position 776 of SEQ ID NO: 2; (j) an insertion of the amino acid sequence GSP between positions V777 and G778 of SEQ ID NO: 2; (k) a substitution of the amino acid sequence PS for the LRE between positions 755 and 757 of SEQ ID NO: 2; (l) a substitution of the amino acid sequence CPGSP for the SP between positions 779 and 780 of SEQ ID NO: 2; (m) an insertion of the amino acid C between positions V777 and G778 of SEQ ID NO: 2; (n) a substitution of the amino acid sequence VVMA for the AG between positions 775 and 776 of SEQ ID NO: 2; (o) a substitution of the amino acid sequence VV for the G at position 776 of SEQ ID NO: 2; (p) a substitution of the amino acid sequence AVCV for the GV between positions 776 and 777 of SEQ ID NO: 2; (q) a substitution of the amino acid sequence VCV for the GV between positions 776 and 777 of SEQ ID NO: 2; (r) an insertion of the amino acid G between positions G778 and S779 of SEQ ID NO: 2; (s) a substitution of the amino acid sequence PK for the LRE between positions 755 and 757 of SEQ ID NO: 2; (t) an insertion of the amino acid V between positions A775 and G776 of SEQ ID NO: 2; (u) an insertion of the amino acid sequenceYAMA between positions A775 and G776 of SEQ ID NO: 2; (v) a substitution of the amino acid sequence CV for the G at position 776 of SEQ ID NO: 2: (w) a substitution of the amino acid sequence AVCGG for the GVG between positions 776 and 778 of SEQ ID NO: 2; (x) a substitution of the amino acid sequence CVCG for the GVG between positions 776 and 778 of SEQ ID NO: 2; (y) a substitution of the amino acid sequence VVVG for the GVG between positions 776 and 778 of SEQ ID NO: 2; (z) a substitution of the amino acid sequence SVGG for the GVGS between positions 776 and 779 of SEQ ID NO: 2; (aa) a substitution of the amino acid sequence VVGES for the GVGS between positions 776 and 779 of SEQ ID NO: 2; (bb) a substitution of the amino acid sequence AVGSGV for the CV between positions 776 and 777 of SEQ ID NO: 2; (cc) a substitution of the amino acid sequence CVC for the GV between positions 776 and 777 of SEQ ID NO: 2; (dd) a substitution of the amino acid sequence HVC for the GV between positions 776 and 777 of SEQ ID NO: 2: (ee) a substitution of the amino acid sequence VAAGV for the GV between positions 776 and 777 of SEQ ID NO: 2; (ff) a substitution of the amino acid sequence VAGV for the GV between positions 776 and 777 of SEQ ID NO: 2; (gg) a substitution of the amino acid sequence VVV for the GV between positions 776 and 777 of SEQ ID NO: 2; (hh) an insertion of the amino acid sequence FPG between positions G778 and S779 of SEQ ID NO: 2; (ii) an insertion of the amino acid sequence GS between positions S779 and P780 of SEQ ID NO: 2; (jj) a substitution of the amino acid sequence VPS for the VLRE between positions 754 and 757 of SEQ ID NO: 2; (kk) an insertion of the amino acid E between positions V777 and G778 of SEQ ID NO: 2; (ll) an insertion of the amino acid sequence MAGV between positions V777 and G778 of SEQ ID NO: 2; (mm) an insertion of the amino acid S between positions V777 and G778 of SEQ ID NO: 2; (nn) an insertion of the amino acid sequence SCV between positions V777 and G778 of SEQ ID NO: 2; and (oo) an insertion of the amino acid sequence LMAY between positions Y772 and V773 of SEQ ID NO: 2.

In some embodiments of the compositions for use in the treatment of cancer of the disclosure, including those wherein the cancer or a tumor or a cell thereof expresses an oncogenic variant of a HER-2 receptor and wherein the oncogenic variant of the HER-2 receptor is an allosteric variant of the HER-2 receptor, the oncogenic variant of a HER2 receptor comprises HER2-Δ16, HER2-C311R, HER2-S310F, p95-HER2-M611 or any combination thereof.

In some embodiments, the disclosure provides a use of the composition of the disclosure for treating cancer, comprising administering to a subject a therapeutically-effective amount of the composition, wherein the cancer, a tumor or a cell thereof expresses an oncogenic variant of an epidermal growth factor receptor (EGFR).

In some embodiments of the uses of the compositions of the disclosure for the treatment of cancer, including those wherein the cancer or a tumor or a cell thereof expresses an oncogenic variant of an EGFR, the oncogenic variant of EGFR is an allosteric variant of EGFR.

In some embodiments of the uses of the compositions of the disclosure for the treatment of cancer, including those wherein the cancer or a tumor or a cell thereof expresses an oncogenic variant of an EGFR and wherein the oncogenic variant of EGFR is an allosteric variant of EGFR, the oncogenic variant of an EGFR comprises an EGFR variant III (EGFR-Viii) mutation.

In some embodiments of the uses of the compositions of the disclosure for the treatment of cancer, including those wherein the cancer or a tumor or a cell thereof expresses an oncogenic variant of an EGFR and wherein the oncogenic variant of EGFR is an allosteric variant of EGFR, the oncogenic variant of an EGFR comprises a substitution of a valine (V) for an alanine (A) at position 289 of SEQ ID NO: 1.

In some embodiments of the uses of the compositions of the disclosure for the treatment of cancer, including those wherein the cancer or a tumor or a cell thereof expresses an oncogenic variant of an EGFR and wherein the oncogenic variant of EGFR is an allosteric variant of EGFR, the oncogenic variant of an EGFR comprises a modification of a structure of the EGF R, wherein the oncogenic variant of an EGFR is a capable of forming a covalently linked dimer, wherein the covalently linked dimer is constitutively active and wherein the covalently linked dimer enhances an activity of EGFR when contacted to a Type I ErbB inhibitor. In some embodiments, the modification of the structure of the EGFR comprises a modification of one or more of a nucleic acid sequence, an amino acid sequence, a secondary structure, a tertiary structure, and a quaternary structure. In some embodiments, the oncogenic variant comprises a mutation, a splicing event, a post-translational process, a conformational change or any combination thereof. In some embodiments, the modification of the structure of the EGFR occurs within a first cysteine rich (CR1) and/or second cysteine rich (CR2) region of EGFR. In some embodiments, the first cysteine rich (CR1) and/or second cysteine rich (CR2) region of EGFR comprises amino acid residues T211-R334 and/or C526-S645 of SEQ ID NO: 1, respectively. In some embodiments, the oncogenic variant of an EGFR generates a physical barrier to formation of a disulfide bond within the CR1 and/or the CR2 region. In some embodiments, the oncogenic variant of an EGFR removes a physical barrier to formation of a disulfide bond within the CR1 and/or the CR2 region. In some embodiments, the oncogenic variant of an EGFR comprises one or more free or unpaired Cysteine (C) residues located at a dimer interface of the EGFR. In some embodiments, the oncogenic variant of an EGFR comprises one or more free or unpaired Cysteine (C) residues at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1. In some embodiments, the modification occurs within 10 angstroms or less of an intramolecular disulfide bond at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579. C582-C591, C595-C617, C620-C628 and C624-C636 according to SEQ ID NO: 1.

In some embodiments of the uses of the compositions of the disclosure for the treatment of cancer, including those wherein the cancer or a tumor or a cell thereof expresses an oncogenic variant of EGFR and the oncogenic variant of EGFR is a mutation of EGFR, a nucleotide sequence encoding the oncogenic variant of an EGFR comprises a deletion or the substitution comprises one or more amino acids that encode an adenosine triphosphate (ATP) binding site. In some embodiments, the ATP binding site comprises amino acids E746 to A750 of SEQ ID NO: 1. In some embodiments, the ATP binding site or the deletion or substitution thereof comprises K858 of SEQ ID NO: 1. In some embodiments, the deletion comprises K858 of SEQ ID NO: 1. In some embodiments, an arginine (R) is substituted for the lysine (K) at position 858 (K858R) of SEQ ID NO: 1. In some embodiments, an arginine (R) is substituted for the leucine (L) at position 858 (L858R) of SEQ ID NO: 1.

In some embodiments of the uses of the compositions of the disclosure for the treatment of cancer, including those wherein the cancer or a tumor or a cell thereof expresses an oncogenic variant of an EGFR and wherein the oncogenic variant of EGFR is an allosteric variant of EGFR, a nucleotide sequence encoding the oncogenic variant of an EGFR comprises an insertion within a sequence encoding exon 20 or a portion thereof. In some embodiments, the sequence encoding exon 20 or a portion thereof comprises a sequence encoding KEILDEAYVMASVDNPHVCAR (SEQ ID NO: 7). In some embodiments, the sequence encoding exon 20 or a portion thereof comprises a sequence encoding a C-helix, a terminal end of the C-helix or a loop following the C-helix. In some embodiments, the insertion comprises the amino acid sequence of ASV, SVD, NPH, or FQEA. In some embodiments, the sequence encoding exon 20 or a portion thereof comprises one or more of: (a) an insertion of the amino acid sequence ASV between positions V769 and D770 of SEQ ID NO: 1; (b) an insertion of the amino acid sequence SVD between positions D770 and N771 of SEQ ID NO: 1; (c) an insertion of the amino acid sequence NPH between positions H773 and V774 of SEQ ID NO: 1; (d) an insertion of the amino acid sequence FQEA between positions A763 and Y764 of SEQ ID NO: 1; (e) an insertion of the amino acid sequence PH between positions H773 and V774 of SEQ ID NO: 1 (f an insertion of the amino acid G between positions D770 and N771 of SEQ ID NO: 1; (g) an insertion of the amino acid H between positions 1H773 and V774 of SEQ ID NO: 1; (h) an insertion of the amino acid sequence HV between positions V774 and C775 of SEQ ID NO: 1; (i) an insertion of the amino acid sequence AH between positions H773 and/774 of SEQ ID NO: 1; (j) an insertion of the amino acid sequence SVA between positions A767 and S768 of SEQ ID NO: 1; (k) a substitution of the amino acid sequence GYN for the DN between positions 770 and 771 of SEQ ID NO: 1; (l) an insertion of the amino acid H between positions N771 and P772 of SEQ ID NO: 1; (m) an insertion of the amino acid Y between positions H773 and V774 of SEQ ID NO: 1; (n) an insertion of the amino acid sequence PHVC between positions C775 and R776 of SEQ ID NO: 1; (o) a substitution of the amino acid sequence YNPY for the H at position 773 of SEQ ID NO: 1; (p) an insertion of the amino acid sequence DNP between positions P772 and H773 of SEQ ID NO: 1; (q) an insertion of the amino acid sequence VDS between positions S768 and V769 of SEQ ID NO: 1; (r) an insertion of the amino acid H between positions D770 and N771 of SEQ ID NO: 1; (s) an insertion of the amino acid N between positions N771 and P772 of SEQ ID NO: 1; (t) an insertion of the amino acid sequence PNP between positions P772 and H773 of SEQ ID NO: 1; (u) a substitution of the amino acid sequence GSVDN for the DN between positions 770 and 771 of SEQ ID NO: 1; (v) a substitution of the amino acid sequence GYP for the NP between positions 771 and 772 of SEQ ID NO: 1; (w) an insertion of the amino acid G between positions N771 and P772 of SEQ ID NO: 1 (x) an insertion of the amino acid sequence GNP between positions P772 and H773 of SEQ ID NO: 1; (v) an insertion of the amino acid sequence GSV between positions V769 and D770 of SEQ ID NO: 1; (z) a substitution of the amino acid sequence GNPHVC for the VC between positions 774 and 775 of SEQ ID NO: 1; (aa) an insertion of the amino acid sequence LQEA between positions A763 and Y764 of SEQ ID NO: 1; (bb) an insertion of the amino acid sequence GL between positions D770 and N77I of SEQ ID NO: 1; (cc) an insertion of the amino acid Y between positions D770 and N771 of SEQ ID NO: 1; (dd) an insertion of the amino acid sequence NPY between positions H773 and V774 of SEQ ID NO: 1; (ee) an insertion of the amino acid sequence TH between positions H773 and V774 of SEQ ID NO: 1; (ff) a substitution of the amino acid sequence KGP for the NP between positions 771 and 772 of SEQ ID NO: 1; (gg) a substitution of the amino acid sequence SVDNP for the NP between positions 771 and 772 of SEQ ID NO: 1; (hh) an insertion of the amino acid sequence NN between positions N771 and P772 of SEQ ID NO: 1; (ii) an insertion of the amino acid T between positions N771 and P772 of SEQ ID NO: 1; and jj) a substitution of the amino acid sequence STLASV for the SV between positions 768 and 769 of SEQ ID NO: 1.

In some embodiments of the uses of the compositions of the disclosure for the treatment of cancer, including those wherein the cancer or a tumor or a cell thereof expresses an oncogenic variant of an EGFR and wherein the oncogenic variant of EGFR is an allosteric variant of EGFR, the oncogenic variant of an EGFR comprises EGFR-Vii, EGFR-Vvi, EGFR-R222C, EGFR-R252C, EGFR-R252P, EGFR-R256Y, EGFR-T263P, EGFR-Y270C, EGFR-A289T, EGFR-A289V, EGFR-A289D, EGFR-H304Y, EGFR-G331R, EGFR-P596S, EGFR-P596L, EGFR-P596R, EGFR-G598V, EGFR-G598A, EGFR-G614D, EGFR-C₆₂₀Y, EGFR-C614W, EGFR-C628F, EGFR-C628Y, EGFR-C636Y, EGFR-G645C, EGFR-Δ660, EGFR-Δ768 or any combination thereof.

In some embodiments, the disclosure provides a use of a composition of the disclosure for treating cancer, comprising administering to a subject a therapeutically-effective amount of the composition, wherein the cancer, a tumor or a cell thereof expresses one or more of: (a) a wild type human epidermal growth factor receptor 2 (HER2) receptor or an oncogenic variant of a HER-2 receptor.

In some embodiments of the uses of the compositions of the disclosure for the treatment of cancer, including those wherein the cancer, a tumor or a cell thereof expresses a wild type HER-2 receptor, the wild type HER2 receptor comprises the amino acid sequence of SEQ ID NO: 2, 3, 4, 5, or 6.

In some embodiments of the uses of the compositions of the disclosure for the treatment of cancer, including those wherein the cancer, a tumor or a cell thereof expresses an oncogenic variant of a HER-2 receptor, the oncogenic variant of a HER2 receptor is an allosteric variant of the HER2 receptor.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of a phenylalanine (F) for a serine (S) at position 310 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of a tyrosine (Y) for a serine (S) at position 310 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of a glutamine (Q) for an arginine (R) at position 678 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of a leucine (L) for a valine (V) at position 777 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of a methionine (M) for a valine (V) at position 777 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of an isoleucine (I) for a valine (V) at position 842 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of an alanine (A) for a leucine (L) at position 755 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises a substitution of a proline (P) for a leucine (L) at position 755 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the I-HER2 receptor, the oncogenic variant of a HER2 receptor comprises a substitution of a serine (S) for a leucine (L) at position 755 of SEQ ID NO: 2 or 5.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, a nucleotide sequence encoding the oncogenic variant of a HER2 receptor comprises an insertion within a sequence encoding exon 20 or a portion thereof. In some embodiments, the sequence encoding exon 20 or a portion thereof comprises a sequence encoding KEILDEAYVMACVGSPYVSR(SEQ ID NO: 8). In some embodiments, the sequence encoding exon 20 or a portion thereof comprises a sequence encoding a C-helix, a terminal end of the C-helix or a loop following the C-helix. In some embodiments, the insertion comprises the amino acid sequence of GSP or YVMA. In some embodiments, the sequence encoding exon 20 or a portion thereof comprises one or more of: (a) an insertion of the amino acid sequence YVMA between positions A775 and G776 of SEQ ID NO: 2; (b) an insertion of the amino acid sequence GSP between positions P780 and Y781 of SEQ ID NO: 2; (c) an insertion of the amino acid sequence YVMA between positions A771 and Y772 of SEQ ID NO: 2; (d) an insertion of the amino acid sequence YVMA between positions A775 and G776 of SEQ ID NO: 2; (e) an insertion of the amino acid V between positions V777 and G778 of SEQ ID NO: 2; (f) an insertion of the amino acid V between positions V777 and G778 of SEQ ID NO: 2; (g) a substitution of the amino acid sequence AVGCV for the GV between positions 776 and 777 of SEQ ID NO: 2; (h) a substitution of the amino acid sequence LC for the G between position 776 of SEQ ID NO: 2; (i) a substitution of the amino acid sequence LCV for the G between position 776 of SEQ ID NO: 2; (j) an insertion of the amino acid sequence ISP between positions V777 and G778 of SEQ ID NO: 2; (k) a substitution of the amino acid sequence PS for the LRE between positions 755 and 757 of SEQ ID NO: 2: (l) a substitution of the amino acid sequence CPGSP for the SP between positions 779 and 780 of SEQ ID NO: 2; (m) an insertion of the amino acid C between positions V777 and G778 of SEQ ID NO: 2; (n) a substitution of the amino acid sequence VVMA for the AG between positions 775 and 776 of SEQ ID NO: 2; (o) a substitution of the amino acid sequence VV for the G at position 776 of SEQ ID NO: 2; (p) a substitution of the amino acid sequence AVCV for the (V between positions 776 and 777 of SEQ ID NO: 2; (q) a substitution of the amino acid sequence VCV for the GV between positions 776 and 777 of SEQ ID NO: 2; (r) an insertion of the amino acid G between positions G778 and S779 of SEQ ID NO: 2; (s) a substitution of the amino acid sequence PK for the LRE between positions 755 and 757 of SEQ ID NO: 2; (t) an insertion of the amino acid V between positions A775 and G776 of SEQ ID NO: 2; (u) an insertion of the amino acid sequenceYAMA between positions A775 and G776 of SEQ ID NO: 2; (v) a substitution of the amino acid sequence CV for the G at position 776 of SEQ ID NO: 2; (w) a substitution of the amino acid sequence AVCGG for the GVG between positions 776 and 778 of SEQ ID NO: 2; (x) a substitution of the amino acid sequence CVCG for the GVG between positions 776 and 778 of SEQ ID NO: 2; (y) a substitution of the amino acid sequence VVVG for the GVG between positions 776 and 778 of SEQ ID NO: 2; (z) a substitution of the amino acid sequence SVGG for the GVGS between positions 776 and 779 of SEQ ID NO: 2; (aa) a substitution of the amino acid sequence VVGES for the GVGS between positions 776 and 779 of SEQ ID NO: 2; (bb) a substitution of the amino acid sequence AVGSGV for the GV between positions 776 and 777 of SEQ ID NO: 2; (cc) a substitution of the amino acid sequence CVC for the GV between positions 776 and 777 of SEQ ID NO: 2; (dd) a substitution of the amino acid sequence HVC for the GV between positions 776 and 777 of SEQ ID NO: 2; (ee) a substitution of the amino acid sequence VAAGV for the GV between positions 776 and 777 of SEQ ID NO: 2; (ff) a substitution of the amino acid sequence VAGV for the GV between positions 776 and 777 of SEQ ID NO: 2; (gg) a substitution of the amino acid sequence VVV for the GV between positions 776 and 777 of SEQ ID NO: 2; (hh) an insertion of the amino acid sequence FPG between positions G778 and S779 of SEQ ID NO: 2; (ii) an insertion of the amino acid sequence GS between positions S779 and P780 of SEQ ID NO: 2; (jj) a substitution of the amino acid sequence VPS for the VLRE between positions 754 and 757 of SEQ ID NO: 2; (kk) an insertion of the amino acid E between positions V777 and G778 of SEQ ID NO: 2; (ll) an insertion of the amino acid sequence MAGV between positions V777 and G778 of SEQ ID NO: 2; (mm) an insertion of the amino acid S between positions V777 and G778 of SEQ ID NO: 2; (nn) an insertion of the amino acid sequence SCV between positions V777 and G778 of SEQ ID NO: 2; and (oo) an insertion of the amino acid sequence LMAY between positions Y772 and V773 of SEQ ID NO: 2.

In some embodiments of the uses of the compositions of the disclosure for the treatment of cancer, including those wherein the cancer, a tumor or a cell thereof expresses an oncogenic variant of a HER-2 receptor and wherein the oncogenic variant of a HER2 receptor is an allosteric variant of the HER2 receptor, the oncogenic variant of a HER2 receptor comprises HER2-Δ16, HER2-C311R, HER2-S310F, p95-HER2-M611 or any combination thereof.

In some embodiments, the disclosure provides a use of a composition of the disclosure the treatment of cancer, including those wherein the cancer, a tumor or a cell thereof expresses an oncogenic variant of a HER-4 receptor. In some embodiments, the oncogenic variant of the HER-4 receptor is an allosteric variant of the HER4 receptor. In some embodiments, the oncogenic variant of a HER4 receptor comprises deletion of exon 16 (HER4-Δ16).

In some embodiments of the uses of the compositions of the disclosure for the treatment of cancer, the composition is suitable for systemic administration. In some embodiments, the composition is suitable for oral administration. In some embodiments, the composition is suitable for intravenous administration

In some embodiments of the uses of the compositions of the disclosure for the treatment of cancer, the composition is suitable for local administration. In some embodiments, the composition is suitable for intratumoral intraocular, intraosseus, intraspinal or intracerebroventricular administration.

In some embodiments of the uses of the compositions of the disclosure for the treatment of cancer, the subject or the cancer is insensitive or resistant to treatment with one or more of gefinitinib, erlotinib, afatinib, osimertinib, and necitunumab. In some embodiments, the subject or the cancer is insensitive or resistant to treatment with one or more of crixotinib, alectinib, and ceritinib. In some embodiments, the subject or the cancer is insensitive or resistant to treatment with one or more of dabrafenib and trametinib. In some embodiments, the subject or the cancer is insensitive or resistant to treatment with crizotinib.

In some embodiments of the uses of the compositions of the disclosure for the treatment of cancer, the cancer, tumor or cell thereof expresses an oncogenic variant of an EGFR, wherein the sequence encoding the oncogenic variant of the EGFR comprises a deletion of exon 20 or a portion thereof and wherein the cancer, tumor or cell thereof does not comprise an oncogenic variation in a sequence encoding one or more of an EGFR kinase domain (KD), BRAF, NTRK, and KRAS or wherein.

In some embodiments of the uses of the compositions of the disclosure for the treatment of cancer, the cancer, tumor or cell thereof comprises an oncogenic variant of an EGFR, wherein the sequence encoding the oncogenic variant of the EGFR comprises a deletion of exon 20 or a portion thereof and wherein the cancer, tumor or cell thereof does not comprise a marker indicating responsiveness to immunotherapy.

In some embodiments, the oncogenic variant (e.g., allosteric variant) or the oncogenic mutation (e.g., allosteric mutation) is detected by a Food and Drug Administration (FDA)-approved diagnosis.

In some embodiments, the subject has an adverse reaction to treatment with a therapeutic agent different from the compound of the present disclosure. In some embodiments, the subject has an adverse reaction to treatment with a Type I inhibitor. In some embodiments, the subject has an adverse reaction to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291. In some embodiments, the adverse reaction is an activation of the oncogenic variant of an EGFR and wherein the oncogenic variant comprises a mutation in an extracellular domain of the receptor. In some embodiments, the adverse reaction is an activation of the oncogenic variant of a HER-2 Receptor and wherein the oncogenic variant comprises a mutation in an extracellular domain of the receptor.

In some embodiments, the method comprises administering to the subject in need thereof a therapeutically effective amount of anon-Type I inhibitor. In some embodiments, the non-Type I inhibitor comprises a small molecule Type II inhibitor.

In some embodiments, the method comprises administering to the subject in need thereof a therapeutically effective amount of anon-Type I inhibitor. In some embodiments, the non-Type I inhibitor comprises a small molecule Type II inhibitor.

In some embodiments, the compound is used in combination with a therapeutically effective amount of a non-Type I inhibitor. In some embodiments, the non-Type I inhibitor comprises a small molecule Type II inhibitor.

In some embodiments, the composition comprises a non-Type I inhibitor. In some embodiments, the non-Type I inhibitor comprises a small molecule Type II inhibitor.

In some embodiments, of the uses of the compositions of the disclosure for the treatment of cancer, the cancer comprises a solid tumor. In some embodiments, the cancer comprises a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC) or any subtype thereof. In some embodiments, the cancer comprises a glioblastoma (GBM). In some embodiments, the cancer comprises a breast cancer. In some embodiments, the cancer comprises a lung cancer.

In some embodiments, of the uses of the compositions of the disclosure for the treatment of cancer, the therapeutically effective amount reduces a severity of a sign or symptom of the cancer. In some embodiments, the sign of the cancer comprises a tumor grade and wherein a reduction of the severity of the sign comprises a decrease of the tumor grade. In some embodiments, the sign of the cancer comprises a tumor metastasis and wherein a reduction of the severity of the sign comprises an elimination of the metastasis or a reduction in the rate or extent the metastasis. In some embodiments, the sign of the cancer comprises a tumor volume and wherein a reduction of the severity of the sign comprises an elimination of the tumor or a reduction in the volume. In some embodiments, the symptom of the cancer comprises pain and wherein a reduction of the severity of the sign comprises an elimination or a reduction in the pain.

In some embodiments, of the uses of the compositions of the disclosure for the treatment of cancer, the therapeutically effective amount induces a period of remission.

In some embodiments, of the uses of the compositions of the disclosure for the treatment of cancer, the therapeutically effective amount improves a prognosis of the subject.

In some embodiments, of the uses of the compositions of the disclosure for the treatment of cancer, the subject is a participant or a candidate for participation in in a clinical trial or protocol thereof. In some embodiments, the subject is excluded from treatment with a Type I inhibitor. In some embodiments, the Type I inhibitor comprises gefinitinib, erlotinib, afatinib, osimertinib, necitunumab, crizotinib, alectinib, ceritinib, dabrafenib, trametinib, afatinib, sapitinib, dacomitinib canertinib, pelitinib WZ4002, WZ8040, WZ3146, CO-1686 or AZD9291.

In some embodiments, of the uses of the compositions of the disclosure for the treatment of cancer, the use comprises treating the subject with a Non-Type I inhibitor.

In some embodiments, of the uses of the compositions of the disclosure for the treatment of cancer, the composition comprises a Non-Type I inhibitor.

In some embodiments, of the uses of the compositions of the disclosure for the treatment of cancer, the Non-Type I inhibitor comprises a Type II small molecule inhibitor. In some embodiments, the Type 11 small molecule inhibitor comprises neratinib, AST-1306, HKI-357, or lapatinib.

In some embodiments, the oncogenic variant is an oncogenic variant in an ErbB receptor.

In some embodiments, the oncogenic variant in the ErbB receptor is an allosteric variant.

In some embodiments, the ErbB receptor is an epidermal growth factor receptor (EGFR) or a human epidermal growth factor receptor 2 (HER2) receptor.

In some embodiments, the ErbB receptor is an epidermal growth factor receptor (EGFR).

In some embodiments, the ErbB receptor is a HER2 receptor.

In some embodiments, the oncogenic variant is an oncogenic variant in an epidermal growth factor receptor (EGFR).

In some embodiments, the oncogenic variant in the EGFR is an allosteric variant.

In some embodiments, the oncogenic variant is an oncogenic variant of a HER2 receptor.

In some embodiments, the oncogenic variant in the HER2 receptor is an allosteric variant.

In some embodiments, the oncogenic variant in the EGFR is an EGFR variant III (EGFR-Viii) variant.

In some embodiments, the oncogenic variant in the EGFR is a substitution of a valine (V) for an alanine (A) at position 289 of SEQ ID NO: 1.

In some embodiments, the oncogenic variant is an oncogenic variant in an EGFR and wherein the oncogenic variant in the EGFR is an allosteric variant in the EGFR, the oncogenic variant in the EGFR is a modification of a structure of the EGFR, wherein the oncogenic variant in the EGFR is capable of forming a covalently linked dimer, wherein the covalently linked dimer is constitutively active and wherein the covalently linked dimer enhances an activity of EGFR when contacted to a Type I ErbB inhibitor. In some embodiments, the modification of the structure of the EGFR comprises a modification of one or more of a nucleic acid sequence, an amino acid sequence, a secondary structure, a tertiary structure, and a quaternary structure. In some embodiments, the modification of the structure of the EGFR occurs within a first cysteine rich (CR1) and/or second cysteine rich (CR2) region of EGFR. In some embodiments, the first cysteine rich (CR1) and/or second cysteine rich (CR2) region of EGFR comprises amino acid residues T211-R334 and/or C526-S645 of SEQ ID NO: 1, respectively. In some embodiments, the oncogenic variant in the EGFR generates a physical barrier to formation of a disulfide bond within the CR1 and/or the CR2 region. In some embodiments, the oncogenic variant in the EGFR removes a physical barrier to formation of a disulfide bond within the CR1 and/or the CR2 region. In some embodiments, the oncogenic variant in the EGFR results into one or more free or unpaired Cysteine (C) residues located at a dimer interface of the EGFR. In some embodiments, the oncogenic variant in the EGFR results into one or more free or unpaired Cysteine (C) residues at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1. In some embodiments, the modification occurs within 10 angstroms or less of an intramolecular disulfide bond at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1.

In some embodiments, the oncogenic variant is an oncogenic variant in an EGFR and wherein the oncogenic variant in the EGFR is an allosteric variant in the EGFR, wherein a nucleotide sequence encoding the EGFR having the oncogenic variant comprises a deletion or the substitution comprises one or more amino acids that encode an adenosine triphosphate (ATP) binding site. In some embodiments, the ATP binding site comprises amino acids E746 to A750 of SEQ ID NO: 1. In some embodiments, the ATP binding site or the deletion or substitution thereof comprises K858 of SEQ ID NO: 1. In some embodiments, the deletion comprises K858 of SEQ ID NO: 1. In some embodiments, an arginine (R) is substituted for the lysine (K) at position 858 (K858R) of SEQ ID NO: 1. In some embodiments, an arginine (R) is substituted for the leucine (L) at position 858 (L858R) of SEQ ID NO: 1.

In some embodiments, the oncogenic variant is an oncogenic variant in an EGFR and wherein the oncogenic variant in the EGFR is an allosteric variant in the EGFR, wherein a nucleotide sequence encoding the EGFR having the oncogenic variant comprises an insertion within a sequence encoding exon 20 or a portion thereof. In some embodiments, the sequence encoding exon 20 or a portion thereof comprises a sequence encoding KEILDEAYVMASVDNPHVCAR (SEQ ID NO: 7). In some embodiments, the sequence encoding exon 20 or a portion thereof comprises a sequence encoding a C-helix, a terminal end of the C-helix or a loop following the C-helix. In some embodiments, the insertion comprises the amino acid sequence of ASV, SVD, NPH, or FQEA. In some embodiments, the sequence encoding exon 20 or a portion thereof comprises one or more of: (a) an insertion of the amino acid sequence ASV between positions V769 and D770 of SEQ ID NO: 1; (b) an insertion of the amino acid sequence SVD between positions D770 and N771 of SEQ ID NO: 1; (c) an insertion of the amino acid sequence NPH between positions H773 and V774 of SEQ ID NO: 1; (d) an insertion of the amino acid sequence FQEA between positions A763 and Y764 of SEQ ID NO: 1; (e) an insertion of the amino acid sequence PH between positions H773 and V774 of SEQ ID NO: 1; (f) an insertion of the amino acid G between positions D770 and N771 of SEQ ID NO: 1; (g) an insertion of the amino acid H between positions H773 and V774 of SEQ ID NO: 1; (h) an insertion of the amino acid sequence I-V between positions V774 and C775 of SEQ ID NO: 1; (i) an insertion of the amino acid sequence AH between positions H773 and V774 of SEQ ID NO: 1; (j) an insertion of the amino acid sequence SVA between positions A767 and S768 of SEQ ID NO: 1; (k) a substitution of the amino acid sequence GYN for the DN between positions 770 and 771 of SEQ ID NO: 1; (l) an insertion of the amino acid H between positions N771 and P772 of SEQ ID NO: 1; (m) an insertion of the amino acid Y between positions H773 and V774 of SEQ ID NO: 1; (n) an insertion of the amino acid sequence PHVC between positions C775 and R776 of SEQ ID NO: 1; (o) a substitution of the amino acid sequence YNPY for the H at position 773 of SEQ ID NO: 1; (p) an insertion of the amino acid sequence DNP between positions P772 and H773 of SEQ ID NO: 1; (q) an insertion of the amino acid sequence VDS between positions S768 and V769 of SEQ ID NO: 1; (r) an insertion of the amino acid H between positions D770 and N771 of SEQ ID NO: 1; (s) an insertion of the amino acid N between positions N771 and P772 of SEQ ID NO: 1; (t) an insertion of the amino acid sequence PNP between positions P772 and H773 of SEQ ID NO: 1; (u) a substitution of the amino acid sequence GSVDN for the DN between positions 770 and 771 of SEQ ID NO: 1; (v) a substitution of the amino acid sequence GYP for the NP between positions 771 and 772 of SEQ ID NO: 1; (w) an insertion of the amino acid G between positions N771 and P772 of SEQ ID NO: 1; (x) an insertion of the amino acid sequence GNP between positions P772 and H773 of SEQ ID NO: 1; (y) an insertion of the amino acid sequence GSV between positions V769 and D770 of SEQ ID NO: 1; (z) a substitution of the amino acid sequence GNPHVC for the VC between positions 774 and 775 of SEQ ID NO: 1; (aa) an insertion of the amino acid sequence LQEA between positions A763 and Y764 of SEQ ID NO: 1; (bb) an insertion of the amino acid sequence GL between positions D770 and N771 of SEQ ID NO: 1; (cc) an insertion of the amino acid Y between positions D770 and N771 of SEQ ID NO: 1; (dd) an insertion of the amino acid sequence NPY between positions H773 and V774 of SEQ ID NO: 1; (ee) an insertion of the amino acid sequence TH between positions H773 and V774 of SEQ ID NO: 1; (ff) a substitution of the amino acid sequence KGP for the NP between positions 771 and 772 of SEQ ID NO: 1; (gg) a substitution of the amino acid sequence SVDNP for the NP between positions 771 and 772 of SEQ ID NO: 1; (hh) an insertion of the amino acid sequence NN between positions N771 and P772 of SEQ ID NO: 1; (ii) an insertion of the amino acid T between positions N771 and P772 of SEQ ID NO: 1; and (jj) a substitution of the amino acid sequence STLASV for the SV between positions 768 and 769 of SEQ ID NO: 1.

In some embodiments, the oncogenic variant is an oncogenic variant in an EGFR and wherein the oncogenic variant in the EGFR is an allosteric variant in the EGFR, the EGFR having the oncogenic variant comprises EGFR-Vii, EGFR-Vvi, EGFR-R222C, EGFR-R252C. EGFR-R252P, EGFR-R256Y, EGFR-T263P, EGFR-Y270C, EGFR-A289T, EGFR-A289V, EGFR-A289D, EGFR-H304Y, EGFR-G331R, EGFR-P596S, EGFR-P596L, EGFR-P596R, EGFR-G598V, EGFR-G598A, EGFR-G614D, EGFR-C620Y, EGFR-C614W, EGFR-C628F, EGFR-C628Y, EGFR-C636Y, EGFR-G645C, EGFR-Δ660, EGFR-Δ768 or any combination thereof.

In some embodiments, the oncogenic variant is an oncogenic variant in a HER-2 receptor.

In some embodiments, the oncogenic variant is an oncogenic variant in a HER-2 receptor, the oncogenic variant in the HER2 receptor is an allosteric variant in the HER2 receptor.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of a phenylalanine (F) for a serine (S) at position 310 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of a tyrosine (Y) for a serine (S) at position 310 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of a glutamine (Q) for an arginine (R) at position 678 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of a leucine (L) for a valine (V) at position 777 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of a methionine (M) for a valine (V) at position 777 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of an isoleucine (I) for a valine (V) at position 842 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of an alanine (A) for a leucine (L) at position 755 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of a proline (P) for a leucine (L) at position 755 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, the oncogenic mutatin in the HER2 receptor comprises a substitution of a serine (S) for a leucine (L) at position 755 of SEQ ID NO: 2 or 5.

In some embodiments, the oncogenic variant is an oncogenic variant in a HER-2 receptor and wherein the oncogenic variant in the HER2 receptor is an allosteric variant in the HER2 receptor, wherein a nucleotide sequence encoding the HER2 receptor having the oncogenic variant comprises an insertion within a sequence encoding exon 20 or a portion thereof. In some embodiments, the sequence encoding exon 20 or a portion thereof comprises a sequence encoding KEILDEAYVMAGVGSPYVSR(SEQ ID NO: 8). In some embodiments, the sequence encoding exon 20 or a portion thereof comprises a sequence encoding a C-helix, a terminal end of the C-helix or a loop following the C-helix. In some embodiments, the insertion comprises the amino acid sequence of GSP or YVMA. In some embodiments, the sequence encoding exon 20 or a portion thereof comprises one or more of: (a) an insertion of the amino acid sequence YVMA between positions A775 and G776 of SEQ ID NO: 2; (b) an insertion of the amino acid sequence GSP between positions P780 and Y781 of SEQ ID NO: 2; (c) an insertion of the amino acid sequence YVMA between positions A771 and Y772 of SEQ ID NO: 2; (d) an insertion of the amino acid sequence YVMA between positions 775 and G776 of SEQ ID NO: 2; (e) an insertion of the amino acid V between positions V777 and G778 of SEQ ID NO: 2; (f) an insertion of the amino acid V between positions V777 and 0778 of SEQ ID NO: 2; (g) a substitution of the amino acid sequence AVGCV for the GV between positions 776 and 777 of SEQ ID NO: 2; (h) a substitution of the amino acid sequence LC for the G between position 776 of SEQ ID NO: 2; (i) a substitution of the amino acid sequence LCV for the G between position 776 of SEQ ID NO: 2; (j) an insertion of the amino acid sequence GSP between positions V777 and G778 of SEQ ID NO: 2; (k) a substitution of the amino acid sequence PS for the LRE between positions 755 and 757 of SEQ ID NO: 2; (l) a substitution of the amino acid sequence CPGSP for the SP between positions 779 and 780 of SEQ ID NO: 2; (m) an insertion of the amino acid C between positions V777 and G778 of SEQ ID NO: 2; (n) a substitution of the amino acid sequence VVMA for the AG between positions 775 and 776 of SEQ ID NO: 2; (o) a substitution of the amino acid sequence VV for the G at position 776 of SEQ ID NO: 2; (p) a substitution of the amino acid sequence AVCV for the GV between positions 776 and 777 of SEQ ID NO: 2; (q) a substitution of the amino acid sequence VCV for the GV between positions 776 and 777 of SEQ ID NO: 2; (r) an insertion of the amino acid G between positions G778 and S779 of SEQ ID NO: 2; (s) a substitution of the amino acid sequence PK for the LRE between positions 755 and 757 of SEQ ID NO: 2; (t) an insertion of the amino acid V between positions A775 and G776 of SEQ ID NO: 2; (u) an insertion of the amino acid sequenceYAMA between positions A775 and G776 of SEQ ID NO: 2; (v) a substitution of the amino acid sequence CV for the G at position 776 of SEQ ID NO: 2; (w) a substitution of the amino acid sequence AVCGG for the CVG between positions 776 and 778 of SEQ ID NO: 2; (x) a substitution of the amino acid sequence CVCG for the GVG between positions 776 and 778 of SEQ ID NO: 2; (y) a substitution of the amino acid sequence VVVG for the GVG between positions 776 and 778 of SEQ ID NO: 2; (z) a substitution of the amino acid sequence SVGG for the GVGS between positions 776 and 779 of SEQ ID NO: 2; (aa) a substitution of the amino acid sequence VVGES for the GVGS between positions 776 and 779 of SEQ ID NO: 2; (bb) a substitution of the amino acid sequence AVGSGV for the GV between positions 776 and 777 of SEQ ID NO: 2; (cc) a substitution of the amino acid sequence CVC for the GV between positions 776 and 777 of SEQ ID NO: 2; (dd) a substitution of the amino acid sequence HVC for the GV between positions 776 and 777 of SEQ ID NO: 2; (ee) a substitution of the amino acid sequence VAAGV for the GV between positions 776 and 777 of SEQ ID NO: 2; (ff) a substitution of the amino acid sequence VAGV for the GV between positions 776 and 777 of SEQ ID NO: 2; (gg) a substitution of the amino acid sequence VVV for the GV between positions 776 and 777 of SEQ ID NO: 2; (hh) an insertion of the amino acid sequence FPG between positions G778 and S779 of SEQ ID NO: 2; (ii) an insertion of the amino acid sequence GS between positions S779 and P780 of SEQ ID NO: 2; (jj) a substitution of the amino acid sequence VPS for the VLRE between positions 754 and 757 of SEQ ID NO: 2; (kk) an insertion of the amino acid E between positions V777 and G778 of SEQ ID NO: 2; (ll) an insertion of the amino acid sequence MAGV between positions V777 and G778 of SEQ ID NO: 2; (mm) an insertion of the amino acid S between positions V777 and G778 of SEQ ID NO: 2; (nn) an insertion of the amino acid sequence SCV between positions V777 and G778 of SEQ ID NO: 2; and (oo) an insertion of the amino acid sequence LMAY between positions Y772 and V773 of SEQ ID NO: 2.

In some embodiments, the oncogenic variant is an oncogenic variant in a HER-2 receptor and wherein the oncogenic variant in the HER2 receptor is an allosteric variant in the HER2 receptor, the HER2 receptor having the oncogenic variant comprises HER2-Δ16, HER2-C311R, HER2-S310F, p95-HER2-M611 or any combination thereof.

In some embodiments, the oncogenic variant is an oncogenic variant in a HER-4 receptor. In some embodiments, the oncogenic variant in the HER-4 receptor is an allosteric variant in the HER4 receptor. In some embodiments, the oncogenic variant in the HER4 receptor results into the deletion of exon 16 (HER4-Δ16).

In some embodiments, the oncogenic variant is an oncogenic variant in an EGFR, wherein the sequence encoding the EGFR having the oncogenic variant comprises a deletion of exon 20 or a portion thereof and wherein the cancer, the tumor or the cell thereof does not comprise a second oncogenic variant in a sequence other than exon 20 of EGFR. In some embodiments, the second oncogenic variation comprises a sequence encoding one or more of an EGFR kinase domain (KD), BRAF, NTRK, and KRAS.

In some embodiments, the oncogenic variant is an oncogenic variant in an EGFR, wherein the sequence encoding the EGFR having the oncogenic variant comprises a deletion of exon 20 or a portion thereof and wherein the cancer, the tumor or the cell thereof does not comprise a marker indicating responsiveness to immunotherapy.

### EXAMPLES

### Example 1. Synthesis of Exemplary Compounds of the Present Disclosure

### General Procedure A:

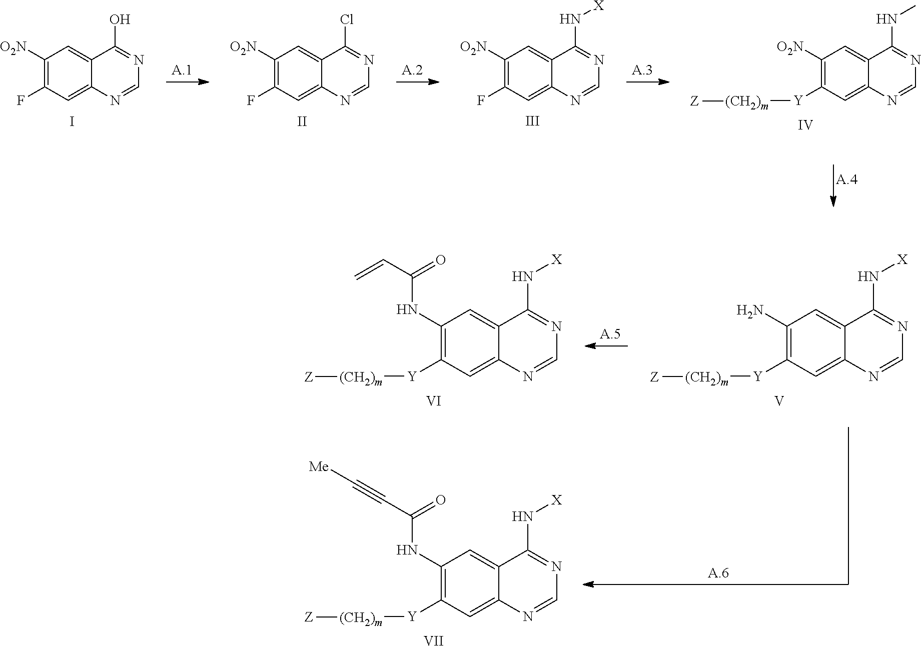

### Step A.1:

A solution of 7-fluoro-6-nitro-quinazolin-4-ol (5.00 g, 23.9 mmol, 1.00 eq) in thionyl chloride (20.0 mL) was added dimethyl formamide (174 mg, 2.39 mmol, 183 uL, 0.10 eq). The reaction was stirred at 80° C. for 10 h. The reaction mixture was concentrated under reduced pressure to give 4-chloro-7-fluoro-6-nitroquinazoline (6.00 g, crude) as an off-white solid. The product was taken to next step without purification.

### Step A.2:

A mixture of 4-chloro-7-fluoro-6-nitroquinazoline (2.4 g, 10.55 mmol, 1 eq) and the free amine H₂N—X (1 eq) in isopropyl alcohol was heated at 80° C. for 1 h. The reaction mixture was concentrated under reduced pressure to give a residue. The residue was triturated with ethyl acetate to give amine III.

### Step A.3:

To a solution of amine III (1 eq) and the NH or OH nucleophile Z—(CH₂)ₘ—YH (1.1 eq) in acetonitrile was added cesium carbonate (2 eq) or DBU (2 eq) and optionally potassium iodide (1 eq). Then the mixture was stirred at 80-110° C. for 12 h. The reaction mixture was quenched by addition of water and then extracted with ethyl acetate. The combined organic layers were washed with brine dried over sodium sulfate, filtered and concentrated under reduced pressure to give a residue. The residue was purified by flash silica gel chromatography to give IV.

### Step A.4:

Variant i): A mixture of IV (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine V.

Variant ii): A mixture of IV (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine V.

### Step A5:

Variant i): To a solution of V (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide VI.

Variant ii): To a solution of V (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide VI.

Variant iii): To a solution of V (1.0 eq) in dimethylformamide was added triethylamine (3.00 eq) and acryloyl chloride (1.20 eq) at 0° C. The reaction mixture was stirred at 0° C. for 1 h and subsequently filtered. The filtrate was purified by prep-HPLC to give acrylamide VI.

### Step A.6:

To a solution of V (1.0 eq), 1-(3-dimethylaminopropyl)-3-ethylcarbodiimide hydrochloride (5.00 eq) and pyridine (5.00 eq) in N,N-dimethylformamide was added but-2-ynoic acid (10.0 eq). The mixture was stirred at 50° C. for 2 h and subsequently concentrated in vacuum. The mixture was purified by prep-HPLC to give ynamide VII.

### General Procedure B:

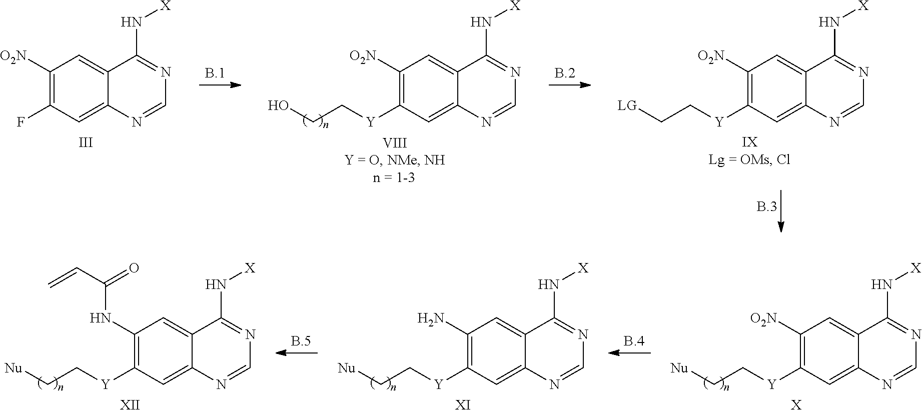

### Step B.1:

To a solution of III, obtained in step A.2 (1.00 eq) and potassium tert-butoxide (4.00 eq) in dimethylsulfoxide (10.0 mL) was added the corresponding diol of aminoalcohol (6.00 eq) dropwise at 20° C. The mixture was stirred at 20° C. for 12 h. The mixture was diluted with water and extracted with ethyl acetate. The combined organic layer was washed with brine and dried over sodium sulfate filtered and concentrated to give crude product. The crude product was purified by silica gel chromatography to give alcohol VIII.

### Step B.2:

Variant i): To a solution of VII (1 eq) and triethylamine (4.00 eq) in dichloromethane and dimethylsulfoxide (6:1) was added MsCl (4.00 eq) dropwise at 0° C. The mixture was stirred at 20° C. for 2 hr. The mixture was diluted with water and extracted with dichloromethane.

The combined organic layer was washed with brine and dried over sodium sulfate, filtered and concentrated to give Mesylate IX.

Variant ii): To a solution of VIII (1.0 eq) in thionyl chloride was added N,N-dimethylformamide (0.1 eq). The mixture was stirred at 90° C. for 3 h. The mixture was cooled to 25° C. and then concentrated in vacuum. The mixture was partitioned between and ethyl acetate. The organic phase was washed with brine, dried with anhydrous sodium sulfate, filtered and concentrated in vacuum. The residue was purified by silica gel chromatography to afford chloride IX.

### Step B.3:

To a solution of IX (1.0 eq) and potassium carbonate (4.00 eq) in dimethylsulfoxide was the corresponding N—H nucleophile (2.0 eq) in one portion at 20° C. The mixture was stirred at 50° C. for 12 h. The mixture was diluted with water and extracted with ethyl acetate. The combined organic layer was washed with brine and dried over sodium sulfate, filtered and concentrated to give crude product. The crude product was purified by prep-HPLC to give X.

### Step B.4:

Variant i): A mixture of X (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine XI.

Variant ii): A mixture of X (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine XI.

### Step B.5:

Variant i): To a solution of XI (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide XII.

Variant ii): To a solution of XI (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide XII.

Variant iii): To a solution of XI (1.0 eq) in dimethylformamide was added triethylamine (3.00 eq) and acryloyl chloride (1.20 eq) at 0° C. The reaction mixture was stirred at 0° C. for 1 h and subsequently filtered. The filtrate was purified by prep-HPLC to give acrylamide XII.

### General Procedure C:

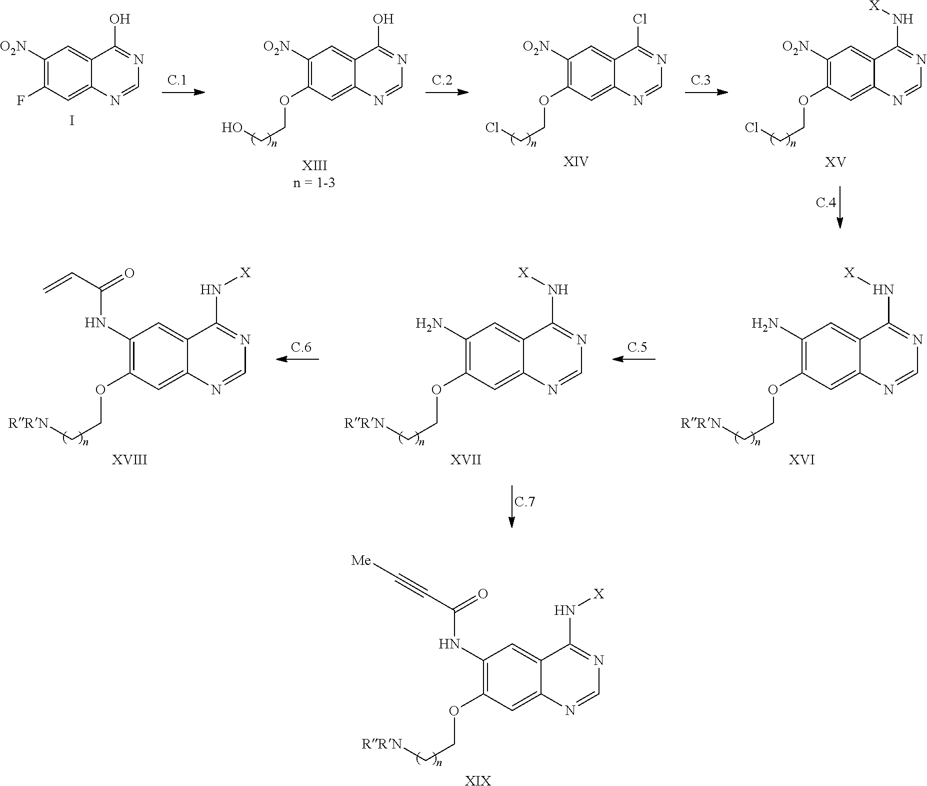

### Step C.1:

Sodium (3.0 eq) was added to the corresponding diol (18.7 eq) at 25° C. The suspension was stirred at 25° C. for 0.5 h. Alcohol I (1.0 eq) was added to the above suspension. The mixture was heated to 70° C. and stirred at 70° C. for 1.5 h. The mixture was cooled to 25° C. and then adjusted to pH=7 with hydrochloric acid (3 M). After filtration, the filter cake was dried under reduced pressure to afford diol XIII.

### Step C.2:

To a solution of diol XIII (1.00 eq) in thionyl chloride (10.0 ml) was added N,N-dimethylformamide (0.1 eq). The mixture was stirred at 90° C. for 3 h. The mixture was cooled to 25° C. and then concentrated in vacuum. The mixture was partitioned between water and ethyl acetate. The organic phase was washed with brine, dried with anhydrous sodium sulfate, filtered and concentrated in vacuum. The residue was purified by silica gel chromatography to afford dichloride XIV.

### Step C.3:

A solution of dichloride XIV (1.0 eq) and H₂N—X (1.50 eq) in propan-2-ol was stirred at 90° C. for 12 h. The mixture was cooled to 25° C. and then concentrated in vacuum. The residue was triturated with methanol, then filtered and dried under reduced pressure to afford XV.

### Step C.4:

To a solution of XV (1.0 eq), potassium iodide (0.1 eq) and tetrabutylammonium iodide (0.1 eq) in toluene was added HNR′R″ (3.00 eq). The mixture was stirred at 110° C. for 12 h. The mixture was cooled to 25° C. and then concentrated in vacuum. The residue was triturated with water and filtered, the filter cake was dried in vacuum to afford XVI.

### Step C.5:

Variant i): A mixture of XVI (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine XVII.

Variant ii): A mixture of XVI (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine XVII.

### Step C.6:

Variant i): To a solution of XVII (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide XVIII.

Variant ii): To a solution of XVII (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide XVIII.

Variant iii): To a solution of XVII (1.0 eq) in dimethylformamide was added triethylamine (3.00 eq) and acryloyl chloride (1.20 eq) at 0 CC. The reaction mixture was stirred at 0 CC for 1 h and subsequently filtered. The filtrate was purified by prep-HPLC to give acrylamide XVII.

### Steps C.7:

To a solution of XVII (1.0 eq), 1-(3-dimethylaminopropyl)-3-ethylcarbodiimide hydrochloride (5.00 eq) and pyridine (5.00 eq) in N,N-dimethylformamide was added but-2-ynoic acid (10.0 eq). The mixture was stirred at 50° C. for 2 h and subsequently concentrated in vacuum. The mixture was purified by prep-HPLC to give ynamide XIX.

### General Procedure D:

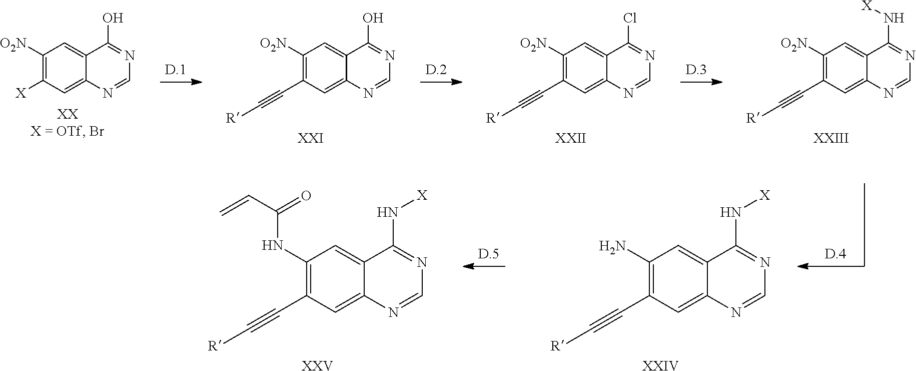

### Step D.1:

To a solution of bromide or triflate XX (1.00 eq) in dimethylsulfoxide was added the corresponding alkyne (1.50 eq), triethylamine (3.00 eq), copper (1) iodide (0.5 eq), tetrakis(triphenylphosphine)palladium (0.05 eq) at 20° C. The mixture was degassed with nitrogen and stirred at 20° C. for 12 h under nitrogen. The mixture was added methanol and filtered, the filter cake was concentrated to give alkyne XXI.

### Step D.2:

To a suspension of alkyne XXI (1.00 eq) in thionyl chloride was added N,N-dimethylformamide (2.0 eq) at 20° C. The mixture was stirred at 90° C. for 0.5 h until the suspension turned to homogenous solution. The solution was concentrated to give chloride XXII.

### Step D.3:

A suspension of chloride XXII (1.0 eq) and H₂N—X in propan-2-ol was stirred at 80° C. for 12 h. The mixture was concentrated to give a residue. And the residue was purified by reverse phase chromatography to give XXIII.

### Step D.4:

Variant i): A mixture of XXIII (1 eq) and nickel(ii) chloride hexahydrate (2 eq) in dichloromethane and methanol (1:1) was added sodium borohydride (4 eq) at 0° C. and then the mixture was stirred at 0° C. for 12 h. The reaction mixture was filtered and the filtrate was concentrated to give a residue. The residue was purified by reversed phase column chromatography to give amine XXIV.

Variant ii): A mixture of XXIII (1 eq), iron (3 eq) and ammonium chloride (5 eq) in methanol and water (4:1) was stirred at 70° C. for 12 h. The reaction mixture was filtered and the filtrate was concentrated under reduced pressure to give a residue. The residue was purified by Reverse-MPLC to give amine XXIV.

### Step D.5:

Variant i): To a solution of XXIV (1 eq), 4-dimethylaminopyridine (1.5 eq) and acrylic acid (1.2 eq) in dimethyl formamide was added 1-(3-dimethylaminopropyl)-3-ethylcarbodiimide (2 eq) and then the solution was stirred at 25° C. for 1 h. The reaction mixture was filtered. The filtrate was purified by prep-HPLC to give acrylamide XXV.

Variant ii): To a solution of XXIV (1 eq) and triethylamine (4 eq) in dimethyl formamide was added acrylic anhydride (1.2 eq) and then the solution was stirred at 25° C. for 0.5 h. The reaction mixture was filtered. The filtrate was purified by prep-HPLC to give acrylamide XXV.

Variant iii): To a solution of XXIV (1.0 eq) in dimethylformamide was added triethylamine (3.00 eq) and acryloyl chloride (1.20 eq) at 0° C. The reaction mixture was stirred at 0° C. for 1 h and subsequently filtered. The filtrate was purified by prep-HPLC to give acrylamide XXV.

| Compound | No. |
|---|---|
| 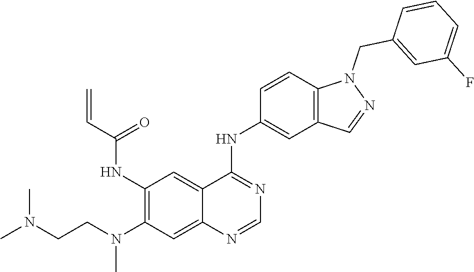 [SMILES: C=CC(=O)Nc1cc2c(Nc3ccc4c(cnn4Cc4cccc(F)c4)c3)ncnc2cc1N(C)CCN(C)C] | 1 |
| 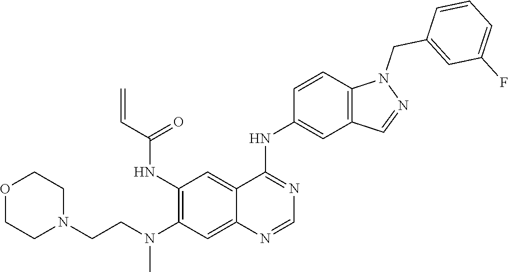 [SMILES: C=CC(=O)Nc1cc2c(Nc3ccc4c(cnn4Cc4cccc(F)c4)c3)ncnc2cc1N(C)CCN1CCOCC1] | 2 |
| 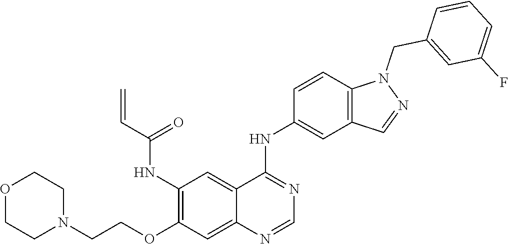 [SMILES: C=CC(=O)Nc1cc2c(Nc3ccc4c(cnn4Cc4cccc(F)c4)c3)ncnc2cc1OCCN1CCOCC1] | 3 |
| 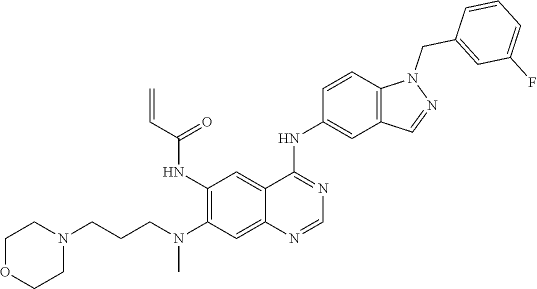 [SMILES: C=CC(=O)Nc1cc2c(Nc3ccc4c(cnn4Cc4cccc(F)c4)c3)ncnc2cc1N(C)CCCN1CCOCC1] | 4 |
| 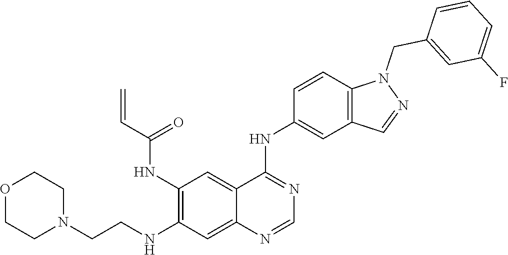 [SMILES: C=CC(=O)Nc1cc2c(Nc3ccc4c(cnn4Cc4cccc(F)c4)c3)ncnc2cc1NCCN1CCOCC1] | 5 |
| 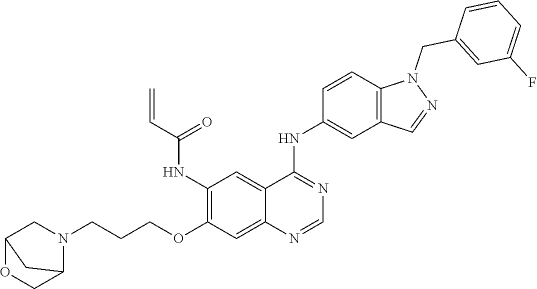 [SMILES: C=CC(=O)Nc1cc2c(Nc3ccc4c(cnn4Cc4cccc(F)c4)c3)ncnc2cc1OCCCN1CC2CC1CO2] | 6 |
| 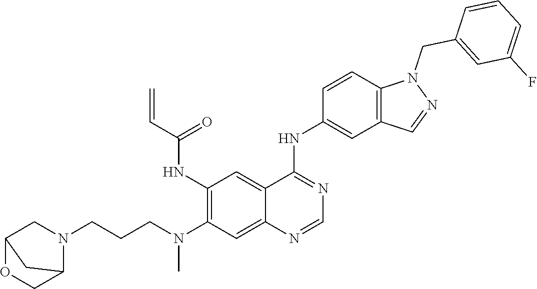 [SMILES: C=CC(=O)Nc1cc2c(Nc3ccc4c(cnn4Cc4cccc(F)c4)c3)ncnc2cc1N(C)CCCN1CC2CC1CO2] | 7 |
| 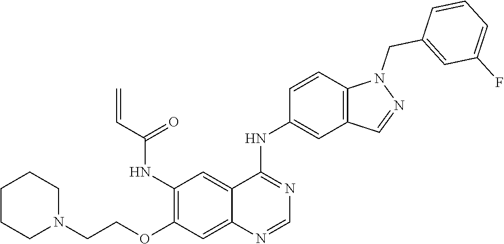 [SMILES: C=CC(=O)Nc1cc2c(Nc3ccc4c(cnn4Cc4cccc(F)c4)c3)ncnc2cc1OCCN1CCCCC1] | 8 |
| 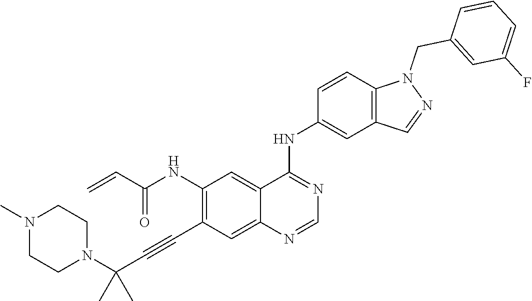 [SMILES: C=CC(=O)Nc1cc2c(Nc3ccc4c(cnn4Cc4cccc(F)c4)c3)ncnc2cc1C#CC(C)(C)N1CCN(C)CC1] | 9 |
| 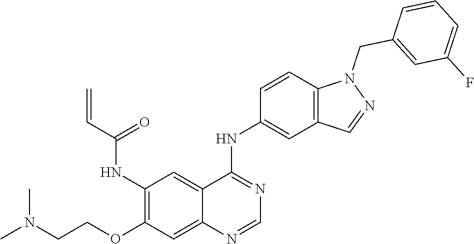 [SMILES: C=CC(=O)Nc1cc2c(Nc3ccc4c(cnn4Cc4cccc(F)c4)c3)ncnc2cc1OCCN(C)C] | 10 |

1: Synthesized according to general procedure A, wherein in step A.2 H₂N—X is 1-(3-fluorobenzyl)-1H-indazol-5-amine (2.54 g, 10.55 mmol); in step A.3 the NH nucleophile is N¹,N¹,N²-trimethylethane-1,2-diamine (181.96 mg, 1.78 mmol) variant i) was used in step A.4; and variant i) was used in step A.5; and 6% overall yield from II. ¹H NMR (400 MHz, CDCl₃) δ=10.51 (s, 1H), 9.29 (s, 1H), 8.64 (s, 1H), 8.16 (d, J=1.5 Hz, 1H), 8.08 (d, J=0.8 Hz, 2H), 7.64 (s, 1H), 7.62 (dd, J=2.0, 9.0 Hz, 1H), 7.37 (d, J=8.8 Hz, 1H), 7.33-7.29 (m, 1H), 7.02-6.90 (m, 3H), 6.58-6.51 (m, 1H), 6.49-6.41 (m, 1H), 5.86-5.81 (m, 1H), 5.62 (s, 2H), 2.96-2.92 (m, 21H), 2.88 (s, 31H), 2.54-2.51 (m, 2H), 2.36 (s, 6H). MS (EST) m/z 539.5 [M+H]⁺.

2: Synthesized according to general procedure A starting from intermediate III (600 mg, 1.4 mmol) obtained in 1, wherein in step A.3 the NH nucleophile is N-methyl-2-morpholinoethanamine (400.24 mg, 2.78 mmol); variant i) was used in step A.4; and variant ii) was used in step A.5; and 40% overall yield from III. ¹H NMR (400 MHz, CDCl₃) δ=9.46 (s, 1H), 9.14 (s, 1H), 8.64 (s, 1H), 8.21 (s, 1H), 8.16 (s, 1H), 8.08 (s, 1H), 7.66 (s, 1H), 7.62 (dd, J=1.6, 8.8 Hz, 1H), 7.37 (d, J=8.8 Hz, 11H), 7.34-7.29 (m, 1H), 7.05-6.94 (m, 2H), 6.91 (br d, J=9.4 Hz, 1H), 6.60-6.48 (m, 2H), 5.90 (dd, J=4.2, 7.4 Hz, 1H), 5.61 (s, 2H), 3.77-3.66 (m, 4H), 3.16 (br t, J=5.8 Hz, 2H), 2.82 (s, 3H), 2.54-2.44 (m, 6H). MS (ESI) m/z 581.3 [M+H]⁺.

3: Synthesized according to general procedure A starting from intermediate III (600 mg, 1.4 mmol) obtained in 1, wherein in step A.3 the OH nucleophile is 2-morpholinoethanol (218 mg, 1.67 mmol, 204 uL); variant i) was used in step A.4; and variant i) was used in step A.5; and 11% overall yield from III. ¹H NMR (400 MHz, DMSO-d₆) δ=8.29 (s, 1H), 8.22 (s, 1H), 8.12 (s, 1H), 7.68 (s, 2H), 7.45 (s, 1H), 7.39-7.32 (m, 1H), 7.12-7.01 (m, 4H), 5.69 (s, 2H), 4.28 (br t, J=5.6 Hz, 2H), 3.63-3.59 (m, 4H), 2.87-2.81 (m, 2H), 2.58-2.52 (m, 4H). MS (ESI) m/z 514.0 [M+H]⁺.

4: Synthesized according to general procedure A starting from intermediate III (600 mg, 1.39 mmol) obtained in 1, wherein in step A.3 the NH nucleophile is N-methyl-3-morpholinopropan-1-amine (439 mg, 2.78 mmol); variant i) was used in step A.4; and variant ii) was used in step A.5; and 8% overall yield from III. ¹H NMR (300 MHz, DMSO-d₆) δ=9.75 (s, 1H), 9.67 (s, 1H), 8.64 (s, 1H), 8.42 (s, 1H), 8.22-8.11 (m, 2H), 7.80-7.59 (m, 2H), 7.46-7.29 (m, 1H), 7.25 (s, 1H), 7.16-7.09 (n, 1H), 7.05 (br d, J=7.4 Hz, 2H), 6.70 (br dd, J=16.6, 9.8 Hz, 1H), 6.33 (dd, J=17.0, 1.8 Hz, 1H), 5.81 (br d, J=11.8 Hz, 11H), 5.69 (s, 2H), 3.51 (br t, 3=4.4 Hz, 4H), 3.09-2.97 (m, 2H), 2.83 (s, 3H), 2.29-2.19 (m, 6H), 1.73 (br d, J=6.8 Hz, 2H). MS (ESI) m/z 595.5 [M+H]⁺.

5: Synthesized according to general procedure A starting from intermediate III (600 mg, 1.39 mmol) obtained in 1, wherein in step A.3 the NH nucleophile is 2-morpholinoethanamine (541 mg, 4.16 mmol); variant i) was used in step A.4; and variant i) was used in step A.5; and 16% overall yield from III. ¹H NMR (400 MHz, DMSO-d₆) δ=9.80 (s, 1H), 9.49 (s, 1H), 8.36 (s, 1H), 8.28 (s, 1H), 8.21 (s, 1H), 8.12 (s, 1H), 7.68 (s, 2H), 7.39-7.33 (m, 11H), 7.12-7.07 (m, 1H), 7.04 (br d, J=7.2 Hz, 2H), 6.74 (s, 1H), 6.55 (dd, J=16.8, 14.4 Hz, 1H), 6.30 (br d, J=16.8 Hz, 1H), 5.85-5.78 (m, 2H). 5.68 (s, 2H), 3.58 (br s, 4H), 3.48 (br s, 1H), 2.60 (br s, 4H), 2.43-2.43 (m, 2H). ¹H NMR (400 MHz, CDCl₃) δ=8.57 (S, 1H), 8.22 (br s, 1H), 8.05 (s, 2H), 7.79 (br s, 1H), 7.62 (br s, 1H), 7.53 (dd, J=8.8, 2.0 Hz, 1H), 7.35-7.28 (m, 2H), 7.00-6.94 (m, 2H), 6.92-6.86 (m, 2H), 6.58-6.51 (m, 1H), 6.45 (br d, J=10.4 Hz, 1H), 5.89 (br d, J=10.4 Hz, 1H), 5.58 (s, 2H), 5.00 (br s, 1H), 3.71 (br s, 4H), 3.23 (br s, 2H), 2.70 (br t, J=6.0 Hz, 2H), 2.49 (br s, 4H). MS (EST) m/z 567.5 [M+H]⁺.

6: Synthesized according to general procedure B, wherein in step B.1 propane-1,3-diol (2.11 g, 27.8 mmol) was used; in step B.2 variant i) was used, in step B.3 the nucleophile is 2-oxa-5-azabicyclo[2.2.1]heptane hydrochloride (622 mg, 4.59 mmol), in step B.4 variant i) was used and variant i) was used in step B.5; and 4% overall yield from III. ¹H NMR (400 MHz, CDCl₃) δ=9.18 (s, 1H), 8.64 (s, 1H), 8.23 (s, 1H), 8.14 (s, 1H), 8.09 (s, 1H), 7.63 (s, 1H), 7.59 (br d, J=8.6 Hz, 1H), 7.38 (d, J=9.0 Hz, 1H), 7.33-7.29 (m, 2H), 7.04-6.96 (m, 2H), 6.92 (br d, J=9.4 Hz, 1H), 6.55-6.48 (m, 1H), 6.45-6.35 (m, 1H), 5.90 (d, J=10.0 Hz, 1H), 5.62 (s, 2H), 4.45 (s, 1H), 4.37 (t, J=6.4 Hz, 2H), 4.07 (d, J=7.8 Hz, 1H), 3.67 (d, J=7.6 Hz, 1H), 3.51 (s, 1H), 2.99 (d, J=9.8 Hz, 1H), 2.93-2.84 (m, 1H), 2.83-2.73 (m, 1H), 2.58 (d, J=9.8 Hz, 1H), 2.11 (quin, J=6.6 Hz, 2H), 1.93-1.87 (m, 1H), 1.79 (br d, J=9.4 Hz, 1H). MS (ESI) m/z 594.5 [M+H]⁻.

7: Synthesized according to general procedure B, wherein in step B.1 3-(methylamino)propan-1-ol (928 ng, 10.4 mmol) was used; in step B.2 variant i) was used, in step B.3 the nucleophile is 2-oxa-5-azabicyclo[2.2.1]heptane hydrochloride (947 mg, 6.99 mmol), in step B.4 variant i) was used and variant ii) was used in step B.5; and 0.5% overall yield from III. ¹H NMR (400 MHz, CDCl₃) δ=9.24 (br s, 1H), 9.16 (s, 1H), 8.61 (s, 1H), 8.45 (br s, 2H), 8.09 (br d, J=9.6 Hz, 2H), 7.64 (s, 1H), 7.58 (br d, J=8.6 Hz, 1H), 7.37 (br d, J=9.0 Hz, 1H), 7.33-7.29 (m, 1H), 7.05-6.95 (m, 2H), 6.95-6.84 (m, 2H), 6.54 (br d, J=16.8 Hz, 1H), 5.88 (br d, J=10.2 Hz, 1H), 5.61 (s, 2H), 4.57 (br s, 1H), 4.20-4.09 (m, 2H), 3.75 (br d, J=9.6 Hz, 1H), 3.33 (br d, J=10.4 Hz, 1H), 3.27-3.14 (m, 3H), 3.09-3.01 (m, 1H), 2.95 (br d, J=11.2 Hz, 1H), 2.71 (s, 3H), 2.21 (br d, J=11.2 Hz, 1H), 2.04 (br d, J=11.2 Hz, 1H), 1.99-1.87 (m, 2H). MS (ESI) m/z 607.6 [M+H]⁺.

8: Synthesized according to general procedure A starting from intermediate III (600 mg, 1.39 mmol) obtained in 1, wherein in step A.3 the NH nucleophile is 2-(1-piperidyl)ethanol (537 mg, 4.16 mmol); variant i) was used in step A.4; and variant i) was used in step A.5; and 14% overall yield from III. ¹H NMR (400 MHz, DMSO-d₆) δ=9.76 (s, 1H), 9.59 (s, 1H), 8.88 (s, 1H), 8.43 (s, 1H), 8.15 (s, 1H), 8.13 (s, 1H), 7.72-7.65 (m, 2H), 7.40-7.33 (m, 1H), 7.29 (s, 1H), 7.13-7.07 (m, 1H), 7.07-7.03 (m, 2H), 6.68 (dd, J=16.8, 10.0 Hz, 1H), 6.30 (dd, J=16.8, 1.2 Hz, 1H), 5.83-5.78 (m, 1H), 5.69 (s, 2H), 4.31 (t, J=6.0 Hz, 2H), 2.78 (t, J=6.0 Hz, 2H), 2.52 (d, J=2.0 Hz, 4H), 1.49 (q, J=5.6 Hz, 4H), 1.40-1.33 (m, 2H). MS (ESI) m/z 566.5 [M+H]⁺.

9: Synthesized according to general procedure D, wherein ins step D.1 the alkyne is 1-methyl-4-(2-methylbut-3-yn-2-yl)piperazine (5.00 g, 30.1 mmol); and in step D.3 H₂N—X is 1-(3-fluorobenzyl)-1H-indazol-5-amine (645 mg, 2.67 mmol); in step D.4 variant ii) was used; in step D.5 variant iii) was used and 0.5% overall yield from XX. ¹H NMR (400 MHz, MeOD) δ=8.70 (s, 1H), 8.44 (s, 1H), 8.16-8.08 (m, 2H), 7.86 (s, 1H), 7.70-7.63 (m, 1H), 7.61-7.55 (m, 1H), 7.38-7.29 (m, 1H), 7.07-6.96 (m, 2H), 6.91 (br d, J=10.0 Hz, 1H), 6.64-6.54 (m, 1H), 6.52-6.44 (m, 1H), 5.90 (dd, J=1.6, 10.0 Hz, 1H), 5.69 (s, 21H), 2.85 (br m, 4H), 2.55 (br m, 4H), 2.30 (s, 3H), 1.54 (s, 6H). MS (ESI) m/z 603.3 [M+H]⁺.

10: Synthesized according to general procedure A starting from intermediate III (1.00 g, 2.31 mmol) obtained in 1, wherein in step A.3 the OH nucleophile is 2-(dimethylamino)ethanol (247 mg, 2.78 mmol); variant i) was used in step A.4; and variant i) was used in step A.5; and 7% overall yield from III. ¹H NMR (400 MHz, DMSO-d₆) δ=9.76 (br s, 1H). 9.64 (br s, 1H), 8.90 (s, 1H), 8.43 (s, 1H), 8.15 (s, 1H), 8.13 (s, 1H), 7.71-7.66 (m, 2H), 7.40-7.34 (m, 1H), 7.30 (s, 1H), 7.13-7.03 (m, 3H), 6.67 (dd, J=17.2, 10.0 Hz, 1H), 6.31 (dd, J=17.2, 2.0 Hz, 1H), 5.84-5.79 (m, 1H), 5.69 (s, 2H), 4.30 (t, J=5.6 Hz, 2H), 2.75 (1, J=5.6 Hz, 2H), 2.26 (s, 6H). MS (EST) m/z 526.4 [M+H4].

### Example 2. Inhibition Activity of Exemplary Compounds of the Present Disclosure

Retroviral Production: EGFR mutants were subcloned into pMXs-IRES-Blasticidin (RTV-016, Cell Biolabs, San Diego, Calif.). Retroviral expression vector retrovirus was produced by transient transfection of HEK 293T cells with the retroviral EGFR mutant expression vector pMXs-IRES-Blasticidin (RTV-016, Cell Biolabs), pCMV-Gag-Pol vector and pCMV-VSV-G-Envelope vector. HEK 293T/17 cells were plated in 100 mm collagen coated plate (354450, Corning Life Sciences, Tewksbury, Mass.) (4×10⁵ per plate) and incubated overnight. The next day, retroviral plasmids (3 μg of EGFR mutant, 1.0 μg of pCMV-Gag-Pol and 0.5 μg pCMV-VSV-G) were mixed in 500 μl of Optimem (31985, Life Technologies). The mixture was incubated at room temperature for 5 min and then added to Optimem containing transfection reagent Lipofectamine (11668, Invitrogen) and incubated for 20 minutes. Mixture was then added dropwise to HEK 293T cells. The next day the medium was replaced with fresh culture medium and retrovirus was harvested @ 24 and 48 hrs. Generation of EGFR mutant stable cell lines: BaF3 cells (1.5E5 cells) were infected with 1 ml of viral supernatant supplemented with 8 μg/ml polybrene by centrifuging for 30 min at 1000 rpm. Cells were placed in a 37° C. incubator overnight. Cells were then spun for 5 minutes to pellet the cells. Supernatant was removed and cells re-infected a fresh 1 m1 of viral supernatant supplemented with 8 μg/ml polybrene by centrifuging for 30 min at 1000 rpm. Cells were placed in 37° C. incubator overnight. Cells were then maintained in RPMI containing 10% Heat Inactivated FBS, 2% L-glutamine containing 10 ng/ml IL-3. After 48 hours cells were selected for retroviral infection in 10 μg/ml Blasticidin for one week. Blasticidin resistant populations were washed twice in phosphate buffered saline before plating in media lacking IL-3 to select for IL-3 independent growth.

Assay for cell proliferation: BaF3 cell lines were resuspended at 1.3E5 c/ml in RPMI containing 10% Heat Inactivated FBS, 2% L-glutamine and 1% Pen/Strep and dispensed in triplicate (17.5E4 c/well) into 96 well plates. To determine the effect of drug on cell proliferation, cells incubated for 3 days in the presence of vehicle control or test drug at varying concentrations. Inhibition of cell growth was determined by luminescent quantification of intracellular ATP content using CellTiterGlo (Promega), according to the protocol provided by the manufacturer. Comparison of cell number on day 0 versus 72 hours post drug treatment was used to plot dose-response curves. The number of viable cells was determined and normalized to vehicle-treated controls. Inhibition of proliferation, relative to vehicle-treated controls was expressed as a fraction of 1 and graphed using PRISM® software (Graphpad Software, San Diego, Calif.). EC₅₀ values were determined with the same application.

Cellular protein analysis: Cell extracts were prepared by detergent lysis (RIPA, R0278, Sigma, St Louis, Mo.) containing 10 mM Iodoacetamide (786-228, G-Biosciences, St, Louis, Mo.), protease inhibitor (P8340, Sigma, St. Louis, Mo.) and phosphatase inhibitors (P5726, P0044, Sigma, St. Louis, Mo.) cocktails. The soluble protein concentration was determined by micro-BSA assay (Pierce, Rockford Ill.). Protein immunodetection was performed by electrophoretic transfer of SDS-PAGE separated proteins to nitrocellulose, incubation with antibody, and chemiluminescent second step detection. Nitrocellulose membranes were blocked with 5% nonfat dry milk in TBS and incubated overnight with primary antibody in 5% bovine serum albumin. The following primary antibodies from Cell Signaling Technology were used at 1:1000 dilution: phospho-EGFR[Y1173] and total EGFR. β-Actin antibody, used as a control for protein loading, was purchased from Sigma Chemicals. Horseradish peroxidase-conjugated secondary antibodies were obtained from Cell Signaling Technology and used at 1:5000 dilution. Horseradish peroxidase-conjugated secondary antibodies were incubated in nonfat dry milk for 1 hour. SuperSignal chemiluminescent reagent (Pierce Biotechnology) was used according to the manufacturer's directions and blots were imaged using the Alpha Innotech image analyzer and AlphaEaseFC software (Alpha Innotech, San Leandro Calif.).

Tables A and B assign each compound a potency code: A, B, C, D, E, F, G, H, H, I, J or K. According to the code, A represents an IC50 value ≤5 nM. B represents an IC50 value >5 nM and ≤10 nM. C represents an IC50 value >10 nM and ≤20 nM. D represents an IC50 value >20 nM and ≤30 nM. E represents an IC50 value >30 nM and ≤50 nM. F represents an IC50 value >50 nM and ≤100 nM. G represents an IC50 value >100 nM and ≤200 nM. H represents an IC50 value >200 nM and ≤300 nM. I represents an IC50 value >300 nM and ≤500 nM. J represents an IC50 value >500 nM and ≤1000 nM. K represents an IC50 value >1000 nM.

TABLE A
Activity for Inhibiting EGFR

| Compound No. | EGFR WT | EGFR V3 | EGFR NPH | EGFR SVD |
|---|---|---|---|---|
| 1 | J | F | | |
| 2 | K | E | | |
| 3 | H | C | C | C |
| 4 | J | E | | |
| 5 | H | G | | |
| 6 | G | E | | |
| 7 | I | G | | |
| 8 | G | D | E | E |
| 9 | I | E | E | E |
| 10 | G | D | E | E |

TABLE B
Activity for Inhibiting HER2

| Compound No. | HER2 WT | HER2 S310F | HER2 YVMA |
|---|---|---|---|
| 1 | D | G | |
| 2 | D | G | |
| 3 | A | B | D |
| 4 | C | G | |
| 5 | B | G | |
| 6 | A | C | |
| 7 | C | G | |
| 8 | B | C | G |
| 9 | B | C | F |
| 10 | A | D | F |

### Example 3. Treatment of Mice Bearing HER2 Mutant Tumors

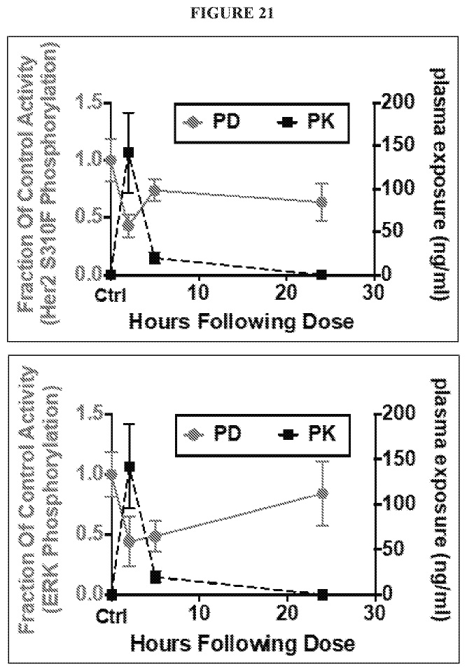

A mouse tumor model containing the HER2 S310F mutation was used to determine the ability of Compound No. 3 to inhibit tumor growth and induce tumor regression in vivo. Athymic nude mice from Charles River Labs bearing HER2 S310F BaF3 tumors were treated with two day acute oral dosing of Compound No. 3 at 15 mg/kg. Following the second dose, tumors were collected at 2 hours, 5 hours, 12 hours and 24 hours, and analyzed for both pHER2 activity and pERK activity via AlphaLisa. Plasma was also collected at these time points and analayzed for the presence of Compound No. 3 to determine pharmacokinetic profile. The tumor tissue was cut and homogenized using the Precellys Soft Tissue Homogenizing kit (KT03961-1-00.3.2) containing T-PER tissue protein extraction reagent (Thermo Scientific #78510), supplemented with Protease Inhibitor (Sigma P8340), and Phosphatase Inhibitors II and III (Sigma P5726 and P0044). Tissue samples were homogenized in the Precellys machine by spinning two times for one minute each. Sample tubes were centrifuged for 5 min at 15,000 rpm at 4° C. The supernatant was transferred to a fresh microtube and spun again for 5 minutes at 15,000 rpm at 4° C. Supernatant was then transferred to a fresh microtube and placed on ice. The protein concentration of the supernatant was measured using the BCA reagent Kit (Thermo Scientific #23225). Tumor tissue-derived lysates were analyzed for either HER2 activity or EGFR activity by detection of pHER2 (Tyr1221/1222) or pERK (Thr202/Tyr204) phosphosites, respectively, via AlphaLisa. Briefly, tumor Lysates were diluted to 0.5 ug/ul in 1× diluted SureFire Ultra Kit Lysis Buffer (5× supplied stock) supplemented with Protease Inhibitor (Sigma P8340) and Phosphatase Inhibitor II and III (Sigma P5726 and P0044). 10 ul of total tumor lysate was added per well in triplicate to a 384-well Opti-plate (Perkin Elmer #6007290). Activation Buffer was diluted 25-fold in combined Reaction Buffer I and Reaction Buffer 2, and acceptor beads were diluted 50-fold in the combined Reaction Buffers. 5 ul/well of the Acceptor bead:Reaction buffer mixture was added to each well. The plate was covered and shaken for 5 minutes on a plate shaker and then incubated at room temperature in the dark for 90 minutes before reading. pHer2 AlphaLisa (Perkin Elmer #ALSU-PEB2-A10K) was used to quantify phosphorylation of Her2 (Tyr1221/1222) or pERK AlphLisA (Perkin Elmer #ALSUPERK-A10K) was used to quantify phosphorylation of ERK1/2 (Thr202/Tyr204) in the control and Compound No. 3 treated tumor samples. As shown in FIG. 21, the administration of Compound No. 3 resulted in a reduction of pHER2 and pERK1/2 at peak plasma levels, indicating that Compound No. 3 inhibits target HER2 S310F mutant kinase activity.

### EQUIVALENTS

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference.

The foregoing description has been presented only for the purposes of illustration and is not intended to limit the disclosure to the precise form disclosed, but by the claims appended hereto.

1. A compound or pharmaceutically acceptable salts or stereoisomers thereof with formula I 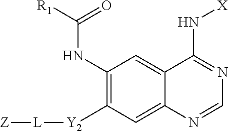
wherein L is a covalent bond, straight chain or branched C₁₋₄ alkyl or 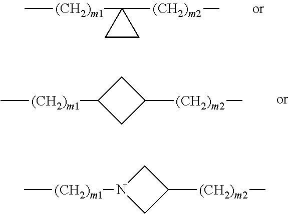
wherein m1, n2 are independently of each other 0, 1, 2, 3, or 4;
Y² is a covalent bond, —O—, —NH—, —NCH₃, —C≡C—;
Z is —(NR⁴R⁵), wherein R⁴ and R⁵ are independently of each other H, C₁₋₆ alkyl, cyclopropyl, cylobutyl, 3 to 6-membered heterocycloalkyl, or —(NR⁶R⁷), —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged- or spirobicycle or a combination thereof and is unsubstituted or substituted with C₁₋₄ alkyl, hal, —OR′, —NR′R″, wherein R′, R″ are independently of each other H or —C₁₋₄ alkyl;
R¹ is —CR_{b}═CHRₐ, —C≡CH or —C≡C—CH₃; wherein Rₐ, R_{b} are independently of each other H, hal, —CH₂—O—CH₃; and
X is a group of formula (i)a 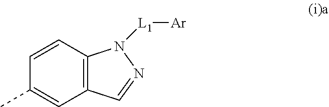
wherein
Ar is 6 membered aryl or N-heteroaryl, which is unsubstituted or substituted with one or more of a group selected from halogen, C₁₋₆alkyl, C₁₋₆alkoxy, —CF₃ or —OCF₃;
L¹ is a covalent bond or straight chain or branched C₁₋₃alkyl, which is unsubstituted or substituted with hal, preferably a covalent bond or —CH₂—.

2. The compound of claim 1 or pharmaceutically acceptable salts or stereoisomers thereof of formula I, wherein L¹ is selected from a covalent bond, —CH₂—, —CH(CH₃)—, CH(hal)-, —CH₂—CH₂— or —CH₂—CH(CH₃)—, —CH₂—CH(hal)-, preferably —CH₂—, —CH₂—CH₂—, more preferably —CH₂—.

3. The compound of claim 1 or pharmaceutically acceptable salts or stereoisomers thereof of formula I, wherein Z is is —(NR⁴R⁵), wherein R⁴ and R⁵ are independently of each other H, C₁₋₄ alkyl, or —(NR⁶R⁷), wherein R⁶ and R⁷ form together with the atom to which they are attached to 3 to 6-membered, preferably 5-membered heteroaryl or 3 to 9-membered, preferably 6-8-membered heterocycloalkyl, wherein the 3 to 9-membered heterocycloalkyl is a monocycle or a bridged bicycle and is unsubstituted or substituted with C₁₋₄ alkyl.

4. The compound of any of the preceding claims or pharmaceutically acceptable salts or stereoisomers thereof of formula I, wherein Ar of the compound of formula Ia (and I) or pharmaceutically acceptable salts or stereoisomers thereof is a group of formula (i)a 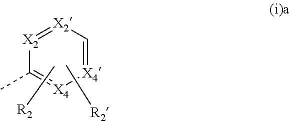
wherein X², X^{2′}, X⁴, X^{2′} are independently of each other —N═ or —CH═;
R², R^{2′} are independently of each other H, C₁₋₆ alkyl, hal, —CF₃, —OCF₃, with the proviso that at least two of X², X^{2′}, X⁴, X^{2′} are —CH═.

5. The compound of claim 4 or pharmaceutically acceptable salts or stereoisomers thereof of formula I, wherein (i) X² and X^{2′} are —CH═ or (ii) X² is —CH═ and X^{2′} is —N═ or X^{2′} is —CH═ and X² is —N═ or (iii) or X² and X^{2′} are —N═.

6. The compound of claim 4 or 5 or pharmaceutically acceptable salts or stereoisomers thereof of formula I, wherein (i) X⁴ and X^{4′} are —CH— or (ii) X⁴ is —N═ and X^{2′} is —CH═ or X^{2′} is —N— and X⁴ is —CH═ or (iii) X⁴ and X^{4′} are —N═.

7. The compound of any of the preceding claims or pharmaceutically acceptable salts or stereoisomers thereof wherein X is a group of formula (ii)b, preferably (ii)c or (ii)c′ 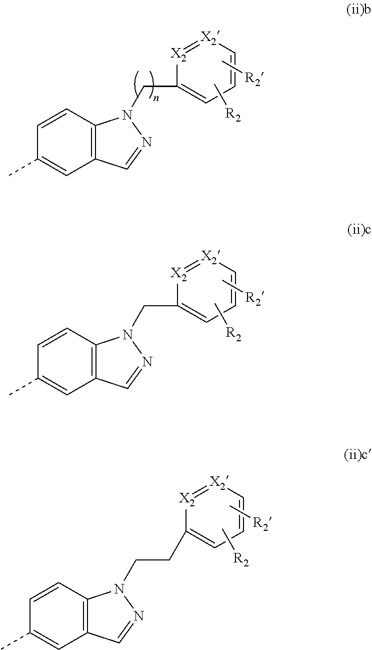
wherein
X², X^{2′} are independently of each other —N═, —CH═;
R², R^{2′} are independently of each other H, C₁₋₆ alkyl, hal, —CF₃, —OCF₃, and
n is 1 or 2.

8. The compound of any of the preceding claims or pharmaceutically acceptable salts or stereoisomers thereof wherein X has the following formula IId, IIe, IIf 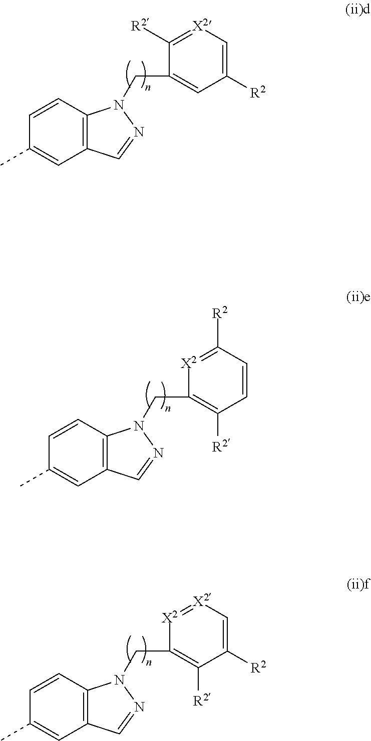
wherein X², X^{2′} are independently of each other —N═ or —CH═;
R², R^{2′} are independently of each other H, C₁₋₆ alkyl, hal, —CF₃, or —OCF₃,
n is 1 or 2.

9. The compound of any of the preceding claims or pharmaceutically acceptable salts or stereoisomers thereof of formula I, wherein X is 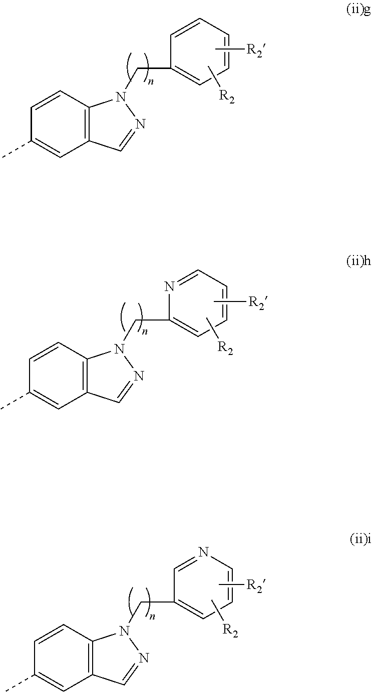
wherein
R², R^{2′} are independently of each other H, C₁₋₆ alkyl, hal, preferably H, —CH₃, F, Cl;
n is 1 or 2.

10. The compound of any of the preceding claims or pharmaceutically acceptable salts or stereoisomers thereof of formula I, wherein —(NR⁶R⁷), —(CR⁶R⁷) are selected from 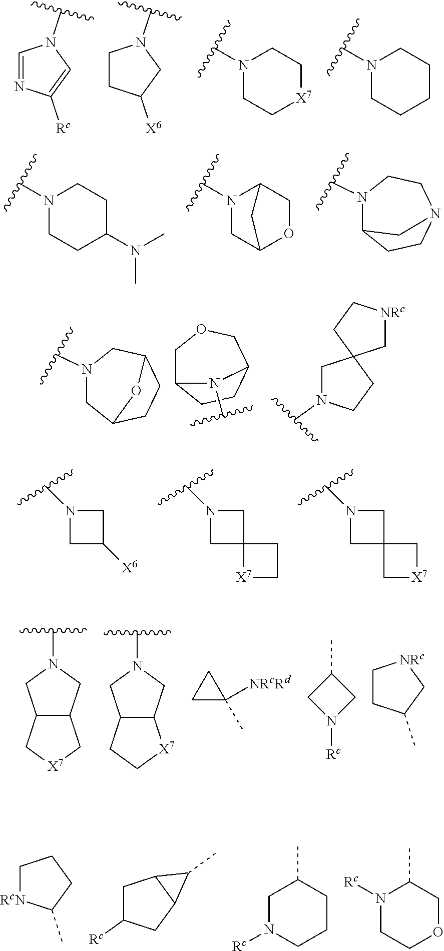
wherein
R^{c}, is H, C₁₋₄ alkyl, oxetane, and R^{d} is H, C₁₋₄ alkyl;
X⁶ is H, —CH₃, —OH, —OCH₃, —OCF₃, —N(CH₃)₂, F, Cl,
X⁷ is —O—, —NH— or —N(CH₃)—.

11. The compound any of the preceding claims or pharmaceutically acceptable salts or stereoisomers thereof of formula I, wherein —(NR⁶R⁷) is selected from 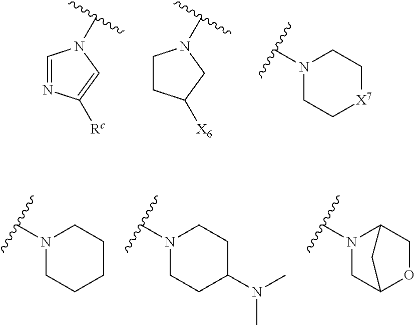
wherein
R^{c} is H, C₁₋₄ alkyl, oxetane,
X⁶ is H, —CH₃, —OH, —OCH₃, —OCF₃, —N(CH₃)₂, F, Cl, preferably H, —CH₃;
X⁷ is —O—, —NH— or —N(CH₃)—.

12. The compound of claim 1 or pharmaceutically acceptable salts or stereoisomers thereof having formula II or III 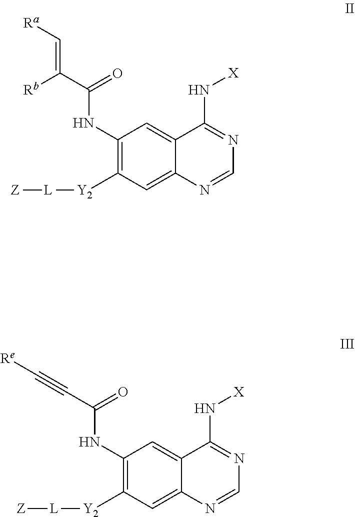
wherein
L is a covalent bond, straight chain or branched C₁₋₄ alkyl or 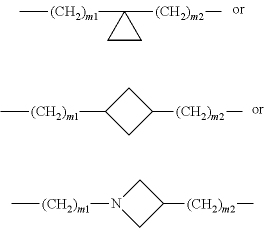
wherein m1, m2 are independently of each other 0, 1, 2, 3, or 4, preferably a covalent bond, straight chain or branched C₁₋₄ alkyl;
Y² is a covalent bond, —O—, —NH—, —NCH₃—, —C≡C—;
Z is —(NR⁴R⁵), wherein R⁴ and R⁵ are independently of each other H, C₁₋₆ alkyl cyclopropyl, cylobutyl, 3 to 6-membered heterocycloalkyl, or —(NR⁶R⁷), —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged- or spirobicycle or a combination thereof and is unsubstituted or substituted with C₁₋₄ alkyl, hal, —OR′, —NR′R″, wherein R′, R″ are independently of each other H or —C₁₋₄ alkyl;
Rₐ, R_{b} are independently of each other H, hal, or —CH₂—O—CH₃, preferably H, and Rₑ is H or methyl; and X is a group of formula (ii)a 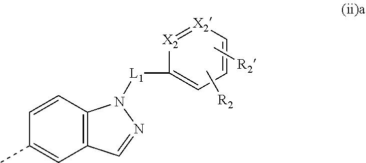
wherein
X², X^{2′} are independently of each other —N═, —CH═;
L¹ is a covalent bond or straight chain or branched C₁₋₃alkyl, which is unsubstituted or substituted with hal;
R², R^{2′} are independently of each other H, C₁₋₆ alkyl, hal, —CF₃, —OCF₃, preferably H, hal.

13. The compound of claim 1 or pharmaceutically acceptable salts or stereoisomers thereof having the formula IV 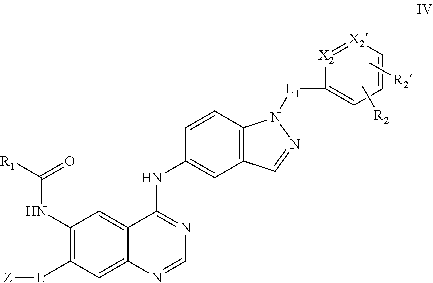
wherein
L¹ is a covalent bond or straight chain or branched C₁₋₃alkyl, which is unsubstituted or substituted with hal;
X², X^{2′} are independently of each other —N═, —CH═;
R¹ is —CR_{b}═CHRₐ, —C≡CH or —C≡C—CH₃, wherein Rₐ, R_{b} are independently of each other H, hal, —CH₂—O—CH₃;
R², R^{2′} are independently of each other H, C₁₋₆ alkyl, hal, —CF₃, —OCF₃;
L is a covalent bond, straight chain or branched C₁₋₄ alkyl or 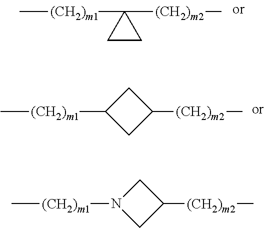
wherein m1, m2 are independently of each other 0, 1, 2, 3, or 4, preferably L is a covalent bond, straight chain or branched C₁₋₄ alkyl;
Z is —(NR⁴R⁵), wherein R⁴ and R⁵ are independently of each other H, C₁₋₆ alkyl, cyclopropyl, cylobutyl, 3 to 6-membered heterocycloalkyl, or —(NR⁶R⁷), —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged- or spirobicycle or a combination thereof and is unsubstituted or substituted with C₁₋₄ alkyl, hal, —OR′, —NR′R″, wherein R′, R″ are independently of each other H or —C₁₋₄ alkyl.

14. The compound of claim 1 or pharmaceutically acceptable salts or stereoisomers thereof having the formula VII 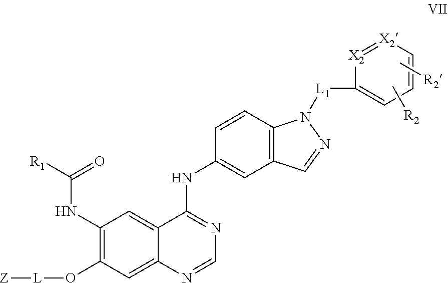
wherein
L¹ is a covalent bond or straight chain or branched C₁₋₃alkyl, which is unsubstituted or substituted with hal;
X², X^{2′} are independently of each other —N═, —CH═;
R¹ is —CR_{b}═CHRₐ, —C≡CH or —C≡C—CH₃, wherein Rₐ, R_{b} are independently of each other H, hal, —CH₂—O—CH₃;
R², R^{2′} are independently of each other H, C₁₋₆ alkyl, hal, —CF₃, —OCF₃;
L is a covalent bond, straight chain or branched C₁₋₄ alkyl or 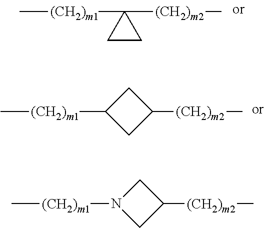
wherein m1, m2 are independently of each other 0, 1, 2, 3, or 4, preferably L is a covalent bond, straight chain or branched C₁₋₄ alkyl;
Z is —(NR⁴R⁵), wherein R⁴ and R⁵ are independently of each other H, C₁₋₆ alkyl, cyclopropyl, cylobutyl, 3 to 6-membered heterocycloalkyl, or —(NR⁶R⁷), —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged- or spirobicycle or a combination thereof and is unsubstituted or substituted with C₁₋₄ alkyl, hal, —OR′, —NR′R″, wherein R′, R″ are independently of each other H or —C₁₋₄ alkyl.

15. The compound of claim 1 or pharmaceutically acceptable salts or stereoisomers thereof having the formula X 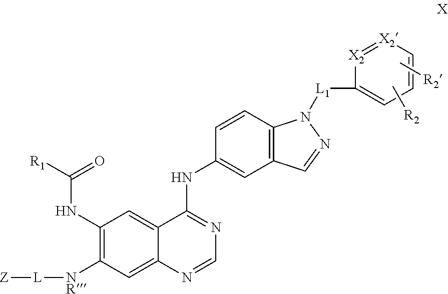
wherein
L¹ is a covalent bond or straight chain or branched C₁₋₃alkyl, which is unsubstituted or substituted with hal;
X², X^{2′} are independently of each other —N═, —CH═;
R¹ is —CR_{b}═CHRₐ, —C≡CH or —C≡C—CH₃, wherein Rₐ, R_{b} are independently of each other H, hal, —CH₂—O—CH₃;
R², R^{2′} are independently of each other H, C₁₋₆ alkyl, hal, —CF₃, —OCF₃;
L is a covalent bond, straight chain or branched C₁₋₄ alkyl or 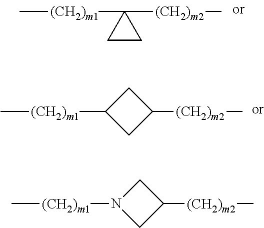
wherein m1, m2 are independently of each other 0, 1, 2, 3, or 4, preferably L is a covalent bond, straight chain or branched C₁₋₄ alkyl;
R′″ is H or —CH₃;
Z is —(NR⁴R⁵), wherein R⁴ and R⁵ are independently of each other H, C₁₋₆ alkyl, cyclopropyl, cylobutyl, 3 to 6-membered heterocycloalkyl, or —(NR⁶R⁷), —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged- or spirobicycle or a combination thereof and is unsubstituted or substituted with C₁₋₄ alkyl, hal, —OR′, —NR′R″, wherein R′, R″ are independently of each other H or —C₁₋₄ alkyl.

16. The compound or pharmaceutically acceptable salts or stereoisomers thereof having the following formula XIII 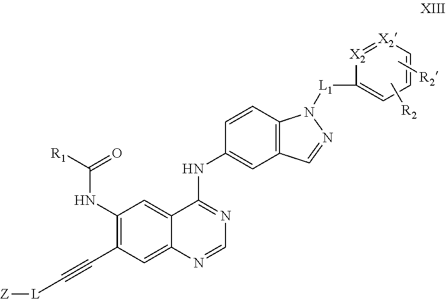
wherein
L¹ is a covalent bond or straight chain or branched C₁₋₃alkyl, which is unsubstituted or substituted with hal;
X², X^{2′} are independently of each other —N═, —OCF₃;
R′ is —CR_{b}═CHRₐ, —C≡CH or —C≡C—CH₃, wherein Rₐ, R_{b} are independently of each other H, hal, —CH₂—O—CH₃;
R², R^{2′} are independently of each other H, C₁₋₆ alkyl, hal, —CF₃, —OCF₃;
L is a covalent bond, straight chain or branched C₁₋₄ alkyl or 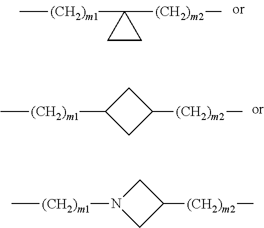
wherein m1, m2 are independently of each other 0, 1, 2, 3, or 4, preferably L is a covalent bond, straight chain or branched C₁₋₄ alkyl;
Z is —(NR⁴R⁵), wherein R⁴ and R⁵ are independently of each other H, C₁₋₆ alkyl, cyclopropyl, cylobutyl, 3 to 6-membered heterocycloalkyl, or —(NR⁶R⁷), —(CHR⁶R⁷), wherein R⁶ and R⁷ form together with the atom to which they are attached to 3 to 6-membered heteroaryl or 3 to 9-membered heterocycloalkyl, wherein the 3 to 9-membered heterocycloalkyl is a monocycle or a fused-, bridged- or spirobicycle or a combination thereof and is unsubstituted or substituted with C₁₋₄ alkyl, hal, —OR′, —NR′R″, wherein R′, R″ are independently of each other H or —C₁₋₄ alkyl.

17. The compound of any of the preceding claims or pharmaceutically acceptable salts or stereoisomers thereof of formula I, wherein —(NR⁶R⁷), —(CHR⁶R⁷) are selected from 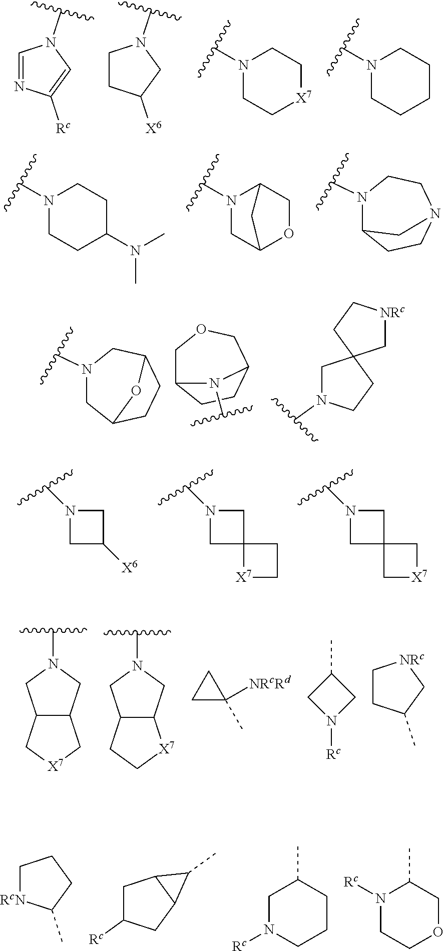
wherein
R^{c} is H, C₁₋₄ alkyl, oxetane;
X⁶ is H, —CH₃, —OH, —OCH₃, —OCF₃, —N(CH₃)₂, F, Cl,
X⁷ is —O—, —NH— or —N(CH₃)—; and
R^{d} is H, C₁₋₄ alkyl.

18. The compound of any one of the preceding claims, being selected from the compounds described in Table I and pharmaceutically acceptable salts thereof.

19. The compound of any one of the preceding claims, being selected from the compounds described in Table I.

20. A composition comprising a compound according to any one of claims 1-19 or pharmaceutically acceptable salts or stereoisomers thereof.

21. The composition of claim 20, further comprising a pharmaceutically acceptable carrier.

22. The composition of claim 20 or 21, further comprising a second therapeutically active agent.

23. The composition of any one of claims 20-22 for use in the treatment of cancer.

24. A method of inhibiting an oncogenic variant of an ErbB receptor, comprising administering the subject in need thereof a therapeutically effective amount of the compound of any one of the preceding claims.

25. A method of inhibiting an oncogenic variant of an ErbB receptor, comprising administering the subject in need thereof the composition of any one of the preceding claims.

26. A method of preventing or treating cancer, comprising administering the subject in need thereof a therapeutically effective amount of the compound of any one of the preceding claims.

27. A method of preventing or treating cancer, comprising administering the subject in need thereof the composition of any one of the preceding claims.

28. A method of preventing or treating cancer, comprising: i) identifying a subject candidate as the subject in need of the treatment when that at least one oncogenic variant of an ErbB receptor is present in the subject; and ii) administering the subject in need of the treatment a therapeutically effective amount of the compound of any one of the preceding claims.

29. A method of preventing or treating cancer, comprising: i) identifying a subject candidate as the subject in need of the treatment when that at least one oncogenic variant of an ErbB receptor is present in the subject; and ii) administering the subject in need of the treatment the composition of any one of the preceding claims.

30. A method of preventing or treating cancer, comprising: i) identifying a subject candidate as the subject in need of the treatment when that at least one oncogenic variant of an ErbB receptor is present in a biological sample from the subject; and ii) administering the subject in need of the treatment a therapeutically effective amount of the compound of any one of the preceding claims.

31. A method of preventing or treating cancer, comprising: i) identifying a subject candidate as the subject in need of the treatment when that at least one oncogenic variant of an ErbB receptor is present in a biological sample from the subject; and ii) administering the subject in need of the treatment the composition of any one of the preceding claims.

32. A method of preventing or treating cancer, comprising administering the subject in need thereof a therapeutically effective amount of the compound of any one of the preceding claims when that at least one oncogenic variant of an ErbB receptor is identified as being present in the subject.

33. A method of preventing or treating cancer, comprising administering the subject in need thereof the compound of any one of the preceding claims when that at least one oncogenic variant of an ErbB receptor is identified as being present in the subject.

34. A method of preventing or treating cancer, comprising administering the subject in need thereof a therapeutically effective amount of the compound of any one of the preceding claims when that at least one oncogenic variant of an ErbB receptor is identified as being present in a biological sample from the subject.

35. A method of preventing or treating cancer, comprising administering the subject in need thereof the composition of any one of the preceding claims when that at least one oncogenic variant of an ErbB receptor is identified as being present in a biological sample from the subject.

36. The compound of any one of the preceding claims for use in the inhibition of an oncogenic variant of an ErbB receptor.

37. The compound of any one of the preceding claims for use in the prevention or treatment of cancer.

38. The composition of any one of the preceding claims for use in the inhibition of an oncogenic variant of an ErbB receptor.

39. The composition of any one of the preceding claims for use in the prevention or treatment of cancer.

40. The compound of any one of the preceding claims for use in the prevention or treatment of cancer in a subject, wherein at least one oncogenic variant of an ErbB receptor is present in the subject.

41. The composition of any one of the preceding claims for use in the prevention or treatment of cancer in a subject, wherein at least one oncogenic variant of an ErbB receptor is present in the subject.

42. The compound of any one of the preceding claims for use in the prevention or treatment of cancer in a subject, wherein at least one oncogenic variant of an ErbB receptor is present in a biological sample from the subject.

43. The composition of any one of the preceding claims for use in the prevention or treatment of cancer in a subject, wherein at least one oncogenic variant of an ErbB receptor is present in a biological sample from the subject.

44. Use of the compound of any one of the preceding claims in the manufacture of a medicament for inhibiting an oncogenic variant of an ErbB receptor.

45. Use of the compound of any one of the preceding claims in the manufacture of a medicament for preventing or treating cancer.

46. The method, the compound for use, or the composition for use of any one of the preceding claims, wherein the cancer, a tumor or a cell thereof expresses the oncogenic variant of an EGFR.

47. The method, the compound for use, or the composition for use of any one of the preceding claims, wherein the oncogenic variant of EGFR is an allosteric variant of EGFR.

48. The method, the compound for use, or the composition for use of any one of the preceding claims, wherein the oncogenic variant of an EGFR comprises an EGFR variant III (EGFR-Viii) mutation.

49. The method, the compound for use, or the composition for use of any one of the preceding claims, wherein the oncogenic variant of an EGFR comprises a substitution of a valine (V) for an alanine (A) at position 289 of SEQ ID NO: 1.

50. The method, the compound for use, or the composition for use of any one of the preceding claims, wherein the oncogenic variant of an EGFR comprises a modification of a structure of the EGFR, wherein the oncogenic variant of an EGFR is a capable of forming a covalently linked dimer, wherein the covalently linked dimer is constitutively active and wherein the covalently linked dimer enhances an activity of EGFR when contacted to a Type I ErbB inhibitor.

51. The method, the compound for use, or the composition for use of any one of the preceding claims, wherein the modification of the structure of the EGFR comprises a modification of one or more of a nucleic acid sequence, an amino acid sequence, a secondary structure, a tertiary structure, and a quaternary structure.

52. The method, the compound for use, or the composition for use of any one of the preceding claims, wherein the oncogenic variant comprises a mutation, a splicing event, a post-translational process, a conformational change or any combination thereof.

53. The method, the compound for use, or the composition for use of any one of the preceding claims, wherein the modification of the structure of the EGFR occurs within a first cysteine rich (CR1) and/or second cysteine rich (CR2) region of EGFR.

54. The method, the compound for use, or the composition for use of any one of the preceding claims, wherein the first cysteine rich (CR1) and/or second cysteine rich (CR2) region of EGFR comprises amino acid residues T211-R334 and/or C526-S645 of SEQ ID NO: 1, respectively.

55. The method, the compound for use, or the composition for use of any one of the preceding claims, wherein the oncogenic variant of an EGFR generates a physical barrier to formation of a disulfide bond within the CR1 and/or the CR2 region.

56. The method, the compound for use, or the composition for use of any one of the preceding claims, wherein the oncogenic variant of an EGFR removes a physical barrier to formation of a disulfide bond within the CR1 and/or the CR2 region.

57. The method, the compound for use, or the composition for use of any one of the preceding claims, wherein the oncogenic variant of an EGFR comprises one or more free or unpaired Cysteine (C) residues located at a dimer interface of the EGFR.

58. The method, the compound for use, or the composition for use of any one of the preceding claims, wherein the oncogenic variant of an EGFR comprises one or more free or unpaired Cysteine (C) residues at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1.

59. The method, the compound for use, or the composition for use of any one of the preceding claims, wherein the modification occurs within 10 angstroms or less of an intramolecular disulfide bond at a site selected from the group consisting of C190-C199, C194-C207, C215-C223, C219-C231, C232-C240, C236-C248, C251-C260, C264-C291, C295-C307, C311-C326, C329-C333, C506-C515, C510-C523, C526-C535, C539-C555, C558-C571, C562-C579, C582-C591, C595-C617, C620-C628 and C624-C636 according to SEQ ID NO: 1.

60. The method, the compound for use, or the composition for use of any one of the preceding claims, wherein a nucleotide sequence encoding the oncogenic variant of an EGFR comprises a deletion of a sequence encoding exon 19 or a portion thereof.

61. The method, the compound for use, or the composition for use of any one of the preceding claims, wherein the deletion encodes an adenosine triphosphate binding (ATP) site.

62. The method, the compound for use, or the composition for use of any one of the preceding claims, wherein the ATP binding site comprises E746-A750 of SEQ ID NO: 1.

63. The method, the compound for use, or the composition for use of any one of the preceding claims, wherein a nucleotide sequence encoding the oncogenic variant of an EGFR comprises an insertion within a sequence encoding exon 20 or a portion thereof.

64. The method, the compound for use, or the composition for use of any one of the preceding claims, wherein the sequence encoding exon 20 or a portion thereof comprises a sequence encoding KEILDEAYVMASVDNPHVCAR (SEQ ID NO: 7).

65. The method, the compound for use, or the composition for use of any one of the preceding claims, wherein the sequence encoding exon 20 or a portion thereof comprises a sequence encoding a C-helix, a terminal end of the C-helix or a loop following the C-helix.

66. The method, the compound for use, or the composition for use of any one of the preceding claims, wherein the insertion comprises the amino acid sequence of ASV, SVD, NPH, or FQEA.

67. The method, the compound for use, or the composition for use of any one of the preceding claims, wherein the sequence encoding exon 20 or a portion thereof comprises one or more of:
(a) an insertion of the amino acid sequence ASV between positions V769 and D770 of SEQ ID NO: 1;
(b) an insertion of the amino acid sequence SVD between positions D770 and N771 of SEQ ID NO: 1;
(c) an insertion of the amino acid sequence NPH between positions H773 and V774 of SEQ ID NO: 1;
(d) an insertion of the amino acid sequence FQEA between positions A763 and Y764 of SEQ ID NO: 1;
(e) an insertion of the amino acid sequence PH between positions H773 and V774 of SEQ ID NO: 1;
(f) an insertion of the amino acid G between positions D770 and N771 of SEQ ID NO: 1;
(g) an insertion of the amino acid H between positions H773 and V774 of SEQ ID NO: 1;
(h) an insertion of the amino acid sequence HV between positions V774 and C775 of SEQ ID NO: 1;
(i) an insertion of the amino acid sequence AH between positions H773 and V774 of SEQ ID NO: 1;
(j) an insertion of the amino acid sequence SVA between positions A767 and S768 of SEQ ID NO: 1;
(k) a substitution of the amino acid sequence GYN for the DN between positions 770 and 771 of SEQ ID NO: 1;
(l) an insertion of the amino acid H between positions N771 and P772 of SEQ ID NO: 1;
(m) an insertion of the amino acid Y between positions H773 and V774 of SEQ ID NO: 1;
(n) an insertion of the amino acid sequence PHVC between positions C775 and R776 of SEQ ID NO: 1;
(o) a substitution of the amino acid sequence YNPY for the H at position 773 of SEQ ID NO: 1;
(p) an insertion of the amino acid sequence DNP between positions P772 and H773 of SEQ ID NO: 1;
(q) an insertion of the amino acid sequence VDS between positions S768 and V769 of SEQ ID NO: 1;
(r) an insertion of the amino acid H between positions D770 and N771 of SEQ ID NO: 1;
(s) an insertion of the amino acid N between positions N771 and P772 of SEQ ID NO: 1;
(t) an insertion of the amino acid sequence PNP between positions P772 and H773 of SEQ ID NO: 1;
(u) a substitution of the amino acid sequence GSVDN for the DN between positions 770 and 771 of SEQ ID NO: 1;
(v) a substitution of the amino acid sequence GYP for the NP between positions 771 and 772 of SEQ ID NO: 1;
(w) an insertion of the amino acid G between positions N771 and P772 of SEQ ID NO: 1;
(x) an insertion of the amino acid sequence GNP between positions P772 and H773 of SEQ ID NO: 1;
(y) an insertion of the amino acid sequence GSV between positions V769 and D770 of SEQ ID NO: 1;
(z) a substitution of the amino acid sequence GNPHVC for the VC between positions 774 and 775 of SEQ ID NO: 1;
(aa) an insertion of the amino acid sequence LQEA between positions A763 and Y764 of SEQ ID NO: 1;
(bb) an insertion of the amino acid sequence GL between positions D770 and N771 of SEQ ID NO: 1;
(cc) an insertion of the amino acid Y between positions D770 and N771 of SEQ ID NO: 1;
(dd) an insertion of the amino acid sequence NPY between positions H773 and V774 of SEQ ID NO: 1;
(ee) an insertion of the amino acid sequence TH between positions H773 and V774 of SEQ ID NO: 1;
(ff) a substitution of the amino acid sequence KGP for the NP between positions 771 and 772 of SEQ ID NO: 1;
(gg) a substitution of the amino acid sequence SVDNP for the NP between positions 771 and 772 of SEQ ID NO: 1;
(hh) an insertion of the amino acid sequence NN between positions N771 and P772 of SEQ ID NO: 1;
(ii) an insertion of the amino acid T between positions N771 and P772 of SEQ ID NO: 1; and
(jj) a substitution of the amino acid sequence STLASV for the SV between positions 768 and 769 of SEQ ID NO: 1.

68. The method, the compound for use, or the composition for use of any one of the preceding claims, wherein the oncogenic variant of an EGFR comprises EGFR-Vii, EGFR-Vvi, EGFR-R222C, EGFR-R252C, EGFR-R252P, EGFR-R256Y, EGFR-T263P, EGFR-Y270C, EGFR-S289T, EGFR-S289V, EGFR-A289D, EGFR-H304Y, EGFR-G331R, EGFR-P596S, EGFR-P596L, EGFR-P596R, EGFR-G598V, EGFR-G598A, EGFR-G614D, EGFR-C620Y, EGFR-C614W, EGFR-C628F, EGFR-C628Y, EGFR-C636Y, EGFR-G645C, EGFR-Δ660, EGFR-Δ768 or any combination thereof.

69. A method of treating cancer in a subject, comprising administering to a subject a therapeutically effective amount of the composition of any one of claims 20-22, wherein the cancer is characterized by expression of one or more of:
(a) a wild type human epidermal growth factor receptor 2 (HER2) receptor or
(b) an oncogenic variant of a HER-2 receptor.

70. The method, the compound for use, or the composition for use of any one of the preceding claims, wherein the cancer, a tumor or a cell thereof expresses one or more of:
(a) a wild type human epidermal growth factor receptor 2 (HER2) receptor or
(b) an oncogenic variant of a HER-2 receptor.

71. The method, the compound for use, or the composition for use of any one of the preceding claims, wherein the wild type HER2 receptor comprises the amino acid sequence of SEQ ID NO: 2, 3, 4, 5, or 6.

72. The method, the compound for use, or the composition for use of any one of the preceding claims, wherein the oncogenic variant of a HER2 receptor is an allosteric variant of the HER2 receptor.

73. The method, the compound for use, or the composition for use of any one of the preceding claims, wherein the oncogenic variant of a HER2 receptor comprises a substitution of a phenylalanine (F) for a serine (S) at position 310 of SEQ ID NO: 2 or 5.

74. The method, the compound for use, or the composition for use of any one of the preceding claims, wherein the oncogenic variant of a HER2 receptor comprises a substitution of a tyrosine (Y) for a serine (S) at position 310 of SEQ ID NO: 2 or 5.

75. The method, the compound for use, or the composition for use of any one of the preceding claims, wherein the oncogenic variant of a HER2 receptor comprises a substitution of a glutamine (Q) for an arginine (R) at position 678 of SEQ ID NO: 2 or 5.

76. The method, the compound for use, or the composition for use of any one of the preceding claims, wherein the oncogenic variant of a HER2 receptor comprises a substitution of a leucine (L) for a valine (V) at position 777 of SEQ ID NO: 2 or 5.

77. The method, the compound for use, or the composition for use of any one of the preceding claims, wherein the oncogenic variant of a HER2 receptor comprises a substitution of a methionine (M) for a valine (V) at position 777 of SEQ ID NO: 2 or 5.

78. The method, the compound for use, or the composition for use of any one of the preceding claims, wherein the oncogenic variant of a HER2 receptor comprises a substitution of an isoleucine (I) for a valine (V) at position 842 of SEQ ID NO: 2 or 5.

79. The method, the compound for use, or the composition for use of any one of the preceding claims, wherein the oncogenic variant of a HER2 receptor comprises a substitution of an alanine (A) for a leucine (L) at position 755 of SEQ ID NO: 2 or 5.

80. The method, the compound for use, or the composition for use of any one of the preceding claims, wherein the oncogenic variant of a HER2 receptor comprises a substitution of a proline (P) for a leucine (L) at position 755 of SEQ ID NO: 2 or 5.

81. The method, the compound for use, or the composition for use of any one of the preceding claims, wherein the oncogenic variant of a HER2 receptor comprises a substitution of a serine (S) for a leucine (L) at position 755 of SEQ ID NO: 2 or 5.

82. The method, the compound for use, or the composition for use of any one of the preceding claims, wherein a nucleotide sequence encoding the oncogenic variant of a HER2 receptor comprises an insertion within a sequence encoding exon 20 or a portion thereof.

83. The method, the compound for use, or the composition for use of any one of the preceding claims, wherein the sequence encoding exon 20 or a portion thereof comprises a sequence encoding KEILDEAYVMAGVGSPYVSR(SEQ ID NO: 8).

84. The method, the compound for use, or the composition for use of any one of the preceding claims, wherein the sequence encoding exon 20 or a portion thereof comprises a sequence encoding a C-helix, a terminal end of the C-helix or a loop following the C-helix.

85. The method, the compound for use, or the composition for use of any one of the preceding claims, wherein the insertion comprises the amino acid sequence of GSP or YVMA.

86. The method, the compound for use, or the composition for use of any one of the preceding claims, wherein the sequence encoding exon 20 or a portion thereof comprises one or more of:
(a) an insertion of the amino acid sequence YVMA between positions A775 and G776 of SEQ ID NO: 2;
(b) an insertion of the amino acid sequence GSP between positions P780 and Y781 of SEQ ID NO: 2;
(c) an insertion of the amino acid sequence YVMA between positions A771 and Y772 of SEQ ID NO: 2;
(d) an insertion of the amino acid sequence YVMA between positions A775 and G776 of SEQ ID NO: 2;
(e) an insertion of the amino acid V between positions V777 and G778 of SEQ ID NO: 2;
(f) an insertion of the amino acid V between positions V777 and G778 of SEQ ID NO: 2;
(g) a substitution of the amino acid sequence AVGCV for the GV between positions 776 and 777 of SEQ ID NO: 2;
(h) a substitution of the amino acid sequence LC for the G between position 776 of SEQ ID NO: 2;
(i) a substitution of the amino acid sequence LCV for the G between position 776 of SEQ ID NO: 2;
(j) an insertion of the amino acid sequence GSP between positions V777 and G778 of SEQ ID NO: 2;
(k) a substitution of the amino acid sequence PS for the LRE between positions 755 and 757 of SEQ ID NO: 2;
(l) a substitution of the amino acid sequence CPGSP for the SP between positions 779 and 780 of SEQ ID NO: 2;
(n) an insertion of the amino acid C between positions V777 and G778 of SEQ ID NO: 2;
(n) a substitution of the amino acid sequence VVMA for the AG between positions 775 and 776 of SEQ ID NO: 2;
(o) a substitution of the amino acid sequence VV for the G at position 776 of SEQ ID NO: 2;
(p) a substitution of the amino acid sequence AVCV for the GV between positions 776 and 777 of SEQ ID NO: 2;
(q) a substitution of the amino acid sequence VCV for the GV between positions 776 and 777 of SEQ ID NO: 2;
(r) an insertion of the amino acid G between positions G778 and S779 of SEQ ID NO: 2;
(s) a substitution of the amino acid sequence PK for the LRE between positions 755 and 757 of SEQ ID NO: 2;
(t) an insertion of the amino acid V between positions A775 and G776 of SEQ ID NO: 2;
(u) an insertion of the amino acid sequenceYAMA between positions A775 and G776 of SEQ ID NO: 2;
(v) a substitution of the amino acid sequence CV for the G at position 776 of SEQ ID NO: 2;
(w) a substitution of the amino acid sequence AVCGG for the CVG between positions 776 and 778 of SEQ ID NO: 2;
(x) a substitution of the amino acid sequence CVCG for the GVG between positions 776 and 778 of SEQ ID NO: 2;
(y) a substitution of the amino acid sequence VVVG for the GVG between positions 776 and 778 of SEQ ID NO: 2;
(z) a substitution of the amino acid sequence SVGG for the GVGS between positions 776 and 779 of SEQ ID NO: 2;
(aa) a substitution of the amino acid sequence VVGES for the GVGS between positions 776 and 779 of SEQ ID NO: 2;
(bb) a substitution of the amino acid sequence AVGSGV for the GV between positions 776 and 777 of SEQ ID NO: 2;
(cc) a substitution of the amino acid sequence CVC for the GV between positions 776 and 777 of SEQ ID NO: 2;
(dd) a substitution of the amino acid sequence HVC for the GV between positions 776 and 777 of SEQ ID NO: 2;
(ee) a substitution of the amino acid sequence VAAGV for the GV between positions 776 and 777 of SEQ ID NO: 2;
(ff) a substitution of the amino acid sequence VAGV for the GV between positions 776 and 777 of SEQ ID NO: 2;
(gg) a substitution of the amino acid sequence VVV for the GV between positions 776 and 777 of SEQ ID NO: 2;
(hh) an insertion of the amino acid sequence FPG between positions G778 and S779 of SEQ ID NO: 2;
(ii) an insertion of the amino acid sequence GS between positions S779 and P780 of SEQ ID NO: 2;
(jj) a substitution of the amino acid sequence VPS for the VLRE between positions 754 and 757 of SEQ ID NO: 2;
(kk) an insertion of the amino acid E between positions V777 and G778 of SEQ ID NO: 2;
(ll) an insertion of the amino acid sequence MAGV between positions V777 and G778 of SEQ ID NO: 2;
(mm) an insertion of the amino acid S between positions V777 and G778 of SEQ ID NO: 2;
(nn) an insertion of the amino acid sequence SCV between positions V777 and G778 of SEQ ID NO: 2; and
(oo) an insertion of the amino acid sequence LMAY between positions Y772 and V773 of SEQ ID NO: 2.

87. The method, the compound for use, or the composition for use of any one of the preceding claims, wherein the oncogenic variant of a HER2 receptor comprises HER2-Δ16, HER2-C311R, HER2-S310F, p95-HER2-M611 or any combination thereof.

88. A method of treating cancer in a subject, comprising administering to a subject a therapeutically effective amount of the composition of any one of claims 20-22, wherein the cancer is characterized by expression of an oncogenic variant of a HER-4 receptor.

89. The method, the compound for use, or the composition for use of any one of the preceding claims, wherein the oncogenic variant of the HER-4 receptor is an allosteric variant of the HER4 receptor.

90. The method, the compound for use, or the composition for use of any one of the preceding claims, wherein the oncogenic variant of a HER4 receptor comprises deletion of exon 16 (HER4-Δ16).

91. The method, the compound for use, or the composition for use of any one of the preceding claims, wherein the administration is systemic.

92. The method, the compound for use, or the composition for use of any one of the preceding claims, wherein the administration oral.

93. The method, the compound for use, or the composition for use of any one of the preceding claims, wherein the administration is intravenous.

94. The method, the compound for use, or the composition for use of any one of the preceding claims, wherein the administration is local.

95. The method, the compound for use, or the composition for use of any one of the preceding claims, wherein the administration intratumoral, intraocular, intraosseus, intraspinal or intracerebroventricular.

96. The method, the compound for use, or the composition for use of any one of the preceding claims, wherein the oncogenic variant or the oncogenic mutation is detected by a Food and Drug Administration (FDA)-approved diagnosis.

97. The method, the compound for use, or the composition for use of any one of the preceding claims, wherein prior to the treatment with the compound of the present disclosure, the subject is treated with a therapeutic agent different from the compound of any one of the preceding claims.

98. The method, the compound for use, or the composition for use of any one of the preceding claims, wherein the cancer, or a tumor or a cell thereof, is insensitive or resistant to treatment with the therapeutic agent different from the compound of any one of the preceding claims.

99. The method, the compound for use, or the composition for use of any one of the preceding claims, wherein the subject has an adverse reaction to treatment with a therapeutic agent different from the compound of any one of the preceding claims.

100. The method, the compound for use, or the composition for use of any one of the preceding claims, wherein the cancer, a tumor or a cell thereof is insensitive or resistant to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.

101. The method, the compound for use, or the composition for use of any one of the preceding claims, wherein the subject has an adverse reaction to treatment with one or more of gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 and AZD9291.

102. The method, the compound for use, or the composition for use of any one of the preceding claims, wherein the adverse reaction is an activation of the oncogenic variant of an EGFR and wherein the oncogenic variant comprises a mutation in an extracellular domain of the receptor.

103. The method, the compound for use, or the composition for use of any one of the preceding claims, wherein the adverse reaction is an activation of the oncogenic variant of a HER-2 Receptor and wherein the oncogenic variant comprises a mutation in an extracellular domain of the receptor.

104. The method, the compound for use, or the composition for use of any one of the preceding claims, wherein the cancer, a tumor or a cell thereof expresses an oncogenic variant of an EGFR, wherein the sequence encoding the oncogenic variant of the EGFR comprises a deletion of exon 20 or a portion thereof and wherein the the cancer, the tumor or the cell thereof does not comprise a second oncogenic variation in a sequence other than exon 20 of EGFR.

105. The method, the compound for use, or the composition for use of any one of the preceding claims, wherein the second oncogenic variation comprises a sequence encoding one or more of an EGFR kinase domain (KD), BRAF, NTRK, and KRAS.

106. The method, the compound for use, or the composition for use of any one of the preceding claims, wherein the cancer, a tumor or a cell thereof expresses an oncogenic variant of an EGFR, wherein the sequence encoding the oncogenic variant of the EGFR comprises a deletion of exon 20 or a portion thereof and wherein the the cancer, the tumor or the cell thereof does not comprise a marker indicating responsiveness to immunotherapy.

107. The method, the compound for use, or the composition for use of any one of the preceding claims, wherein the cancer comprises a solid tumor.

108. The method, the compound for use, or the composition for use of any one of the preceding claims, wherein the cancer is a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC) or any subtype thereof.

109. The method, the compound for use, or the composition for use of any one of the preceding claims, wherein the cancer is a glioblastoma (GBM) or any subtype thereof.

110. The method, the compound for use, or the composition for use of any one of the preceding claims, wherein the cancer is a breast cancer or any subtype thereof.

111. The method, the compound for use, or the composition for use of any one of the preceding claims, wherein the cancer is a lung cancer or any subtype thereof.

112. The method, the compound for use, or the composition for use of any one of the preceding claims, wherein the therapeutically effective amount reduces a severity of a sign or symptom of the cancer.

113. The method, the compound for use, or the composition for use of any one of the preceding claims, wherein the sign of the cancer comprises a tumor grade and wherein a reduction of the severity of the sign comprises a decrease of the tumor grade.

114. The method, the compound for use, or the composition for use of any one of the preceding claims, wherein the sign of the cancer comprises a tumor metastasis and wherein a reduction of the severity of the sign comprises an elimination of the metastasis or a reduction in the rate or extent the metastasis.

115. The method, the compound for use, or the composition for use of any one of the preceding claims, wherein the sign of the cancer comprises a tumor volume and wherein a reduction of the severity of the sign comprises an elimination of the tumor or a reduction in the volume.

116. The method, the compound for use, or the composition for use of any one of the preceding claims, wherein the symptom of the cancer comprises pain and wherein a reduction of the seventy of the sign comprises an elimination or a reduction in the pain.

117. The method, the compound for use, or the composition for use of any one of the preceding claims, wherein the therapeutically effective amount induces a period of remission.

118. The method, the compound for use, or the composition for use of any one of the preceding claims, wherein the therapeutically effective amount improves a prognosis of the subject.

119. The method, the compound for use, or the composition for use of any one of the preceding claims, wherein the subject is a participant or a candidate for participation in in a clinical trial or protocol thereof.

120. The method, the compound for use, or the composition for use of any one of the preceding claims, wherein the subject is excluded from treatment with a Type I inhibitor.

121. The method, the compound for use, or the composition for use of any one of the preceding claims, wherein the Type I inhibitor comprises gefinitinib, erlotinib, afatinib, osimertinib, necitunumab, crizotinib, alectinib, ceritinib, dabrafenib, trametinib, afatinib, sapitinib, dacomitinib, canertinib, pelitinib, WZ4002, WZ8040, WZ3146, CO-1686 or AZD9291.

122. The method, the compound for use, or the composition for use of any one of the preceding claims, wherein the method comprises treating the subject with a Non-Type I inhibitor.

123. The method, the compound for use, or the composition for use of any one of the preceding claims, wherein the composition comprises a Non-Type I inhibitor.

124. The method, the compound for use, or the composition for use of any one of the preceding claims, wherein the Non-Type I inhibitor comprises a Type II small molecule inhibitor.

125. The method, the compound for use, or the composition for use of any one of the preceding claims, wherein the Type II small molecule inhibitor comprises neratinib, AST-1306, HKI-357, or lapatinib.

126. A method of treating cancer in a subject comprising administering to the subject a Non-Type I inhibitor or a potent Type I inhibitor, wherein the subject comprises an allosteric variant of an EGFR, an allosteric variant of a HER2-receptor or an allosteric variant of a HER4-receptor and wherein the allosteric variant comprises a mutation in an extracellular domain of the receptor.

127. The method, the compound for use, or the composition for use of any one of the preceding claims, wherein the Non-Type I ErbB inhibitor comprises a Type II small molecule inhibitor.

128. The method, the compound for use, or the composition for use of any one of the preceding claims, wherein the Non-Type I ErbB inhibitor or potent Type I inhibitor comprises AMG-595, rindopepimut, sapitinib, afatinib, neratinib, AST-1306, HKI-357, or lapatinib.

129. The method, the compound for use, or the composition for use of any one of the preceding claims, wherein the cancer comprises a solid cancer.

130. The method, the compound for use, or the composition for use of any one of the preceding claims, wherein the cancer comprises a bladder cancer, a breast cancer, a cervical cancer, a colorectal cancer, an endometrial cancer, a gastric cancer, a glioblastoma (GBM), a head and neck cancer, a lung cancer, a non-small cell lung cancer (NSCLC) or any subtype thereof.

131. The method, the compound for use, or the composition for use of any one of the preceding claims, wherein the cancer comprises a glioblastoma (GBM) or any subtype thereof.

132. The method, the compound for use, or the composition for use of any one of the preceding claims, wherein the cancer comprises a breast cancer or any subtype thereof.

133. The method, the compound for use, or the composition for use of any one of the preceding claims, wherein the cancer comprises a lung cancer or any subtype thereof.

134. The method, the compound for use, or the composition for use of any one of the preceding claims, wherein the compound is selected from the compounds described in Table I and pharmaceutically acceptable salts thereof.

135. The method, the compound for use, or the composition for use of any one of the preceding claims, wherein the compound is selected from the compounds described in Table I.


SEQUENCE LISTING (17 sequences)

SEQ ID NO: 1 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 2 - protein, 1255 aa, Homo sapiens [sequence omitted]

SEQ ID NO: 3 - protein, 1225 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 4 - protein, 1240 aa, Homo sapiens
MPRGSWKPQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 5 - protein, 1055 aa, Homo sapiens
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRNM

SEQ ID NO: 6 - protein, 603 aa, Homo sapiens
MKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHS

SEQ ID NO: 7 - protein, 21 aa, Artificial sequence
  EGFR exon 20
KEILDEAYVMASVDNPHVCAR

SEQ ID NO: 8 - protein, 20 aa, Artificial
  HER2 exon 20
KEILDEAYVMAGVGSPYVSR

SEQ ID NO: 9 - DNA, 17 bp, Artificial sequence
  EGFRVIII forward
tcgggctctggaggaaa

SEQ ID NO: 10 - DNA, 24 bp, Artificial sequence
  EGFRVIII probe
atgtggtgacagatcacggctcgt

SEQ ID NO: 11 - DNA, 21 bp, Artificial sequence
  EGFRVIII reverse
ccgtcttcctccatctcatag

SEQ ID NO: 12 - DNA, 19 bp, Artificial sequence
  EGFRVII forward
cttctggagggatgcactg

SEQ ID NO: 13 - DNA, 24 bp, Artificial sequence
  EGFRVII probe
caacgaatgggcctaagatcccgt

SEQ ID NO: 14 - DNA, 17 bp, Artificial sequence
  EGFRVII reverse
caccaccagcagcaaga

SEQ ID NO: 15 - DNA, 22 bp, Artificial sequence
  EGFRVvi forward
ccatgcctttgagaacctagaa

SEQ ID NO: 16 - DNA, 24 bp, Artificial sequence
  EGFRVvi probe
acatgagccaagggagtttgtgga

SEQ ID NO: 17 - DNA, 19 bp, Artificial sequence
  EGFRVvi reverse
ctctgggtggcactgtatg